(19) United States
(12) Patent Application Publication
(10) Pub. No.: US 2024/0317672 A1
(43) Pub. Date: Sep. 26, 2024

(54) POLYCARBOXYLATE COMPOUND, RESIST COMPOSITION COMPRISING THE SAME AND METHOD OF FORMING PATTERN USING THE SAME

(71) Applicant: Samsung Electronics Co., Ltd., Suwon-si (KR)

(72) Inventors: Dmitry ANDROSOV, Suwon-si (KR); Cheol KANG, Suwon-si (KR); Haengdeog KOH, Suwon-si (KR); Yoonhyun KWAK, Suwon-si (KR); Beomseok KIM, Suwon-si (KR); Hana KIM, Suwon-si (KR); Hoyoon PARK, Suwon-si (KR)

(73) Assignee: Samsung Electronics Co., Ltd., Suwon-si (KR)

(21) Appl. No.: 18/483,911

(22) Filed: Oct. 10, 2023

(30) Foreign Application Priority Data
Mar. 24, 2023 (KR) 10-2023-0038993
Mar. 28, 2023 (KR) 10-2023-0040761

(51) Int. Cl. C07C 69/94; G03F 7/038; G03F 7/039

(52) U.S. Cl. CPC C07C 69/94; G03F 7/0385; G03F 7/039; C07C 2601/08; C07C 2603/54

(57) ABSTRACT

Disclosed are a polycarboxylate compound represented by Formula 1 below, a resist composition including the same, and a method of forming a pattern using the same.

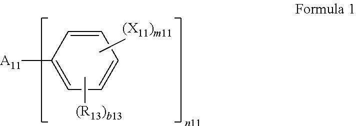

In Formula 1, A₁₁, X₁₁, m11, n11, R₁₃ and b13 are as described in the specification.

### CROSS-REFERENCE TO RELATED APPLICATION

This application is based on and claims priority under 35 U.S.C. § 119 to Korean Patent Application Nos. 10-2023-0038993, filed on Mar. 24, 2023, and 10-2023-0040761, filed on Mar. 28, 2023, in the Korean Intellectual Property Office, the disclosures of each of which are incorporated by reference herein in their entirety.

### BACKGROUND

### 1. Field

The disclosure relates to a polycarboxylate compound, a resist composition including the same, and/or a method of forming a pattern by using the same.

### 2. Description of Related Art

In semiconductor manufacturing, resists that have physical properties that change in response to light may be used to form fine patterns. Among them, chemically amplified resists have been widely used. A chemically amplified resist enables patterning because a base resin of the chemically amplified resist reacts with an acid generated via reaction between light and a photoacid generator, and thus solubility of the base resin in a developer is changed.

Recently, in order to overcome the limits of chemically amplified resists, attempts have been made to develop materials, physical properties of which are changed by exposure to light.

### SUMMARY

Therefore, provided are a polycarboxylate compound, physical properties of which are changed by exposure to even a small dose of light and providing a pattern with an improved resolution, a resist composition including the same, and a method of forming a pattern using the same.

Additional aspects will be set forth in part in the description which follows and, in part, will be apparent from the description, or may be learned by practice of the presented embodiments of the disclosure.

According to an embodiment of the disclosure, a polycarboxylate compound may be represented by Formula 1 below.

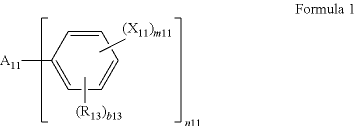

In Formula 1,
A₁₁ may be a single bond, a substituted or unsubstituted carbon atom, or a substituted or unsubstituted silicon atom, or an n11-valent binding unit; and
X₁₁ may be represented by Formula 2 below.

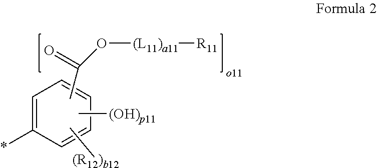

In Formulae 1 and 2,
L₁₁ may be (CRₐR_{b})_{q11}, O, CO, CO₂, or any combination thereof,
q11 may be an integer from 1 to 6,
a11 may be an integer from 0 to 3,
R₁₁ may be a linear, branched, or cyclic C₁-C₃₀ monovalent hydrocarbon group optionally including a hetero atom,
Rₐ, R_{b}, R₁₂ and R₁₃ are each independently hydrogen, deuterium, a halogen atom, a hydroxyl group, a cyano group, a nitro group, a linear, branched, or cyclic C₁-C₃₀ monovalent hydrocarbon group optionally including a hetero atom, or —CO₂(Q₁),
Q₁ may be hydrogen, deuterium, or a linear, branched, or cyclic C₁-C₃₀ monovalent hydrocarbon group optionally including a hetero atom,
b12 may be an integer from 0 to 3,
b13 may be an integer from 0 to 4,
o11 may be an integer from 1 to 4,
p11 may be an integer from 1 to 4,
a sum of b12, o11, and p11 may be 5,
m11 may be an integer from 1 to 5,
a sum of m11 and b13 may be 5,
n11 may be an integer from 1 to 12,
A₁₁ may optionally bind to R₁₃ to form a ring,
among a plurality of R₁₃'s, two adjacent ones may optionally bind to each other to form a ring, and
* may be a binding site with an adjacent atom.

According to another embodiment, a resist composition may include the above-described polycarboxylate compound, a photoacid generator, and an organic solvent.

According to another embodiment, a method of forming a pattern may include forming a resist film by applying the above-described resist composition, exposing at least one portion of the resist film to high-energy rays to provide an exposed resist film, and developing the exposed resist film using a developer.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a flowchart illustrating a method of forming a pattern, according to an embodiment.

FIG. 2A to 2C are a side cross-sectional view for describing a method of forming a pattern, according to an embodiment.

FIGS. 3A to 13B show ¹H-NMR data of Synthesis Examples 1 to 11, respectively.

FIGS. 14A to 14E are side cross-sectional views illustrating a method of forming a patterned structure according to an embodiment; and

FIGS. 15A to 15E are side cross-sectional views illustrating a method of forming a semiconductor device according to an embodiment.

### DETAILED DESCRIPTION

Reference will now be made in detail to embodiments, examples of which are illustrated in the accompanying drawings, wherein like reference numerals refer to like elements throughout. In this regard, the presented embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list. For example, “at least one of A, B, and C,” and similar language (e.g., “at least one selected from the group consisting of A, B, and C”) may be construed as A only, B only, C only, or any combination of two or more of A, B, and C, such as, for instance, ABC, AB, BC, and AC.

When the terms “about” or “substantially” are used in this specification in connection with a numerical value, it is intended that the associated numerical value includes a manufacturing or operational tolerance (e.g., ±10%) around the stated numerical value. Moreover, when the words “generally” and “substantially” are used in connection with geometric shapes, it is intended that precision of the geometric shape is not required but that latitude for the shape is within the scope of the disclosure. Further, regardless of whether numerical values or shapes are modified as “about” or “substantially,” it will be understood that these values and shapes should be construed as including a manufacturing or operational tolerance (e.g., ±10%) around the stated numerical values or shapes. When ranges are specified, the range includes all values therebetween such as increments of 0.1%.

As used herein, when specific definition is not otherwise provided, an “unsubstituted carbon atom” may refer to a carbon atom with separate single bonds to three hydrogen atoms. An “substituted carbon atom” may refer to a carbon atom that is the same as an “unsubstituted carbon atom” except one of the hydrogens has been replaced with a hetero atom (e.g., oxygen, sulfur, nitrogen), a halogen atom, a moiety including a hetero atom (e.g., a hydroxyl group, a cyano group, a carbonyl group, a carboxyl group, an ether bond, an ester bond, a sulfonate ester bond, a carbonate, a lactone ring, a sultone ring, a carboxylic anhydride moiety), or a haloalkyl moiety. An “unsubstituted silicon atom” may refer to a silicon atom with single bonds to three hydrogen atoms. An “substituted silicon atom” may refer to a silicon atom that is the same as an “unsubstituted silicon atom” except one of the hydrogens has been replaced with a hetero atom, a halogen atom, a moiety including a hetero atom, or a haloalkyl moiety. However, example embodiments are not limited thereto.

As the disclosure below allows for various changes and numerous embodiments, particular embodiments will be illustrated in the drawings and described in detail in the written description. However, this is not intended to limit the disclosure to particular modes of practice, and it is to be appreciated that all modifications, equivalents, and substitutes that do not depart from the spirit and technical scope of the disclosure are encompassed in the disclosure. In the following description of the disclosure, a detailed description of known functions and configurations incorporated herein will be omitted when it may obscure the subject matter of the disclosure.

Although the terms “first”, “second”, “third”, and the like may be used herein to describe various elements, these terms are only used to distinguish one element from another and the order, type, or the like of the elements are not limited thereby.

Throughout the specification, it will be understood that when one element such as layer, film, region, or plate, is referred to as being “on” another element, it may be directly on the other element or intervening elements may also be present therebetween.

An expression used in the singular encompasses the expression of the plural, unless it has a clearly different meaning in the context. In the present specification, it is to be understood that the terms such as “including” or “having,” etc., are intended to indicate the existence of the features, numbers, operations, elements, parts, components, materials, or combinations thereof disclosed in the specification, and are not intended to preclude the possibility that one or more other features, numbers, operations, elements, parts, components, materials, or combinations thereof may exist or may be added, unless otherwise stated.

Whenever a range of values is recited, the range includes all values corresponding to the range as clearly recited and also includes boundaries of the range. Therefore, a range “X to Y” includes all numbers between X and Y and also includes X and Y.

As used herein, the term “Cₓ-C_{y}” means that the number of carbon atoms constituting a substituent is from x to y. For example, the term “C₁-C₆” means that the number of carbon atoms constituting a substituent is from 1 to 6, and the term “C₆-C₂₀” means that the number of carbon atoms constituting a substituent is from 6 to 20.

As used herein, the term “monovalent hydrocarbon group” refers to a monovalent moiety derived from an organic compound including carbon and hydrogen or derivatives thereof, and examples thereof may include a linear or branched alkyl group (e.g. a methyl group, an ethyl group, a propyl group, an isopropyl group, a butyl group, an isobutyl group, a sec-butyl group, a tert-butyl group, a pentyl group, a neopentyl group, a hexyl group, a heptyl group, a 2-ethylhexyl group, and a nonyl group); a monovalent saturated cycloaliphatic hydrocarbon group (cycloalkyl group, e.g., a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a 1-adamantyl group, a 2-adamantyl group, a 1-adamantylethyl group, a norbornyl group, a norbornylmethyl group, a tricyclodecanyl group, a tetracyclododecanyl group, a tetracyclododecanylmethyl group, and a dicyclohexylmethyl group); a monovalent unsaturated aliphatic hydrocarbon group (an alkenyl group or an alkynyl group, e.g., an allyl group); a monovalent unsaturated cycloaliphatic hydrocarbon group (cycloalkenyl group, e.g., a 3-cyclohexenyl group); an aryl group (e.g., a phenyl group, a 1-naphthyl group, and a 2-naphthyl group); an arylalkyl group (e.g., a benzyl group and a diphenylmethyl group); a hetero atom-containing monovalent hydrocarbon group (e.g., a tetrahydrofuranyl group, a methoxymethyl group, an ethoxymethyl group, a methylthiomethyl group, an acetamidomethyl group, a trifluoroethyl group, a 2-(methoxyethoxy)methyl group, an acetoxymethyl group, a 2-carboxyl-1-cyclohexyl group, a 2-oxopropyl group, a 4-oxo-1-adamantyl group, and a 3-oxocyclohexyl group), or any combination thereof. Also, because, in these groups, some hydrogen atoms may be substituted with a moiety including a hetero atom, such as oxygen, sulfur, nitrogen, or a halogen atom, or some carbon atoms may be substituted with a moiety including a hetero atom, such as oxygen, sulfur, or nitrogen, the groups may include a hydroxyl group, a cyano group, a carbonyl group, a carboxyl group, an ether bond, an ester bond, a sulfonate ester bond, a carbonate, a lactone ring, a sultone ring, a carboxylic anhydride moiety, or a haloalkyl moiety.

As used herein, the term “divalent hydrocarbon group” refers to a divalent moiety prepared by substituting at least one hydrogen atom of the monovalent hydrocarbon group with a binding site with an adjacent atom. The divalent hydrocarbon group may include, for example, a linear or branched alkylene group, a cycloalkylene group, an alkenylene group, an alkynylene group, a cycloalkylene group, an arylene group, and those in which some carbon atoms are replaced with a hetero atom.

As used herein, the term “alkyl group” refers to a linear or branched monovalent saturated aliphatic hydrocarbon group, and examples thereof include a methyl group, an ethyl group, a propyl group, an isobutyl group, a sec-butyl group, a tert-butyl group, a pentyl group, an iso-amyl group, and a hexyl group. As used herein, the term “alkylene group” refers to a linear or branched divalent saturated aliphatic hydrocarbon group, and examples thereof include a methylene group, an ethylene group, a propylene group, a butylene group, an isobutylene group.

As used herein, the term “halogenated alkyl group” refers to a group in which at least one substituent of the alkyl group is substituted with a halogen atom, and examples thereof include CF₃. In this regard, the halogen atom is F, Cl, Br or I.

As used herein, the term “alkoxy group” refers to a monovalent group represented by formula -OA₁₀₁, where A₁₀₁ is an alkyl group. Examples thereof include a methoxy group, an ethoxy group, and an isopropyloxy group.

As used herein, the term “alkylthio group” refers to a monovalent group represented by formula -SA₁₀₁, where A₁₀₁ is an alkyl group.

As used herein, the term “halogenated alkoxy group” refers to an alkoxy group, at least one hydrogen atom of which is substituted with a halogen atom, and examples thereof include —OCF₃.

As used herein, the term “halogenated alkylthio group” refers to an alkylthio group, at least one hydrogen atom of which is substituted with a halogen atom, and examples thereof include —SCF₃.

As used herein, the term “cycloalkyl group” refers to a monovalent saturated hydrocarbon cyclic group, and examples thereof include a monocyclic group such as a cyclopropyl group, a cyclobutyl group, a cyclopentyl group, a cyclohexyl group, and a cycloheptyl group and a condensed polycyclic group such as a norbornyl group and an adamantly group. As used herein, the term “cycloalkylene group” refers a divalent saturated hydrocarbon cyclic group, and examples thereof include a cyclopentylene group, a cyclohexylene group, an adamantylene group, an adamantylmethylene group, a norbornylene group, a norbornylmethylene group, a tricyclodecanylene group, a tetracyclododecanylene group, a tetracyclododecanylmethylene group, and a dicyclohexylmethylene group.

As used herein, the term “cycloalkoxy group” refers to a monovalent group represented by formula -OA₁₀₂, where A₁₀₂ is a cycloalkyl group. Examples thereof include a cyclopropoxy group and a cyclobutoxy group.

As used herein, the term “cycloalkylthio group” refers to a monovalent group represented by formula -SA₁₀₂, where A₁₀₂ is a cycloalkyl group.

As used herein, the term “heterocycloalkyl group” refers to a cycloalkyl group in which some carbon atoms are substituted with a moiety including a hetero atom, such as oxygen, sulfur, or nitrogen, and the heterocycloalkyl group may specifically include an ether bond, an ester bond, a sulfonate ester bond, a carbonate, a lactone ring, a sultone ring, or a carboxylic anhydride moiety. As used herein, the term “heterocycloalkylene group” refers to a cycloalkylene group in which some carbon atoms are substituted with a moiety including a hetero atom such as oxygen, sulfur, or nitrogen.

As used herein, the term “heterocycloalkoxy group” refers to a monovalent group represented by formula -OA₁₀₃, where A₁₀₃ is a heterocycloalkyl group.

As used herein, the term “alkenyl group” refers to a linear or branched monovalent unsaturated aliphatic hydrocarbon group including at least one carbon-carbon double bond. As used herein, the term “alkenylene group” refers to a linear or branched divalent saturated aliphatic hydrocarbon group including at least one carbon-carbon double bond.

As used herein, the term “cycloalkenyl group” refers to a monovalent unsaturated hydrocarbon cyclic group including at least one carbon-carbon double bond. As used herein, the term “cycloalkenylene group” refers to a divalent unsaturated hydrocarbon cyclic group including at least one carbon-carbon double bond.

As used herein, the term “heterocycloalkenyl group” refers to a cycloalkenyl group in which some carbon atoms are substituted with a moiety including a hetero atom, such as oxygen, sulfur, or nitrogen. As used herein, the term “heterocycloalkenylene group” refers to a cycloalkenylene group in which some carbon atoms are substituted with a moiety including a hetero atom, such as oxygen, sulfur, or nitrogen.

As used herein, the term “alkynyl group” refers to a linear or branched monovalent unsaturated aliphatic hydrocarbon group including at least one carbon-carbon triple bond.

As used herein, the term “aryl group” refers to a monovalent group including a carbocyclic aromatic system, and examples thereof include a phenyl group, a naphthyl group, an anthracenyl group, a phenanthrenyl group, a pyrenyl group, and a chrysenyl group. As used herein, the term “arylene group” refers to a divalent group including a carbocyclic aromatic system.

As used herein, the term “heteroaryl group” refers to a monovalent group including a heterocyclic aromatic system, and examples thereof include a pyridinyl group, a pyrimidinyl group, and a pyrazinyl group. As used herein, the term “heteroarylene group” refers to a divalent group including a heterocyclic aromatic system.

As used herein, the term “substituent” includes deuterium, a halogen atom, a hydroxyl group, a cyano group, a nitro group, a carbonyl group, a carboxylic acid group, an amino group, an ether moiety, an ester moiety, a sulfonate ester moiety, a carbonate moiety, an amide moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₁-C₂₀ alkylthio group, a C₁-C₂₀ halogenated alkoxy group, a C₁-C₂₀ halogenated alkylthio group, a C₃-C₂₀ cycloalkyl group, a C₃-C₂₀ cycloalkoxy group, a C₃-C₂₀ cycloalkylthio group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, or a C₁-C₂₀ heteroarylthio group; and a C₁-C₂₀ alkyl group, a C₁-C₂₀ halogenated alkyl group, a C₁-C₂₀ alkoxy group, a C₁-C₂₀ alkylthio group, a C₁-C₂₀ halogenated alkoxy group, a C₁-C₂₀ halogenated alkylthio group, a C₃-C₂₀ cycloalkyl group, a C₃-C₂₀ cycloalkoxy group, a C₃-C₂₀ cycloalkylthio group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, and a C₁-C₂₀ heteroarylthio group; and any combination thereof, which are unsubstituted or substituted with deuterium, a halogen atom, a hydroxyl group, a cyano group, a nitro group, a carbonyl group, a carboxylic acid group, an amino group, an ether moiety, an ester moiety, a sulfonate ester moiety, a carbonate moiety, an amide moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₁-C₂₀ alkylthio group, a C₁-C₂₀ halogenated alkoxy group, a C₁-C₂₀ halogenated alkylthio group, a C₃-C₂₀ cycloalkyl group, a C₃-C₂₀ cycloalkoxy group, a C₃-C₂₀ cycloalkylthio group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, a C₁-C₂₀ heteroarylthio group, and any combination thereof.

Hereinafter, embodiments will be described in detail with reference to the accompanying drawings. In the drawings, like reference numerals denote like elements or components having substantially same functions, and duplicate descriptions will be omitted. In the drawings, thicknesses of various layers and regions may be enlarged for clarity. In the drawings, for convenience of explanation, thicknesses of some layers and regions are exaggerated for clarity. Meanwhile, it should be understood that embodiments described hereinafter are merely for illustrative purposes various changes in form from the embodiments.

### [Polycarboxylate Compound]

A polycarboxylate compound according to embodiments may be represented by Formula 1 below.

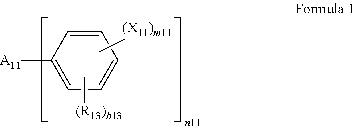

In Formula 1,
A₁₁ is a single bond, a substituted or unsubstituted carbon atom, or a substituted or unsubstituted silicon atom, or an n11-valent binding unit; and
X₁₁ is represented by Formula 2 below.

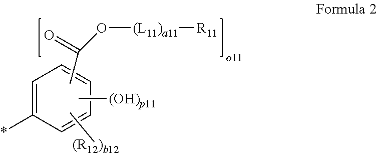

In Formulae 1 and 2,
L₁₁ is (CRₐR_{b})_{q11}, O, CO, CO₂, or any combination thereof,
q11 is an integer from 1 to 6,
a11 is an integer from 0 to 3,
R₁₁ is a linear, branched, or cyclic C₁-C₃₀ monovalent hydrocarbon group optionally including a hetero atom,
Rₐ, R_{b}, R₁₂ and R₁₃ are each independently hydrogen, deuterium, a halogen atom, a hydroxyl group, a cyano group, a nitro group, a linear, branched, or cyclic C₁-C₃₀ monovalent hydrocarbon group optionally including a hetero atom, or —CO₂(Q₁),
Q₁ is hydrogen, deuterium, or a linear, branched, or cyclic C₁-C₃₀ monovalent hydrocarbon group optionally including a hetero atom,
b12 is an integer from 0 to 3,
b13 is an integer from 0 to 4,
o11 is an integer from 1 to 4,
p11 is an integer from 1 to 4,
a sum of b12, o11, and p11 is 5,
m11 is an integer from 1 to 5,
a sum of m11 and b13 is 5,
n11 is an integer from 1 to 12,
A₁₁ may optionally bind to R₁₃ to form a ring,
among a plurality of R₁₃'s, two adjacent ones may optionally bind to each other to form a ring, and
* is a binding site with an adjacent atom.

For example, in Formula 2, L₁₁ may be (CRₐR_{b})_{q11}, O, CO₂, or any combination thereof.

For example, in Formula 2, q11 may be an integer from 1 to 3.

Specifically, in Formula 2, q11 may be 1 or 2.

More specifically, in Formula 2, q11 may be 1.

For example, in Formula 2, a11 may be an integer from 0 to 2.

Specifically, in Formula 2, (L₁₁)ₐ₁₁ may be a single bond, CRₐR_{b}O, or CRₐR_{b}CO₂.

For example, in Formula 1, R₁₁ may be a substituted or unsubstituted C₁-C₃₀ alkyl group, a substituted or unsubstituted C₃-C₃₀ cycloalkyl group, a substituted or unsubstituted C₂-C₃₀ alkenyl group, a substituted or unsubstituted C₃-C₃₀ cycloalkenyl group, a substituted or unsubstituted C₂-C₃₀ alkynyl group, a substituted or unsubstituted C₆-C₃₀ aryl group, or a substituted or unsubstituted C₇-C₃₀ arylalkyl group.

Specifically, in Formula 1, R₁₁ may be selected from a C₁-C₃₀ alkyl group, a C₃-C₃₀ cycloalkyl group, a C₂-C₃₀ alkenyl group, a C₃-C₃₀ cycloalkenyl group, a C₂-C₃₀ alkynyl group, a C₆-C₃₀ aryl group, and a C₇-C₃₀ arylalkyl group, which are unsubstituted or substituted with deuterium, a halogen atom, a hydroxyl group, a cyano group, a nitro group, an amino group, a carbonyl group, a carboxylic acid group, an ether moiety, an ester moiety, a sulfonate ester moiety, a carbonate moiety, an amide moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₁-C₂₀ halogenated alkoxy group, a C₃-C₂₀ cycloalkyl group, a C₃-C₂₀ cycloalkoxy group, a C₂-C₂₀ alkenyl group, a C₆-C₂₀ aryl group, a C₇-C₃₀ arylalkyl group, or any combination thereof.

More specifically, in Formula 1, R₁₁ may be selected from a C₁-C₃₀ alkyl group, a C₃-C₃₀ cycloalkyl group, a C₆-C₃₀ aryl group and a C₇-C₃₀ arylalkyl group, which are unsubstituted or substituted with deuterium, a halogen atom, a hydroxyl group, a cyano group, a nitro group, an amino group, a carbonyl group, a carboxylic acid group, an ether moiety, an ester moiety, a sulfonate ester moiety, a carbonate moiety, an amide moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₁-C₂₀ halogenated alkoxy group, a C₃-C₂₀ cycloalkyl group, a C₃-C₂₀ cycloalkoxy group, a C₆-C₂₀ aryl group, a C₇-C₃₀ arylalkyl group or any combination thereof.

Particularly, in Formula 1, R₁₁ may be selected from compounds represented by Formulae 3-1 to 3-18 below:

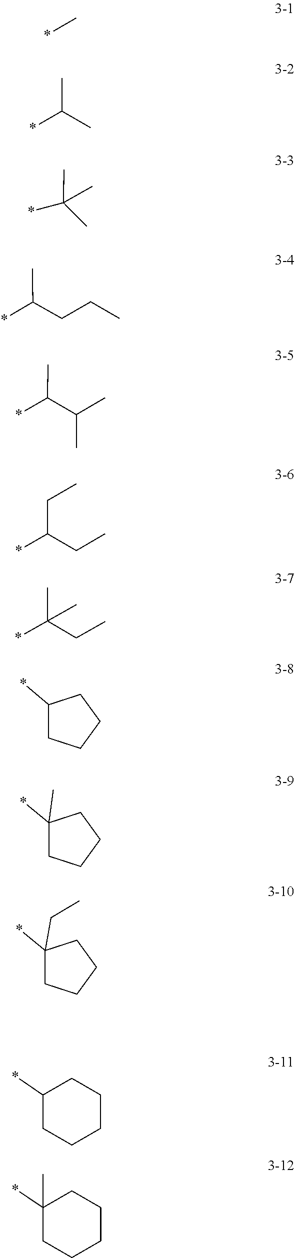

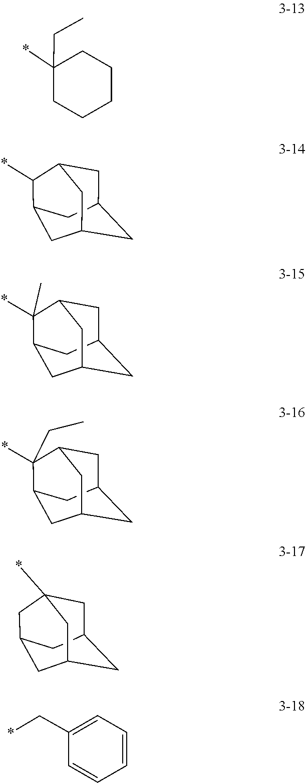

In Formulae 3-1 to 3-18,
* is a binding site with an adjacent atom.

For example, in Formula 1, Rₐ and R_{b} may be each independently hydrogen, deuterium, a halogen atom, a hydroxyl group, a cyano group, a nitro group, a substituted or unsubstituted C₁-C₃₀ alkyl group, a substituted or unsubstituted C₁-C₃₀ alkoxy group, a substituted or unsubstituted C₃-C₃₀ cycloalkyl group, a substituted or unsubstituted C₃-C₃₀ cycloalkoxy group, a substituted or unsubstituted C₂-C₃₀ alkenyl group, a substituted or unsubstituted C₃-C₃₀ cycloalkenyl group, a substituted or unsubstituted C₂-C₃₀ alkynyl group, a substituted or unsubstituted C₆-C₃₀ aryl group, or a substituted or unsubstituted C₇-C₃₀ arylalkyl group.

Specifically, in Formula 1, Rₐ and R_{b} may be each independently hydrogen, deuterium, a halogen atom, a hydroxyl group, a C₁-C₃₀ alkyl group, a C₁-C₃₀ alkoxy group, a C₃-C₃₀ cycloalkyl group, a C₃-C₃₀ cycloalkoxy group, a C₂-C₃₀ alkenyl group, a C₃-C₃₀ cycloalkenyl group, a C₂-C₃₀ alkynyl group, a C₆-C₃₀ aryl group, or a C₇-C₃₀ arylalkyl group.

More specifically, in Formula 1, Rₐ and R_{b} may be each independently hydrogen, deuterium, a halogen atom, a hydroxyl group, C₁-C₃₀ alkyl group, a C₁-C₃₀ alkoxy group, a C₃-C₃₀ cycloalkyl group, a C₃-C₃₀ cycloalkoxy group, a C₆-C₃₀ aryl group, or a C₇-C₃₀ arylalkyl group.

According to an embodiment, in Formula 1, (L₁₁)ₐ₁₁ may be a single bond, CRₐR_{b}O, or CRₐR_{b}CO₂, Rₐ and R_{b} may be each independently hydrogen, deuterium, a halogen atom, a hydroxyl group, a C₁-C₃₀ alkyl group, a C₁-C₃₀ alkoxy group, a C₃-C₃₀ cycloalkyl group, a C₃-C₃₀ cycloalkoxy group, a C₆-C₃₀ aryl group, or a C₇-C₃₀ arylalkyl group, and R₁₁ may be selected from compounds represented by Formulae 3-1 to 3-18 described above.

For example, in Formulae 1 and 2, R₁₂ and R₁₃ may be each independently selected from hydrogen; deuterium; a halogen atom; a hydroxyl group; a cyano group; a nitro group; CO₂(Q₁); and a C₁-C₂₀ alkyl group, a C₅-C₂₀ cycloalkyl group, a C₆-C₂₀ aryl group and a C₇-C₃₀ arylalkyl group, which are unsubstituted or substituted with deuterium, a halogen atom, a hydroxyl group, a cyano group, a nitro group, a carboxylic acid group, an ester moiety, a sulfonate ester moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, a C₇-C₃₀ arylalkyl group, or any combination thereof. Q₁ may be selected from hydrogen; deuterium; and a C₁-C₂₀ alkyl group, a C₃-C₂₀ cycloalkyl group, a C₆-C₂₀ aryl group, and a C₇-C₃₀ arylalkyl group, which are unsubstituted or substituted with deuterium, a halogen atom, a hydroxyl group, a cyano group, a nitro group, a carboxylic acid group, an ester moiety, a sulfonate ester moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, a C₇-C₃₀ arylalkyl group, or any combination thereof.

Specifically, in Formulae 1 and 2, R₁₂ and R₁₃ may be each independently selected from hydrogen; deuterium; a halogen atom; a hydroxyl group; CO₂(Q₁); and a C₁-C₂₀ alkyl group, a C₅-C₂₀ cycloalkyl group, a C₆-C₂₀ aryl group, and a C₇-C₃₀ arylalkyl group, which are unsubstituted or substituted with deuterium, a halogen atom, a hydroxyl group, a carboxylic acid group, an ester moiety, a carbonate moiety, a lactone moiety, a carboxylic anhydride moiety, a C₁-C₂₀ alkyl group, a C₁-C₂₀ alkoxy group, a C₃-C₂₀ cycloalkyl group, a C₃-C₂₀ cycloalkoxy group, a C₆-C₂₀ aryl group, a C₇-C₃₀ arylalkyl group, or any combination thereof. Q₁ may be selected from hydrogen; deuterium; and a C₁-C₂₀ alkyl group, a C₅-C₂₀ cycloalkyl group, a C₆-C₂₀ aryl group, and a C₇-C₃₀ arylalkyl group, which are unsubstituted or substituted with deuterium, a halogen atom, a hydroxyl group, a carboxylic acid group, an ester moiety, a sulfonate ester moiety, a carbonate moiety, a lactone moiety, a sultone moiety, a carboxylic anhydride moiety, a C₁-C₂₀ alkyl group, a C₁-C₂₀ alkoxy group, a C₃-C₂₀ cycloalkyl group, a C₃-C₂₀ cycloalkoxy group, a C₆-C₂₀ aryl group, a C₇-C₃₀ arylalkyl group or any combination thereof.

For example, in Formula 2, o11 and p11 may be each independently an integer from 1 to 3.

Specifically, in Formula 2, o11 and p11 may be each independently an integer from 1 to 2.

More specifically, in Formula 2, o11 and p11 may be 1, respectively.

For example, in Formula 1, m11 may be an integer from 1 to 3.

Specifically, in Formula 1, m11 may be an integer from 1 to 2.

For example, in Formula 1, n11 may be an integer from 1 to 10.

According to an embodiment, in Formula 1, X₁₁ may be represented by one Formulae 2-1 to 2-6 below:

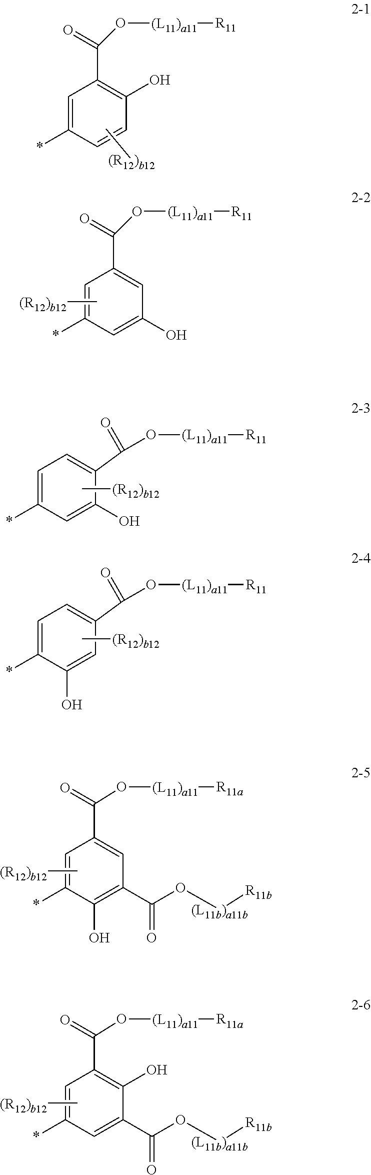

In Formulae 2-1 to 2-6,
for descriptions of L₁₁, a11, R₁₁, R₁₂ and b12, refer to those of Formula 2 above,
for descriptions of L₁₁a and L₁₁b, refer to those of L₁₁ of Formula 2 above,
for descriptions of a11a and a11b, refer to those of a11 of Formula 2 above,
for descriptions of R₁₁ₐ and R_{11b}, refer to those of R₁₁ of Formula 2 above, and
* is a binding site with an adjacent atom.

According to an embodiment, in Formula 1, X₁₁ may be represented by one of Formulae 2-11 to 2-76 below:

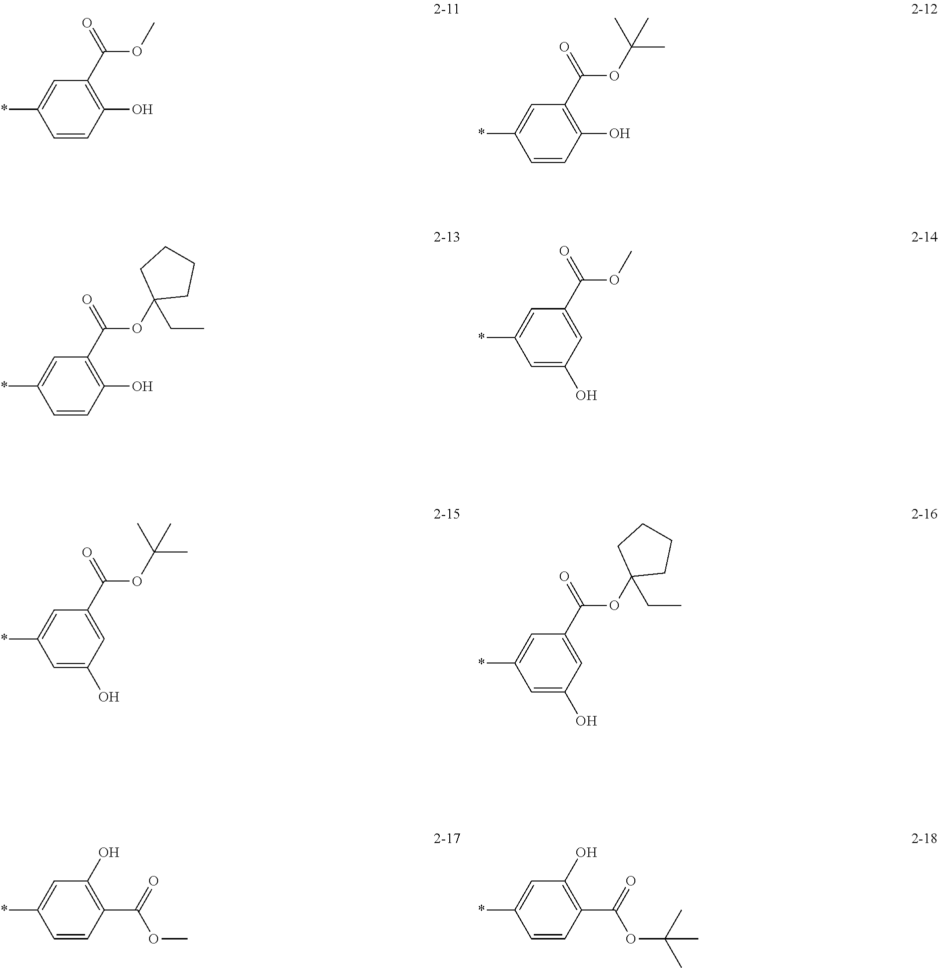

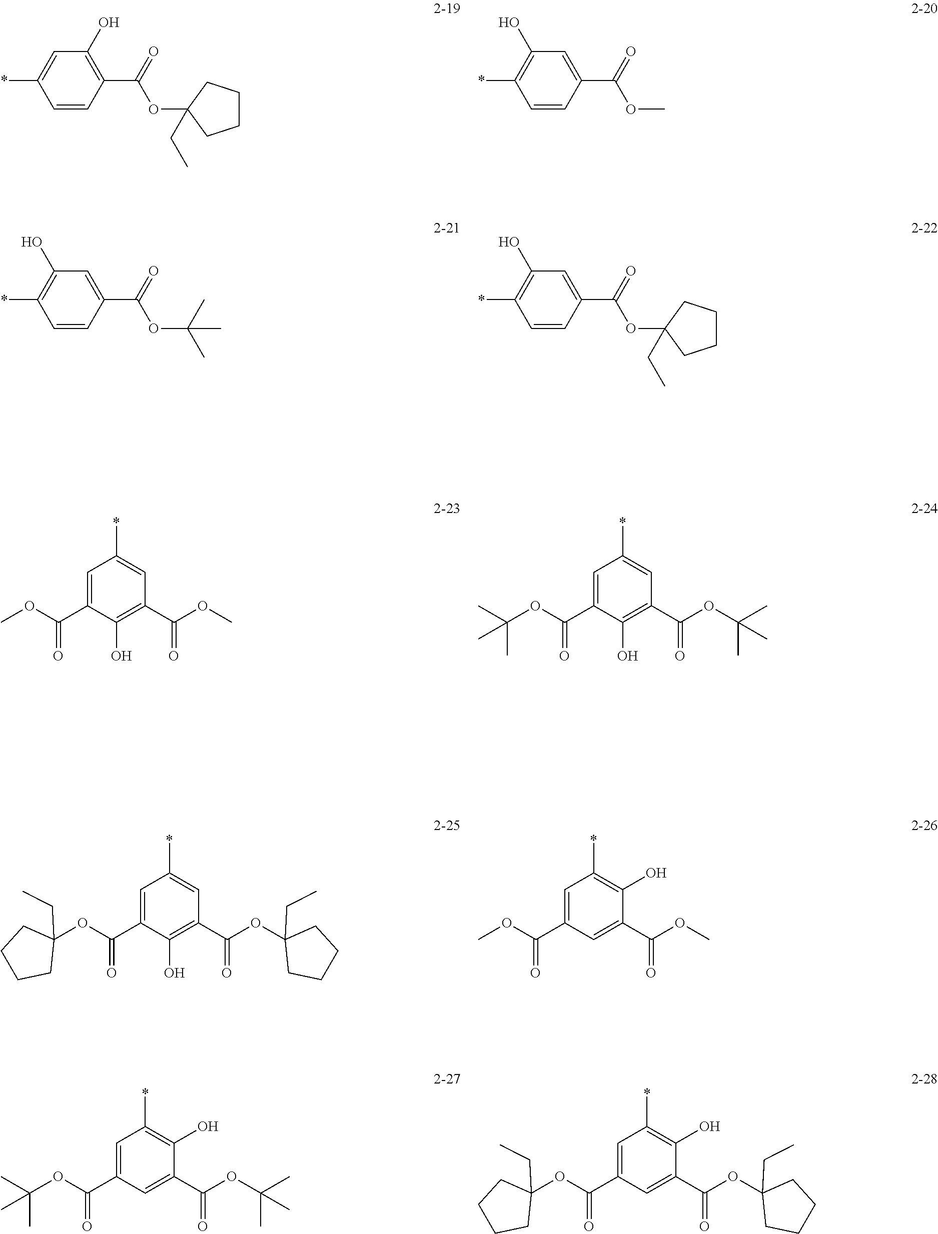

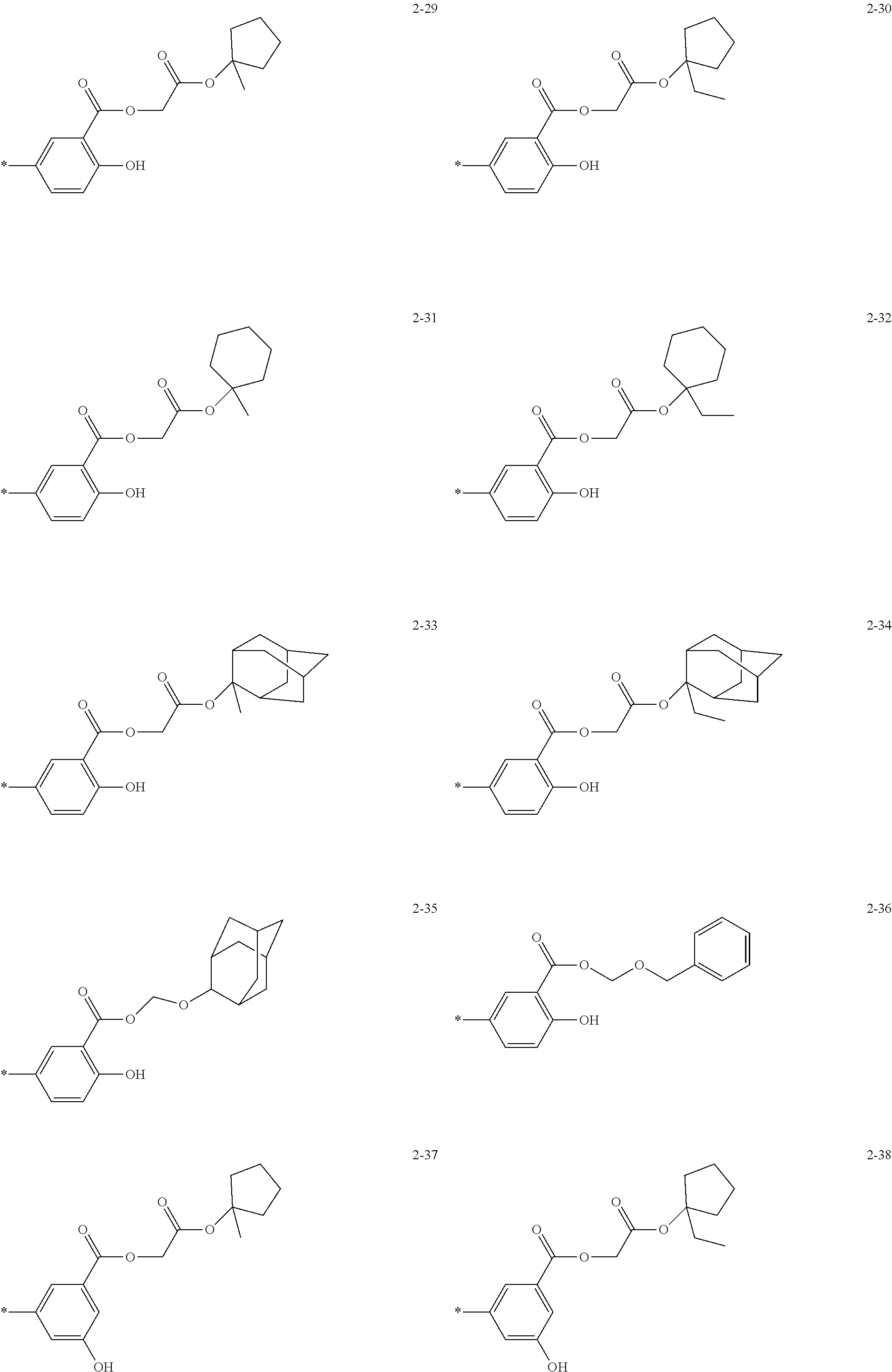

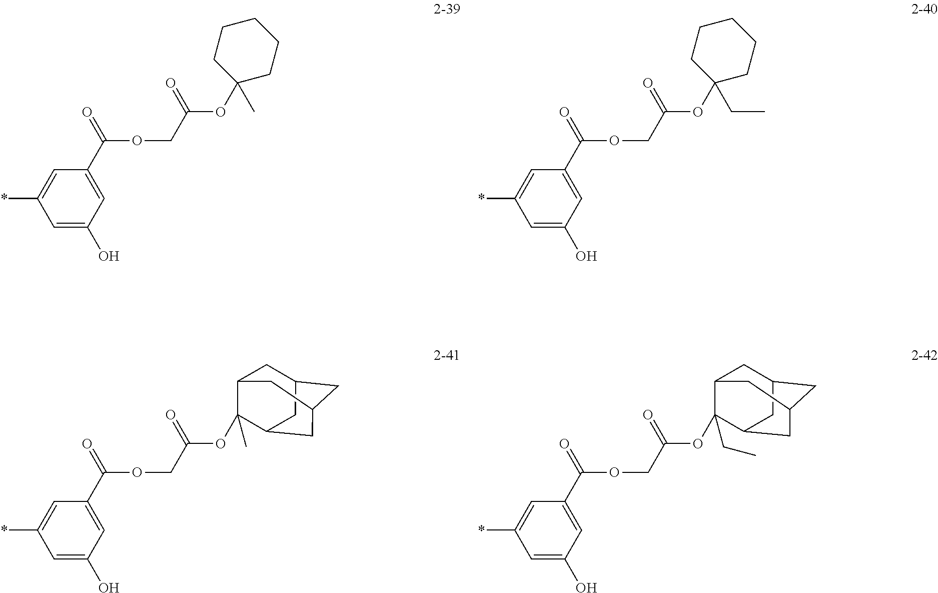 [SMILES: *c1cc(O)cc(C(=O)OCC(=O)OC2(C)CCCCC2)c1 ; *c1cc(O)cc(C(=O)OCC(=O)OC2(CC)CCCCC2)c1 ; *c1cc(O)cc(C(=O)OCC(=O)OC2(C)C3CC4CC(C3)CC2C4)c1 ; *c1cc(O)cc(C(=O)OCC(=O)OC2(CC)C3CC4CC(C3)CC2C4)c1]

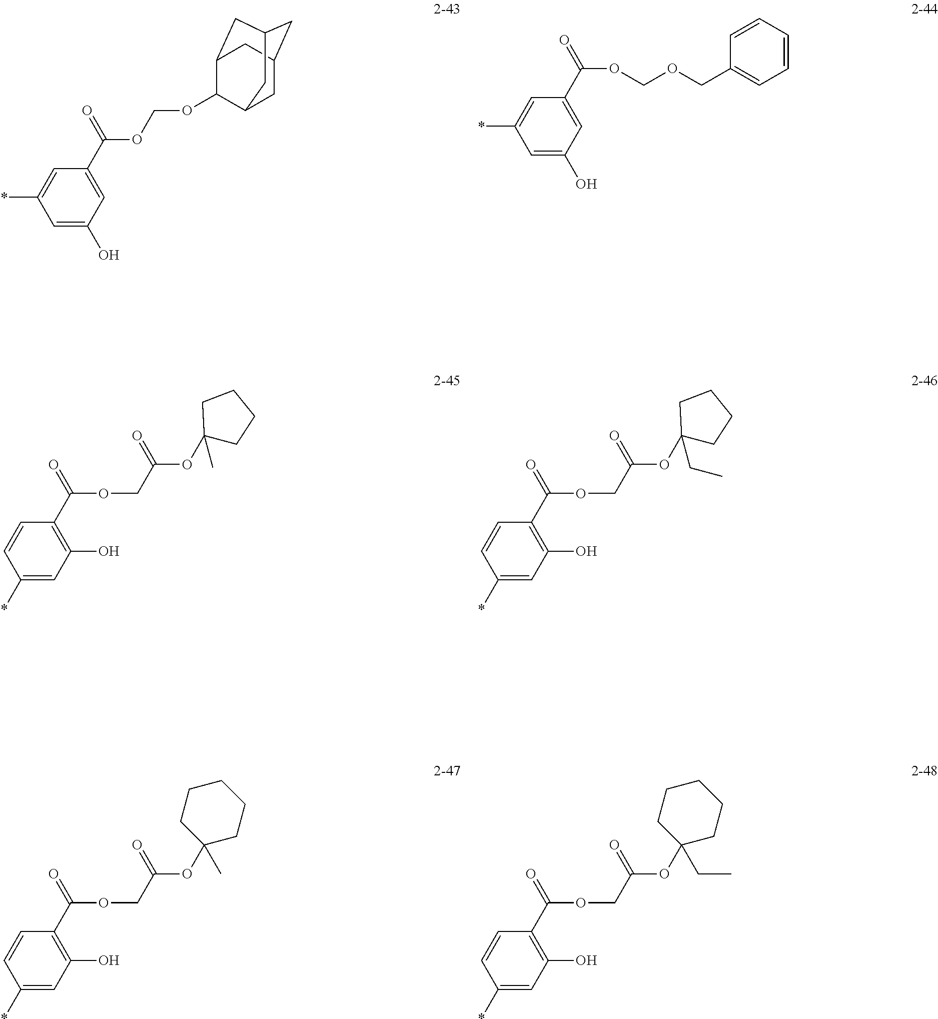 [SMILES: *c1cc(O)cc(C(=O)OCOC2C3CC4CC(C3)CC2C4)c1 ; *c1cc(O)cc(C(=O)OCOCc2ccccc2)c1 ; *c1ccc(C(=O)OCC(=O)OC2(C)CCCC2)c(O)c1 ; *c1ccc(C(=O)OCC(=O)OC2(CC)CCCC2)c(O)c1 ; *c1ccc(C(=O)OCC(=O)OC2(C)CCCCC2)c(O)c1 ; *c1ccc(C(=O)OCC(=O)OC2(CC)CCCCC2)c(O)c1]

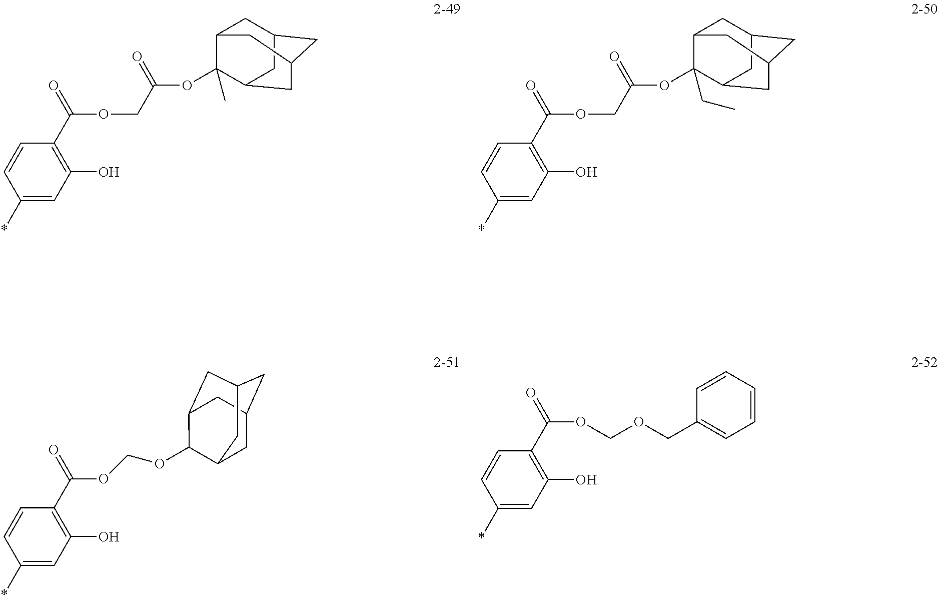 [SMILES: *c1ccc(C(=O)OCC(=O)OC2(C)C3CC4CC(C3)CC2C4)c(O)c1 ; *c1ccc(C(=O)OCC(=O)OC2(CC)C3CC4CC(C3)CC2C4)c(O)c1 ; *c1ccc(C(=O)OCOC2C3CC4CC(C3)CC2C4)c(O)c1 ; *c1ccc(C(=O)OCOCc2ccccc2)c(O)c1]

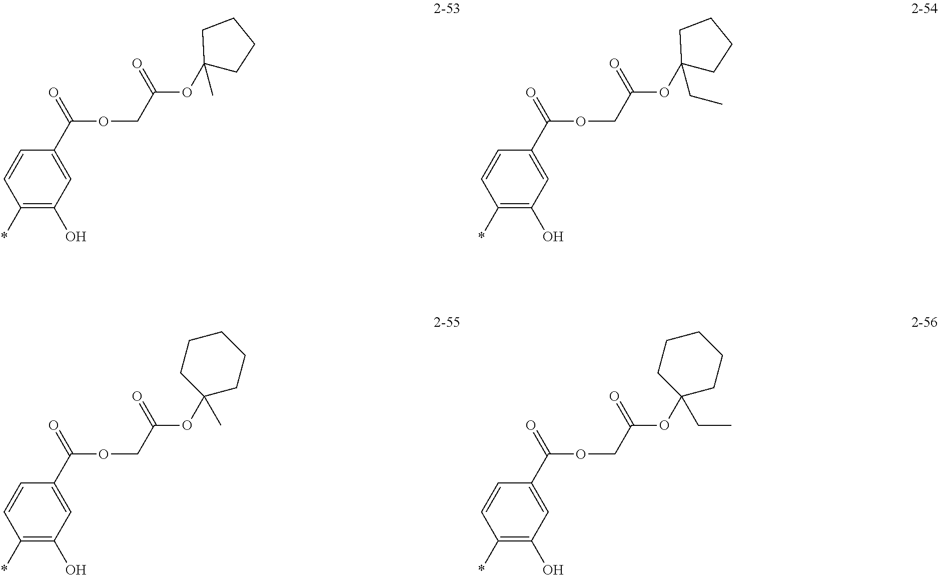 [SMILES: *c1ccc(C(=O)OCC(=O)OC2(C)CCCC2)cc1O ; *c1ccc(C(=O)OCC(=O)OC2(CC)CCCC2)cc1O ; *c1ccc(C(=O)OCC(=O)OC2(C)CCCCC2)cc1O ; *c1ccc(C(=O)OCC(=O)OC2(CC)CCCCC2)cc1O]

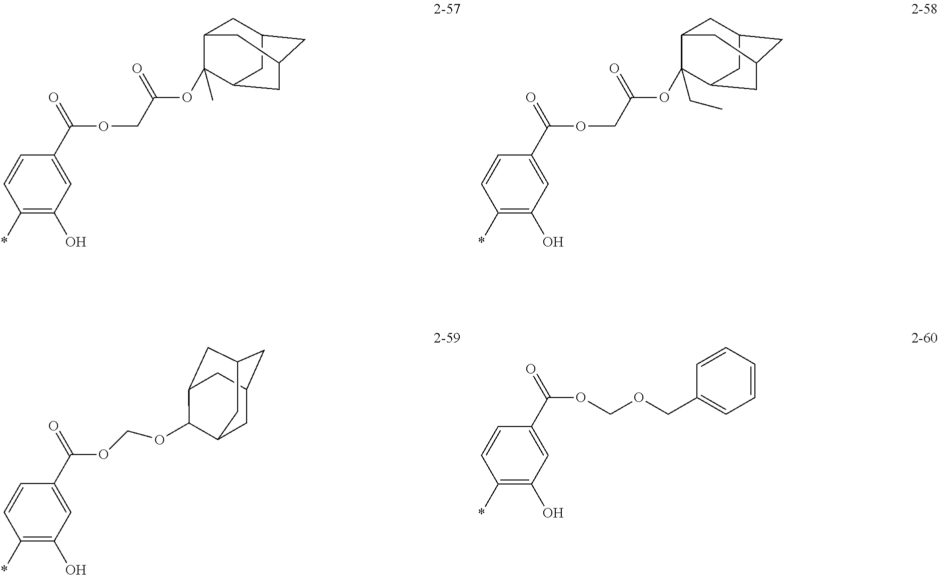 [SMILES: *c1ccc(C(=O)OCC(=O)OC2(C)C3CC4CC(C3)CC2C4)cc1O ; *c1ccc(C(=O)OCC(=O)OC2(CC)C3CC4CC(C3)CC2C4)cc1O ; *c1ccc(C(=O)OCOC2C3CC4CC(C3)CC2C4)cc1O ; *c1ccc(C(=O)OCOCc2ccccc2)cc1O]

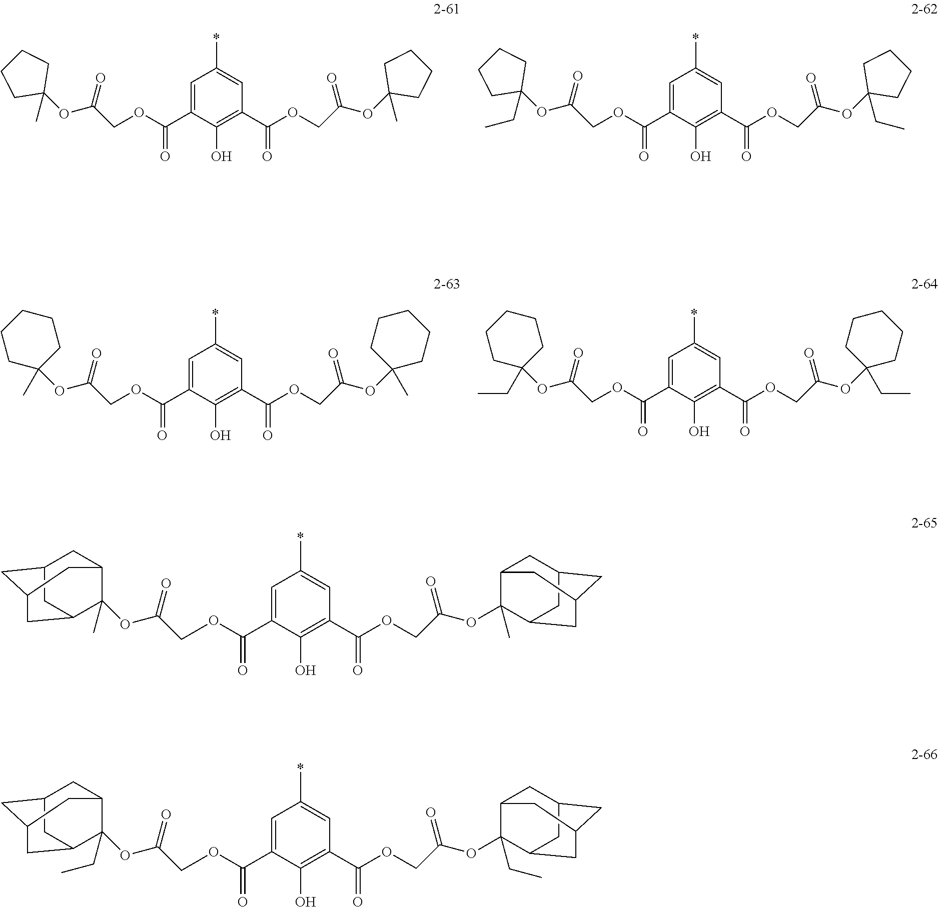 [SMILES: *c1cc(C(=O)OCC(=O)OC2(C)CCCC2)c(O)c(C(=O)OCC(=O)OC2(C)CCCC2)c1 ; *c1cc(C(=O)OCC(=O)OC2(CC)CCCC2)c(O)c(C(=O)OCC(=O)OC2(CC)CCCC2)c1 ; *c1cc(C(=O)OCC(=O)OC2(C)CCCCC2)c(O)c(C(=O)OCC(=O)OC2(C)CCCCC2)c1 ; *c1cc(C(=O)OCC(=O)OC2(CC)CCCCC2)c(O)c(C(=O)OCC(=O)OC2(CC)CCCCC2)c1 ; *c1cc(C(=O)OCC(=O)OC2(C)C3CC4CC(C3)CC2C4)c(O)c(C(=O)OCC(=O)OC2(C)C3CC4CC(C3)CC2C4)c1 ; *c1cc(C(=O)OCC(=O)OC2(CC)C3CC4CC(C3)CC2C4)c(O)c(C(=O)OCC(=O)OC2(CC)C3CC4CC(C3)CC2C4)c1]

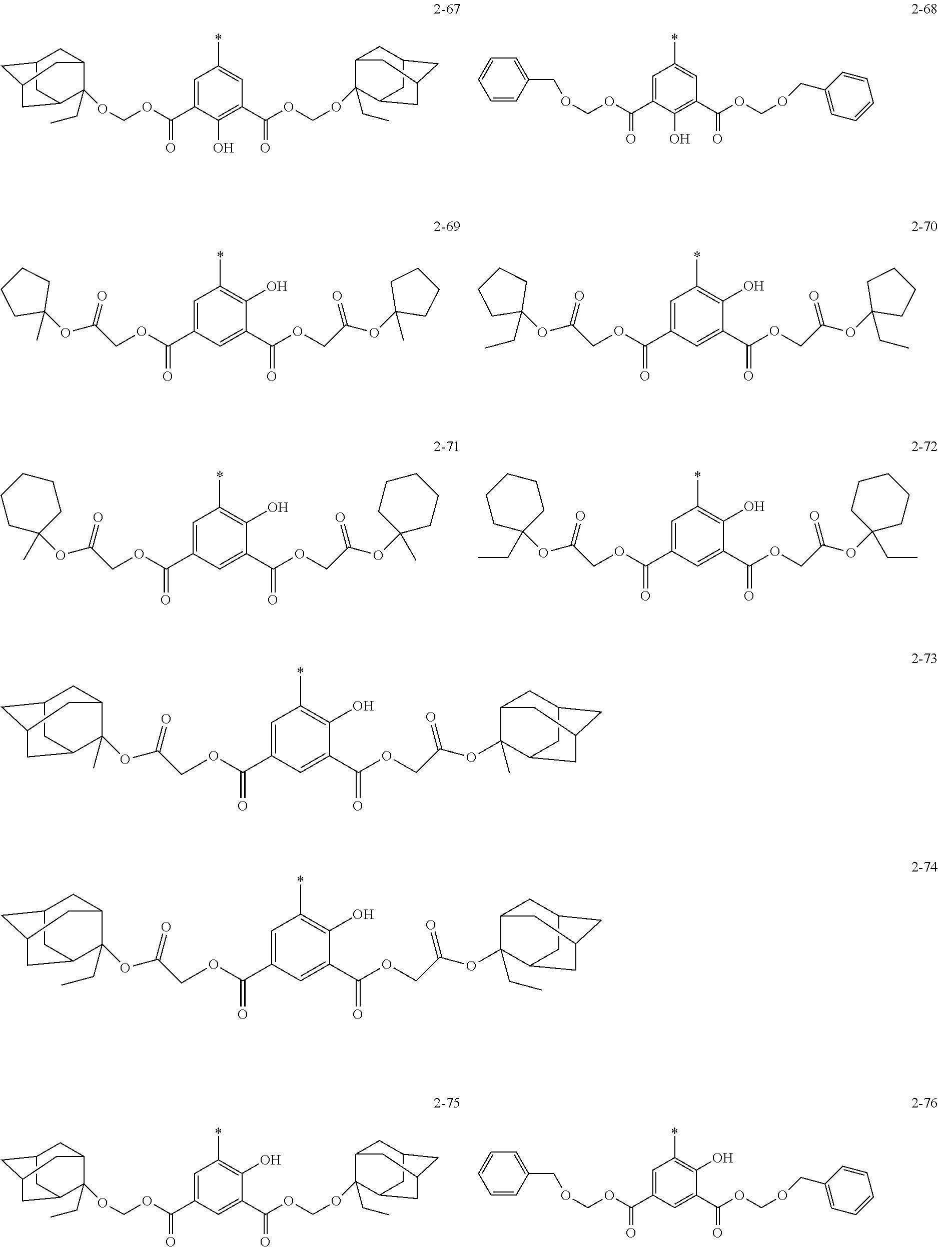

In Formulae 2-11 to 2-76,
* is a binding site with an adjacent atom.

In an embodiment, the polycarboxylate compound represented by Formula 1 may be represented by one of Formulae 1-1 to 1-51 below:

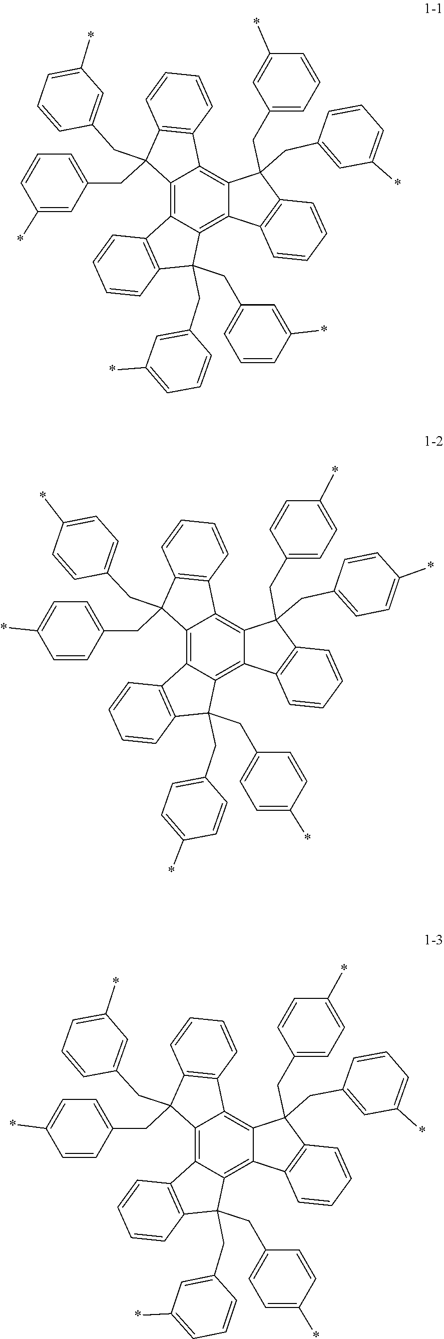 [SMILES: *c1cccc(CC2(Cc3cccc(*)c3)c3ccccc3-c3c2c2c(c4c3C(Cc3cccc(*)c3)(Cc3cccc(*)c3)c3ccccc3-4)C(Cc3cccc(*)c3)(Cc3cccc(*)c3)c3ccccc3-2)c1 ; *c1ccc(CC2(Cc3ccc(*)cc3)c3ccccc3-c3c2c2c(c4c3C(Cc3ccc(*)cc3)(Cc3ccc(*)cc3)c3ccccc3-4)C(Cc3ccc(*)cc3)(Cc3ccc(*)cc3)c3ccccc3-2)cc1 ; *c1ccc(CC2(Cc3cccc(*)c3)c3ccccc3-c3c2c2c(c4c3C(Cc3ccc(*)cc3)(Cc3cccc(*)c3)c3ccccc3-4)C(Cc3ccc(*)cc3)(Cc3cccc(*)c3)c3ccccc3-2)cc1]

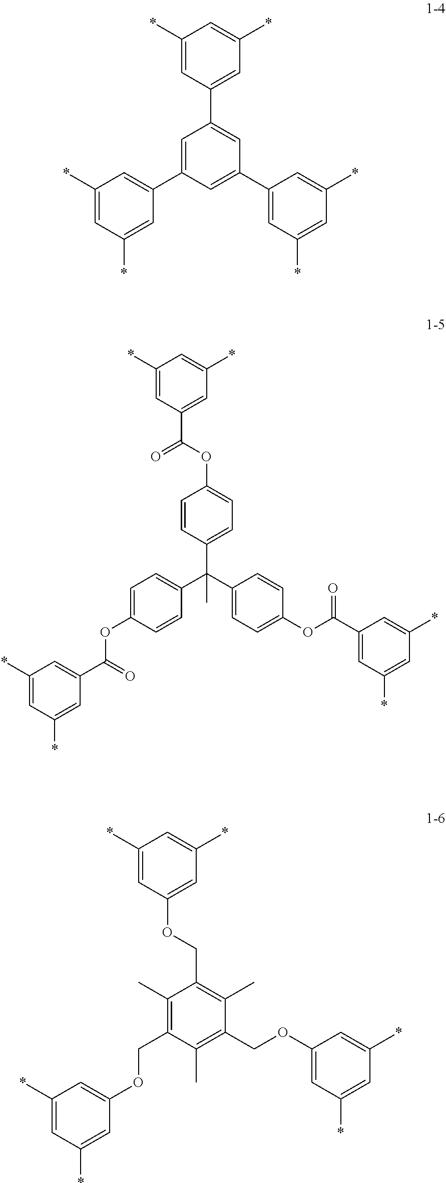 [SMILES: *c1cc(*)cc(-c2cc(-c3cc(*)cc(*)c3)cc(-c3cc(*)cc(*)c3)c2)c1 ; *c1cc(*)cc(C(=O)Oc2ccc(C(C)(c3ccc(OC(=O)c4cc(*)cc(*)c4)cc3)c3ccc(OC(=O)c4cc(*)cc(*)c4)cc3)cc2)c1 ; *c1cc(*)cc(OCc2c(C)c(COc3cc(*)cc(*)c3)c(C)c(COc3cc(*)cc(*)c3)c2C)c1]

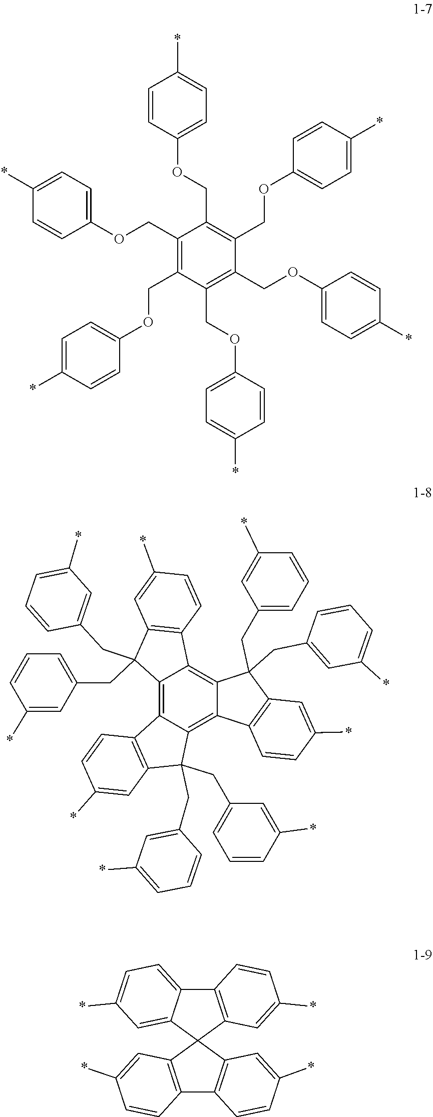 [SMILES: *c1ccc(OCc2c(COc3ccc(*)cc3)c(COc3ccc(*)cc3)c(COc3ccc(*)cc3)c(COc3ccc(*)cc3)c2COc2ccc(*)cc2)cc1 ; *c1cccc(CC2(Cc3cccc(*)c3)c3cc(*)ccc3-c3c2c2c(c4c3C(Cc3cccc(*)c3)(Cc3cccc(*)c3)c3cc(*)ccc3-4)C(Cc3cccc(*)c3)(Cc3cccc(*)c3)c3cc(*)ccc3-2)c1 ; *c1ccc2c(c1)C1(c3cc(*)ccc3-2)c2cc(*)ccc2-c2ccc(*)cc21]

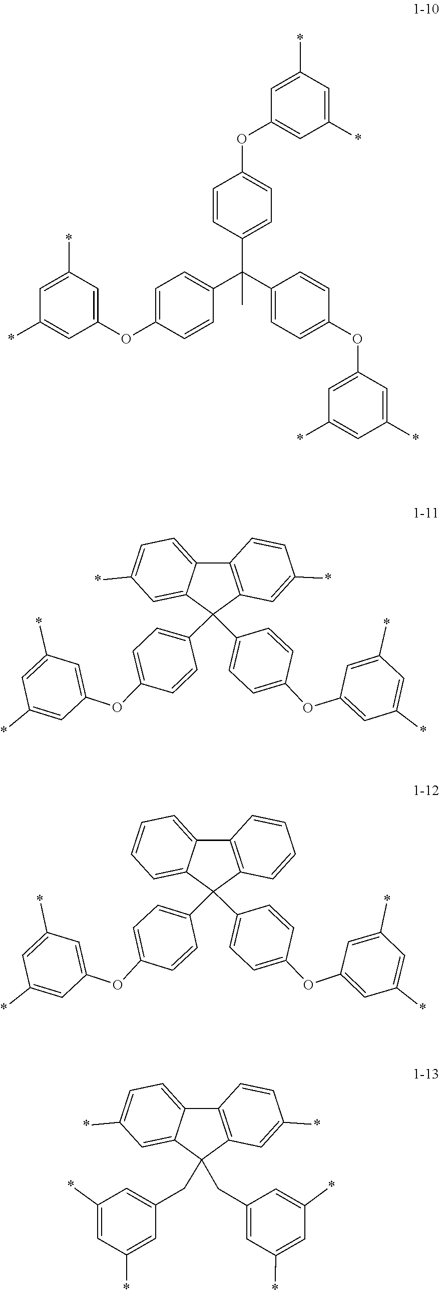 [SMILES: *c1cc(*)cc(Oc2ccc(C(C)(c3ccc(Oc4cc(*)cc(*)c4)cc3)c3ccc(Oc4cc(*)cc(*)c4)cc3)cc2)c1 ; *c1cc(*)cc(Oc2ccc(C3(c4ccc(Oc5cc(*)cc(*)c5)cc4)c4cc(*)ccc4-c4ccc(*)cc43)cc2)c1 ; *c1cc(*)cc(Oc2ccc(C3(c4ccc(Oc5cc(*)cc(*)c5)cc4)c4ccccc4-c4ccccc43)cc2)c1 ; *c1cc(*)cc(CC2(Cc3cc(*)cc(*)c3)c3cc(*)ccc3-c3ccc(*)cc32)c1]

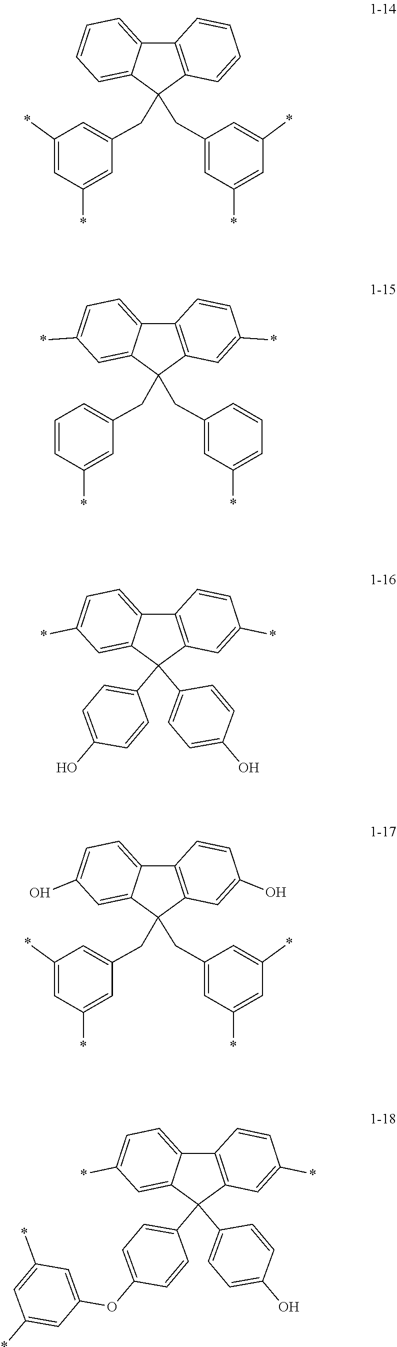 [SMILES: *c1cc(*)cc(CC2(Cc3cc(*)cc(*)c3)c3ccccc3-c3ccccc32)c1 ; *c1cccc(CC2(Cc3cccc(*)c3)c3cc(*)ccc3-c3ccc(*)cc32)c1 ; *c1ccc2c(c1)C(c1ccc(O)cc1)(c1ccc(O)cc1)c1cc(*)ccc1-2 ; *c1cc(*)cc(CC2(Cc3cc(*)cc(*)c3)c3cc(O)ccc3-c3ccc(O)cc32)c1 ; *c1cc(*)cc(Oc2ccc(C3(c4ccc(O)cc4)c4cc(*)ccc4-c4ccc(*)cc43)cc2)c1]

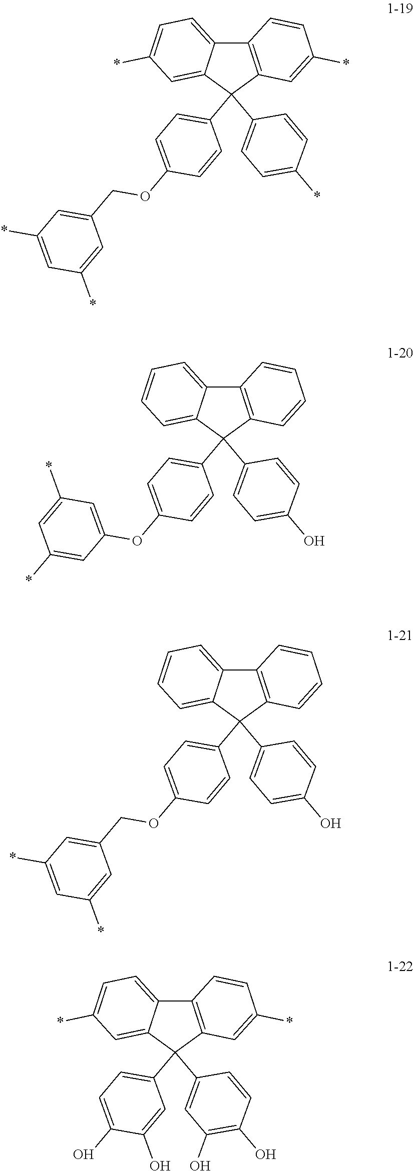 [SMILES: *c1ccc(C2(c3ccc(OCc4cc(*)cc(*)c4)cc3)c3cc(*)ccc3-c3ccc(*)cc32)cc1 ; *c1cc(*)cc(Oc2ccc(C3(c4ccc(O)cc4)c4ccccc4-c4ccccc43)cc2)c1 ; *c1cc(*)cc(COc2ccc(C3(c4ccc(O)cc4)c4ccccc4-c4ccccc43)cc2)c1 ; *c1ccc2c(c1)C(c1ccc(O)c(O)c1)(c1ccc(O)c(O)c1)c1cc(*)ccc1-2]

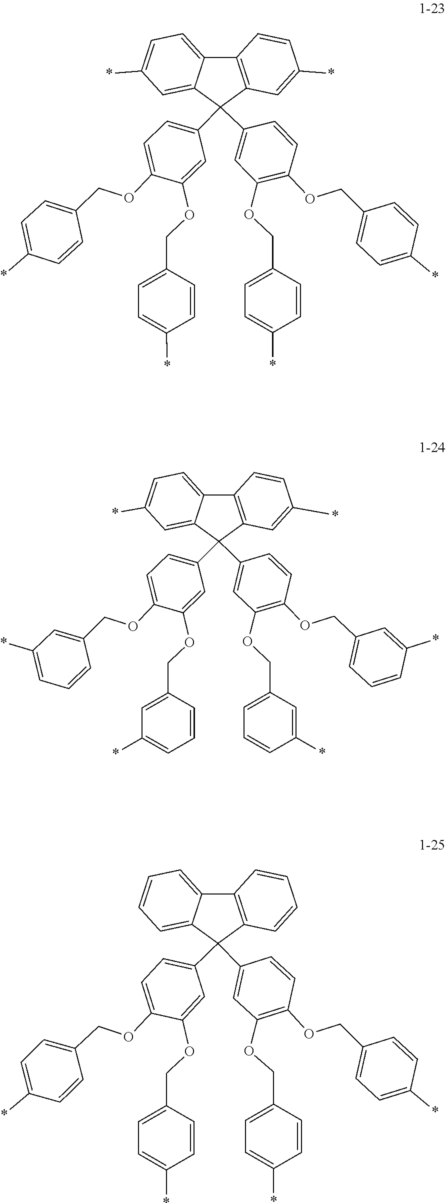 [SMILES: *c1ccc(COc2ccc(C3(c4ccc(OCc5ccc(*)cc5)c(OCc5ccc(*)cc5)c4)c4cc(*)ccc4-c4ccc(*)cc43)cc2OCc2ccc(*)cc2)cc1 ; *c1cccc(COc2ccc(C3(c4ccc(OCc5cccc(*)c5)c(OCc5cccc(*)c5)c4)c4cc(*)ccc4-c4ccc(*)cc43)cc2OCc2cccc(*)c2)c1 ; *c1ccc(COc2ccc(C3(c4ccc(OCc5ccc(*)cc5)c(OCc5ccc(*)cc5)c4)c4ccccc4-c4ccccc43)cc2OCc2ccc(*)cc2)cc1]

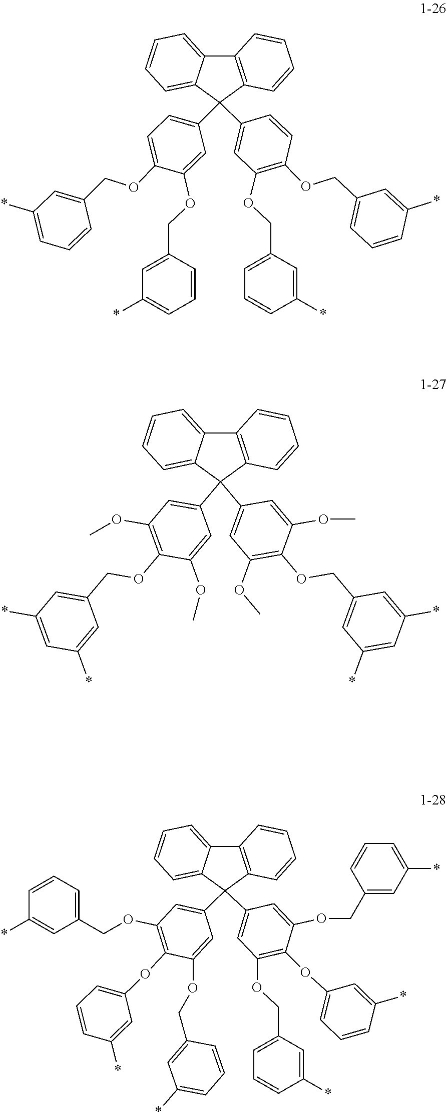 [SMILES: *c1cccc(COc2ccc(C3(c4ccc(OCc5cccc(*)c5)c(OCc5cccc(*)c5)c4)c4ccccc4-c4ccccc43)cc2OCc2cccc(*)c2)c1 ; *c1cc(*)cc(COc2c(OC)cc(C3(c4cc(OC)c(OCc5cc(*)cc(*)c5)c(OC)c4)c4ccccc4-c4ccccc43)cc2OC)c1 ; *c1cccc(COc2cc(C3(c4cc(OCc5cccc(*)c5)c(Oc5cccc(*)c5)c(OCc5cccc(*)c5)c4)c4ccccc4-c4ccccc43)cc(OCc3cccc(*)c3)c2Oc2cccc(*)c2)c1]

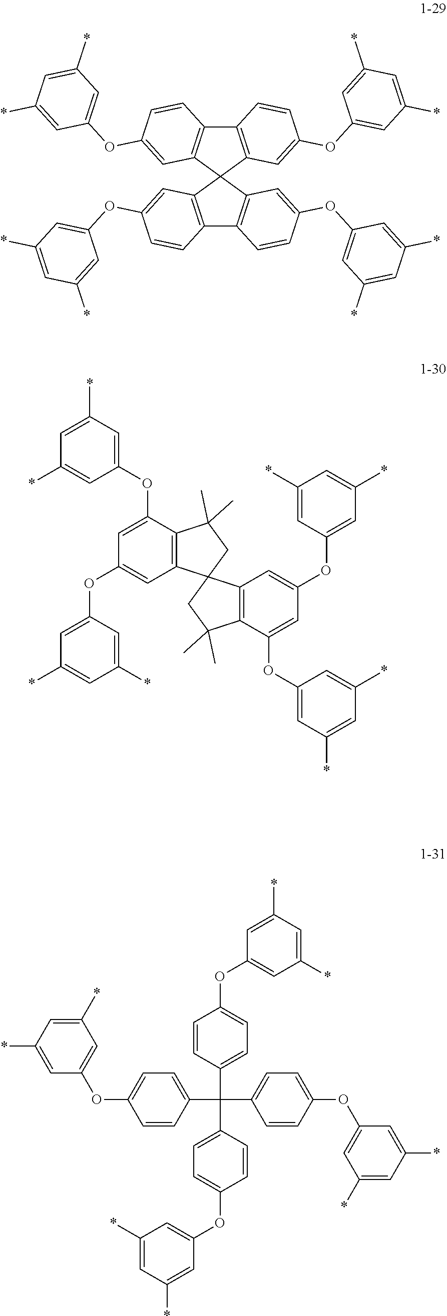 [SMILES: *c1cc(*)cc(Oc2ccc3c(c2)C2(c4cc(Oc5cc(*)cc(*)c5)ccc4-3)c3cc(Oc4cc(*)cc(*)c4)ccc3-c3ccc(Oc4cc(*)cc(*)c4)cc32)c1 ; *c1cc(*)cc(Oc2cc(Oc3cc(*)cc(*)c3)c3c(c2)C2(CC3(C)C)CC(C)(C)c3c(Oc4cc(*)cc(*)c4)cc(Oc4cc(*)cc(*)c4)cc32)c1 ; *c1cc(*)cc(Oc2ccc(C(c3ccc(Oc4cc(*)cc(*)c4)cc3)(c3ccc(Oc4cc(*)cc(*)c4)cc3)c3ccc(Oc4cc(*)cc(*)c4)cc3)cc2)c1]

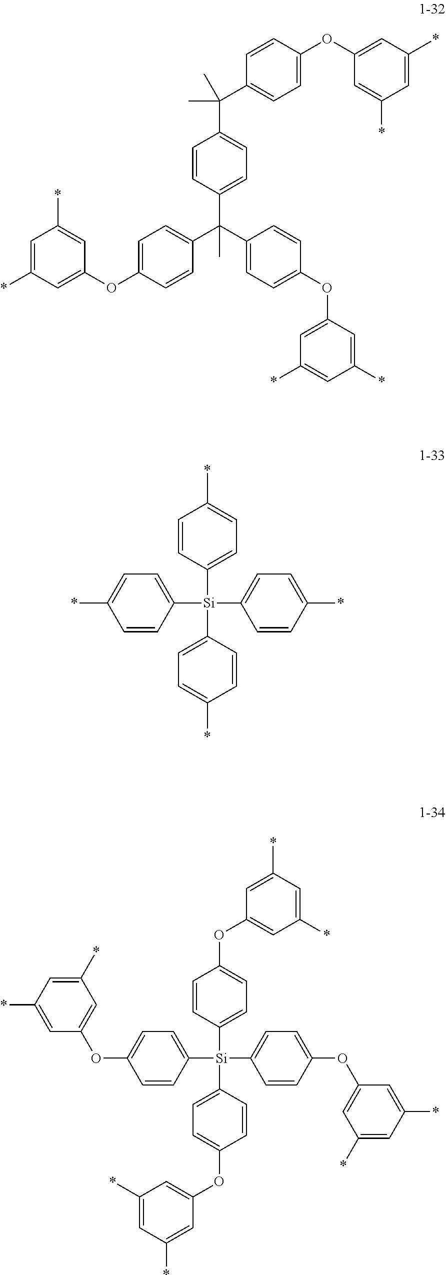 [SMILES: *c1cc(*)cc(Oc2ccc(C(C)(C)c3ccc(C(C)(c4ccc(Oc5cc(*)cc(*)c5)cc4)c4ccc(Oc5cc(*)cc(*)c5)cc4)cc3)cc2)c1 ; *c1ccc([Si](c2ccc(*)cc2)(c2ccc(*)cc2)c2ccc(*)cc2)cc1 ; *c1cc(*)cc(Oc2ccc([Si](c3ccc(Oc4cc(*)cc(*)c4)cc3)(c3ccc(Oc4cc(*)cc(*)c4)cc3)c3ccc(Oc4cc(*)cc(*)c4)cc3)cc2)c1]

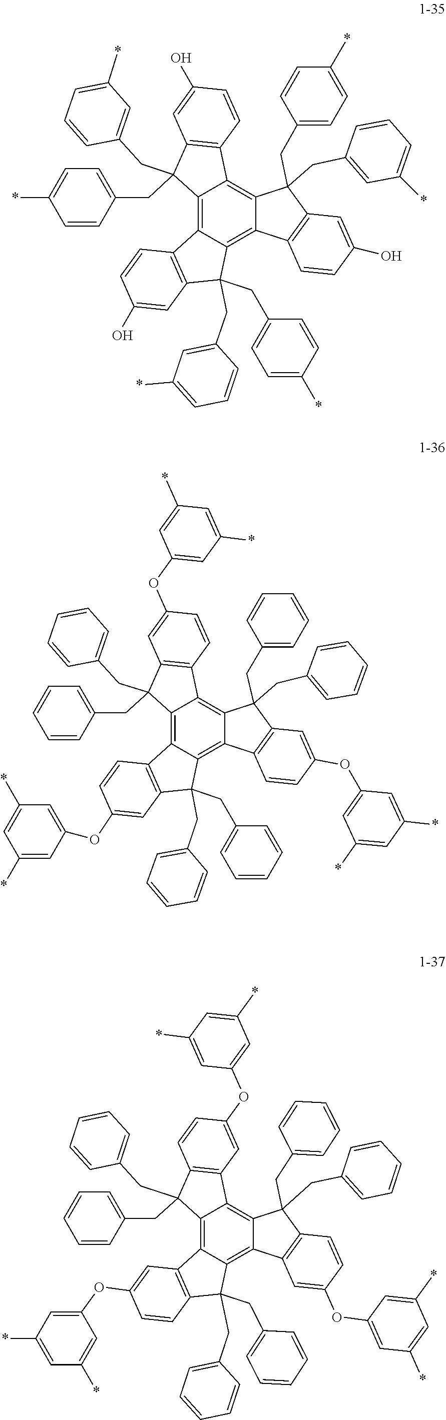 [SMILES: *c1ccc(CC2(Cc3cccc(*)c3)c3cc(O)ccc3-c3c2c2c(c4c3C(Cc3ccc(*)cc3)(Cc3cccc(*)c3)c3cc(O)ccc3-4)C(Cc3ccc(*)cc3)(Cc3cccc(*)c3)c3cc(O)ccc3-2)cc1 ; *c1cc(*)cc(Oc2ccc3c(c2)C(Cc2ccccc2)(Cc2ccccc2)c2c-3c3c(c4c2-c2ccc(Oc5cc(*)cc(*)c5)cc2C4(Cc2ccccc2)Cc2ccccc2)-c2ccc(Oc4cc(*)cc(*)c4)cc2C3(Cc2ccccc2)Cc2ccccc2)c1 ; *c1cc(*)cc(Oc2ccc3c(c2)-c2c(c4c(c5c2C(Cc2ccccc2)(Cc2ccccc2)c2ccc(Oc6cc(*)cc(*)c6)cc2-5)C(Cc2ccccc2)(Cc2ccccc2)c2ccc(Oc5cc(*)cc(*)c5)cc2-4)C3(Cc2ccccc2)Cc2ccccc2)c1]

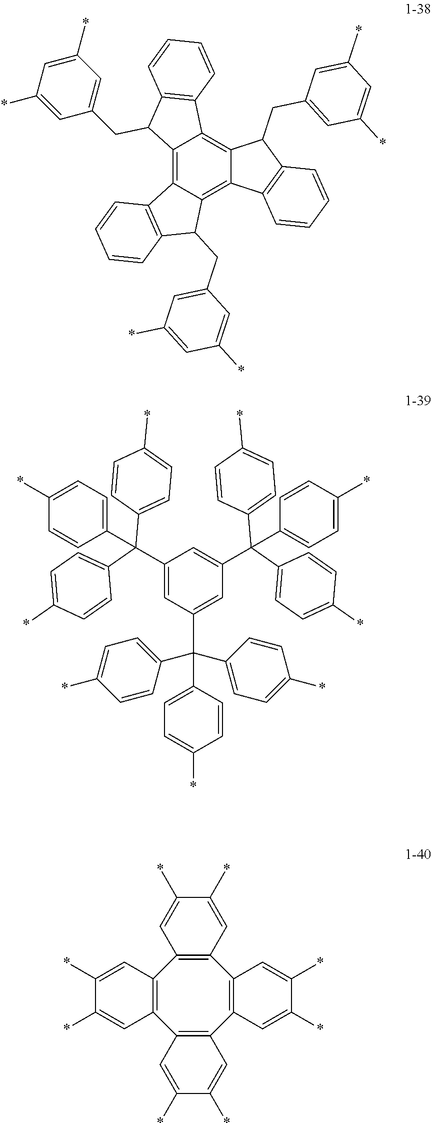

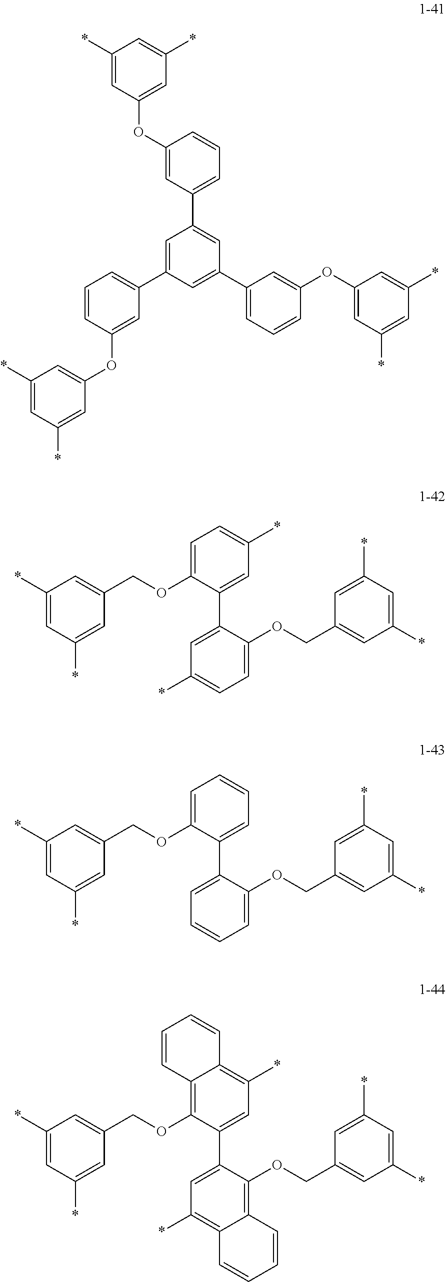 [SMILES: *c1cc(*)cc(Oc2cccc(-c3cc(-c4cccc(Oc5cc(*)cc(*)c5)c4)cc(-c4cccc(Oc5cc(*)cc(*)c5)c4)c3)c2)c1 ; *c1cc(*)cc(COc2ccc(*)cc2-c2cc(*)ccc2OCc2cc(*)cc(*)c2)c1 ; *c1cc(*)cc(COc2ccccc2-c2ccccc2OCc2cc(*)cc(*)c2)c1 ; *c1cc(*)cc(COc2c(-c3cc(*)c4ccccc4c3OCc3cc(*)cc(*)c3)cc(*)c3ccccc23)c1]

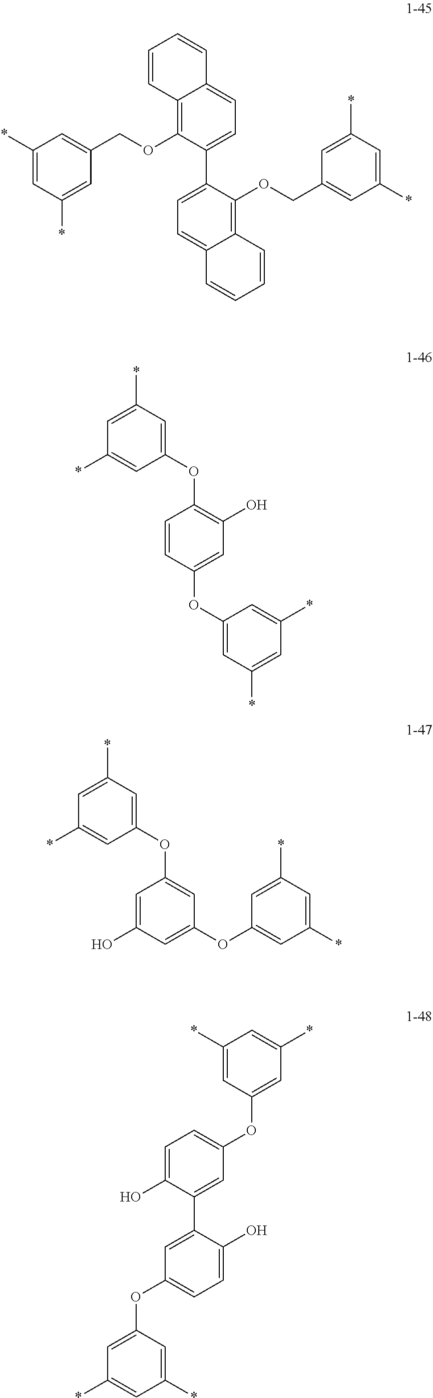 [SMILES: *c1cc(*)cc(COc2c(-c3ccc4ccccc4c3OCc3cc(*)cc(*)c3)ccc3ccccc23)c1 ; *c1cc(*)cc(Oc2ccc(Oc3cc(*)cc(*)c3)c(O)c2)c1 ; *c1cc(*)cc(Oc2cc(O)cc(Oc3cc(*)cc(*)c3)c2)c1 ; *c1cc(*)cc(Oc2ccc(O)c(-c3cc(Oc4cc(*)cc(*)c4)ccc3O)c2)c1]

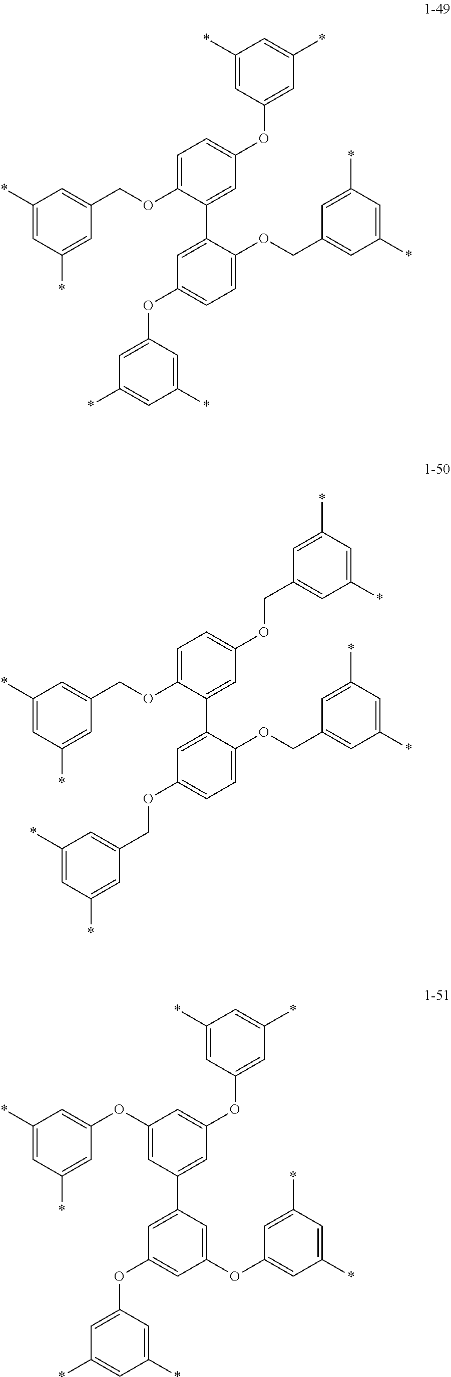 [SMILES: *c1cc(*)cc(COc2ccc(Oc3cc(*)cc(*)c3)cc2-c2cc(Oc3cc(*)cc(*)c3)ccc2OCc2cc(*)cc(*)c2)c1 ; *c1cc(*)cc(COc2ccc(OCc3cc(*)cc(*)c3)c(-c3cc(OCc4cc(*)cc(*)c4)ccc3OCc3cc(*)cc(*)c3)c2)c1 ; *c1cc(*)cc(Oc2cc(Oc3cc(*)cc(*)c3)cc(-c3cc(Oc4cc(*)cc(*)c4)cc(Oc4cc(*)cc(*)c4)c3)c2)c1]

In Formulae 1-1 to 1-51,
a hydrogen may be optionally substituted with a substituent and/or R₁₃, and
* is a binding site with X₁₁.

For example, in Formulae 1-1 to 1-51 above, X₁₁ may be represented by one of Formulae 2-11 to 2-28.

For example, in Formulae 1-1 to 1-51 above, a hydrogen of Formulae 1-1 to 1-51 may be unsubstituted.

According to an embodiment, the polycarboxylate compound represented by Formula 1 may be represented by one of Formulae 1-1 to 1-51. In Formulae 1-1 to 1-51, a hydrogen may be unsubstituted, * is a binding site with X₁₁; and X₁₁ may be represented by one of Formulae 2-11 to 2-28.

In an embodiment, the polycarboxylate compound represented by Formula 1 may be selected from compounds of Group I below:

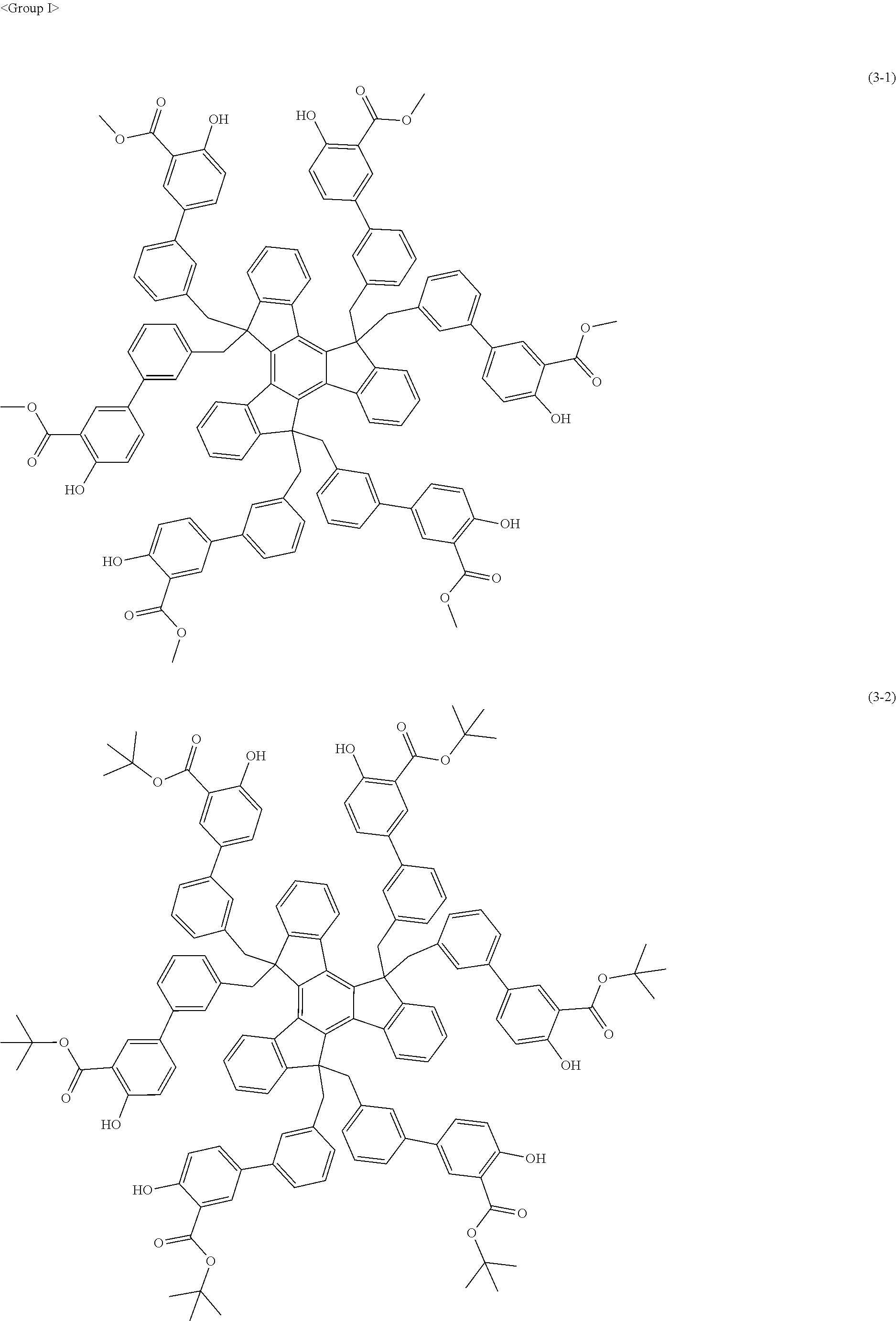

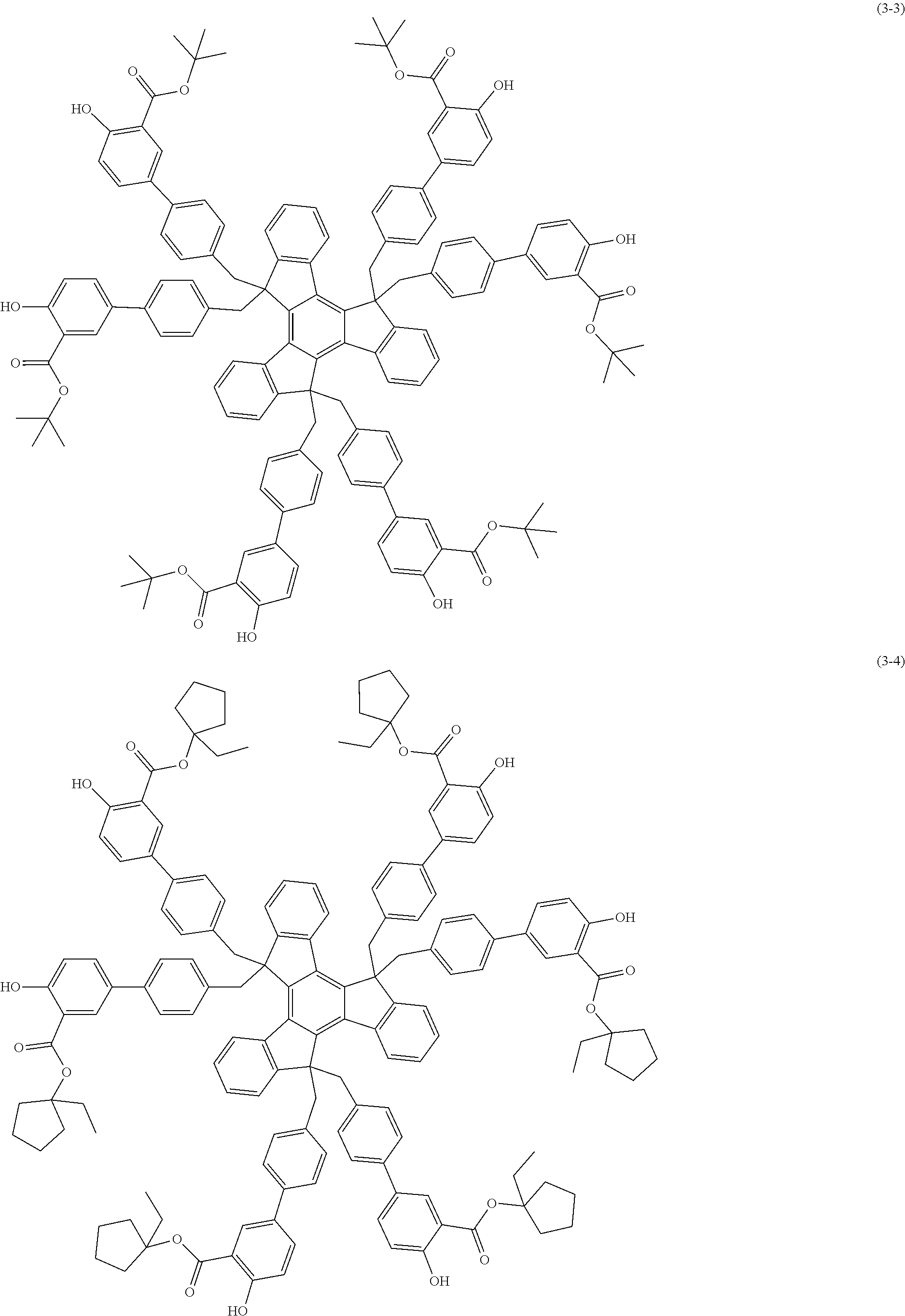

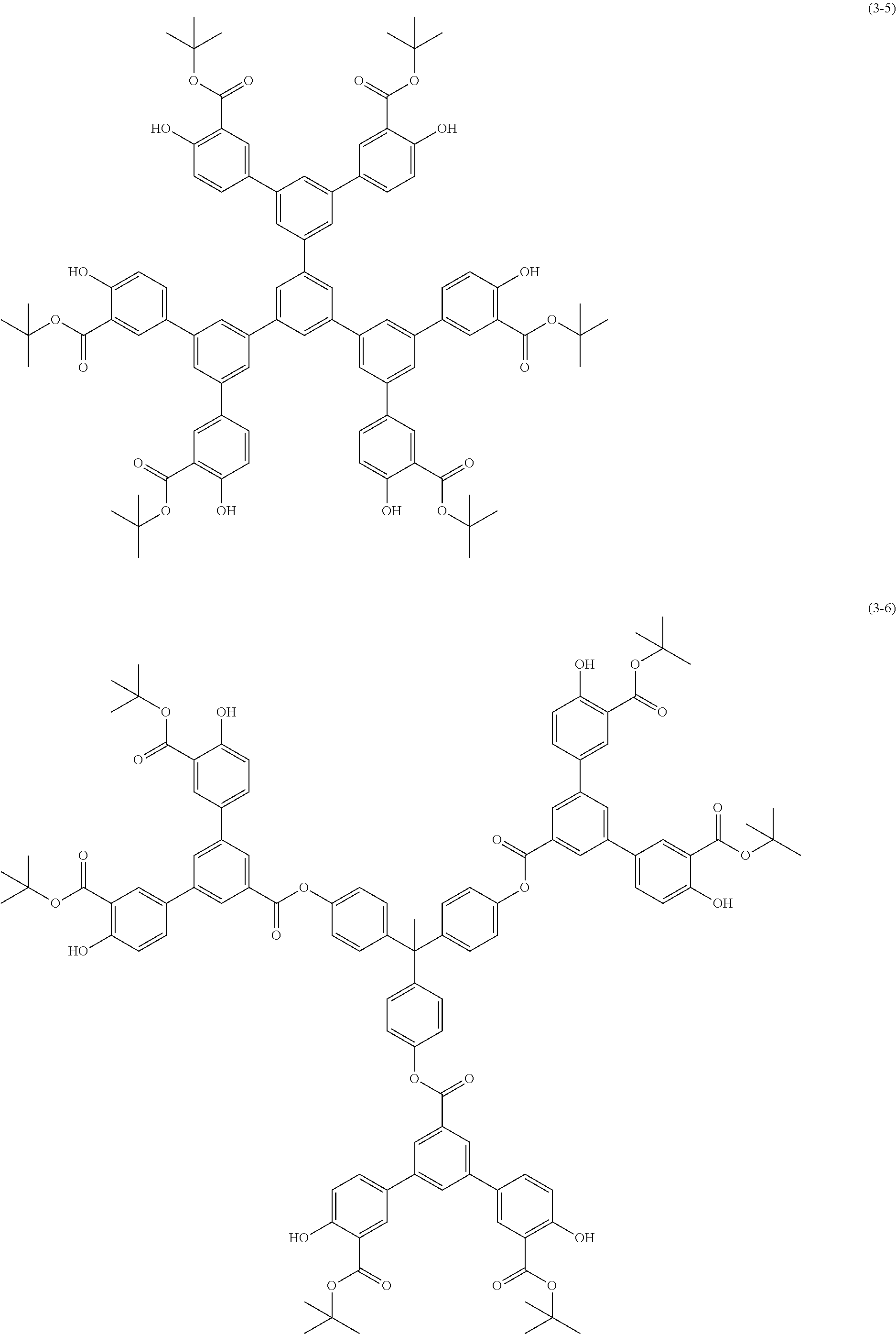

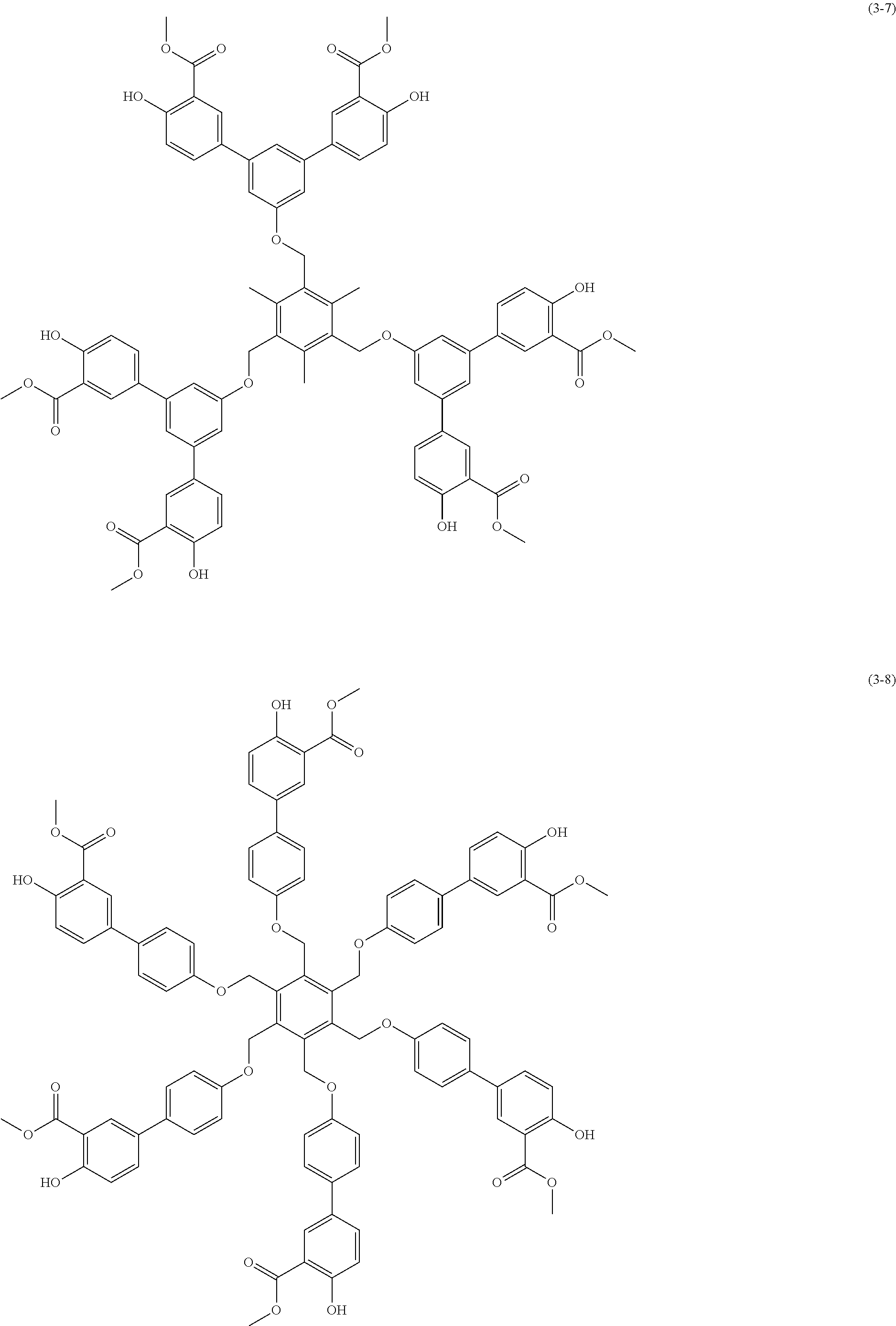

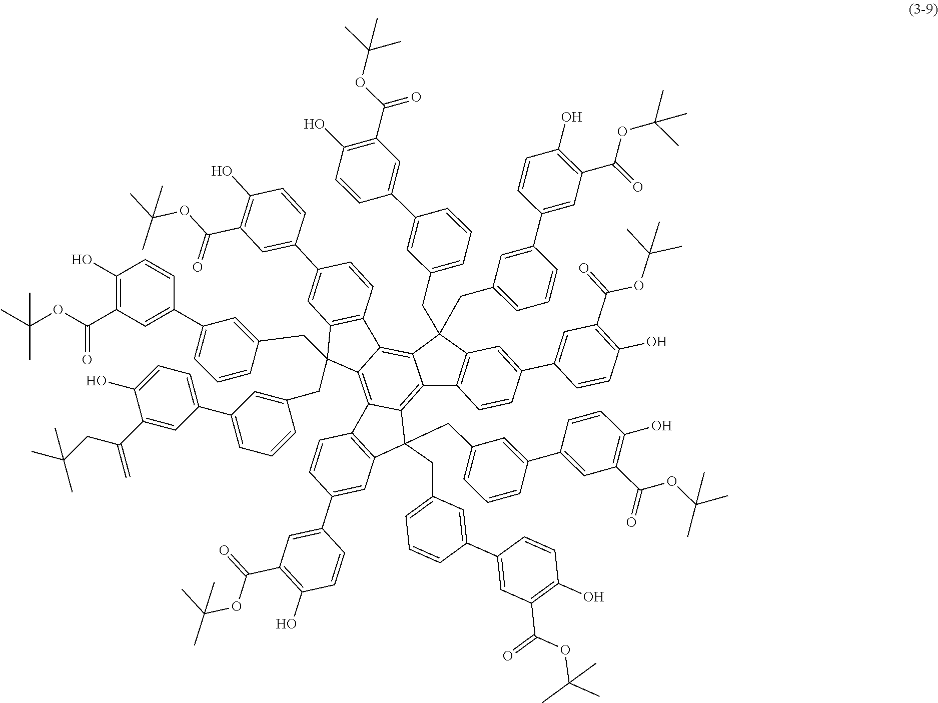 [SMILES: C=C(CC(C)(C)C)c1cc(-c2cccc(CC3(Cc4cccc(-c5ccc(O)c(C(=O)OC(C)(C)C)c5)c4)c4cc(-c5ccc(O)c(C(=O)OC(C)(C)C)c5)ccc4-c4c3c3c(c5c4C(Cc4cccc(-c6ccc(O)c(C(=O)OC(C)(C)C)c6)c4)(Cc4cccc(-c6ccc(O)c(C(=O)OC(C)(C)C)c6)c4)c4cc(-c6ccc(O)c(C(=O)OC(C)(C)C)c6)ccc4-5)C(Cc4cccc(-c5ccc(O)c(C(=O)OC(C)(C)C)c5)c4)(Cc4cccc(-c5ccc(O)c(C(=O)OC(C)(C)C)c5)c4)c4cc(-c5ccc(O)c(C(=O)OC(C)(C)C)c5)ccc4-3)c2)ccc1O]

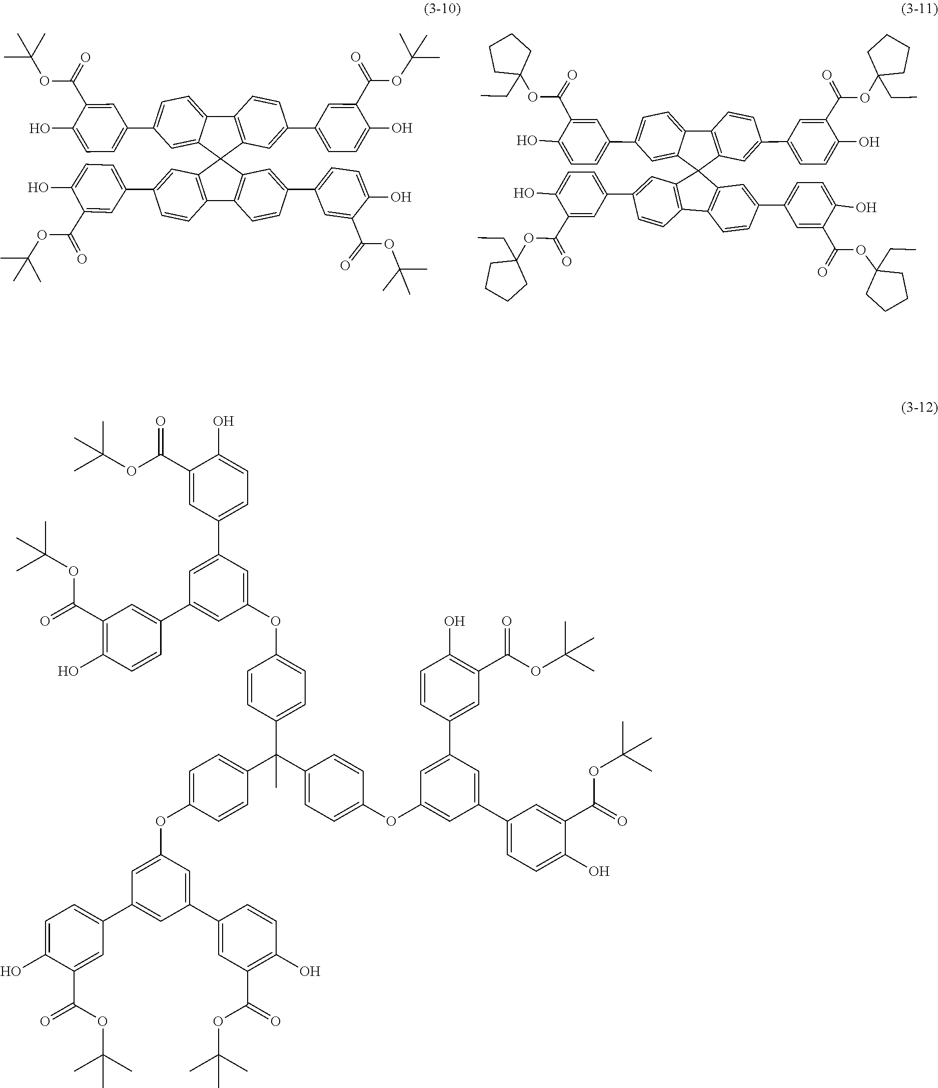

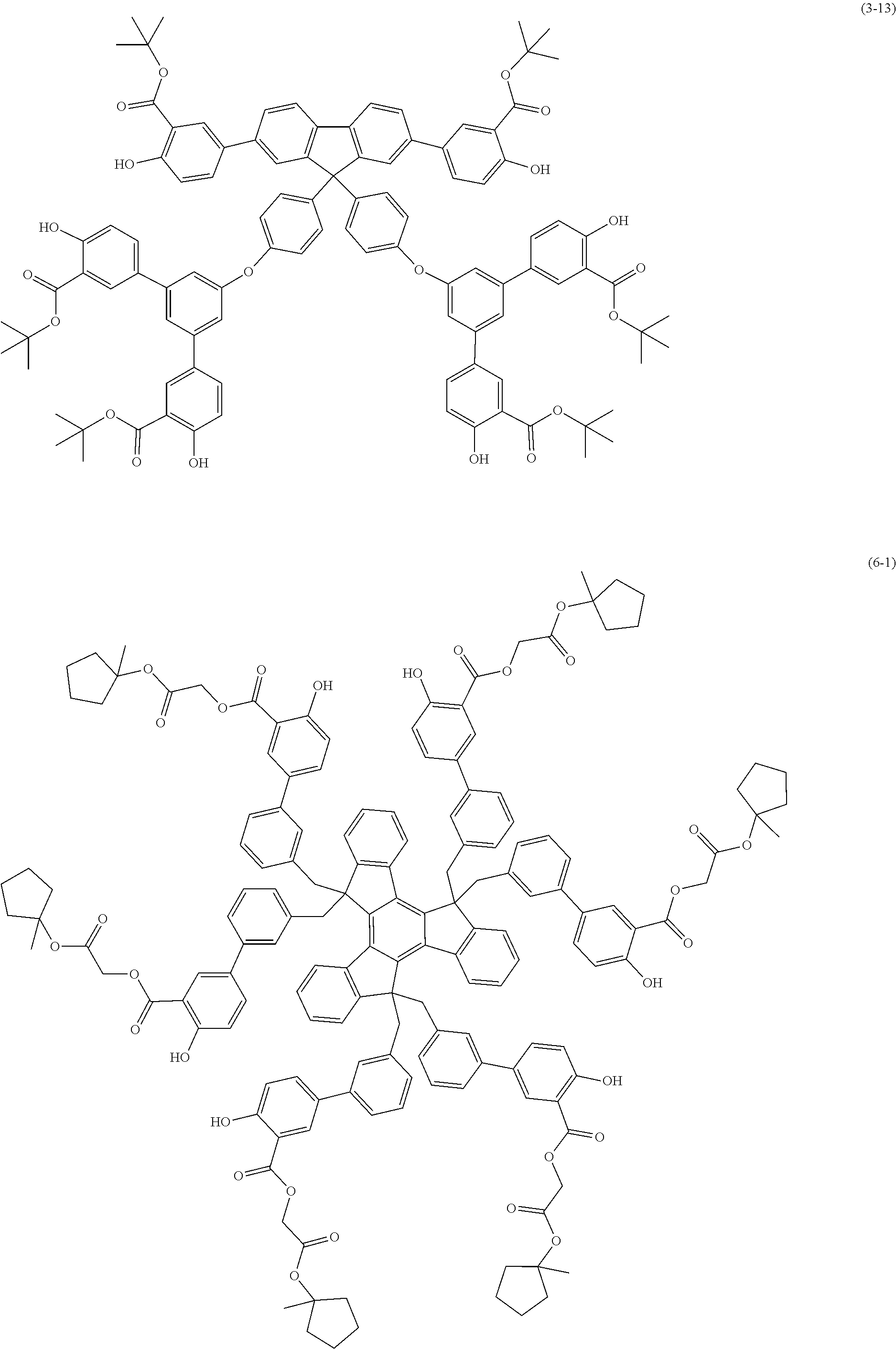

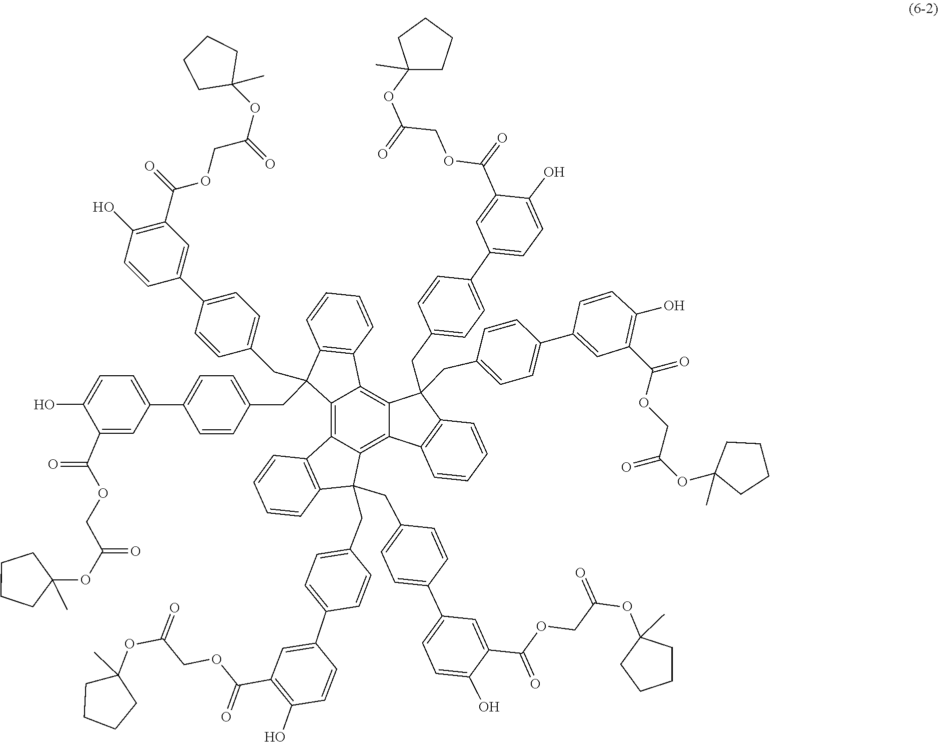 [SMILES: CC1(OC(=O)COC(=O)c2cc(-c3ccc(CC4(Cc5ccc(-c6ccc(O)c(C(=O)OCC(=O)OC7(C)CCCC7)c6)cc5)c5ccccc5-c5c4c4c(c6c5C(Cc5ccc(-c7ccc(O)c(C(=O)OCC(=O)OC8(C)CCCC8)c7)cc5)(Cc5ccc(-c7ccc(O)c(C(=O)OCC(=O)OC8(C)CCCC8)c7)cc5)c5ccccc5-6)C(Cc5ccc(-c6ccc(O)c(C(=O)OCC(=O)OC7(C)CCCC7)c6)cc5)(Cc5ccc(-c6ccc(O)c(C(=O)OCC(=O)OC7(C)CCCC7)c6)cc5)c5ccccc5-4)cc3)ccc2O)CCCC1]

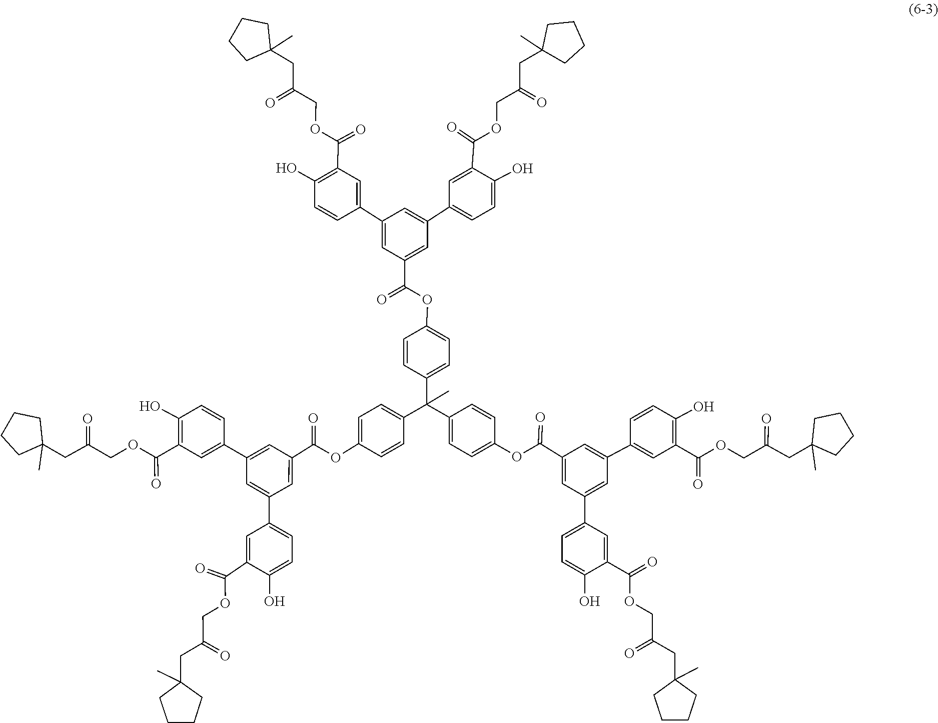 [SMILES: CC1(CC(=O)COC(=O)c2cc(-c3cc(C(=O)Oc4ccc(C(C)(c5ccc(OC(=O)c6cc(-c7ccc(O)c(C(=O)OCC(=O)CC8(C)CCCC8)c7)cc(-c7ccc(O)c(C(=O)OCC(=O)CC8(C)CCCC8)c7)c6)cc5)c5ccc(OC(=O)c6cc(-c7ccc(O)c(C(=O)OCC(=O)CC8(C)CCCC8)c7)cc(-c7ccc(O)c(C(=O)OCC(=O)CC8(C)CCCC8)c7)c6)cc5)cc4)cc(-c4ccc(O)c(C(=O)OCC(=O)CC5(C)CCCC5)c4)c3)ccc2O)CCCC1]

Base resins commonly used in resist compositions may have low line edge roughness (LER) due to polydispersity, large molecular size, chain entanglement, and/or low compatibility with a photoacid generator, and thus it may be difficult to obtain high-resolution patterns thereby.

Meanwhile, unlike polymers, the polycarboxylate compound represented by Formula 1, as an example of molecular glass, have well-defined structure and functional groups and has a relatively small molecular weight. Thus, the polycarboxylate compound represented by Formula 1 may have excellent properties of both polymers and small molecules such as monodispersity, amorphous character, high thermal stability, excellent compatibility with additives, and/or small size.

In addition, because the polycarboxylate compound represented by Formula 1 includes a hydroxyl group, hydrophilicity may be improved and adhesion to a substrate may be increased, and thus a resist film detachment phenomenon may be reduced during development. Because the polycarboxylate compound represented by Formula 1 has improved hydrophilicity, a wet process using an alkaline developer may be performed more easily.

In addition, because the polycarboxylate compound represented by Formula 1 includes a hydroxyl group, the number of intermolecular hydrogen bonds increases and a glass transition temperature (Tg) is raised, and thus breakage of a pattern formed of a resist composition including the polycarboxylate compound may be reduced. In addition, because a temperature range is widened in various processing operations by using a resist composition having a high glass transition temperature, such as the resist composition including the polycarboxylate compound represented by Formula 1, the resist composition may be efficiently applied to photolithography.

A carboxylic moiety is protected by an acid labile group in the polycarboxylate compound represented by Formula 1. In the case where the acid labile group is detached therefrom in the presence of an acid, a polarity of the polycarboxylate compound is changed. In this regard, a change in polarity of the polycarboxylate compound represented by Formula 1 is relatively greater than a change in polarity of a compound having an aromatic hydroxyl group protected by the acid labile group. Therefore, the resist composition including the polycarboxylate compound represented by Formula 1 has relatively improved sensitivity and physical properties thereof may be changed even by a small amount of light exposure.

In addition, the polycarboxylate compound represented by Formula 1 may have improved adhesion to a substrate and a relatively high phase transition temperature because a carboxylic acid group and a hydroxyl group are substituted with one benzene ring.

### [Resist Composition]

According to another embodiment, a resist composition including the above-described polycarboxylate compound, a photoacid generator, and an organic solvent is provided. The resist composition may have improved photosensitivity and/or thermal stability.

Exposure to high-energy rays changes solubility of the resist composition in a developer. The resist composition may be a positive-type resist composition that dissolves and removes an exposed region of a resist film to form a positive-type resist pattern or a negative-type resist composition that dissolves and removes an unexposed region of the resist film to form a negative-type resist pattern. Also, a sensitive resist composition according to an embodiment may be one for an alkaline developing process using an alkaline developer for development while forming a resist pattern or one for a solvent developing process using an organic solvent-containing developer (hereinafter, referred to as organic developer) for development.

In the resist composition, an amount of the polycarboxylate compound may be from 0.1 parts by weight to 50 parts by weight, specifically, 0.2 parts by weight or more, 0.5 parts by weight or more, 1 part by weight or more, or 2 parts by weight or more and 30 parts by weight or less or 20 parts by weight or less based on 100 parts by weight of the composition. In the case where the above-described ranges are satisfied, hydrogen bonds are sufficiently formed between the polycarboxylate compounds while inhibiting side reactions, and thus the resist composition may have improved sensitivity and/or resolution.

### <Organic Solvent>

The resist composition may further include an organic solvent.

The organic solvent included in the resist composition is not particularly limited, as long as the polycarboxylate compound and any component contained therein as needed may be dissolved or dispersed therein. The organic solvent may be used alone or any combination of two or more different organic solvents may also be used.

Examples of the organic solvent may include alcohol-based solvents, ether-based solvents, ketone-based solvents, amide-based solvents, ester-based solvents, sulfoxide-based solvents, and hydrocarbon-based solvents.

More specifically, examples of the alcohol-based solvents may include: a monoalcohol-based solvent such as methanol, ethanol, n-propanol, isopropanol, 1-methoxy-2-propanol, 1-ethoxy-2-propanol, n-butanol, isobutanol, sec-butanol, tert-butanol, n-pentanol, isopentanol, 2-methylbutanol, sec-pentanol, tert-pentanol, 3-methoxybutanol, 3-methyl-3-methoxy butanol, n-hexanol, 2-methylpentanol, sec-hexanol, 2-ethylbutanol, 4-methyl-2-pentanol (MIBC), sec-heptanol, 3-heptanol, n-octanol, 2-ethylhexanol, sec-octanol, n-nonylalcohol, 2,6-dimethyl-4-heptanol, n-decanol, sec-undecyl alcohol, trimethylnonylalcohol, sec-tetradecyl alcohol, sec-heptadecyl alcohol, furfuryl alcohol, phenol, cyclohexanol, methylcyclohexanol, 3,3,5-trimethylcyclohexanol, benzyl alcohol, and diacetone alcohol; a polyalcohol-based solvent such as ethylene glycol, 1,2-propylene glycol, 1,3-butylene glycol, 2,4-pentanediol, 2-methyl-2,4-pentanediol, 2,5-hexanediol, 2,4-heptanediol, 2-ethyl-1,3-hexanediol, diethyleneglycol, dipropyleneglycol, triethylene glycol, and tripropylene glycol; and a polyalcohol-containing ether-based solvent such as ethyleneglycol monomethylether, ethyleneglycol monoethylether, ethyleneglycol monopropylether, ethyleneglycol monobutylether, ethyleneglycol monohexylether, ethyleneglycol monophenylether, ethyleneglycol mono-2-ethylbutylether, diethyleneglycol monomethylether, diethyleneglycol monoethylether, diethyleneglycol monopropylether, diethyleneglycol monobutylether, diethyleneglycol monohexyl ether, diethylene glycol dimethylether, propylene glycol monomethylether, propylene glycol dimethylether, propylene glycol monoethylether, propylene glycol monopropylether, propylene glycol monobutylether, dipropyleneglycol monomethylether, dipropyleneglycol monoethylether, and dipropyleneglycol monopropylether.

Examples of the ether-based solvents may include: a dialkylether-based solvent such as diethylether, dipropylether, and dibutylether; a cyclic ether-based solvent such as tetrahydrofuran and tetrahydropyran; and an aromatic ring-containing ether-based solvent such as diphenylether and anisole.

Examples of the ketone-based solvents may include: a chain-shaped ketone-based solvent such as acetone, methylethylketone, methyl-n-propylketone, methyl-n-butylketone, methyl-n-pentylketone, diethylketone, methylisobutylketone, 2-heptanone, ethyl-n-butylketone, methyl-n-hexylketone, diisobutylketone, and trimethylnonanone; a cyclic ketone-based solvent such as cyclopentanone, cyclo hexanone, cycloheptanone, cyclooctanone, and methylcyclohexanone; and 2,4-pentanedione, acetonylacetone, and acetophenone.

Examples of the amide-based solvents may include: a cyclic amide-based solvent such as N,N′-dimethylimidazolidinone and N-methyl-2-pyrrolidone; and a chain-shaped amide-based solvent such as N-methylformamide, N,N-dimethylformamide, N,N-diethylformamide, acetamide, N-methylacetamide, N,N-dimethylacetamide, and N-methylpropionamide.

Examples of the ester-based solvents include: an acetate ester-based solvent such as methyl acetate, ethyl acetate, n-propyl acetate, isopropyl acetate, n-butyl acetate, isobutyl acetate, sec-butyl acetate, T-butyl acetate, n-pentyl acetate, isopentyl acetate, sec-pentyl acetate, 3-methoxybutyl acetate, methylpentyl acetate, 2-ethylbutyl acetate, 2-ethylhexyl acetate, benzyl acetate, cyclohexyl acetate, methylcyclohexyl acetate, and n-nonyl acetate; a polyalcohol-containing ethercarboxylate-based solvent such as ethyleneglycol monomethylether acetate, ethyleneglycol monoethylether acetate, diethyleneglycol monomethylether acetate, diethyleneglycol monoethylether acetate, diethyleneglycol mono-n-butyl ether acetate, propylene glycol monomethylether acetate (PGMEA), propylene glycol monoethylether acetate, propylene glycol monopropylether acetate, propylene glycol monobutylether acetate, dipropylene glycol monomethylether acetate, and dipropylene glycol monoethylether acetate; a lactone-based solvent such as γ-butyrolactone and δ-valerolactone; a carbonate-based solvent such as dimethyl carbonate, diethyl carbonate, ethylene carbonate, and propylene carbonate; a lactate ester-based solvent such as methyl lactate, ethyl lactate, n-butyl lactate, and n-amyl lactate; and glycoldiacetate, methoxytriglycol acetate, ethyl propionate, n-butyl propionate, isoamyl propionate, diethyloxalate, di-n-butyloxalate, methyl acetoacetate, ethyl acetoacetate, diethyl malonate, dimethyl phthalate, and diethyl phthalate.

Examples of the sulfoxide-based solvents may include dimethyl sulfoxide and diethyl sulfoxide.

Examples of the hydrocarbon-based solvents include: an aliphatic hydrocarbon-based solvent such as n-pentane, isopentane, n-hexane, isohexane, n-heptane, isoheptane, 2,2,4-trimethyl pentane, n-octane, isooctane, cyclohexane, and methylcyclohexane; and an aromatic hydrocarbon-based solvent such as benzene, toluene, xylene, mesitylene, ethylbenzene, trimethylbenzene, methylethylbenzene, n-propylbenzene, isopropylbenzene, diethylbenzene, isobutylbenzene, triethylbenzene, diisopropylbenzene, and n-amylnaphthalene.

Specifically, the organic solvent may be selected from the alcohol-based solvent, the amide-based solvent, the ester-based solvent, the sulfoxide-based solvent, and any combination thereof. More specifically, the solvent may be selected from propylene glycol monomethylether, propylene glycol monoethylether, propylene glycol monomethylether acetate, N-methyl-2-pyrrolidone, N,N-dimethylacetamide, ethyl lactate, dimethylsulfoxide, and any combination thereof.

Meanwhile, in the case of using an acid labile group in the form of acetal, the organic solvent may further include a high-boiling point alcohol, such as diethyleneglycol, propylene glycol, glycerol, 1,4-butanediol, or 1,3-butanediol, to accelerate deprotection reaction of the acetal.

The organic solvent may be included in an amount of 200 parts by weight to 5,000 parts by weight, specifically, 400 parts by weight to 3,000 parts by weight based on 100 parts by weight of the polycarboxylate compound.

### <Photoacid Generator>

The photoacid generator may be any compound capable of generating an acid when exposed to high-energy rays such as UV rays, DUV rays, EBs, EUV rays, X-rays, α-rays, and γ-rays.

The photoacid generator may include a sulfonium salt, an iodonium salt, and any combination thereof.

In an embodiment, the photoacid generator may be represented by Formula 7 below.

B₇₁⁺A₇₁⁻ <Formula 7>

In Formula 7, B₇₁⁺ is represented by Formula 7A below, A₇₁⁻ is represented by one of Formulae 7B to 7D below, and B₇₁⁺ may optionally be linked to A₇₁⁻ by a carbon-carbon covalent bond.

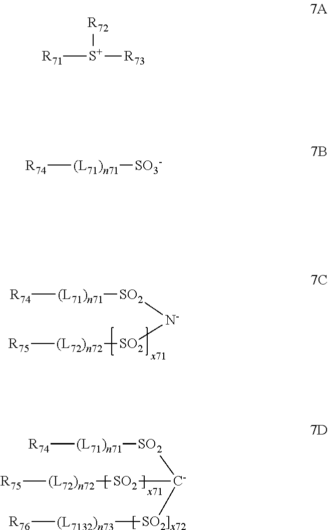

In Formulae 7A to 7D,
L₇₁ to L₇₃ are each independently a single bond or CRR′,
R and R′ are each independently hydrogen, deuterium, a halogen atom, a cyano group, a hydroxyl group, a C₁-C₃₀ alkyl group, a C₁-C₃₀ halogenated alkyl group, a C₁-C₃₀ alkoxy group, a C₃-C₃₀ cycloalkyl group, or a C₃-C₃₀ cycloalkoxy group,
n71 to n73 are each independently 1, 2, or 3,
x71 and x72 are each independently 0 or 1,
R₇₁ to R₇₃ are each independently a linear, branched, or cyclic C₁-C₃₀ monovalent hydrocarbon group,
among R₇₁ to R₇₃, two adjacent ones may optionally bind to each other to form a ring, and
R₇₄ to R₇₆ are each independently hydrogen; a halogen atom; or a linear, branched, or cyclic C₁-C₃₀ monovalent hydrocarbon group optionally including a hetero atom.

For example, in Formula 7, B₇₁⁺ may be represented by Formula 7A and A₇₁⁻ may be represented by Formula 7B. Specifically, in Formula 7A, R₇₁ to R₇₃ may be each independently a phenyl group.

The photoacid generator may be included in an amount of 0 parts by weight to 40 parts by weight, 0.1 parts by weight to 40 parts by weight, 0.1 parts by weight to 20 parts by weight based on 100 parts by weight of the polycarboxylate compound. In the case where the above-described ranges are satisfied, an appropriate resolution may be obtained, and problems related to particles of foreign matter after development or during stripping may be reduced.

The photoacid generator may be used alone or any combination of two or more different photoacid generators may also be used.

### <Quencher>

The quencher may be a salt generating an acid with a lower acidity than an acid generated from the photoacid generator.

The quencher may include an ammonium salt, a sulfonium salt, an iodonium salt, and any combination thereof.

In an embodiment, the quencher may be represented by Formula 8 below.

B₈₁⁺A₈₁⁻ <Formula 8>

In Formula 8, B₈₁⁺ is represented by one of Formulae 8A to 8C below, and A₈₁ is represented by one of Formulae 8D to 8F below, and B₈₁⁺ may optionally be linked to A₈₁⁻ by a carbon-carbon covalent bond.

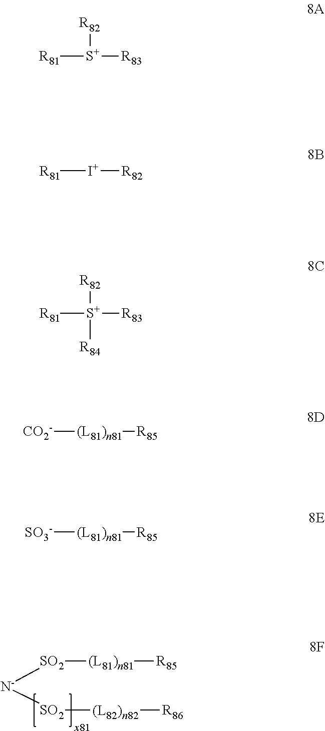

In Formulae 8A to 8F,
L₈₁ and L₈₂ are each independently a single bond or CRR′,
R and R′ are each independently hydrogen, deuterium, a halogen atom, a cyano group, a hydroxyl group, C₁-C₃₀ alkyl group, a C₁-C₂₀ halogenated alkyl group, a C₁-C₃₀ alkoxy group, a C₃-C₃₀ cycloalkyl group, or a C₃-C₃₀ cycloalkoxy group,
n81 and n82 are each independently 1, 2 or 3,
x81 is 0 or 1,
R₈₁ to R₈₄ are each independently a linear, branched, or cyclic C₁-C₃₀ monovalent hydrocarbon group,
among R₈₁ to R₈₄, two adjacent ones may optionally bind to each other to form a ring, and
R₈₅ and R₈₆ are each independently hydrogen; a halogen atom; or a linear, branched, or cyclic C₁-C₃₀ monovalent hydrocarbon group optionally including a hetero atom.

The quencher may be included in an amount of 0.01 parts by weight to 10 parts by weight, 0.05 parts by weight to 5 parts by weight, or 0.1 parts by weight to 3 parts by weight based on 100 parts by weight of the polycarboxylate compound. In the case where the above-described ranges are satisfied, an appropriate resolution may be obtained, and problems related to particles of foreign matter after development or during stripping may be reduced.

The quencher may be used alone or any combination of two or more different quenchers may also be used.

### <Other Components>

The resist composition may further include a surfactant, a crosslinking agent, a leveling agent, a colorant, or any combination thereof, if necessary.

The resist composition may further include a surfactant to improve coatability, developability, and the like. Examples of the surfactant may include a nonionic surfactant such as polyoxyethylene lauryl ether, polyoxyethylene stearyl ether, polyoxyethyleneoleyl ether, polyoxyethylene n-octylphenyl ether, polyoxyethylene n-nonylphenyl ether, polyethyleneglycol dilaurate, and polyethyleneglycol distearate. Any commercially available product or a synthetic product may be used as the surfactant. Examples of the commercially available product may include KP341 (manufactured by Shin-Etsu Chemical Co., Ltd.), Polyflow No. 75 and Polyflow No. 95 (manufactured by Kyoeisha Chemical Co., Ltd.), Eftop EF301, Eftop EF303, and Eftop EF352 (manufactured by Mitsubishi Materials Electronic Chemicals Co., Ltd.), MEGAFACE (registered trademark) F171, MEGAFACE F173, R40, R41, and R43 (manufactured by DIC Corporation), Fluorad (registered trademark) FC430, Fluorad FC431 (manufactured by 3M Co., Ltd.), AsahiGuard AG710 (manufactured by AGC Co., Ltd.), and Surflon (registered trademark) S-382, Surflon SC-101, Surflon SC-102, Surflon SC-103, Surflon SC-104, Surflon SC-105, and Surflon SC-106 (manufactured by AGC Seimi Chemical Co., Ltd).

The surfactant may be included in an amount of 0 parts by weight to 20 parts by weight based on 100 parts by weight of the polycarboxylate compound. The surfactant may be used alone or any mixture of two or more different surfactants may also be used.

A method of manufacturing the resist composition is not particularly limited. For example, a method of mixing the polycarboxylate compound, the photoacid generator, and any other components added, if necessary, in an organic solvent may be used. Temperature or time is not particularly limited during mixing. If necessary, filtration may be performed after mixing.

### [Method of Forming Pattern]

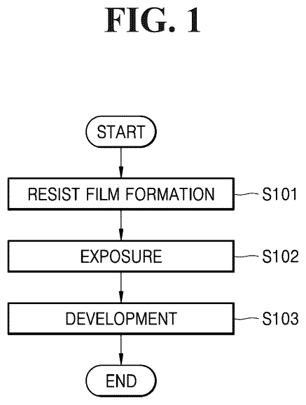

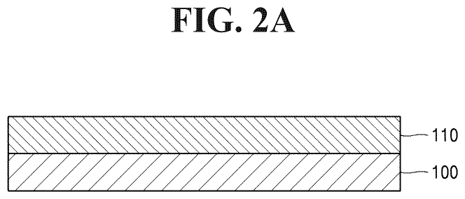

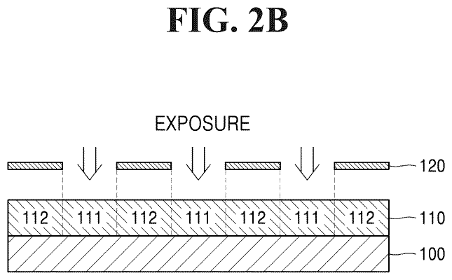

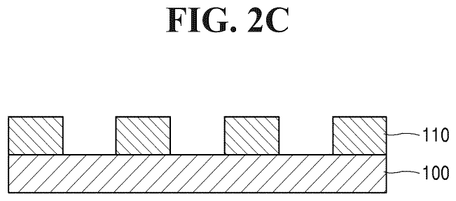

Hereinafter, a method of forming a pattern according to embodiments will be described in more detail with reference to FIGS. 1 and 2A to 2C. FIG. 1 is a flowchart illustrating a method of forming a pattern according to an embodiment. FIG. 2A to 2C are a side cross-sectional view illustrating a method of forming a pattern according to an embodiment. Hereinafter, a method of forming a pattern using a negative resist composition will be described by way of example, but the embodiment is not limited thereto.

Referring to FIG. 1, a method of forming a pattern includes forming a resist film by applying a resist composition (S101), exposing at least one portion of the resist film to high-energy rays (S102), and developing the exposed resist film using a developer (S103). These operations may be omitted or may be performed in a different order, if necessary.

First, a substrate 100 is prepared. The substrate 100 may be a semiconductor substrate such as a silicon substrate and a germanium substrate, or may be formed of glass, quartz, ceramic, copper, or the like. In some embodiments, the substrate 100 may include compounds of Groups 3 to 5, such as GaP, GaAs, and GaSb.

A resist film 110 may be formed on the substrate 100 by applying the resist composition thereto to a desired thickness using a coating method. If necessary, the resist film 110 may be heated (pre-baked, PB) to remove the organic solvent remaining therein.

As the coating method, spin coating, dipping, roller coating, or other common coating methods may be used. Among them, spin coating may particularly be used. The resist film 110 having a desired thickness may be formed by adjusting viscosity, concentration, and/or spin speed of the resist composition. Specifically, the resist film 110 may have a thickness of 10 nm to 300 nm. More specifically, the resist film 110 may have a thickness of 30 nm to 200 nm.

A lower limit of a pre-baking temperature may be 60° C. or higher, specifically, 80° C. or higher. In addition, an upper limit of the pre-baking temperature may be 150° C. or less, specifically, 140° C. or lower. A lower limit of a pre-baking time may be 5 seconds or more, specifically, 10 seconds or more. An upper limit of the pre-baking time may be 600 seconds or less, specifically, 300 seconds or less.

Before applying the resist composition to the substrate 100, a film to be etched (not shown) may be formed on the substrate 100. The film to be etched may refer to a film onto which an image is transferred from a resist pattern and converted into a pattern. In an embodiment, the film to be etched may be formed to include, for example, an insulating material such as a silicon oxide, a silicon nitride, and a silicon oxynitride. In some embodiments, the film to be etched may be formed to include a conductive material such as a metal, a metal nitride, a metal silicide, and a metal silicide nitride film. In some embodiments, the film to be etched may be formed to include a semiconductor material such as polysilicon.

In an embodiment, an anti-reflection film may further be formed on the substrate 100 to maximize efficiency of the resist. The anti-reflection film may be an organic or inorganic anti-reflection film.

In an embodiment, a protective film may further be formed on the resist film 110 to reduce effects of alkaline impurities included during a process. In addition, in the case of performing immersion lithography, a protective film for immersion lithography may be formed on the resist film to avoid direct contact between an immersion medium and the resist film 110.

Subsequently, at least one portion of the resist film 110 may be exposed to high-energy rays. For example, high-energy rays having passed through a mask 120 may reach at least one portion of the resist film 110. Therefore, the resist film 110 may have exposed regions 111 and unexposed regions 112.

The light exposure is performed by emitting high-energy rays through a mask having a particular pattern using a liquid such as water as a medium, if necessary. Examples of the high-energy rays may include electromagnetic waves such as ultraviolet rays, deep ultraviolet rays, extreme ultraviolet (EUV) rays, wavelength of 13.5 nm), X-rays, and γ-rays; and charged particle beams such as electron beams and a particle beams. Irradiation of these high-energy rays may be collectively referred to as “exposure”.

Various light sources may be used for the exposure. For example, a light source emitting laser beams in the UV range, such as a KrF excimer laser (wavelength of 248 nm), an ArF excimer laser (wavelength of 193 nm), and an F2 excimer laser (wavelength of 157 nm), a light source emitting harmonic laser beams in the far ultraviolet or vacuum ultraviolet range by converting wavelengths of laser beams received from a solid laser light source (YAG or semiconductor laser), and a light source emitting electron beams or EUVs may be used. During exposure, a mask corresponding to a desired pattern is commonly used. However, in the case of using electron beams as a light source for exposure, exposure may be directly performed without using a mask.

An integral dose of the high-energy rays may be 2000 mJ/cm² or less, specifically, 500 mJ/cm² or less, in the case of using extreme ultraviolet rays as the high-energy rays. In addition, in the case of using electron beams as the high-energy rays, the integral dose may be 5000 μC/cm² or less, specifically, 1000 μC/cm² or less.

Also, post exposure baking (PEB) may be performed after exposure. A lower limit of a PEB temperature may be 50° C. or higher, specifically, 80° C. or higher. An upper limit of the PEB temperature may be 250° C. or lower, specifically, 200° C. or lower. A lower limit of a PEB time may be, 5 seconds or more, specifically, 10 seconds or more. An upper limit of the PEB time may be 600 seconds of less, specifically, 300 seconds or less.

The exposed resist film 110 may be developed using a developer. The exposed regions 111 may be washed away by the developer, the unexposed regions 112 may remain without being washed by the developer.

As the developer, an alkaline developer, an organic solvent-containing developer (hereinafter, referred to as “organic developer”), and the like may be used. A developing method may be dipping, puddling, spraying, dynamic approach, or the like. A developing temperature may be, for example, from 5° C. to 60° C., and a developing time may be, for example, from 5 seconds to 300 seconds.

Examples of the alkaline developer may include an alkaline aqueous solution include at least one alkaline compound dissolved therein such as sodium hydroxide, potassium hydroxide, sodium carbonate, sodium silicate, sodium metasilicate, aqueous ammonia, ethylamine, n-propylamine, diethylamine, di-n-propyl amine, triethylamine, methyldiethylamine, ethyldimethylamine, triethanolamine, tetramethyl ammonium hydroxide (TMAH), pyrrole, piperidine, choline, 1,8-diazabicyclo[5.4.0]-7-undecene (DBU), and 1,5-diazabicyclo[4.3.0]-5-nonene (DBN). The alkaline developer may further include a surfactant.

A lower limit of an amount of the alkaline compound contained in the alkaline developer may be 0.1 wt % or more, specifically, 0.5 wt % or more, more specifically, 1 wt % or more. In addition, an upper limit of the amount of the alkaline compound contained in the alkaline developer may be 20 wt % or less, specifically, 10 wt % or less, more specifically, 5 wt % or less.

For example, the organic solvent contained in the organic developer may be the same as those described above in the <Organic Solvent> section of the [Resist Composition].

In the organic developer, a lower limit of the amount of the organic solvent may be 80 wt % or more, specifically, 90 wt % or more, more specifically, 95 wt % or more, particularly, 99 wt % or more.

The organic developer may include a surfactant. In addition, the organic developer may include a trace amount of moisture. In addition, development may be stopped during developing by replacing the organic developer with a different type of solvent.

The resist pattern may further be washed after the developing. Ultrapure water, a ringing solution, and the like may be used during washing. The rinsing solution is not particularly limited as long as the resist pattern is not dissolved therein, and any solution including a common organic solvent may be used. For example, the rinsing solution may be an alcohol-based solvent or an ester-based solvent. After washing, the rinsing solution remaining on the substrate and the pattern may be removed. Also, in the case of using ultrapure water, water remaining on the substrate and the pattern may be removed.

In addition, the developer may be used alone or any combination of two or more different developers may also be used.

After the resist pattern is formed as described above, a pattered wiring substrate may be obtained by etching. Any etching methods well known in the art such as dry etching using plasma gas, and wet etching using an alkaline solution, a copper (II) chloride solution, or a ferric chloride solution may be used.

After forming the resist pattern, plating may be performed. A plating method is not particularly limited, but for example, copper plating, solder plating, nickel plating, and gold plating may be used therefor.

The resist pattern remaining after etching may be stripped off using an organic solvent. Examples of the organic solvent may be, but are not limited to, propylene glycol monomethylether acetate (PGMEA), propylene glycol monomethylether (PGME), and ethyl lactate (EL). A peeling method is not particularly limited, but may be, for example, immersing and spraying. In addition, a wiring substrate on which the resist pattern is formed may be a multi-layered wiring substrate or may have a small-diameter through hole.

In an embodiment, the wiring substrate may be formed by a method including forming a resist pattern, depositing a metal in a vacuum, and melting the resist pattern using a solution, e.g., a lift-off method.

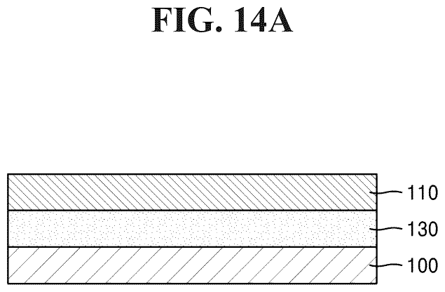

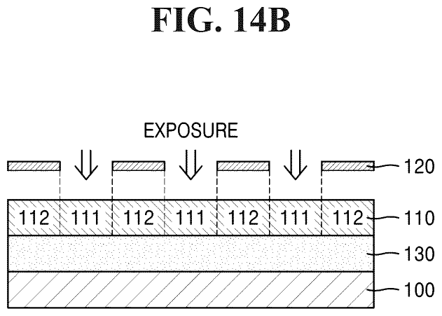

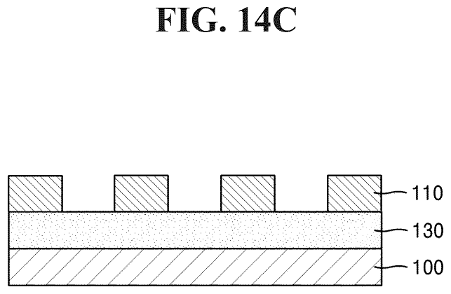

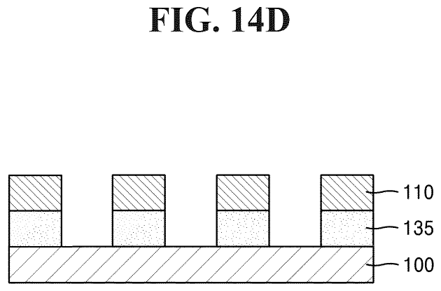

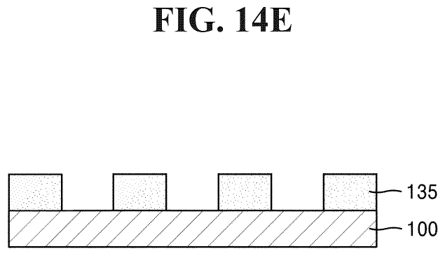

FIGS. 14A to 14E are side cross-sectional views illustrating a method of forming a patterned structure according to an embodiment.

Referring to FIG. 14A, a material layer 130 may be formed on the substrate 100 before forming a resist film 110 on the substrate 100. The resist film 110 may be formed on top of the material layer 130. The material layer 130 may include an insulating material (e.g., silicon oxide, silicon nitride), a semiconductor material (e.g., silicon), a metal (e.g., copper). In some embodiments, the material layer 130 may be a multi-layer structure. A material of the material layer 130 may be different than a material of the substrate 100.

Referring to FIG. 14B, like the operations described in FIGS. 2A to 2B, the resist film 110 may undergo a pre-exposure bake process and may be exposed with high energy rays through a mask 120, after which the resist film 110 may include exposed regions 111 and unexposed regions 112.

Referring to FIG. 14C, like the operation described in FIG. 2C, the exposed resist film 110 may be developed using a developer (e.g., developing solution). The exposed area 111 may be washed away by the developer, whereas the unexposed area 112 may remain without being washed away by the developer.

Referring to FIG. 14D, exposed areas of the material layer 130 may be etched using the resist pattern 110 to form a material pattern 135 on the substrate 100.

Referring to FIG. 14E, the resist pattern 110 may be removed.

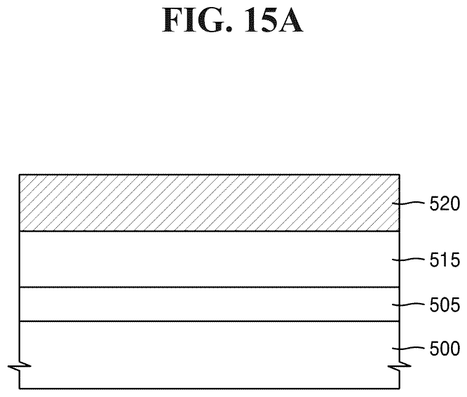

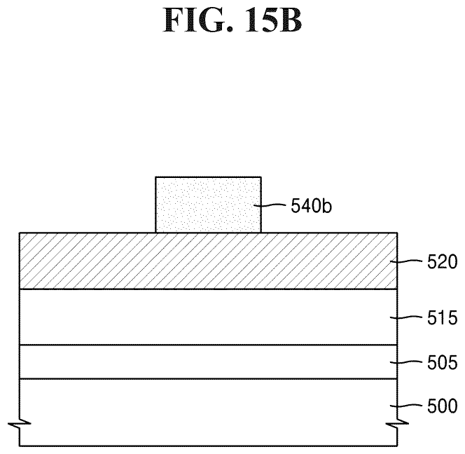

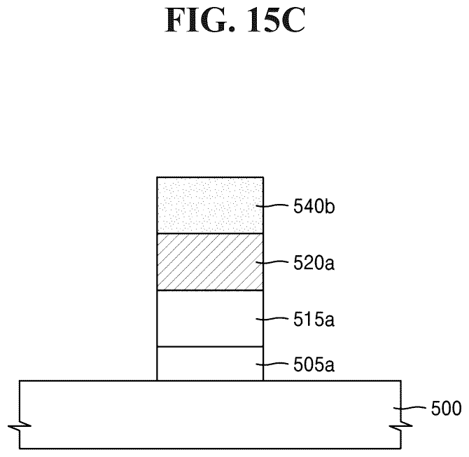

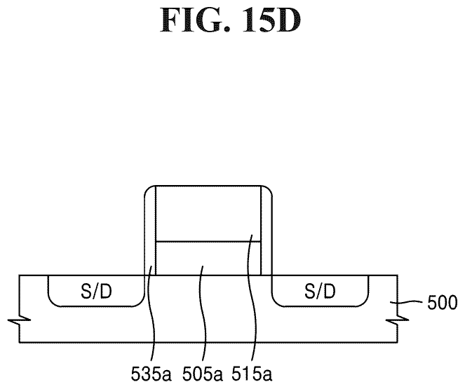

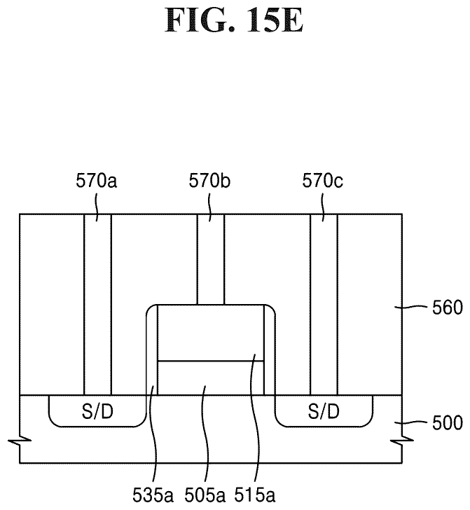

FIGS. 15A to 15E are side cross-sectional views illustrating a method of forming a semiconductor device according to an embodiment.

Referring to FIG. 15A, a gate dielectric 505 (e.g., silicon oxide) may be formed on a substrate 500. The substrate 500 may be a semiconductor substrate, such as a silicon substrate. A gate layer 515 (e.g., doped polysilicon) may be formed on the gate dielectric 505. A hardmask layer 520 may be formed on the gate layer 515.

Referring to FIG. 15B, a resist pattern 540b may be formed on the hardmask layer 520. The resist pattern 540b may be formed using a resist composition including a polycarboxylate compound according to example embodiments. The resist composition may include a photoacid generator and an organic solvent.

Referring to FIG. 15C, the gate layer 515 and the gate dielectric 505 may be etched to form a hardmask pattern 520a, a gate electrode pattern 515a, and a gate dielectric pattern 505a.

Referring to FIG. 15D, a spacer layer may be formed over the gate electrode pattern 515a and the gate dielectric pattern 505a. The spacer layer may be formed using a deposition process (e.g., CVD). The spacer layer may be etched to form spacers 535a (e.g., silicon nitride) on sidewalls of the gate electrode pattern 515a and the gate dielectric pattern 505a. After forming the spacers 535a, ions may be implanted into the substrate 500 to form source/drain impurity regions S/D.

Referring to FIG. 15E, an interlayer insulating layer 560 (e.g., oxide) may be formed on the substrate 500 to cover the gate electrode pattern 515a, gate dielectric pattern 505a, and spacers 535a. Then, electrical contacts 570a, 570b, and 570c may be formed in the interlayer insulating layer 560 to connect to the gate electrode 515a and the S/D regions. The electrical contacts may be formed of a conductive material (e.g., metal). Although not illustrated, a barrier layer may be formed between sidewalls of the interlayer insulating layer 560 and the electrical contacts 570a, 570b, and 570c. While FIGS. 15A to 15E illustrate an example of forming a transistor, inventive concepts are not limited thereto. A resist composition including a polycarboxylate compound according one or more embodiments may be used in a patterning process to form other types of semiconductor devices. The resist composition may include a photoacid generator and an organic solvent.

Hereinafter, the disclosure will be described in more detail according to the following examples and comparative examples. However, the following examples are merely presented as non-limiting examples, and the scope of the disclosure is not limited thereto.

### EXAMPLES

### Synthesis Example 1

### (1) Synthesis of Compound (2-3)

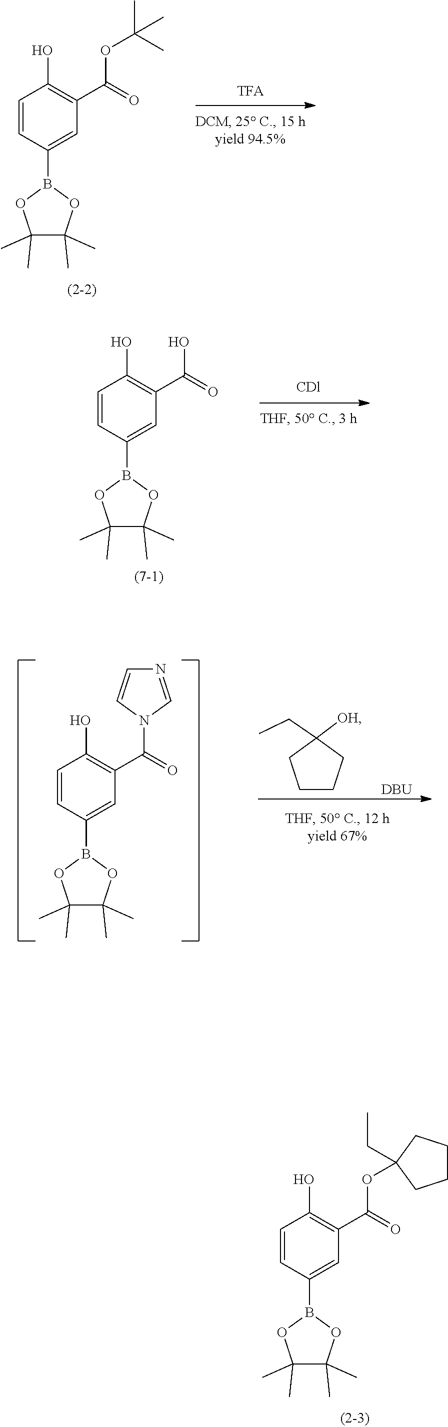

### 1) Synthesis of Compound (2-2)

Referring to J. Am. Chem. Soc. 2021, 143, 27, 10317-10323, Compound (2-2) was synthesized.

### 2) Synthesis of Compound (7-1)

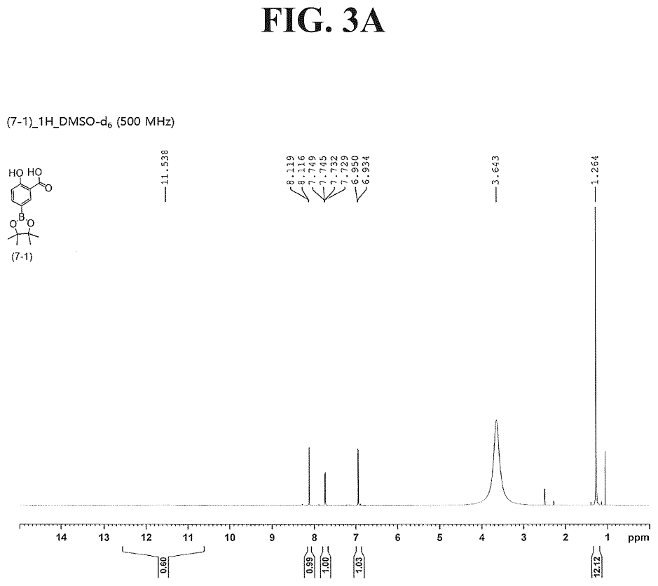

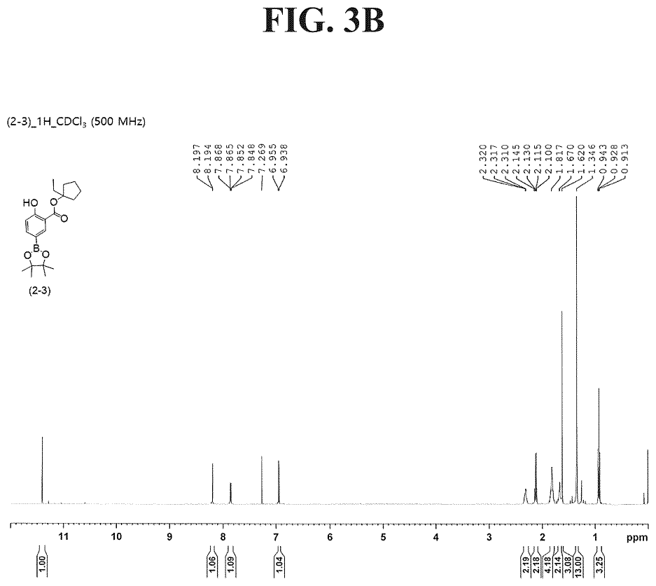

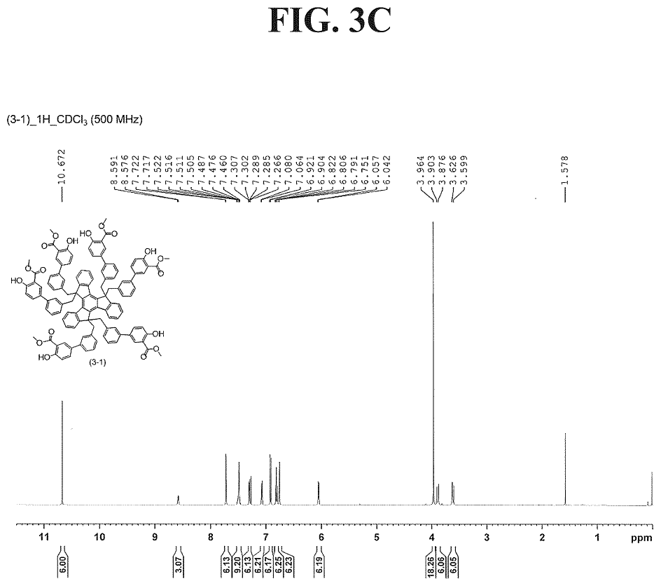

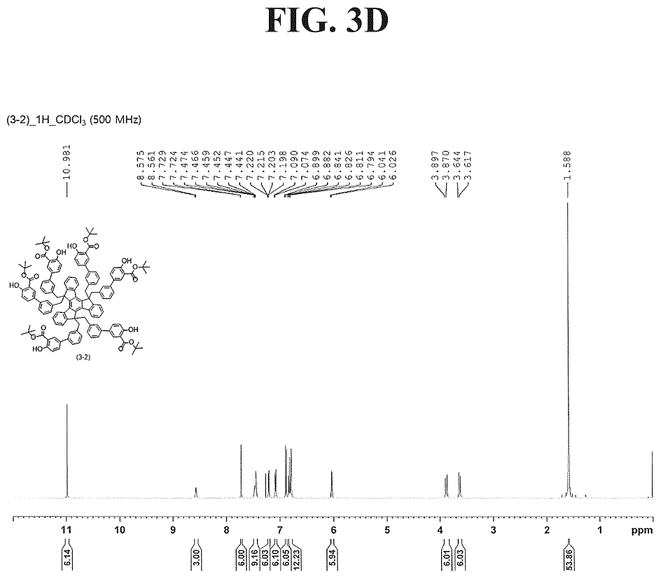

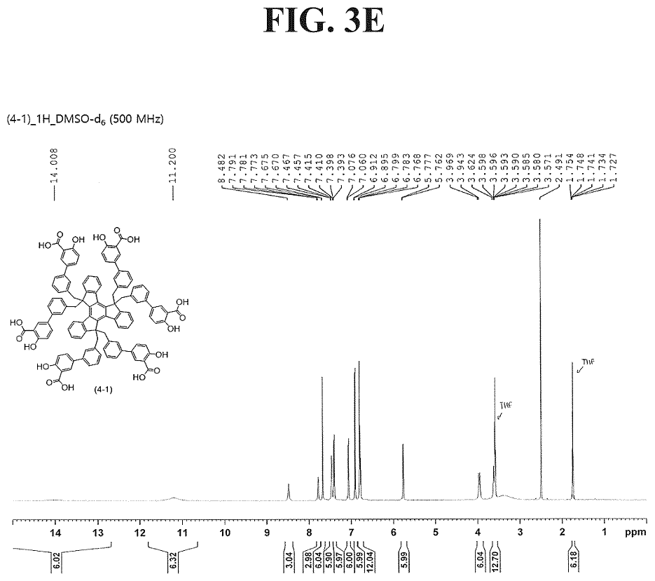

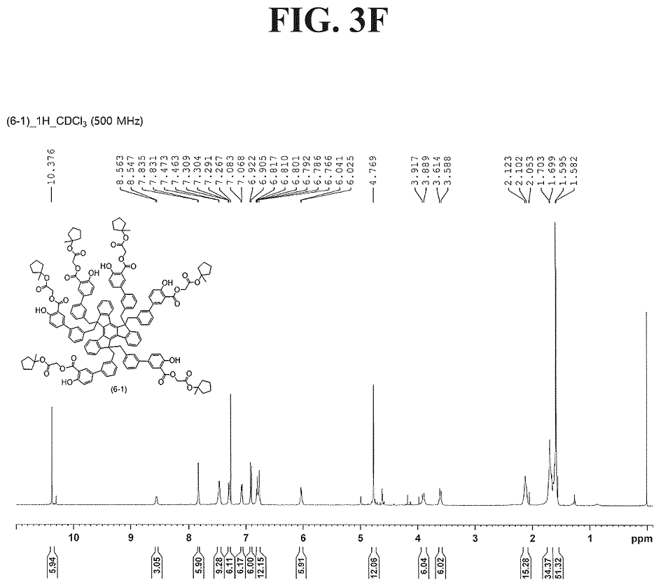

2.48 g (7.77 mmol) of Compound (2-2) was dissolved in 50 mL of DCM, and 17.71 g (0.155 mol) of TFA was added thereto while stirring. A mixture obtained thereby was stirred at 25° C. for 15 hours. Volatile materials were evaporated under reduced pressure. Residual solid materials were suspended in 30 ml of toluene, filtered, and washed with toluene and hexane. A product was dried at 85° C. 1.88 g (92.2%) of the product was a white crystalline material with R_{f}=0.64 (eluent: EtOAc, TLC silica gel 60 F254). ¹H-NMR data of obtained Compound (7-1) are shown in FIG. 3A.

### 3) Synthesis of Compound (2-3)

7.8 g (29.64 mmol) of Compound (7-1) was dissolved in 100 ML of THF, and 7.21 g (44.45 mmol) of 1,1′-carbonyldiimidazole (CDI) was added thereto while stirring. A mixture obtained thereby was heated to 50° C. and stirred for 3 hours, and then cooled to 25° C. and degassed under reduced pressure. 8.46 g (74.1 mmol) of 1-ethylcyclopentanol and 9 g (59.1 mmol) of DBU were sequentially added thereto to obtain a mixture and the mixture was stirred at 50° C. for 12 hours. Upon completion of reaction, the mixture was diluted with EtOAc (0.2 L) and washed twice with 0.2 L of a NH₄Cl aqueous solution, and then dried in the presence of Na₂SO₄. After removing the solvent under reduced pressure, a residue was separated by flash chromatography on SiO₂ using DCM/Hex (1/1 (v/v), 1 L) and DCM (0.3 L). A residue obtained after evaporating the solvent was dried under reduced pressure at 40° C. 7.02 g (yield: 67%) of Compound (2-3) was obtained as a white solid. R_{f}=0.36 (eluent: DCM/Hexane=1/1, TLC silica gel 60 F254). ¹H-NMR data of obtained Compound (2-3) are shown in FIG. 3B.

### (2) Synthesis of Compound (4-1)

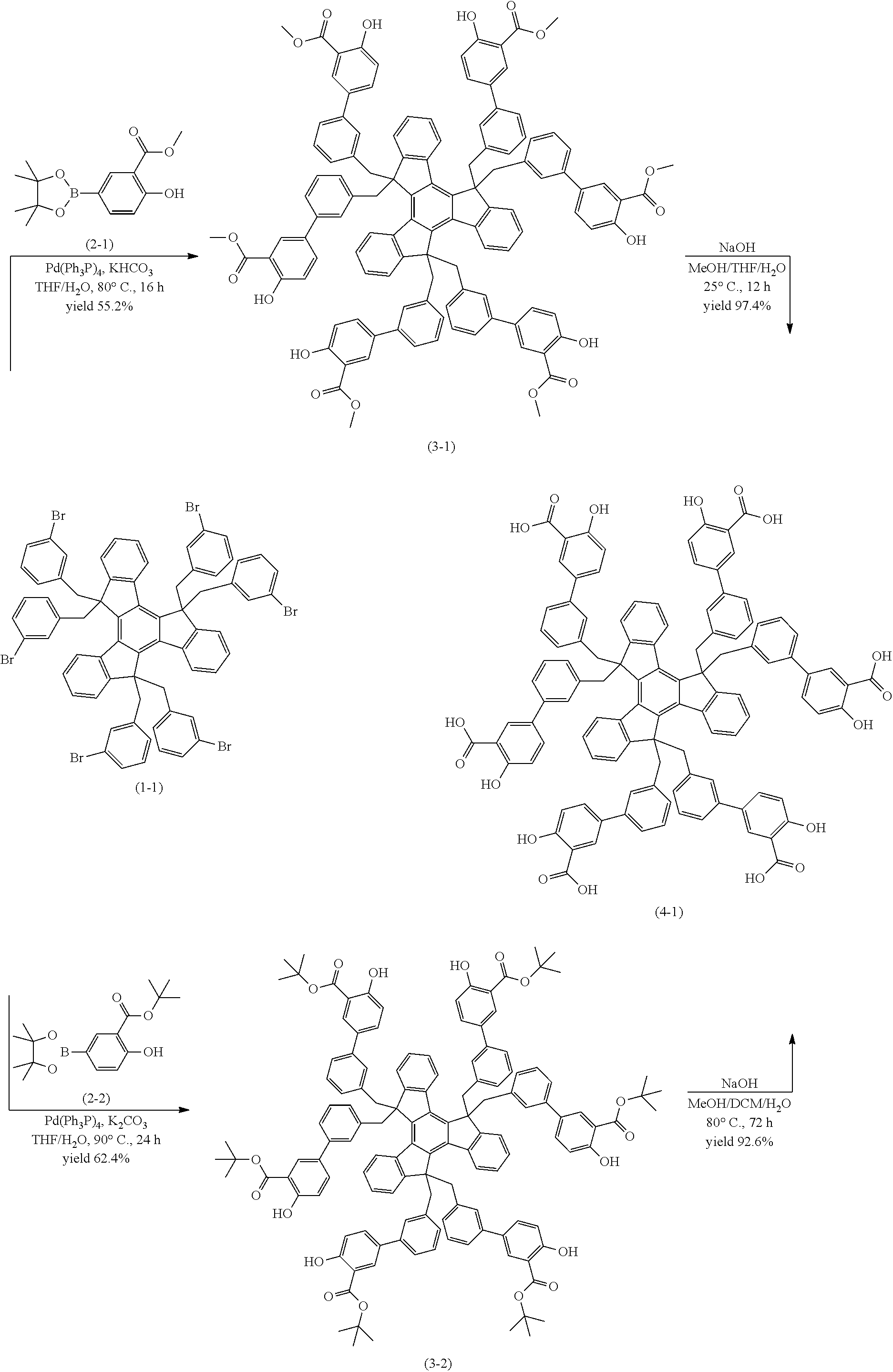

### 1) Synthesis of Compound (1-1)

Referring to European Journal of Organic Chemistry (2005), (19), 4127-4140), Compound (1-1) was synthesized.

### 2) Synthesis of Compound (3-1)

0.678 g (0.5 mmol) of Compound (1-1), 1.25 g (4.5 mmol) of Compound (2-1), 87 mg (0.075 mmol) of Pd(PPh₃)₄, and 1.35 g (13.5 mmol) of KHCO₃ were added to a 100 mL-round bottom flask and 45 mL of a THF/H₂O mixture (2:1 (v:v)) was added thereto. A mixture obtained thereby was degassed and heated to 80° C., and then stirred in a nitrogen atmosphere for 16 hours. Upon completion of reaction, the mixture was diluted with EtOAc (0.2 L) and washed twice with 0.2 L of a NH₄Cl aqueous solution, and then dried in the presence of Na₂SO₄. After removing the solvent by drying under reduced pressure, a residue was separated by chromatography on SiO₂ with DCM/EtOAc (gradient from 100:0 to 95:5) to obtain fragments with an Rr of 0.53 (eluent: DCM, TLC silica gel 60 F254). The residue was treated with a small amount of MeOH and dried under reduced pressure at 60° C. 0.492 g (yield: 55.2%) of Compound (3-1) was obtained as a white solid. ¹H-NMR data of obtained Compound (3-1) are shown in FIG. 3C.

### 3) Synthesis of Compound (3-2)

1.36 g (1 mmol) of Compound (1-1), 2.39 g (7.5 mmol) of Compound (2-2), 173 mg (0.15 mmol) of Pd(PPh₃)₄ and 3.73 g (27 mmol) of K₂CO₃ were added to a 100 mL-round bottom flask and 80 mL of a THF/H₂O mixture (3:1 (v:v)) was added thereto. A mixture obtained thereby was degassed and heated to 80° C., and then stirred for 24 hours. Upon completion of reaction, the mixture was diluted with EtOAc (0.2 L) and washed twice with 0.2 L of a NH₄Cl aqueous solution, and then dried in the presence of Na₂SO₄. After removing the solvent by drying under reduced pressure, a residue was separated by chromatography on SiO₂ with DCM/hexane (gradient from 50:50 to 100:0) to obtain fragments with an Rr of 0.63 (eluent: DCM, TLC silica gel 60 F254). The residue was treated with a small amount of MeOH and dried under reduced pressure at 60° C. 1.267 g (yield: 62.4%) of Compound (3-2) was obtained as a white solid. ¹H-NMR data of obtained Compound (3-2) are shown in FIG. 3D.

### 4) Synthesis of Compound (4-1)

Method A: 0.994 g (0.557 mmol) of Compound (3-1) was dissolved in a MeOH/THF mixture (1:1, 50 mL), and a solution prepared by dissolving 0.669 g (16.72 mmol) of NaOH in 5 mL of H₂O was added thereto. A mixture obtained thereby was stirred at 25° C. for 12 hours and neutralized using an aqueous HCl solution (2N) to a pH of 1. A precipitate obtained by removing the solvent under reduced pressure was filtered and washed with water, and then dried at 85° C. 0.928 g (yield: 97.4%) of a product was obtained as a white solid.

Method B: 1.217 g (0.6 mmol) of Compound (3-2) was dissolved in a MeOH/DCM mixture (5:1, 60 mL), and a solution prepared by dissolving 1.44 g (36 mmol) of NaOH in 10 mL of H₂O was added thereto. A mixture obtained thereby was refluxed for 72 hours and neutralized using a HCl aqueous solution (2N) to a pH of 1. A precipitate obtained by removing the solvent under reduced pressure was filtered and washed with water, and then dried at 85° C. 0.944 g (yield: 92.6%) of a product was obtained as a white solid.

¹H-NMR data of obtained Compound (4-1) are shown in FIG. 3E.

### (3) Synthesis of Compound (6-1)

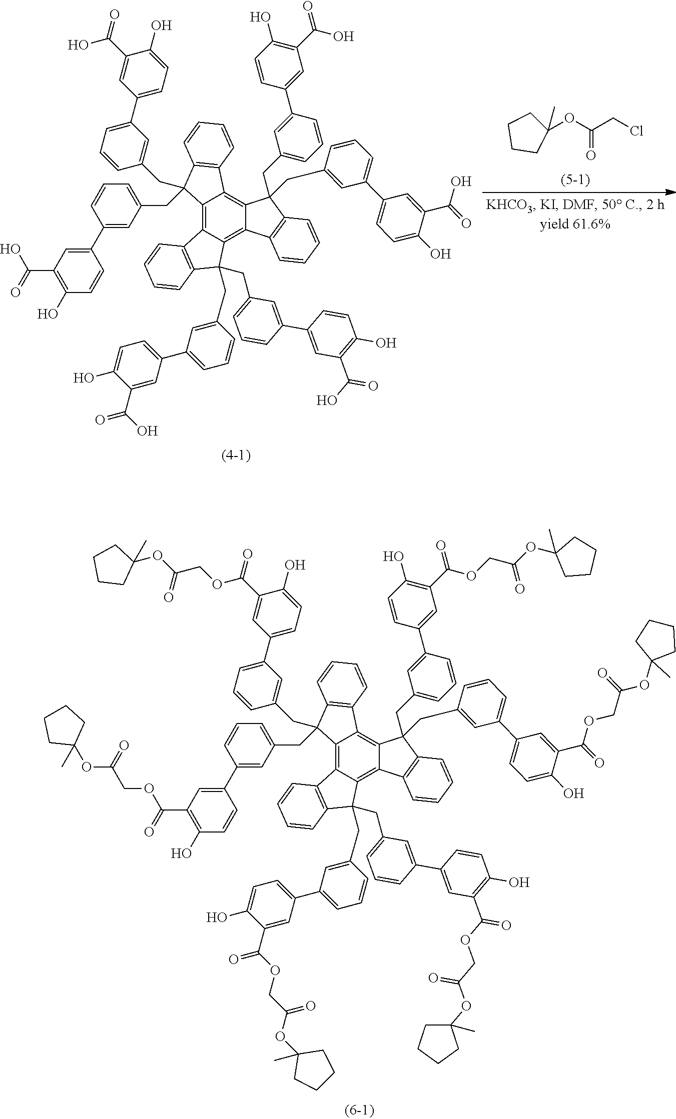

0.49 g (0.288 mmol) of Compound (4-1) was dissolved in 10 mL of DMF, and 0.346 g (3.46 mmol) of KHCO₃ and 48 mg (0.288 mmol) of Kl were added thereto while stirring. A mixture obtained thereby was stirred at 40° C. for 30 minute and 0.611 g (3.46 mmol) of Compound (5-1) was added thereto. A reaction mixture obtained thereby was further stirred at 50° C. for 2 hours. Upon completion of reaction, the mixture was diluted with EtOAc (0.2 L) and washed twice with 0.2 L of a NH₄Cl aqueous solution, and then dried in the presence of Na₂SO₄. After removing the solvent by drying under reduced pressure, a brown residue was separated by flash chromatography on a SiO₂ pad (7 cm) sequentially with a DCM/hexane mixture (1:1, 0.4 L) and a DCM/EtOAc mixture (100:3, 0.4 L). A solid obtained by evaporating the solvent under reduced pressure was dried in a vacuum oven at 40° C. 0.6 g (yield:61.6%) of Compound (6-1) was obtained as a white solid product, and Rt=0.76 (eluent: DCM:EtOAc=100:3, TLC silica gel 60 F₂₅₄). ¹H-NMR data of the obtained Compound (6-1) are shown in FIG. 3F.

### Synthesis Example 2

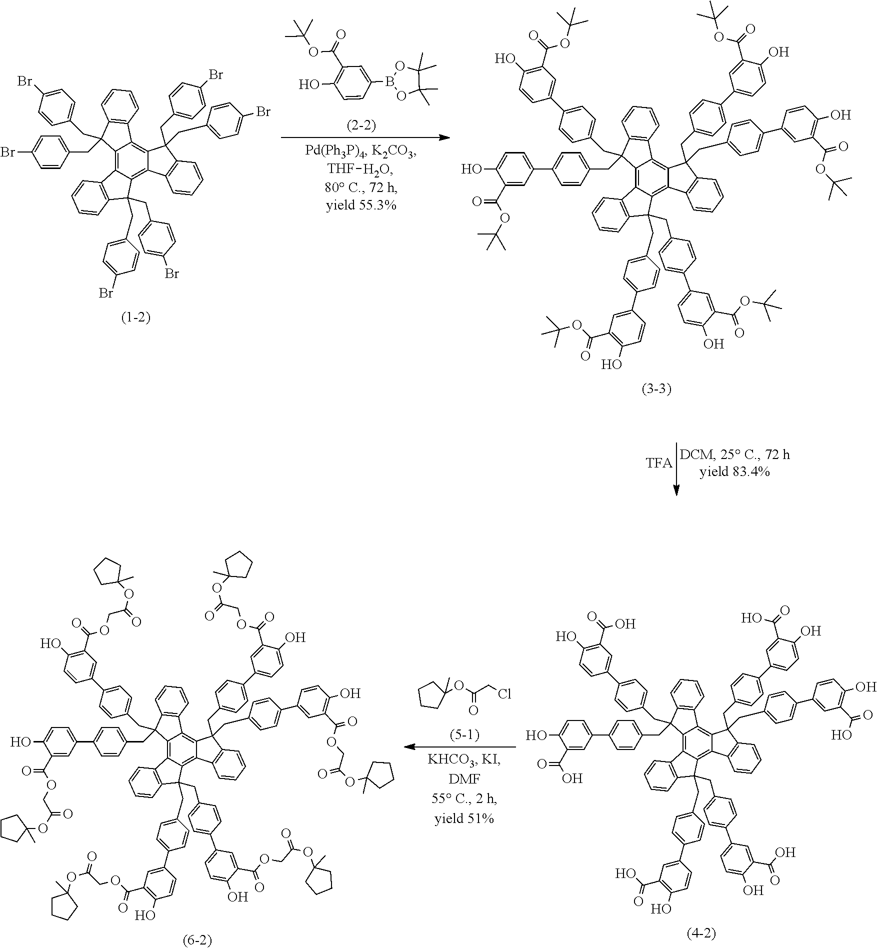

### 1) Synthesis of Compound (1-2)

Referring to European Journal of Organic Chemistry (2005), (19), 4127-4140), Compound (1-2) was synthesized.

### 2) Synthesis of Compound (3-3)

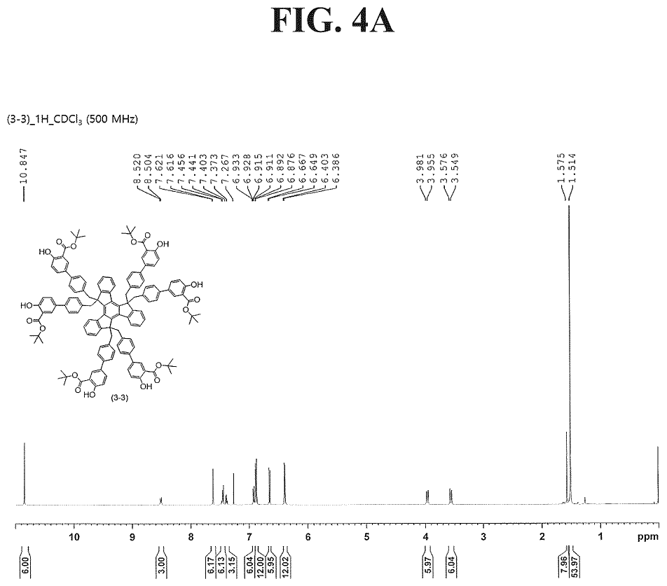

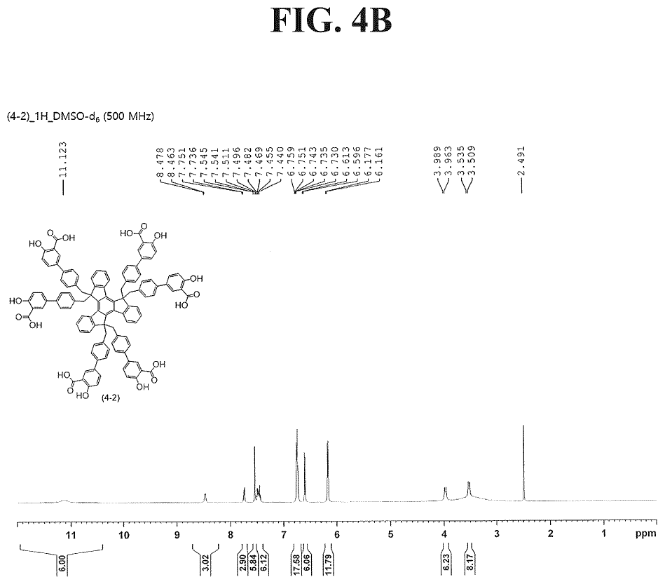

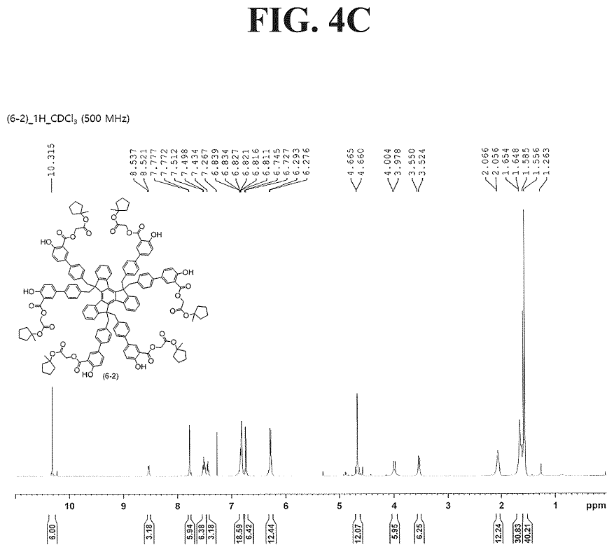

1.36 g (1 mmol) of Compound (1-2), 2.39 g (7.5 mmol) of Compound (2-2), 173 mg (0.15 mmol) of Pd(PPh₃)₄ and 3.73 g (27 mmol) of K₂CO₃ were added to a 100 mL-round bottom flask and 80 mL of a THF/H₂O mixture (3:1) was added thereto. A mixture obtained thereby was degassed and heated to 80° C., and then stirred in a nitrogen atmosphere for 24 hours. Upon completion of reaction, the mixture was diluted with EtOAc (0.2 L) and washed twice with 0.2 L of a NH₄Cl aqueous solution, and then dried in the presence of Na₂SO₄. After removing the solvent by drying under reduced pressure, a residue was separated by chromatography on SiO₂ with DCM/hexane (gradient from 50:50 to 100:0) to obtain fragments with an R_{f} of 0.50 (eluent: DCM, TLC silica gel 60 F₂₅₄). The materials were reprecipitated using DCM/MeOH, filtered, and dried under reduced pressure at 40° C. 1.12 g (yield: 55.3%) of Compound (3-3) was obtained as a light green solid. ¹H-NMR data of obtained Compound (3-3) are shown in FIG. 4A.

### 3) Synthesis of Compound (4-2)

1.357 g (0.668 mmol) of Compound (3-3) was dissolved in 40 mL of DCM and 9.14 g (80.2 mmol) of TFA was added thereto while stirring. A mixture obtained thereby was stirred at 25° C. for 24 hours. A white precipitate was formed. The mixture was diluted using DCM to 70 mL, and a solid material was filtered and washed with DCM, and then dried at 85° C. 0.944 g (83.4%) of a product was obtained as a white solid. ¹H-NMR data of obtained Compound (4-2) are shown in FIG. 4B.

### 4) Synthesis of Compound (6-2)

0.8 g (0.47 mmol) of Compound (4-2) was dissolved in 20 ml of DMF, and 0.565 g (5.65 mmol) of KHCO₃ and 78 mg (0.47 mmol) of Kl were added thereto while stirring. A mixture obtained thereby was stirred at 55° C. for 1 hour and 1 g (5.65 mmol) of Compound (5-1) was added thereto. The reaction mixture obtained thereby was further stirred at 55° C. for 2 hours. Upon completion of reaction, the mixture was diluted with EtOAc (0.2 L) and washed twice with 0.2 L of a NH₄Cl aqueous solution, and then dried in the presence of Na₂SO₄. After removing the solvent under reduced pressure, a brown residue was separated by flash chromatography on a SiO₂ pad (7 cm) sequentially with DCM (0.4 L) and a DCM/EtOAc mixture (100:3, 0.6 L). The solvent was evaporated under reduced pressure, and a residue was reprecipitated using DCM/MeOH. A solid was filtered, washed with MeOH, and dried in a vacuum oven at 40° C. 0.61 g (yield: 51%) of Compound (6-2) was obtained as a white solid product, and Rt=0.50 (eluent: DCM:EtOAc=100:2, TLC silica gel 60 F₂₅₄). ¹H-NMR data of obtained Compound (6-2) are shown in FIG. 4C.

### Synthesis Example 3

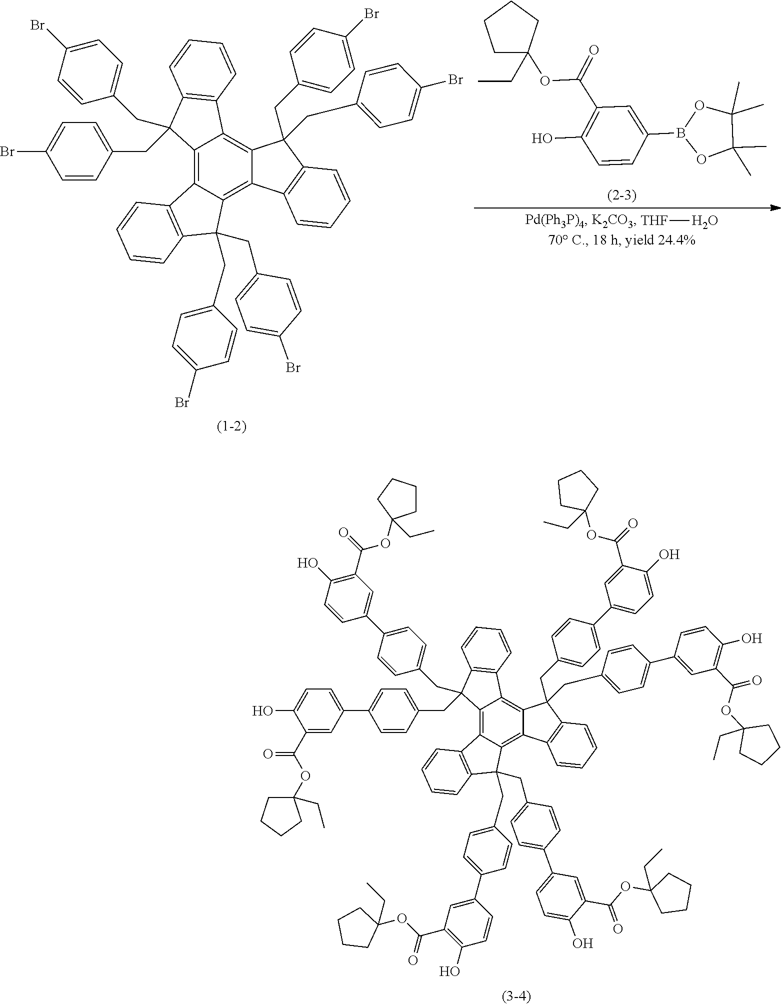

### 1) Synthesis of Compound (3-4)

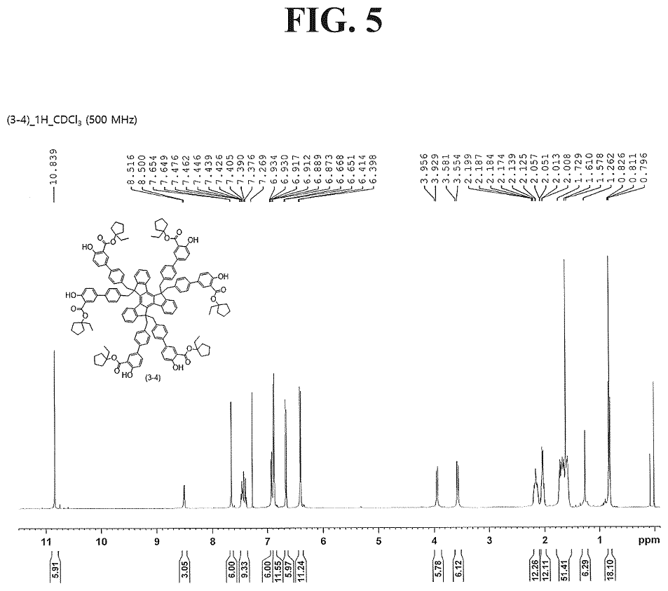

0.755 g (0.557 mmol) of Compound (1-2), 1.5 g (4.17 mmol) of Compound (2-3), 96 mg (0.083 mmol) of Pd(PPh₃)₄, and 2.08 g (15 mmol) of K₂CO₃ were added to a 100 mL-round bottom flask and 80 mL of a THF/H₂O mixture (3:1) was added thereto. A mixture obtained thereby was degassed and heated to 70° C., and then stirred in a nitrogen atmosphere for 16 hours. Upon completion of reaction, the mixture was diluted with EtOAc (0.2 L) and washed twice with 0.2 L of a NH₄Cl aqueous solution, and then dried in the presence of Na₂SO₄. After removing the solvent by drying under reduced pressure, a residue was separated by chromatography on SiO₂ with DCM/hexane (gradient from 12:88 to 100:0) to obtain fragments with an R_{f} of 0.39 (eluent: DCM/Hexane=3/1, TLC silica gel 60 F₂₅₄). After evaporating the solvent under reduced pressure, a residue was reprecipitated using DCM/MeOH, filtered, and dried under reduced pressure at 40° C. 0.31 g (yield: 24.4%) of a product was obtained as a white solid. ¹H-NMR data of obtained Compound (3-4) are shown in FIG. 5.

### Synthesis Example 4

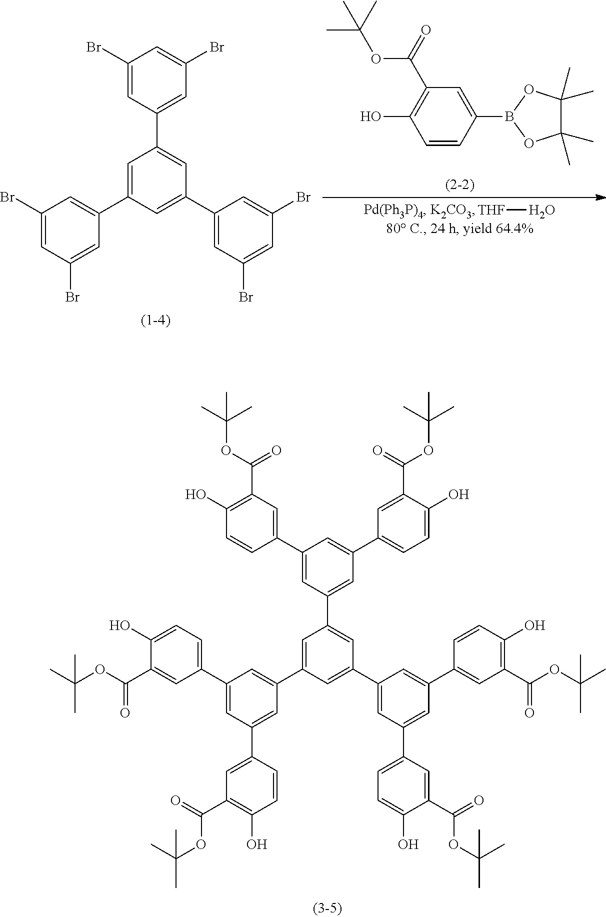

### 1) Synthesis of Compound (1-4)

Referring to ARKIVOC 2013 (iii) 49-60, Compound (1-4) (yield: 48%) was synthesized from 3,5-dibromoacetophenone and p-TsOH·H₂O.

### 2) Synthesis of Compound (3-5)

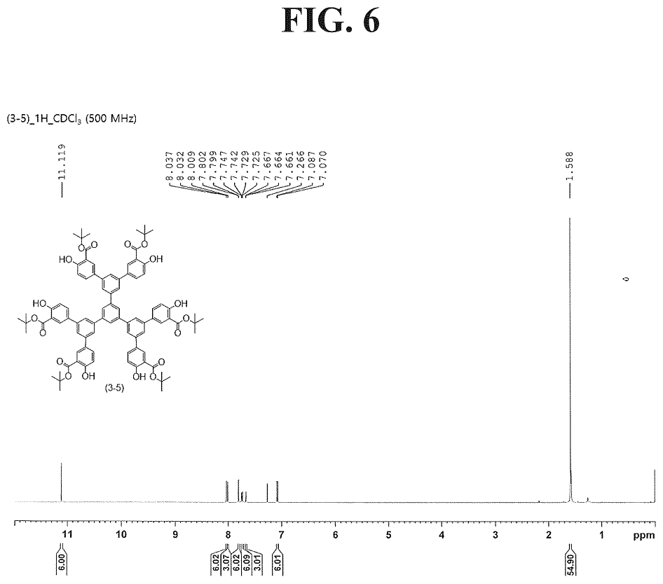

0.39 g (0.5 mmol) of Compound (1-4), 1.2 g (3.75 mmol) of Compound (2-2), 87 mg (0.075 mmol) of Pd(PPh₃)₄ and 1.87 g (13.5 mmol) of K₂CO₃ were added to a 100 mL-round bottom flask and 80 mL of a THF/H₂O mixture (4:1) was added thereto. A mixture obtained thereby was degassed and heated to 80° C., and then stirred in a nitrogen atmosphere for 24 hours. Upon completion of reaction, the mixture was diluted with EtOAc (0.2 L) and washed twice with 0.2 L of a NH₄Cl aqueous solution, and then dried in the presence of Na₂SO₄. After removing the solvent by drying under reduced pressure, a residue was separated by chromatography on SiO₂ with DCM/hexane (gradient from 12:88 to 100:0) to obtain fragments with an R_{f} of 0.39 (eluent: DCM/Hexane=3/1, TLC silica gel 60 F₂₅₄). The residue was reprecipitated using DCM/MeOH, filtered, and dried under reduced pressure at 40° C. 0.47 g (yield: 64.4%) of Compound (3-5) was obtained as a white solid product. ¹H-NMR data of obtained Compound (3-5) are shown in FIG. 6.

### Synthesis Example 5

### (1) Synthesis of Compound (4-3)

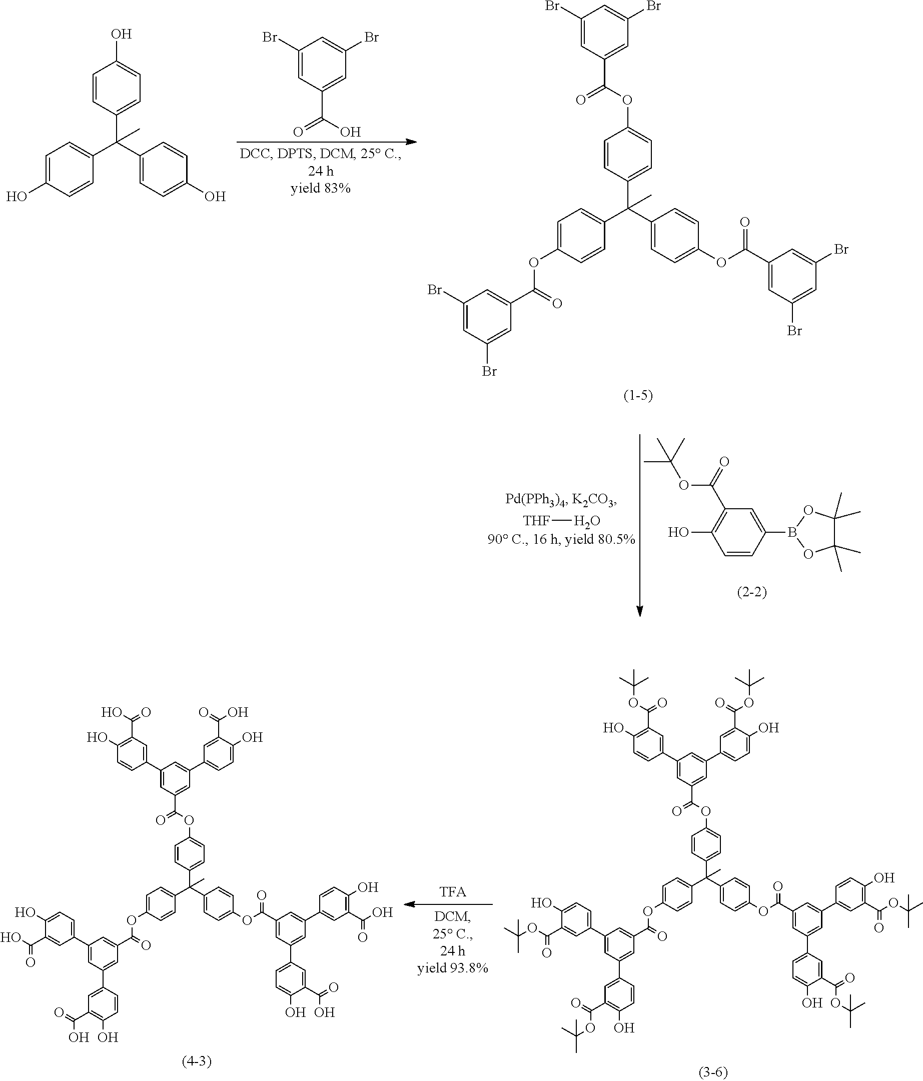

### 1) Synthesis of Compound (1-5)

0.919 g (3 mmol) of 1,1,1-tris(4-hydroxyphenyl)ethane, 2.77 g (9.9 mmol) of 3,5-dibromobenzoic acid, and 3.24 g (11 mmol) of DPTS were added to a 100 mL-round bottom flask and 50 mL of DCM was added thereto. 2.27 g (11 mmol) of DCC was added thereto while stirring and the mixture was stirred at 25° C. for 24 hours. Upon completion of reaction, DCM was evaporated under reduced pressure and a residual solid was suspended in THF (0.2 L) and filtered, and then DPTS, which was not dissolved, was removed. THF was removed under reduced pressure. A residue was suspended in boiling MeOH, cooled to 25° C., and filtered. A solid was washed with MeOH and dried at 85° C. 2.72 g (yield: 83%) of Compound (1-5) was obtained as a white solid product. R_{f}=0.63 (eluent: DCM/Hexane=1/1, TLC silica gel 60 F₂₅₄).

### 2) Synthesis of Compound (3-6)

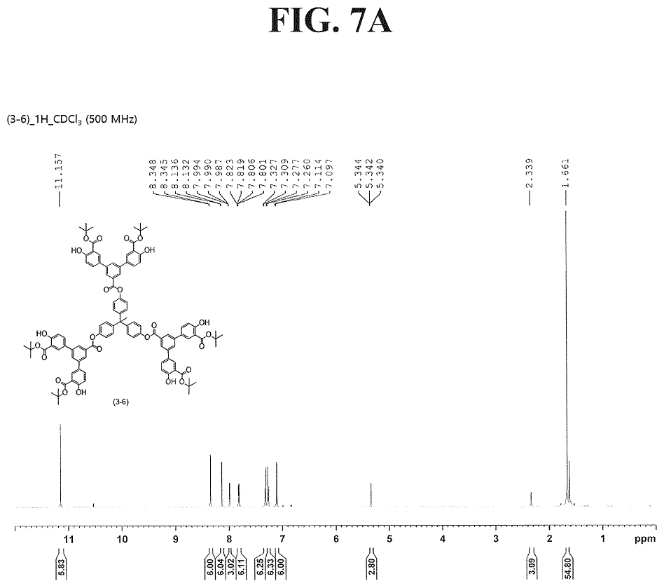

1.55 g (1.42 mmol) of Compound (1-5), 3.4 g (10.64 mmol) of Compound (2-2), 246 mg (0.213 mmol) of Pd(PPh₃)₄ and 5.3 g (38.32 mmol) of K₂CO₃ were added to a 100 mL-round bottom flask and 90 mL of a THF/H₂O mixture (5:1) was added thereto. A mixture obtained thereby was degassed and heated to 80° C., and then stirred in a nitrogen atmosphere for 24 hours. Upon completion of reaction, the mixture was diluted with EtOAc (0.2 L) and washed twice with 0.2 L of a NH₄Cl aqueous solution, and then dried in the presence of Na₂SO₄. After removing the solvent by drying under reduced pressure, a residue was separated by chromatography on SiO₂ with DCM/hexane (gradient from 50:50 to 100:0) to obtain fragments with an R_{f} of 0.63 (eluent: DCM, TLC silica gel 60 F₂₅₄). The residue was reprecipitated using DCM/MeOH, filtered, and dried under reduced pressure at 40° C. 1.896 g (yield: 89.6%) of Compound (3-6) was obtained as a white solid product. ¹H-NMR data of obtained Compound (3-6) are shown in FIG. 7A.

### 3) Synthesis of Compound (4-3)

1.896 g (1.07 mmol) of Compound (3-6) was dissolved in 50 mL of DCM and 18.3 g (0.16 mol) of TFA was added thereto while stirring. A mixture obtained thereby was stirred at 25° C. for 24 hours. A white precipitate was formed. The mixture was diluted using DCM to 150 mL, and a solid material was filtered and washed with DCM, and then dried at 85° C. 1.44 g (yield: 93.8%) of a product was obtained as a white solid.

### (2) Synthesis of Compound (6-3)

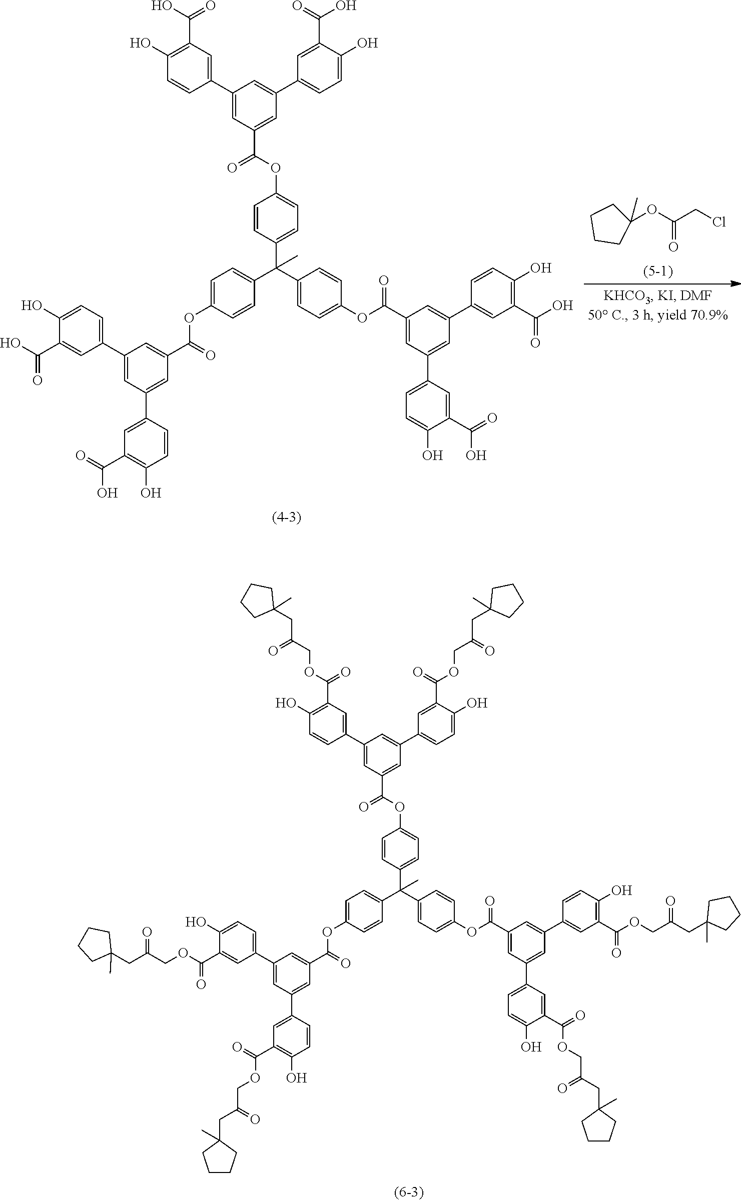

0.8 g (0.557 mmol) of Compound (4-3) was dissolved in 12 mL of DMF, and 0.67 g (6.69 mmol) of KHCO₃ and 93 mg (0.557 mmol) of Kl were added thereto while stirring. A mixture obtained thereby was stirred at 40° C. for 1 hour and 1.19 g (6.69 mmol) of Compound (5-1) was added thereto. The reaction mixture obtained thereby was further stirred at 55° C. for 2 hours. Upon completion of reaction, the mixture was diluted with EtOAc (0.2 L) and washed twice with 0.2 L of a NH₄Cl aqueous solution, and then dried in the presence of Na₂SO₄. After removing the solvent under reduced pressure, a brown residue was separated by flash chromatography on a SiO₂ pad (7 cm) sequentially using a DCM/hexane mixture (1:1, 0.5 L) and a DCM/EtOAc mixture (100:3, 0.6 L). The solvent was evaporated under reduced pressure, and a residual solid was dried in a vacuum oven at 40° C. 0.9 g (yield: 70.9%) of Compound (6-1) was obtained as a dark orange solid, and Rt=0.81 (eluent: DCM:EtOAc=100:5, TLC silica gel 60 F₂₅₄). ¹H-NMR data of obtained Compound (6-3) are shown in FIG. 7B.

### Synthesis Example 6

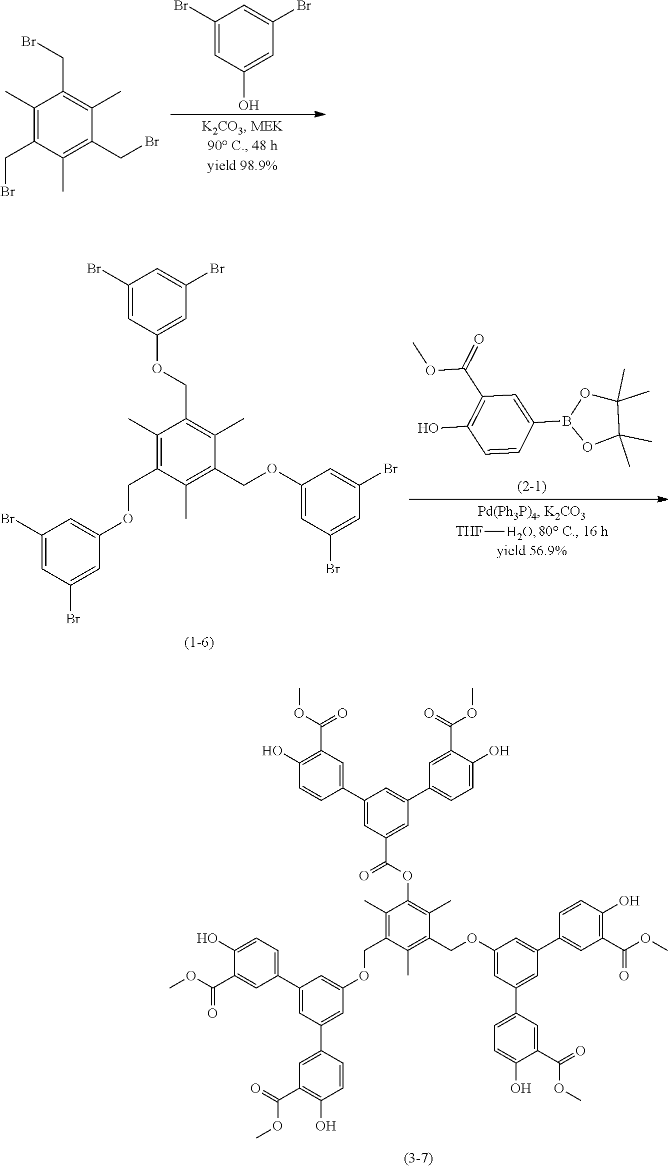

### 1) Synthesis of Compound (1-6)

2.4 g (6.01 mmol) of 1,3,5-tris(bromomethyl)-2,4,6-trimethylbenzene, 5 g (19.85 mmol) of 3,5-dibromophenol, and 4.98 g (36.06 mmol) of K₂CO₃ were added to a 100 mL-round bottom flask, and MEK was added thereto. A mixture obtained thereby was stirred at 90° C. for 24 hours. Upon completion of reaction, the mixture was poured into 0.3 L of water, and a solid was filtered and crystallized with DCM/MeOH, and then dried at 60° C. 5.48 g (yield: 98.9%) of Compound (1-6) was obtained as a white solid.

### 2) Synthesis of Compound (3-7)

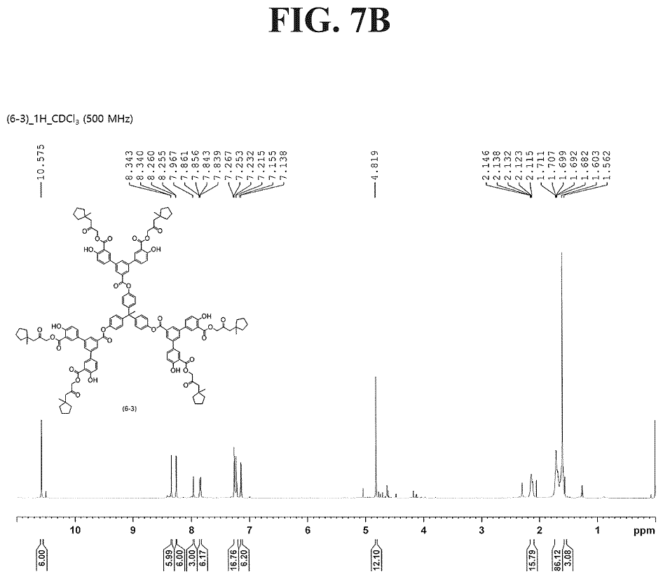

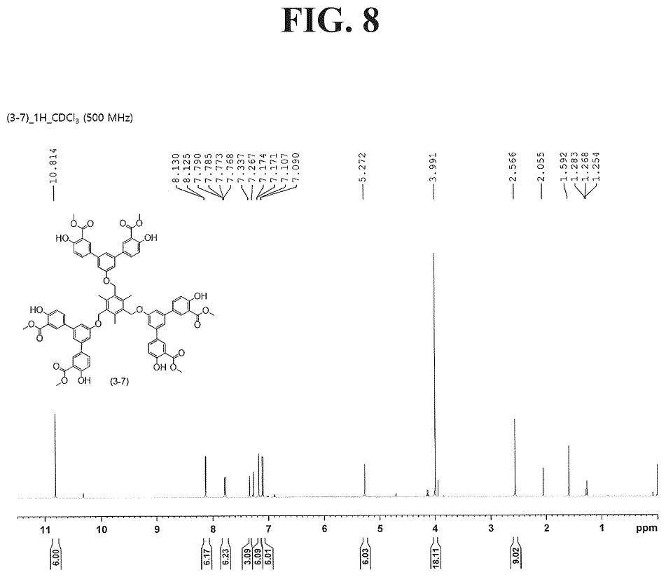

0.5 g (0.548 mmol) of Compound (1-6), 1.37 g (4.94 mmol) of Compound (2-1), 95 mg (0.08 mmol) of Pd(PPh₃)₄, and 2.04 g (14.8 mmol) of K₂CO₃ were added to a 100 mL-round bottom flask and 60 mL of a THF/H₂O mixture (5:1) was added thereto. A mixture obtained thereby was degassed and heated to 80° C., and then stirred in a nitrogen atmosphere for 24 hours. Upon completion of reaction, the mixture was diluted with EtOAc (0.2 L) and washed twice with 0.2 L of a NH₄Cl aqueous solution, and then dried in the presence of Na₂SO₄. After removing the solvent by drying under reduced pressure, a residue was separated by chromatography on SiO₂ with DCM/EtOAc (gradient from 100:0 to 95:5) to obtain fragments with an R_{f} of 0.62 (eluent: DCM:EtOAc=100:3, TLC silica gel 60 F₂₅₄). The residue was reprecipitated using DCM/MeOH, filtered, and dried under reduced pressure at 60° C. 0.434 g (yield: 56.9%) of Compound (3-7) was obtained as a white solid. ¹H-NMR data of obtained Compound (3-7) are shown in FIG. 8.

### Synthesis Example 7

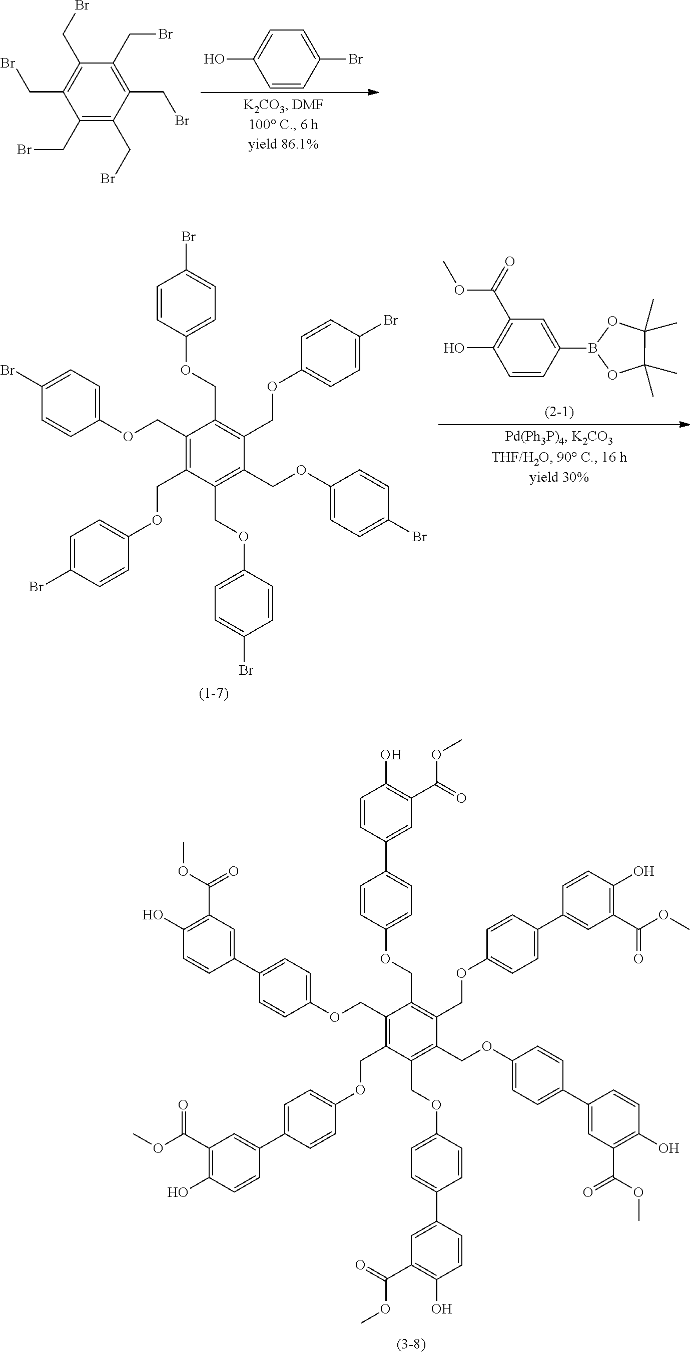

### 1) Synthesis of Compound (1-7)

1.27 g (2 mmol) of hexakis(bromomethyl)benzene, 2.42 g (14 mmol) of 4-bromophenol, and 1.94 g (14 mmol) of K₂CO₃ were added to a 100 mL-round bottom flask and 40 mL of DMF was added thereto. A mixture obtained thereby was stirred at 100° C. for 6 hours. Upon completion of reaction, the mixture was poured into 0.2 L of water, and a solid was filtered, washed with water, and treated with high-temperature MeOH (2×0.15 L) and high-temperature EtOAc (0.15 L), following by filtering and drying at 85° C. 2.05 g (yield: 86.1%) of Compound (1-7) was obtained as white powder.

### 2) Synthesis of Compound (3-8)

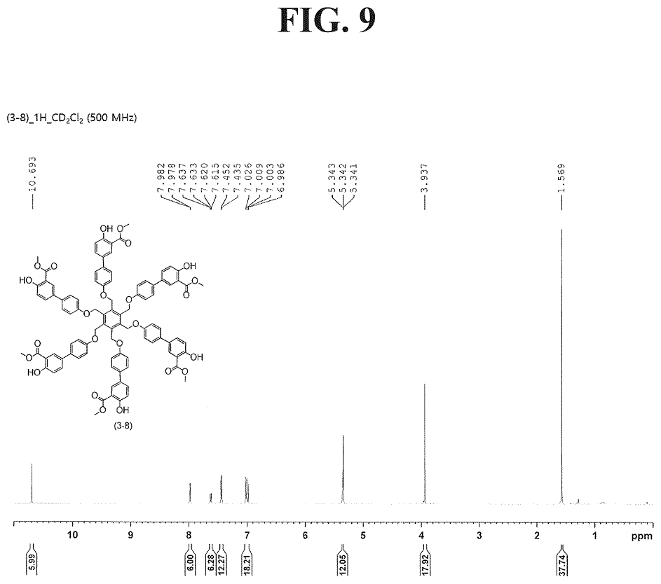

0.594 g (0.5 mmol) of Compound (1-7), 1.25 g (4.5 mmol) of Compound (2-1), 87 mg (0.075 mmol) of Pd(PPh₃)₄ and 1.87 g (13.5 mmol) of K₂CO₃ were added to a 100 mL-round bottom flask and 60 mL of a THF/H₂O mixture (5:1) was added thereto. A mixture obtained thereby was degassed and heated to 90° C., and then stirred in a nitrogen atmosphere for 24 hours. Upon completion of reaction, the mixture was diluted with DCM (0.3 L) and washed twice with 0.2 L of a NH₄Cl aqueous solution, and then dried in the presence of Na₂SO₄. After removing the solvent by drying under reduced pressure, a residue was separated by chromatography on SiO₂ with DCM/EtOAc (gradient from 100:0 to 95:5). The residue was reprecipitated using DCM/MeOH, filtered, and dried under reduced pressure at 60° C. 0.242 g (yield: 30%) of Compound (3-8) was obtained as a white solid. ¹H-NMR data of obtained Compound (3-8) are shown in FIG. 9.

### Synthesis Example 8

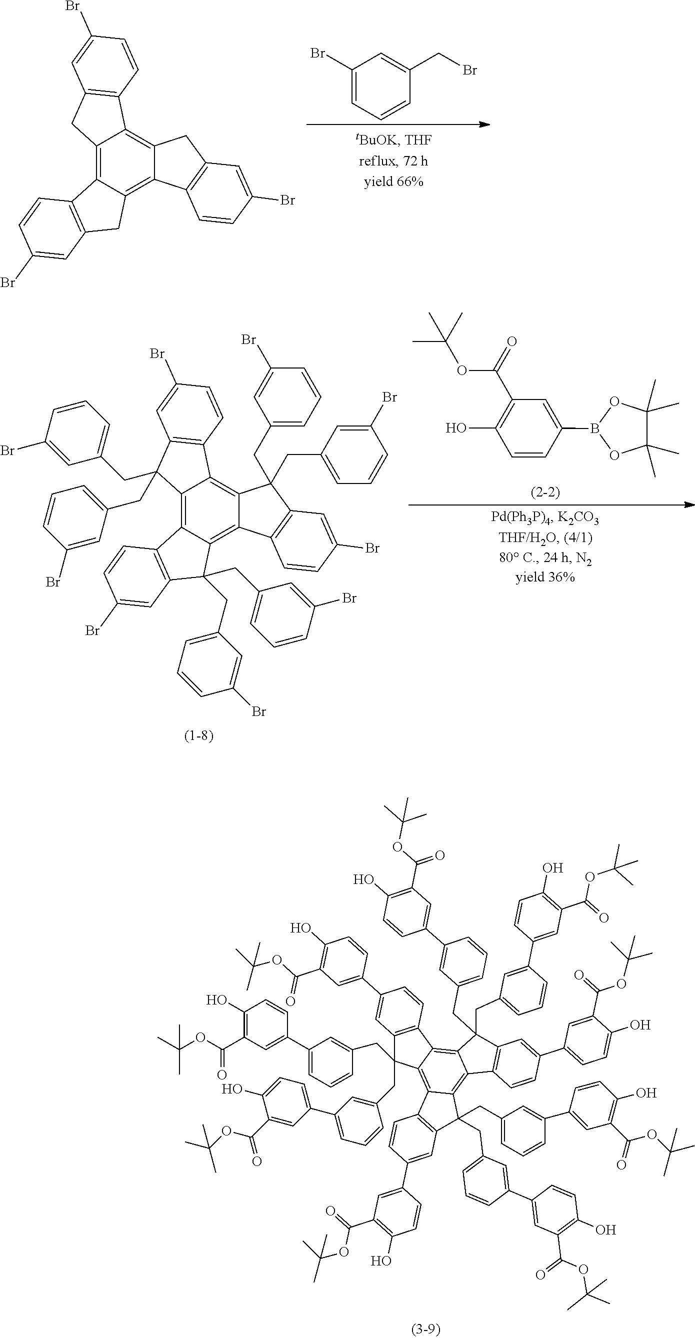

### 1) Synthesis of Compound (1-8)

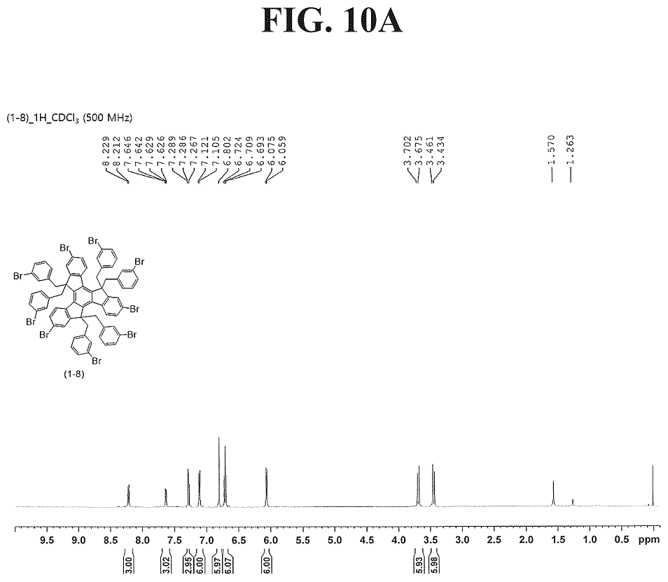

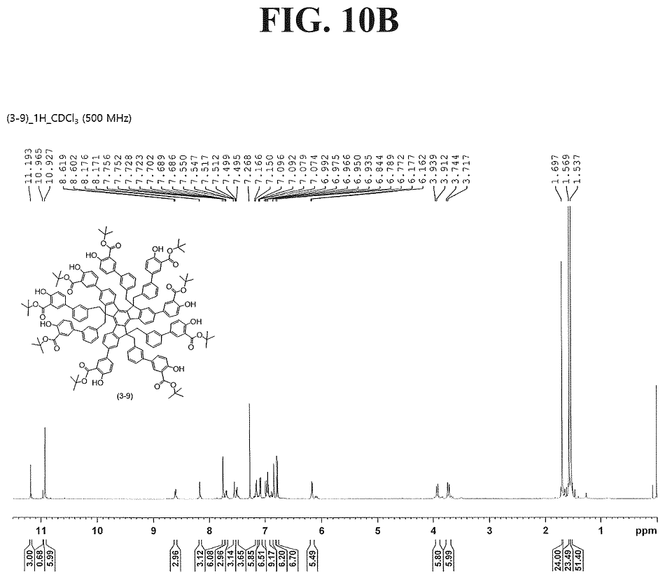

2.26 g (3.9 mmol) of 2,7,12-tribromotruxene and 7.8 g (31 mmol) of 3-bromobenzyl bromide were suspended in THF (80 mL). 3.5 g (31 mmol) of potassium tert-butoxide was gradually added thereto while quickly stirring. A mixture obtained thereby was refluxed for 72 hours. Upon completion of reaction, the mixture was poured into water. A dark green, viscous material was separated and washed with methanol. A crude product was separated by chromatography on SiO₂ with DCM/hexane (gradient from 1:9 to 2:3 to obtain fragment with R_{f}=0.50 (eluent: DCM/Hex=1/1, TLC silica gel 60 F₂₅₄). A material obtained by chromatography was crystallized using DCM/MeCN and dried at 85° C. 4.1 g (yield: 66%) of Compound (1-8) was obtained as a light green solid. ¹H-NMR data of obtained Compound (1-8) are shown in FIG. 10A.

### 2) Synthesis of Compound (3-9)

0.398 g (0.25 mmol) of Compound (1-8), 0.958 g (3 mmol) of Compound (2-2), 65 mg (0.056 mmol) of Pd(PPh₃)₄ and 1.25 g (9 mmol) of K₂CO₃ were added to a 100 mL-round bottom flask and 60 mL of a THF/H₂O mixture (5:1) was added thereto. A mixture obtained thereby was degassed and heated to 80° C., and then stirred in a nitrogen atmosphere for 24 hours. Upon completion of reaction, the mixture was diluted with EtOAc (0.2 L) and washed twice with 0.2 L of a NH₄Cl aqueous solution, and then dried in the presence of Na₂SO₄. After removing the solvent by drying under reduced pressure, a residue was separated by chromatography on SiO₂ with DCM/hexane (gradient from 50:50 to 100:0) to obtain fragments with an R_{f} of 0.67 (eluent: DCM, TLC silica gel 60 F₂₅₄). The residue was reprecipitated using DCM/MeOH, filtered, and dried under reduced pressure at 40° C. 0.235 g (yield: 36%) of Compound (3-9) was obtained as a white solid. ¹H-NMR data of obtained Compound (3-9) are shown in FIG. 10B.

### Synthesis Example 9

### (1) Synthesis of Compound (3-10)

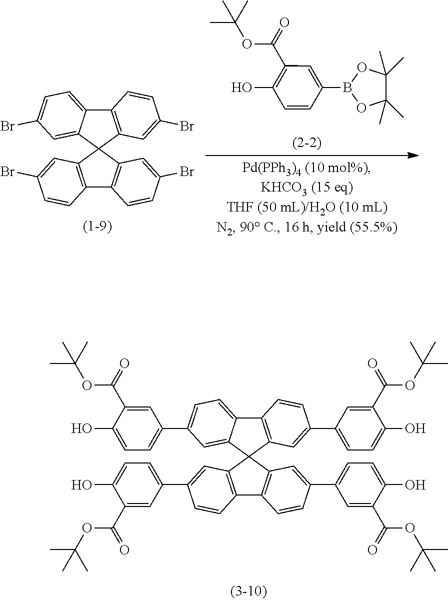 [SMILES: Brc1ccc2c(c1)C1(c3cc(Br)ccc3-2)c2cc(Br)ccc2-c2ccc(Br)cc21 ; CC(C)(C)OC(=O)c1cc(B2OC(C)(C)C(C)(C)O2)ccc1O ; CC(C)(C)OC(=O)c1cc(-c2ccc3c(c2)C2(c4cc(-c5ccc(O)c(C(=O)OC(C)(C)C)c5)ccc4-3)c3cc(-c4ccc(O)c(C(=O)OC(C)(C)C)c4)ccc3-c3ccc(-c4ccc(O)c(C(=O)OC(C)(C)C)c4)cc32)ccc1O]

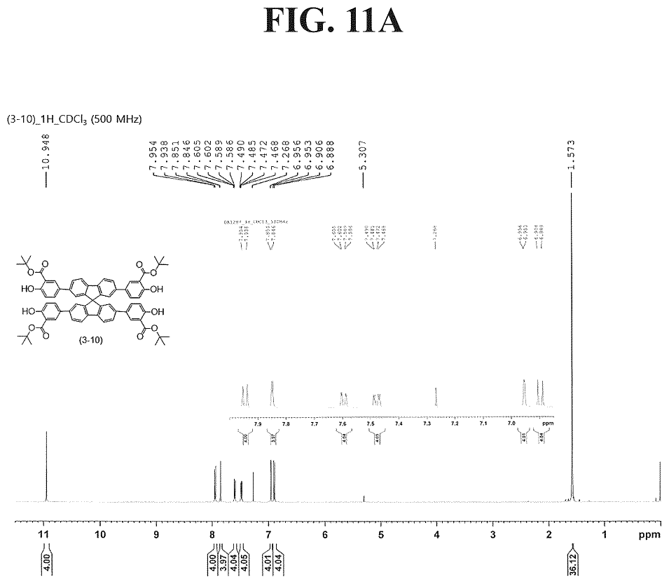

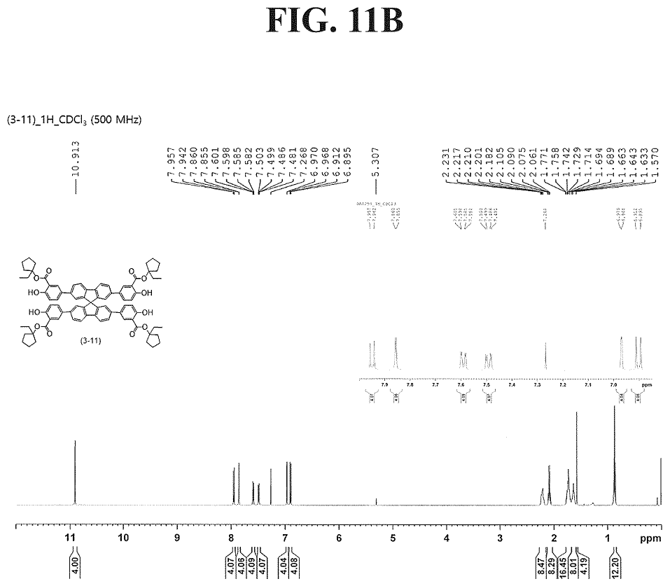

0.38 g (0.6 mmol) of Compound (1-9), 0.96 g (3 mmol) of Compound (2-2), 69 mg (0.06 mmol) of Pd(PPh₃)₄ and 0.9 g (9 mmol) of KHCO₃ were added to a 100 mL-round bottom flask and 60 mL of a THF/H₂O mixture (5:1) was added thereto. A mixture obtained thereby was degassed and heated to 90° C., and then stirred in a nitrogen atmosphere for 16 hours. Upon completion of reaction, the mixture was diluted with EtOAc (0.2 L) and washed twice with 0.2 L of a NH₄Cl aqueous solution, and then dried in the presence of Na₂SO₄. After removing the solvent by drying under reduced pressure, a residue was separated by chromatography on SiO₂ with DCM/hexane (gradient from 50:50 to 100:0) to obtain fragments with an R_{f} of 0.47 (eluent: DCM:hexane=4:1, TLC silica gel 60 F₂₅₄). The solvent was evaporated under reduced pressure, and a residual solid was dried under reduced pressure at 40° C. 0.36 g (yield: 55.5%) of Compound (3-10) was obtained as a white amorphous solid. ¹H-NMR data of obtained Compound (3-10) are shown in FIG. 11A.

### (2) Synthesis of Compound (3-11)

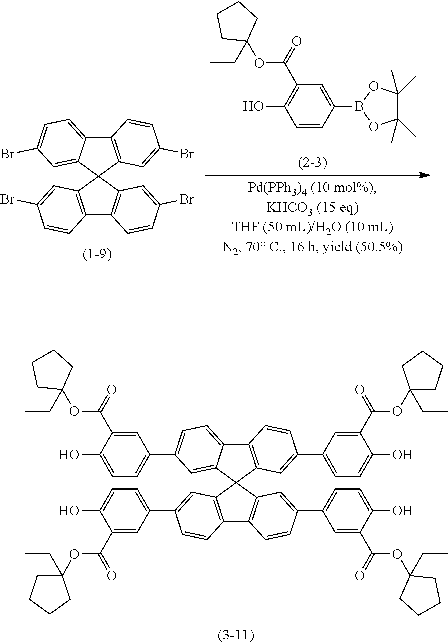 [SMILES: Brc1ccc2c(c1)C1(c3cc(Br)ccc3-2)c2cc(Br)ccc2-c2ccc(Br)cc21 ; CCC1(OC(=O)c2cc(B3OC(C)(C)C(C)(C)O3)ccc2O)CCCC1 ; CCC1(OC(=O)c2cc(-c3ccc4c(c3)C3(c5cc(-c6ccc(O)c(C(=O)OC7(CC)CCCC7)c6)ccc5-4)c4cc(-c5ccc(O)c(C(=O)OC6(CC)CCCC6)c5)ccc4-c4ccc(-c5ccc(O)c(C(=O)OC6(CC)CCCC6)c5)cc43)ccc2O)CCCC1]

0.584 g (0.94 mmol) of Compound (1-9), 1.66 g (6.62 mmol) of Compound (2-3), 107 mg (0.0924 mmol) of Pd(PPh₃)₄ and 1.39 g (13.86 mmol) of KHCO₃ were added to a 100 mL-round bottom flask and 60 ml of a THF/H₂O mixture (5:1) was added thereto. A mixture obtained thereby was degassed and heated to 70° C., and then stirred in a nitrogen atmosphere for 18 hours. Upon completion of reaction, the mixture was diluted with EtOAc (0.2 L) and washed twice with 0.2 L of a NH₄Cl aqueous solution, and then dried in the presence of Na₂SO₄. After removing the solvent by drying under reduced pressure, a residue was separated by chromatography on SiO₂ with DCM/hexane (gradient from 50:50 to 80:20) to obtain fragments with an R_{f} of 0.66 (eluent: DCM:hexane=4:1, TLC silica gel 60 F₂₅₄). The solvent was evaporated under reduced pressure, and a residue was dried under reduced pressure at 40° C. 0.58 g (yield: 50.5%) of Compound (3-11) was obtained as a white amorphous solid. ¹H-NMR data of obtained Compound (3-11) are shown in FIG. 11B.

### Synthesis Example 10

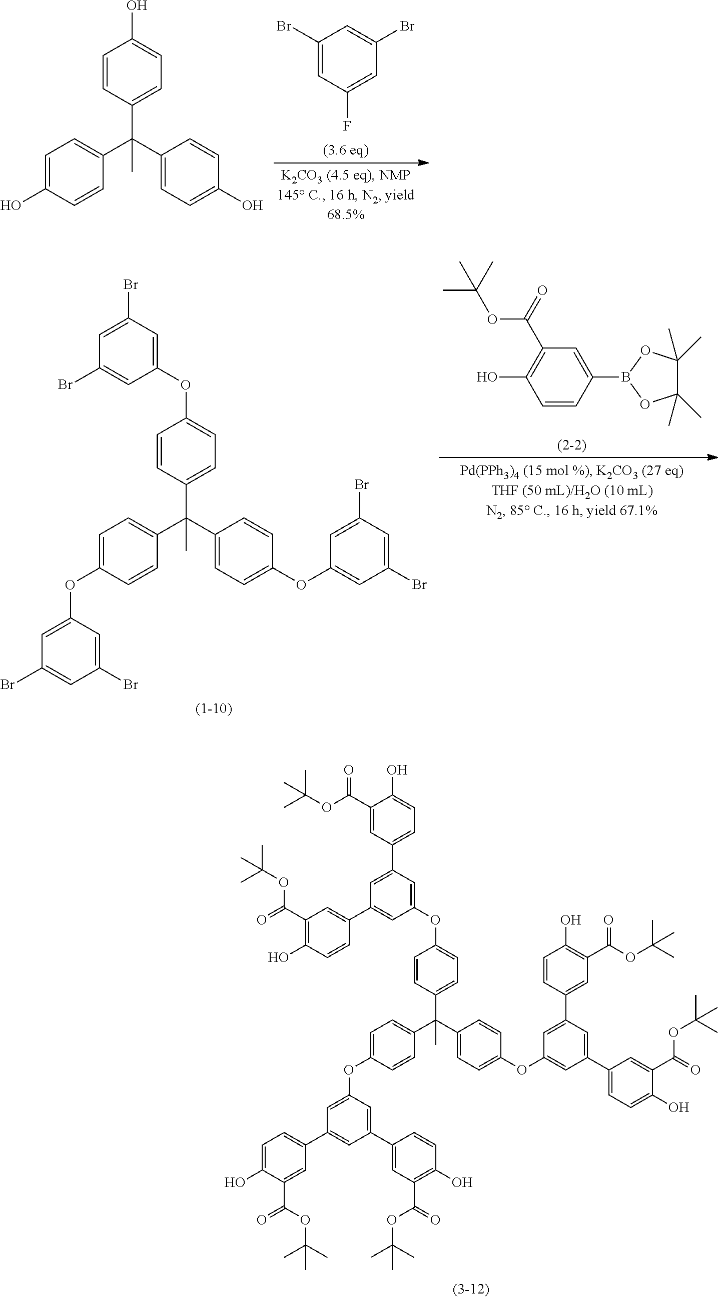 [SMILES: CC(c1ccc(O)cc1)(c1ccc(O)cc1)c1ccc(O)cc1 ; Fc1cc(Br)cc(Br)c1 ; CC(c1ccc(Oc2cc(Br)cc(Br)c2)cc1)(c1ccc(Oc2cc(Br)cc(Br)c2)cc1)c1ccc(Oc2cc(Br)cc(Br)c2)cc1 ; CC(C)(C)OC(=O)c1cc(B2OC(C)(C)C(C)(C)O2)ccc1O ; CC(C)(C)OC(=O)c1cc(-c2cc(Oc3ccc(C(C)(c4ccc(Oc5cc(-c6ccc(O)c(C(=O)OC(C)(C)C)c6)cc(-c6ccc(O)c(C(=O)OC(C)(C)C)c6)c5)cc4)c4ccc(Oc5cc(-c6ccc(O)c(C(=O)OC(C)(C)C)c6)cc(-c6ccc(O)c(C(=O)OC(C)(C)C)c6)c5)cc4)cc3)cc(-c3ccc(O)c(C(=O)OC(C)(C)C)c3)c2)ccc1O]

### 1) Synthesis of Compound (1-10)

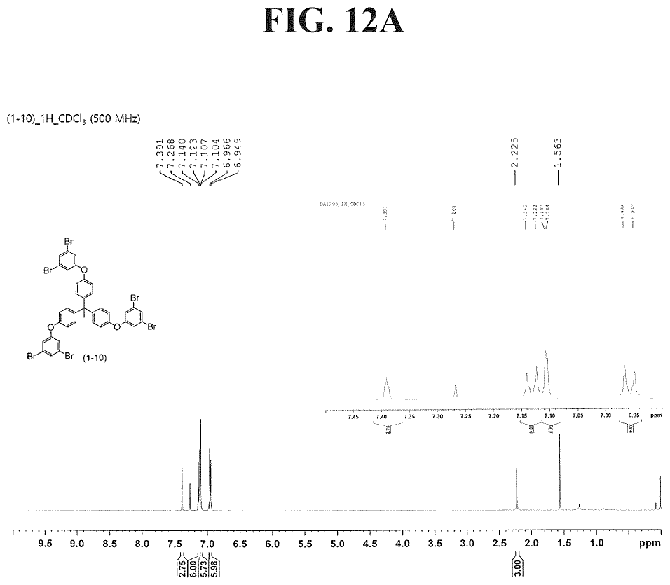

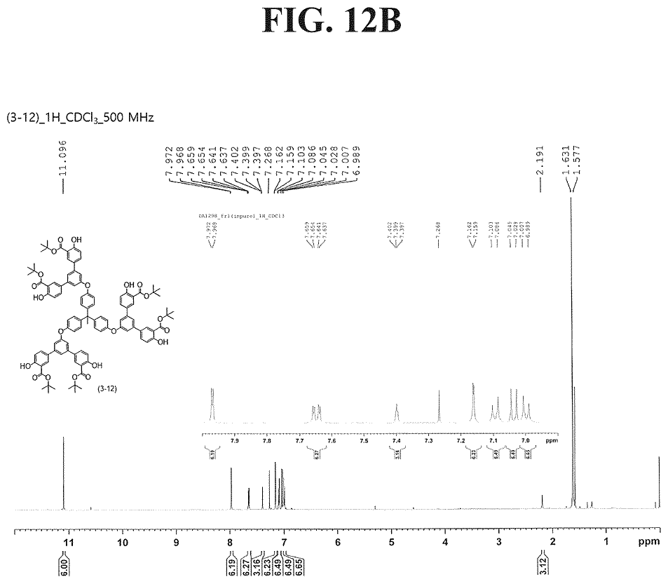

1.53 g (5 mmol) of 1,1,1-tris(4-hydroxyphenyl)ethane, 4.57 g (18 mmol) of 1,3-dibromo-5-fluorobenzene, 3.1 g (22.5 mmol) of K₂CO₃, and 50 mL of NMP were added to a 100 mL-round bottom flask. A mixture obtained thereby was heated to 145° C., and then stirred in a nitrogen atmosphere for 16 hours. Upon completion of reaction, the mixture was poured into water (0.4 L) and stirred for 1 hour. An amorphous brown residue was filtered, washed with water, dissolved in DCM (0.2 L), and dried in the presence of Na₂SO₄. After evaporating the solvent by drying under reduced pressure, a residue was separated by chromatography on SiO₂ with DCM/hexane (25:75) to obtain fragments with an R_{f} of 0.42 (eluent: DCM:hexane=1:3, TLC silica gel 60 F₂₅₄). The solvent was evaporated under reduced pressure, and a residue was dried under reduced pressure at 40° C. 3.45 g (yield: 68.5%) of Compound (1-10) was obtained as a white amorphous solid. ¹H-NMR data of obtained Compound (1-10) are shown in FIG. 12A.

### 2) Synthesis of Compound (3-12)

0.42 g (0.418 mmol) of Compound (1-10), 1.2 g (3.75 mmol) of Compound (2-2), 72 mg (0.063 mmol) of Pd(PPh₃)₄ and 1.56 g (11.28 mmol) of K₂CO₃ were added to a 100 mL-round bottom flask and 60 mL of a THF/H₂O mixture (5:1) was added thereto. A mixture obtained thereby was degassed and heated to 70° C., and then stirred in a nitrogen atmosphere for 18 hours. Upon completion of reaction, the mixture was diluted with EtOAc (0.2 L) and washed twice with 0.2 L of a NH₄Cl aqueous solution, and then dried in the presence of Na₂SO₄. After removing the solvent by drying under reduced pressure, a residue was separated by chromatography on SiO₂ with DCM/hexane (gradient from 50:50 to 100:0) to obtain fragments with an R_{f} of 0.51 (eluent: DCM:hexane=5:1, TLC silica gel 60 F₂₅₄). The solvent was evaporated under reduced pressure, and a residue was dried under reduced pressure at 40° C. 0.47 g (yield: 67.1%) of Compound (3-12) was obtained as a white amorphous solid. ¹H-NMR data of obtained Compound (3-12) are shown in FIG. 12B.

### Synthesis Example 11

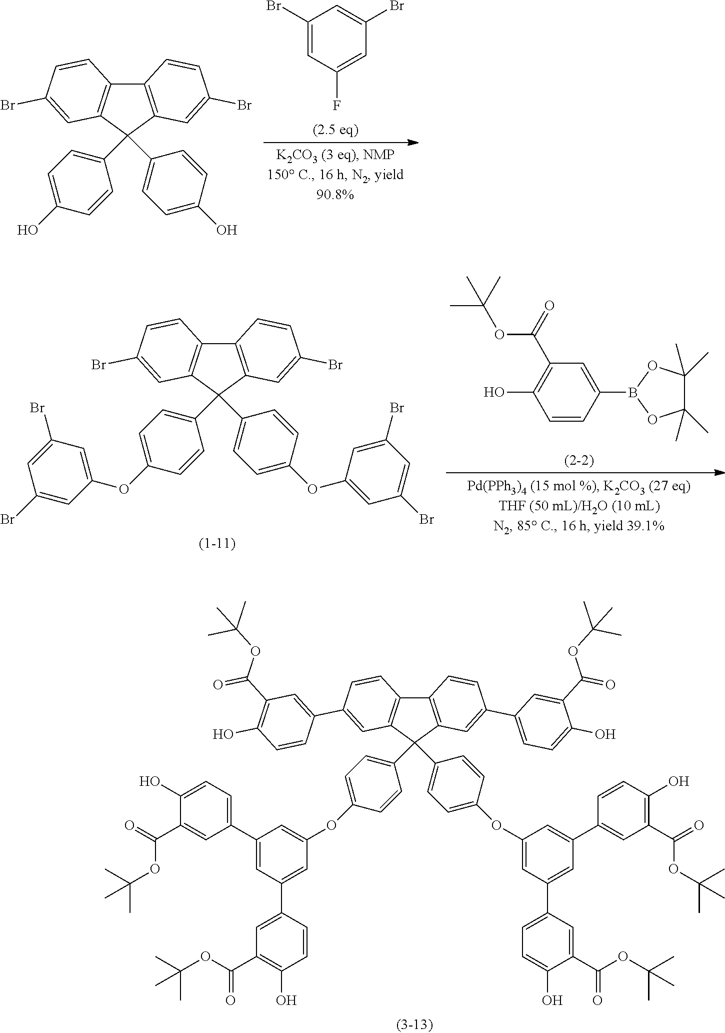 [SMILES: Oc1ccc(C2(c3ccc(O)cc3)c3cc(Br)ccc3-c3ccc(Br)cc32)cc1 ; Fc1cc(Br)cc(Br)c1 ; Brc1cc(Br)cc(Oc2ccc(C3(c4ccc(Oc5cc(Br)cc(Br)c5)cc4)c4cc(Br)ccc4-c4ccc(Br)cc43)cc2)c1 ; CC(C)(C)OC(=O)c1cc(B2OC(C)(C)C(C)(C)O2)ccc1O ; CC(C)(C)OC(=O)c1cc(-c2cc(Oc3ccc(C4(c5ccc(Oc6cc(-c7ccc(O)c(C(=O)OC(C)(C)C)c7)cc(-c7ccc(O)c(C(=O)OC(C)(C)C)c7)c6)cc5)c5cc(-c6ccc(O)c(C(=O)OC(C)(C)C)c6)ccc5-c5ccc(-c6ccc(O)c(C(=O)OC(C)(C)C)c6)cc54)cc3)cc(-c3ccc(O)c(C(=O)OC(C)(C)C)c3)c2)ccc1O]

### 1) Synthesis of Compound (1-11)

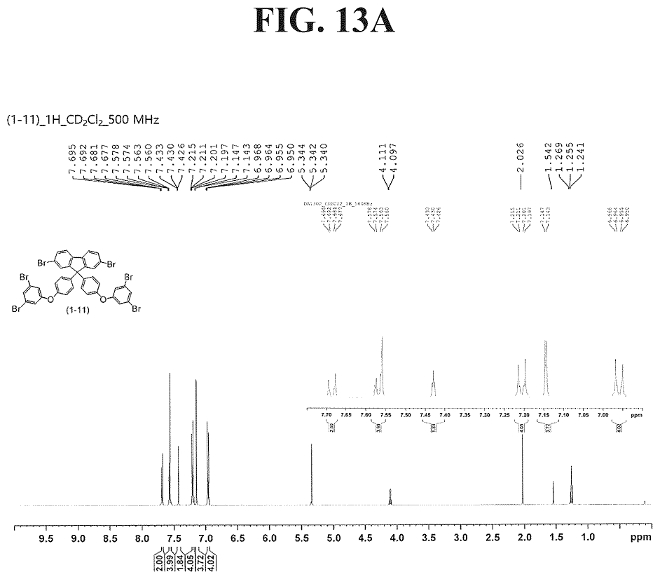

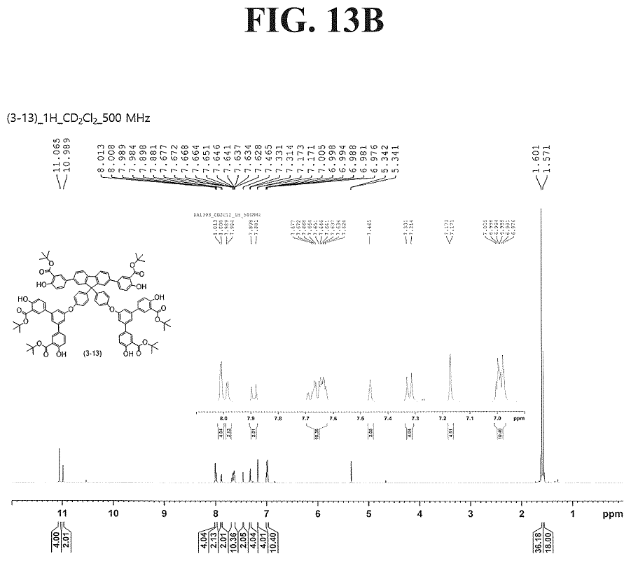

2.54 g (5 mmol) of 2,7-dibromo-9,9-bis(4-hydroxyphenyl)fluorene, 3.77 g (12.5 mmol) of 1,3-dibromo-5-fluorobenzene, 2.07 g (15 mmol) of K₂CO₃ and 40 mL of NMP were added to a 100 mL-round bottom flask. A mixture obtained thereby was heated to 145° C., and then stirred in a nitrogen atmosphere for 16 hours. Upon completion of reaction, the mixture was poured into water (0.4 L), and a diluted HCl aqueous solution was added thereto while stirring to adjust a pH to a slightly acidic state. A beige solid precipitate was filtered, washed with H₂O, suspended in boiling MeOH (0.1 L), and filtered. A solid obtained thereby was dissolved in DCM (0.3 L) and treated with charcoal and SiO₂ while stirring, and filtered. After replacing DCM with MeOH, the resultant was refluxed to obtain a white precipitate. The solid was filtered, washed with MeOH, and dried at 85° C. 4.43 g (yield: 90.8%) of Compound (1-11) was obtained as a white powdery solid. ¹H-NMR data of obtained Compound (1-11) are shown in FIG. 13A.

### 2) Synthesis of Compound (3-13)

0.6 g (0.615 mmol) of Compound (1-11), 1.57 g (4.92 mmol) of Compound (2-2), 107 mg (0.092 mmol) of Pd(PPh₃)₄, and 2.04 g (14.76 mmol) of K₂CO₃ were added to a 100 mL-round bottom flask and 60 mL of a THF/H₂O mixture (5:1) was added thereto. A mixture obtained thereby was degassed and heated to 85° C., and then stirred in a nitrogen atmosphere for 16 hours. Upon completion of reaction, the mixture was diluted with EtOAc (0.2 L) and washed twice with 0.2 L of a NH₄Cl aqueous solution, and then dried in the presence of Na₂SO₄. After removing the solvent by drying under reduced pressure, a residue was separated by chromatography on SiO₂ with DCM/hexane (gradient from 50:50 to 100:0) to obtain fragments with an R_{f} of 0.50 (eluent: DCM:hexane=6:1, TLC silica gel 60 F₂₅₄). The solvent was evaporated under reduced pressure, and a residual solid was dried under reduced pressure at 40° C. 0.395 g (yield: 39.1%) of Compound (3-13) was obtained as a white amorphous solid. ¹H-NMR data of obtained Compound (3-13) are shown in FIG. 13B.

### Evaluation Example 1: Evaluation of Thermal Stability

Thermal analysis was performed on about 5 to about 10 mg of each of the compounds shown in Table 1 below by thermo gravimetric analysis (TGA) and differential scanning calorimetry (DSC) (in a N2 atmosphere, temperature range: from room temperature to 600° C. at a rate of 10° C./min)−TGA and from room temperature to 600° C. at a rate of 10° C./min)−DSC, Pan Type: Pt Pan in disposable Al Pan (TGA) and disposable Al pan (DSC)). The results are shown in Table 1 below. During TGA analysis, a temperature at which a mass of a sample corresponds to 99% of an initial mass was marked by Ta (1%).

TABLE 1

| Compound No. | T_{g} (° C.) | T_{d} (1%, ° C.) |
|---|---|---|
| (3-1) | 347 | 130 |
| (3-2) | 244 | >130 |
| (3-3) | 196 | >140 |
| (3-4) | 138 | >120 |
| (3-5) | 204 | 155 |
| (3-6) | 170 | >130 |
| (3-7) | 145 | 80 |
| (3-8) | 315 | 117 |
| (3-10) | 183 | >130 |
| (3-11) | 147 | 105 |
| (3-12) | 168 | 130 |
| (3-13) | 165 | >120 |
| (6-1) | 164 | >120 |
| (6-2) | 178 | 89 |
| (6-3) | 171 | 68 |
| (C1-4) | 165 | N/A |
| (C2-3) | ~140 | N/A |
| (C3-5) | 175 | 43 |

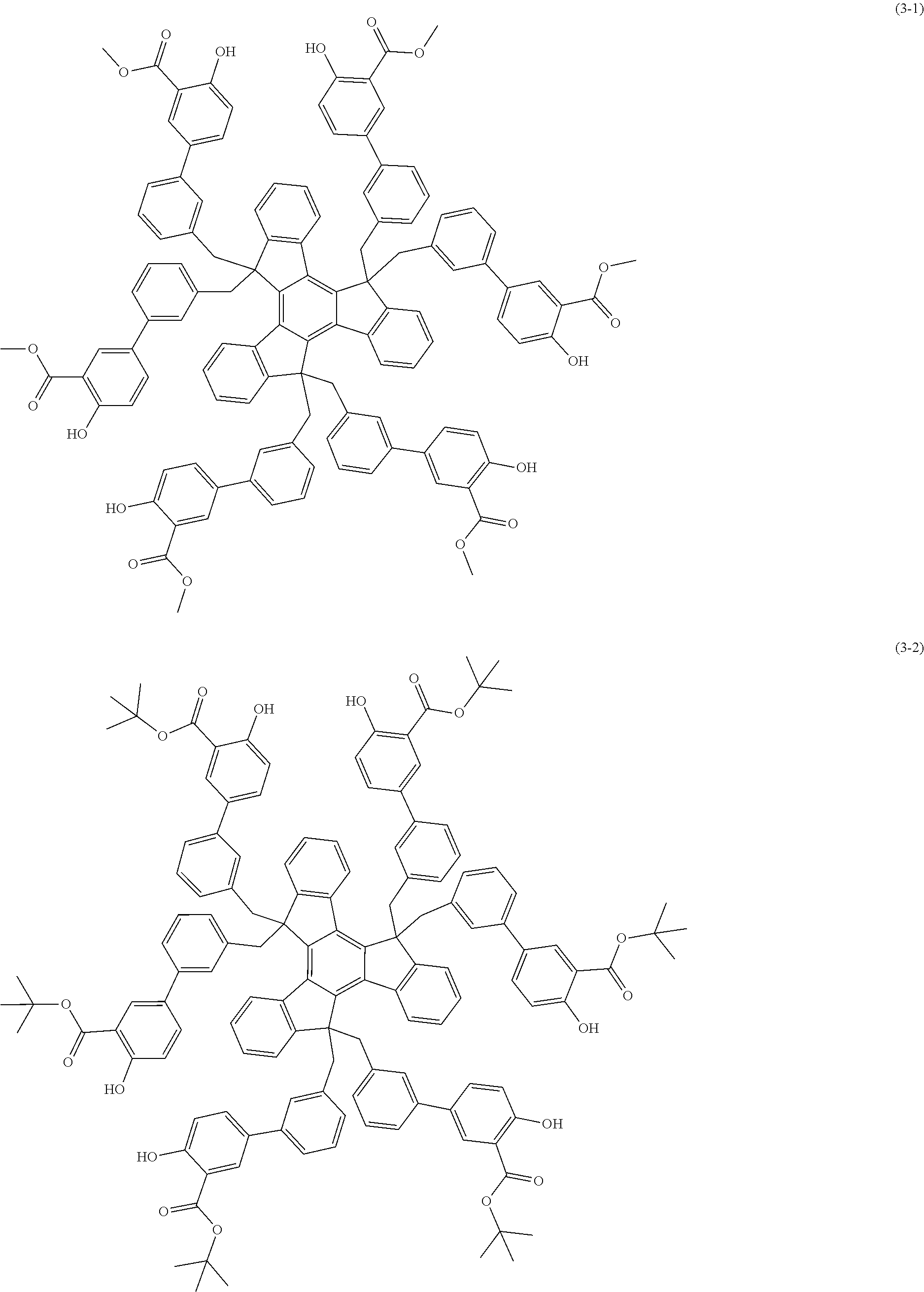

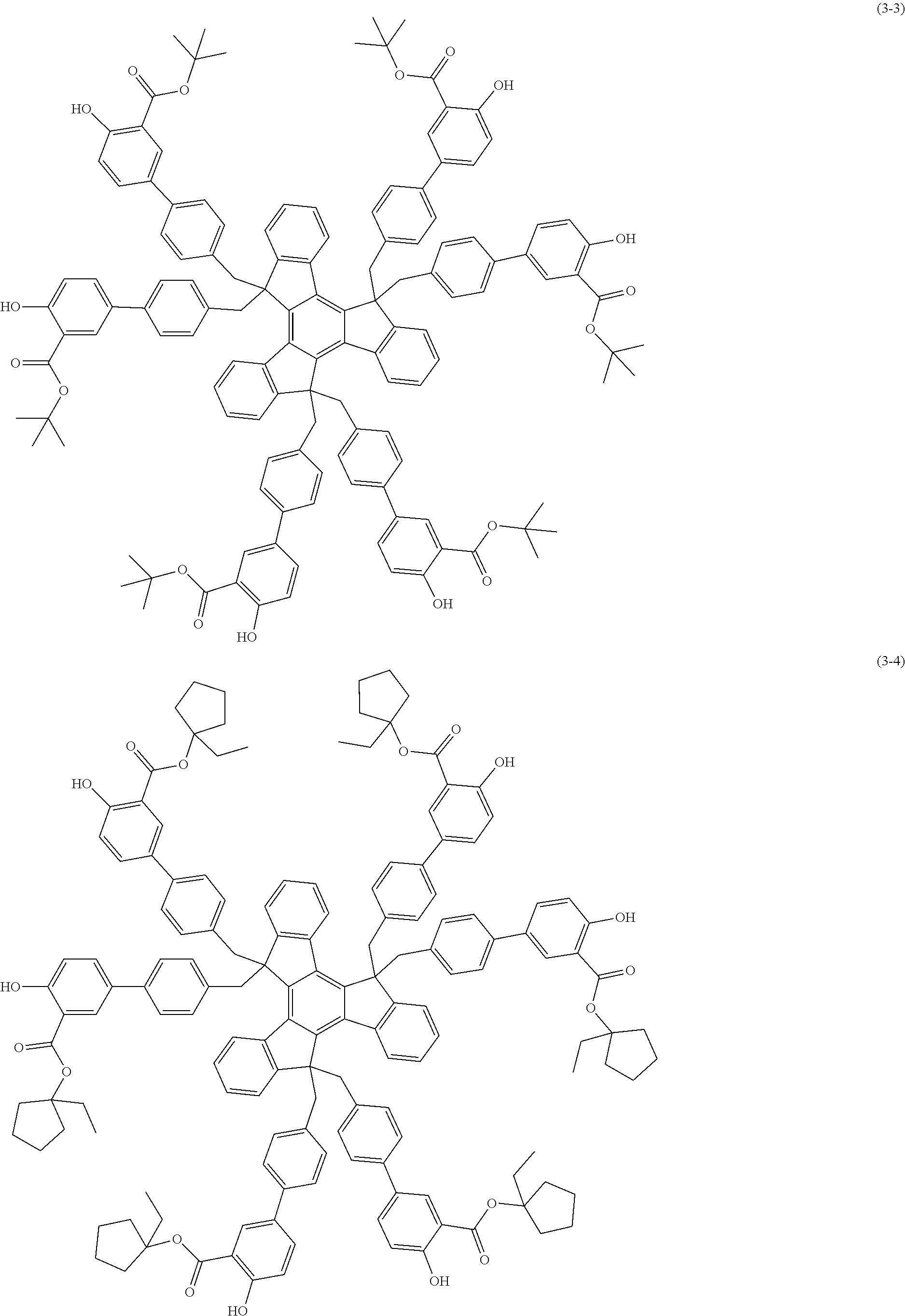

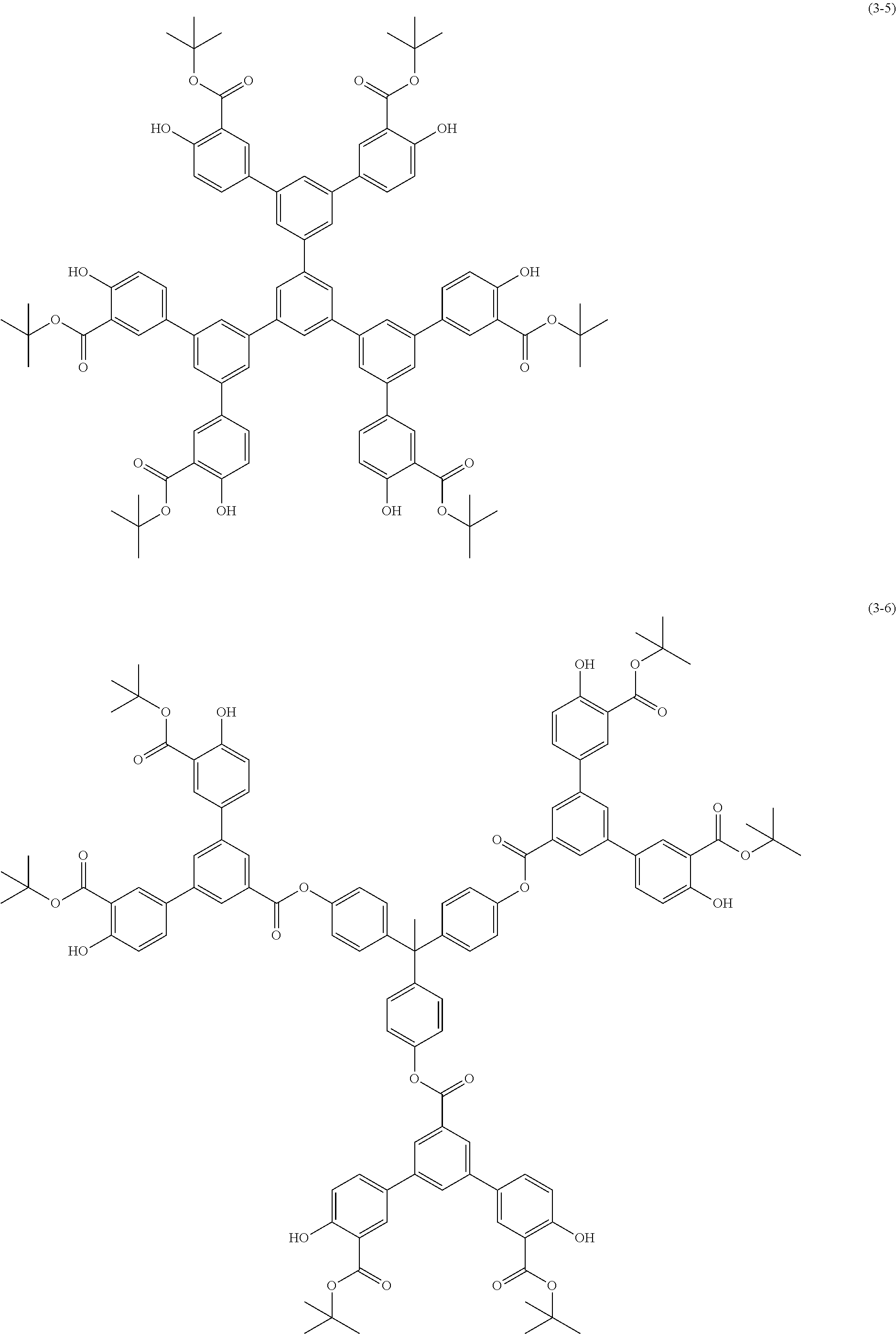

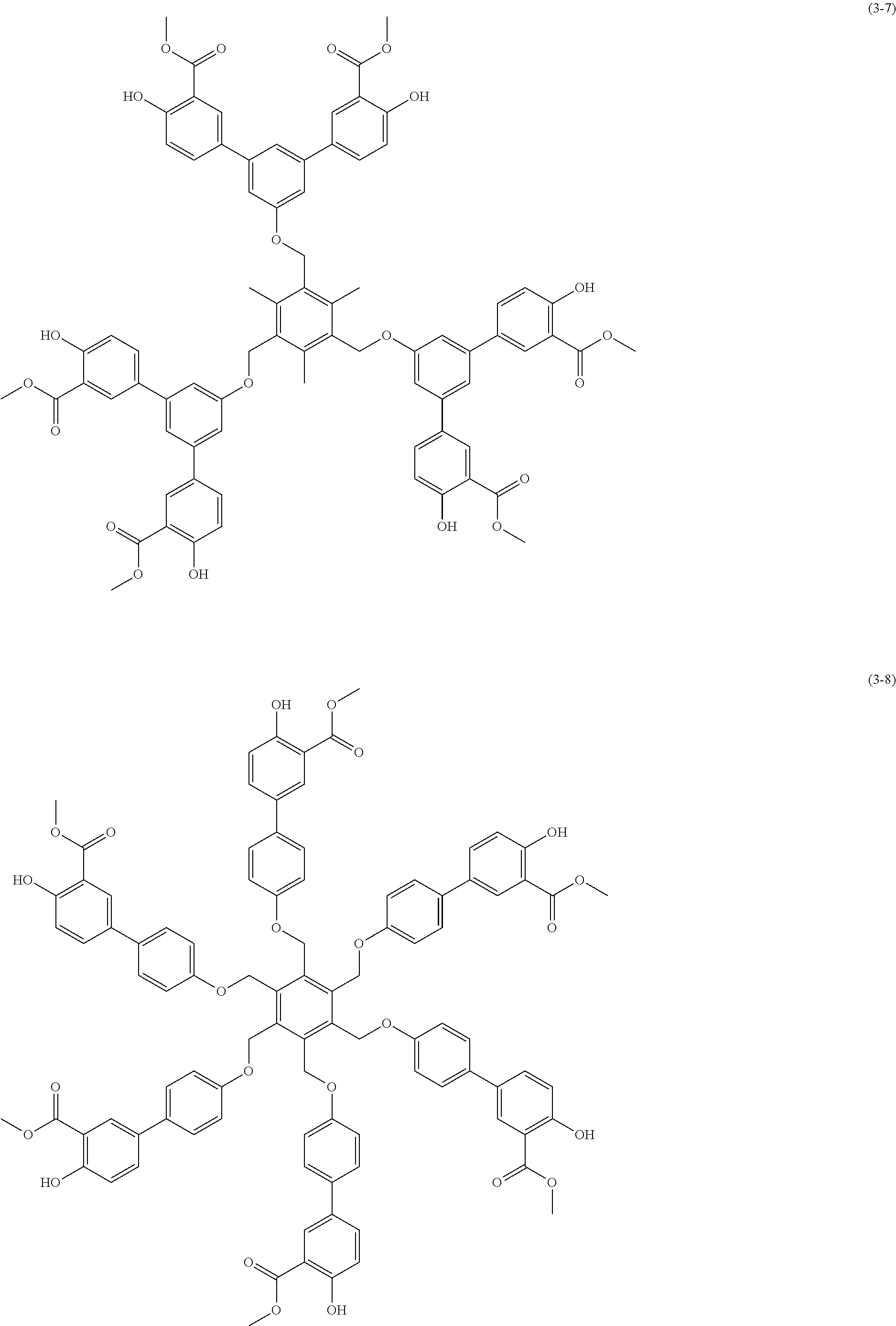

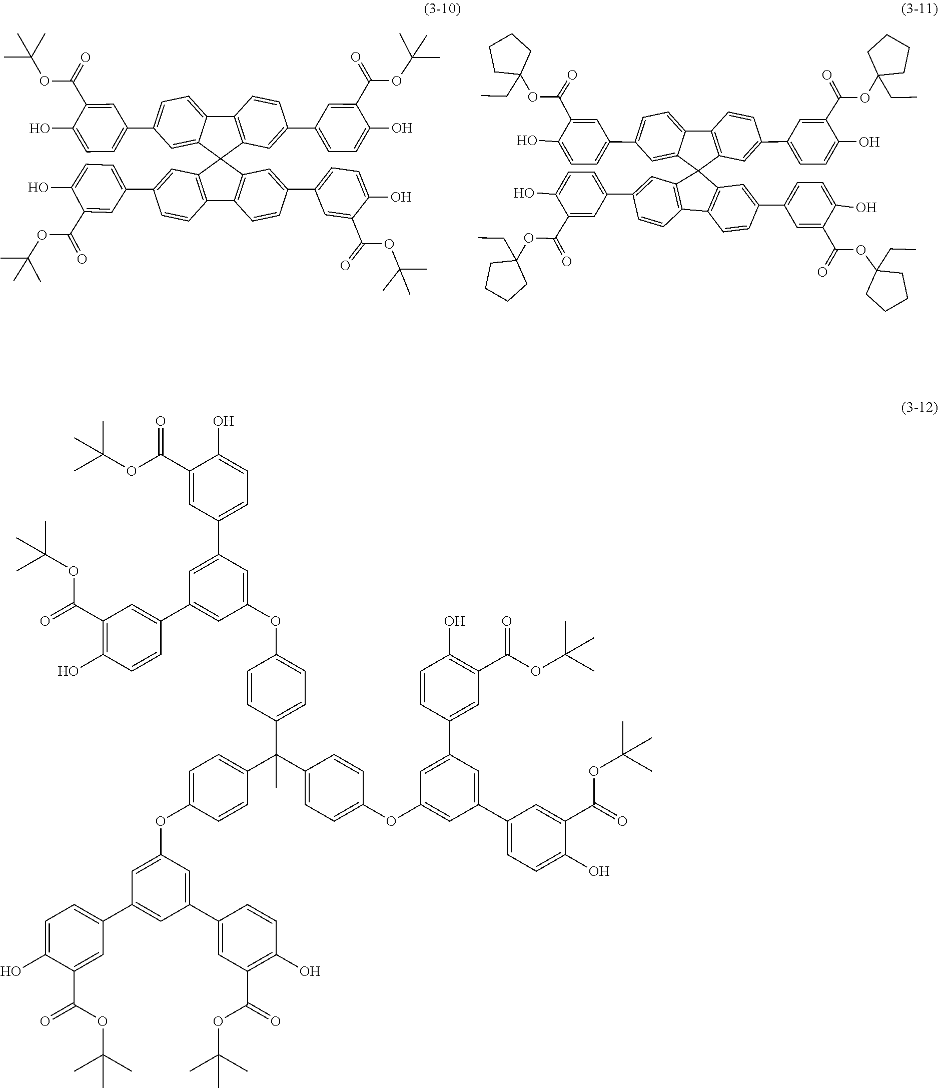

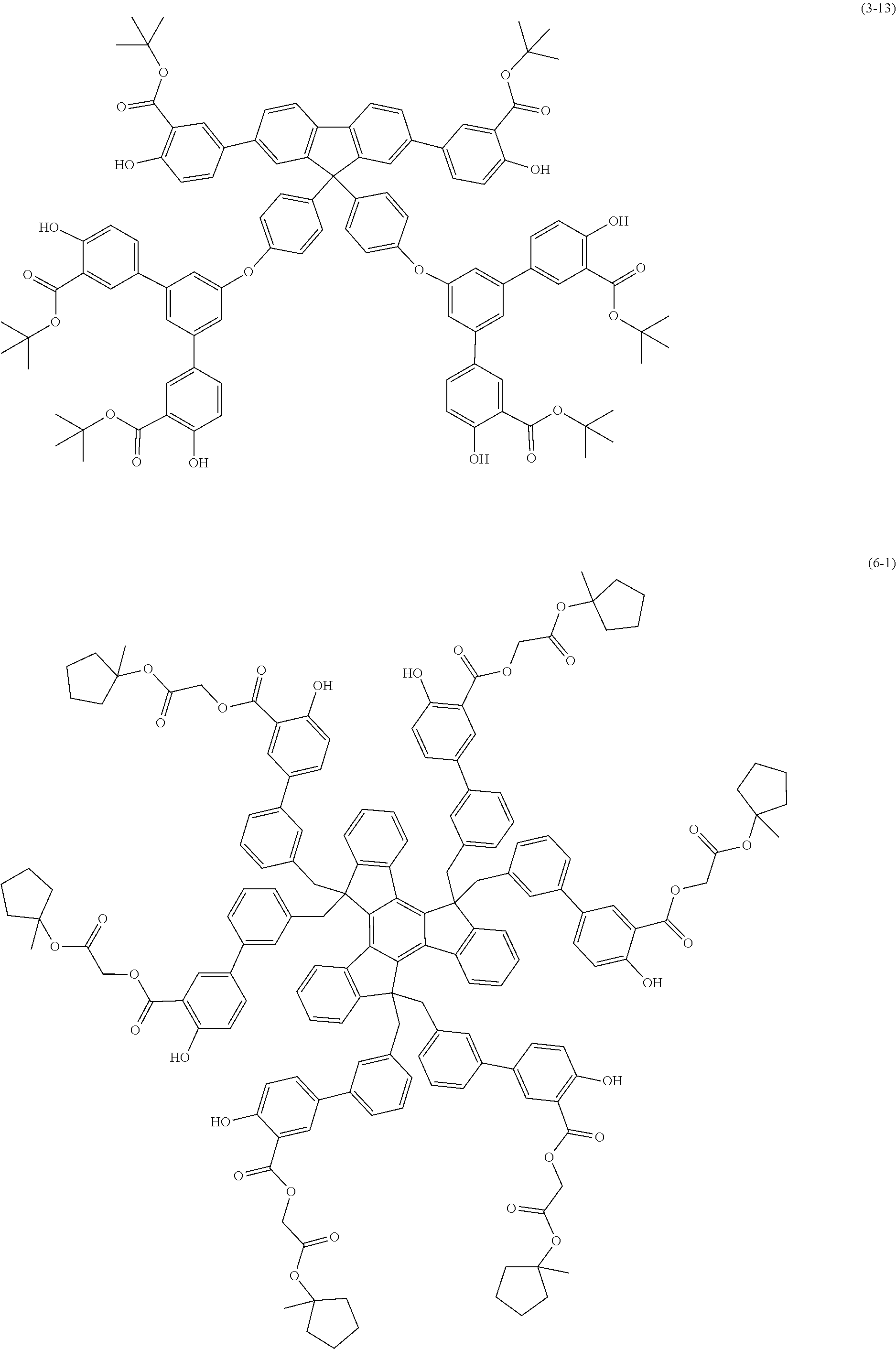

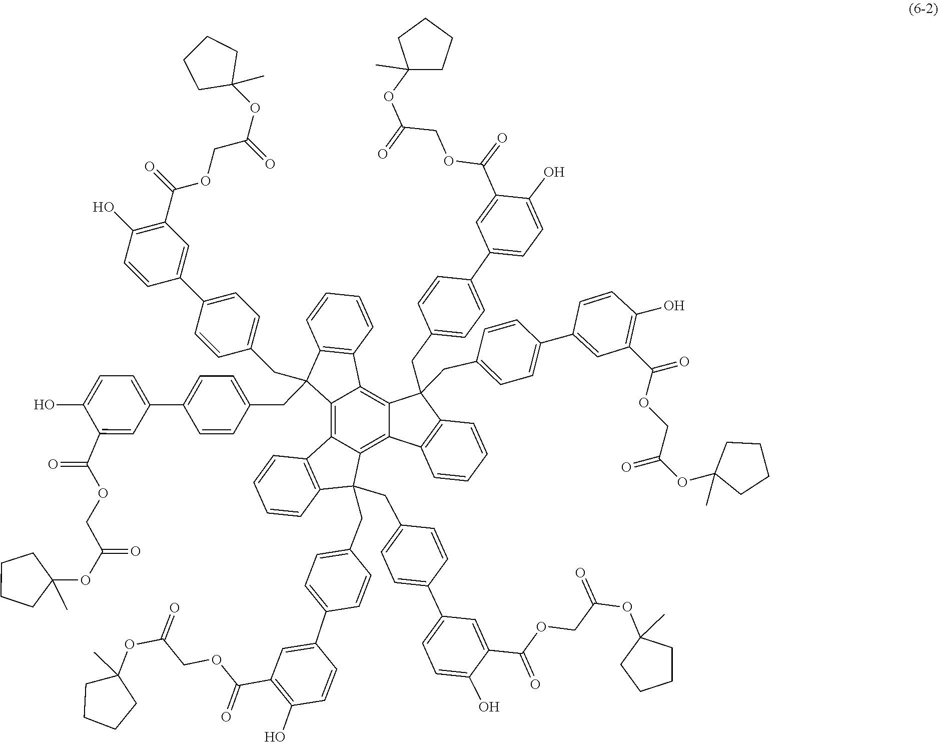 [SMILES: CC1(OC(=O)COC(=O)c2cc(-c3ccc(CC4(Cc5ccc(-c6ccc(O)c(C(=O)OCC(=O)OC7(C)CCCC7)c6)cc5)c5ccccc5-c5c4c4c(c6c5C(Cc5ccc(-c7ccc(O)c(C(=O)OCC(=O)OC8(C)CCCC8)c7)cc5)(Cc5ccc(-c7ccc(O)c(C(=O)OCC(=O)OC8(C)CCCC8)c7)cc5)c5ccccc5-6)C(Cc5ccc(-c6ccc(O)c(C(=O)OCC(=O)OC7(C)CCCC7)c6)cc5)(Cc5ccc(-c6ccc(O)c(C(=O)OCC(=O)OC7(C)CCCC7)c6)cc5)c5ccccc5-4)cc3)ccc2O)CCCC1]

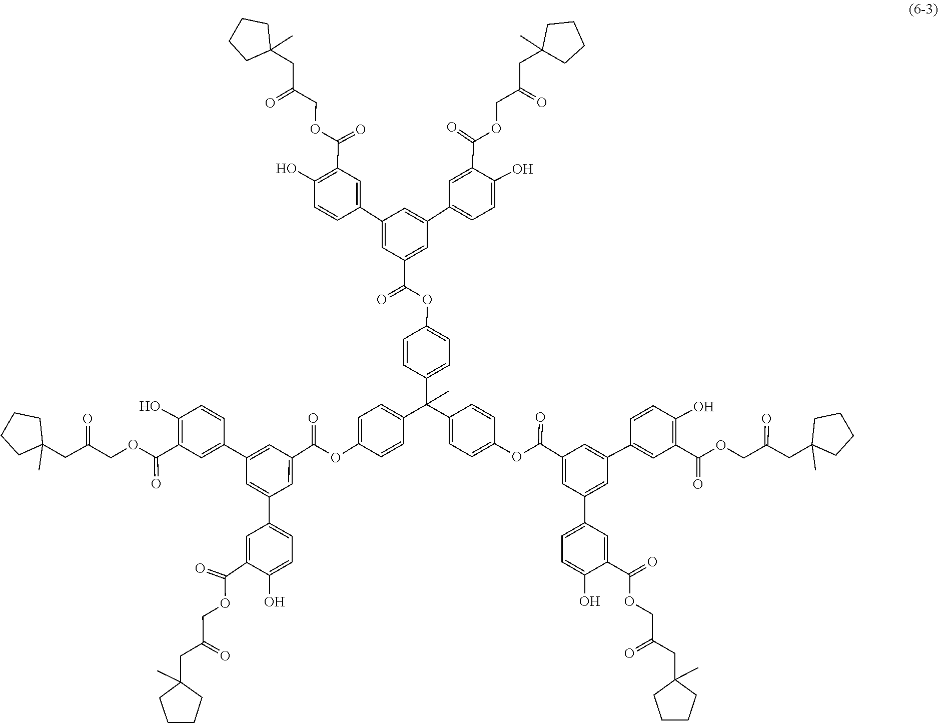 [SMILES: CC1(CC(=O)COC(=O)c2cc(-c3cc(C(=O)Oc4ccc(C(C)(c5ccc(OC(=O)c6cc(-c7ccc(O)c(C(=O)OCC(=O)CC8(C)CCCC8)c7)cc(-c7ccc(O)c(C(=O)OCC(=O)CC8(C)CCCC8)c7)c6)cc5)c5ccc(OC(=O)c6cc(-c7ccc(O)c(C(=O)OCC(=O)CC8(C)CCCC8)c7)cc(-c7ccc(O)c(C(=O)OCC(=O)CC8(C)CCCC8)c7)c6)cc5)cc4)cc(-c4ccc(O)c(C(=O)OCC(=O)CC5(C)CCCC5)c4)c3)ccc2O)CCCC1]

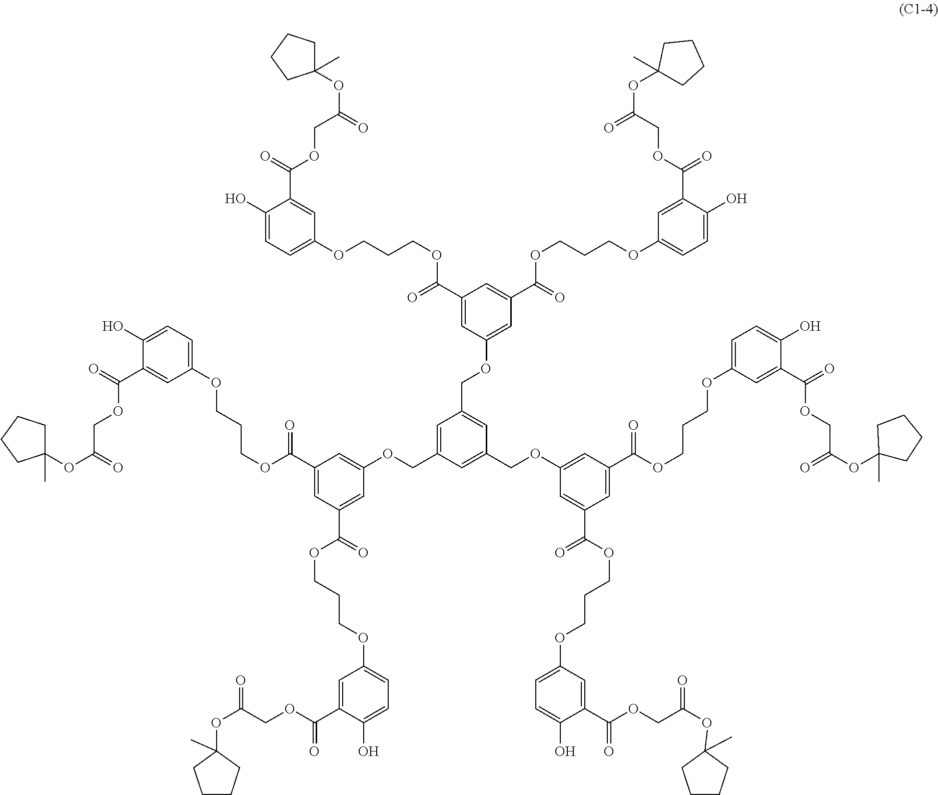 [SMILES: CC1(OC(=O)COC(=O)c2cc(OCCCOC(=O)c3cc(OCc4cc(COc5cc(C(=O)OCCCOc6ccc(O)c(C(=O)OCC(=O)OC7(C)CCCC7)c6)cc(C(=O)OCCCOc6ccc(O)c(C(=O)OCC(=O)OC7(C)CCCC7)c6)c5)cc(COc5cc(C(=O)OCCCOc6ccc(O)c(C(=O)OCC(=O)OC7(C)CCCC7)c6)cc(C(=O)OCCCOc6ccc(O)c(C(=O)OCC(=O)OC7(C)CCCC7)c6)c5)c4)cc(C(=O)OCCCOc4ccc(O)c(C(=O)OCC(=O)OC5(C)CCCC5)c4)c3)ccc2O)CCCC1]

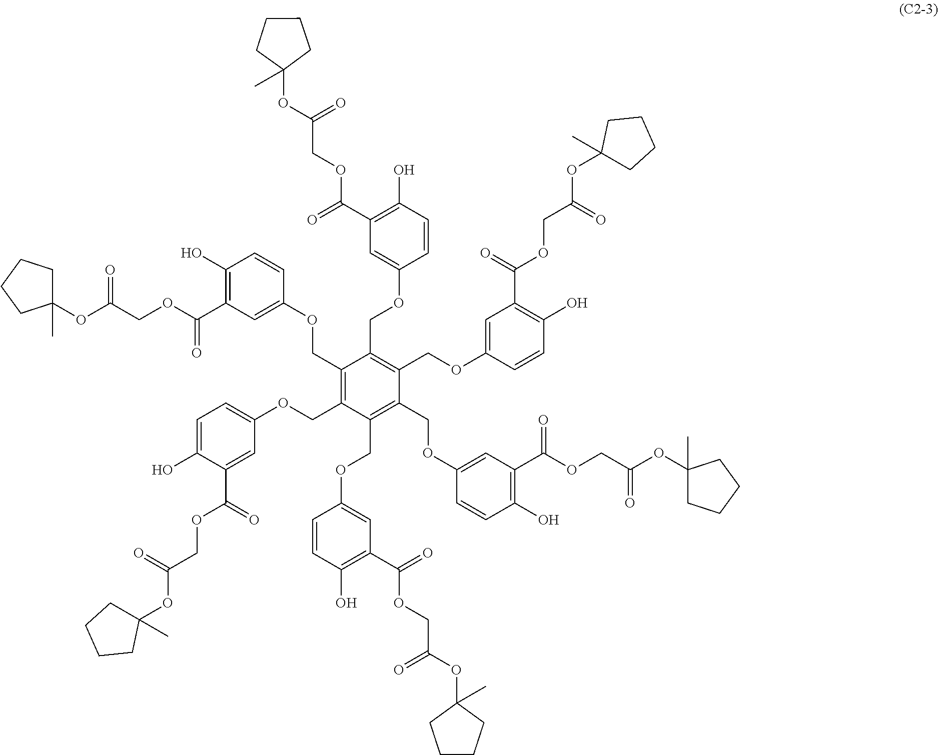 [SMILES: CC1(OC(=O)COC(=O)c2cc(OCc3c(COc4ccc(O)c(C(=O)OCC(=O)OC5(C)CCCC5)c4)c(COc4ccc(O)c(C(=O)OCC(=O)OC5(C)CCCC5)c4)c(COc4ccc(O)c(C(=O)OCC(=O)OC5(C)CCCC5)c4)c(COc4ccc(O)c(C(=O)OCC(=O)OC5(C)CCCC5)c4)c3COc3ccc(O)c(C(=O)OCC(=O)OC4(C)CCCC4)c3)ccc2O)CCCC1]

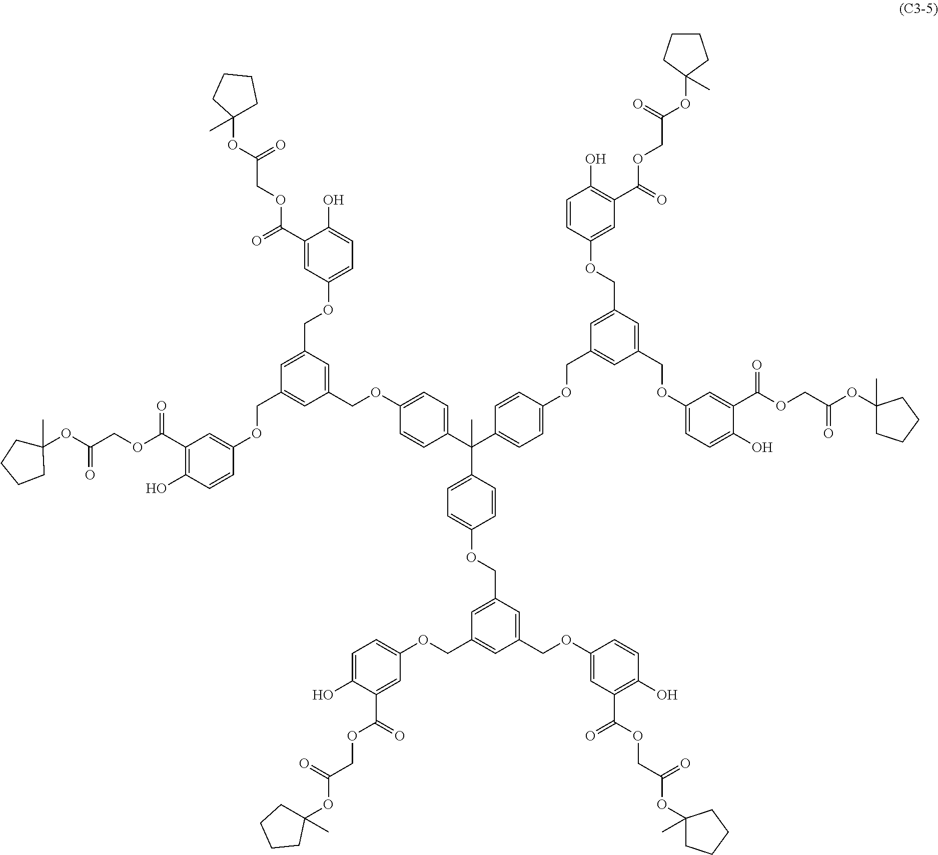 [SMILES: CC1(OC(=O)COC(=O)c2cc(OCc3cc(COc4ccc(C(C)(c5ccc(OCc6cc(COc7ccc(O)c(C(=O)OCC(=O)OC8(C)CCCC8)c7)cc(COc7ccc(O)c(C(=O)OCC(=O)OC8(C)CCCC8)c7)c6)cc5)c5ccc(OCc6cc(COc7ccc(O)c(C(=O)OCC(=O)OC8(C)CCCC8)c7)cc(COc7ccc(O)c(C(=O)OCC(=O)OC8(C)CCCC8)c7)c6)cc5)cc4)cc(COc4ccc(O)c(C(=O)OCC(=O)OC5(C)CCCC5)c4)c3)ccc2O)CCCC1]

In Table 1, N/A indicates that it was impossible to measure.

Referring to Table 1, Compounds (3-1) to (3-8), (3-10) to (3-13) and (6-1) to (6-3) according to embodiments have improved Tg and/or Ta characteristics compared to Compounds (C1-4), (C2-3) and (C3-5) according to the comparative examples.

### Evaluation Example 2: Evaluation of Pattern Formation

Compounds shown in Table 2 below were dissolved in a propylene glycol methyl ether/propylene glycol methyl ether acetate (PGME/PGMEA, 7/3 (wt/wt)) solution to prepare solutions (1 wt % to 4 wt %) 0.043 mmol of triphenyl sulfonium 2-adamantane-1-carbonyl oxy-1,1-difluoroethene-1-sulfonate (PAG) as a photoacid generator and 0.030 mmol of triphenyl sulfonium salicylic acid (PDQ) as a quencher were added thereto, followed by filtering using a 0.2 μm-separator filter. The obtained solution was spin-coated on a silicon wafer at 1500 rpm for 60 seconds to form a coating layer having a thickness of 40 nm. The coating was pre-baked at 130° C. for 60 seconds to remove a residual cast solution. Subsequently, the coating was exposed to DUV with a wavelength of 254 nm at a dose of 0 to 40 mJ/cm². Then, the resultant was subjected to post exposure baking at 90° C. for 60 seconds, immersed in a 2.38 wt % TMAH aqueous solution for 60 seconds, washed with deionized water for 10 seconds to remove regions exposed to DUV light, and dried to form a resist pattern.

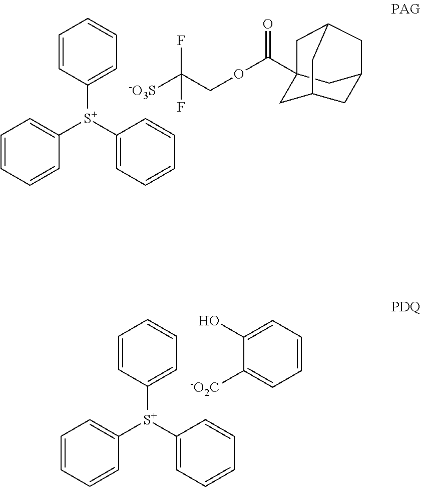

TABLE 2

| Compound No. | Eₜₕ (mJ/cm²) | E₀ (mJ/cm²) | g |
|---|---|---|---|
| (3-3) | 11.8 | 24.0 | 3.2 |
| (3-4) | 5.1 | 13.6 | 2.4 |
| (3-5) | 22.6 | 29.3 | 8.9 |
| (3-10) | 5.3 | 8.9 | 4.5 |
| (6-1) | 5.0 | 6.9 | 6.9 |
| (C3-5) | 13 | 18.6 | 6.38 |

In Table 2, Eth indicates an exposure amount at a time when a thin film starts to develop, and E₀ indicates an exposure amount at a time when the thin film is completely developed. γ, as a contrast curve, is a value calculated by Equation 1 below.

$$γ = {\vert log ( \frac{{E}_{th}}{{E}_{0}} ) \vert}^{- 1} \qquad Equation 1$$

Referring to Table 2, Compounds (3-3) to (3-5), (3-10) and (6-1) according to an embodiment had smaller Eth and/or E₀ values and/or greater values than those of Compound (C3-5) of the comparative example. Based thereon, it may be confirmed that resist compositions including Compounds (3-3) to (3-5), (3-10) and (6-1) have improved photosensitivity compared to a resist composition including Compound (C3-5).

According to example embodiments, a resist composition having improved sensitivity and providing a pattern with increased resolution may be provided.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A polycarboxylate compound represented by Formula 1 below:
Formula 1 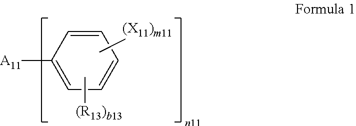
wherein, in Formula 1,
A₁₁ is a single bond, a substituted or unsubstituted carbon atom, a substituted or unsubstituted silicon atom, or an n11-valent binding unit, and
X₁₁ is represented by Formula 2 below, 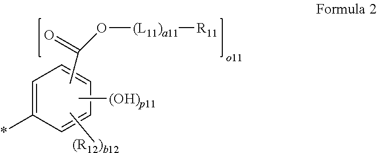
wherein in Formulae 1 and 2,
L₁₁ is (CRₐR_{b})_{q11}, O, CO, CO₂, or any combination thereof,
q11 is an integer from 1 to 6,
a11 is an integer from 0 to 3,
R₁₁ is a linear, branched, or cyclic C₁-C₃₀ monovalent hydrocarbon group optionally including a hetero atom,
Rₐ, R_{b}, R₁₂, and R₁₃ are each independently hydrogen, deuterium, a halogen atom, a hydroxyl group, a cyano group, a nitro group, a linear, branched, or cyclic C₁-C₃₀ monovalent hydrocarbon group optionally including a hetero atom, or —CO₂(Q₁),
Q₁ is hydrogen, deuterium, or a linear, branched, or cyclic C₁-C₃₀ monovalent hydrocarbon group optionally including a hetero atom,
b12 is an integer from 0 to 3,
b13 is an integer from 0 to 4,
o11 is an integer from 1 to 4,
p11 is an integer from 1 to 4,
a sum of b12, o11, and p11 is 5,
m11 is an integer from 1 to 5,
a sum of m11 and b13 is 5,
n11 is an integer from 1 to 12,
A₁₁ optionally binds to R₁₃ to form a ring,
among a plurality of R₁₃, two adjacent ones are optionally bound to each other to form a ring, and
* is a binding site with an adjacent atom.

2. The polycarboxylate compound of claim 1, wherein L₁₁ is (CRₐR_{b})_{q11}, O, CO₂, or any combination thereof.

3. The polycarboxylate compound of claim 1, wherein (L₁₁)ₐ₁₁ is a single bond, CRₐR_{b}O, or CRₐR_{b}CO₂.

4. The polycarboxylate compound of claim 1, wherein
R₁₁ is a substituted or unsubstituted C₁-C₃₀ alkyl group, a substituted or unsubstituted C₃-C₃₀ cycloalkyl group, a substituted or unsubstituted C₂-C₃₀ alkenyl group, a substituted or unsubstituted C₃-C₃₀ cycloalkenyl group, a substituted or unsubstituted C₂-C₃₀ alkynyl group, a substituted or unsubstituted C₆-C₃₀ aryl group, or a substituted or unsubstituted C₇-C₃₀ arylalkyl group,
L₁₁ includes (CRₐR_{b})_{q11},
Rₐ and R_{b} are each independently hydrogen, deuterium, a halogen atom, a hydroxyl group, a cyano group, a nitro group, a substituted or unsubstituted C₁-C₃₀ alkyl group, a substituted or unsubstituted C₁-C₃₀ alkoxy group, a substituted or unsubstituted C₃-C₃₀ cycloalkyl group, a substituted or unsubstituted C₃-C₃₀ cycloalkoxy group, a substituted or unsubstituted C₂-C₃₀ alkenyl group, a substituted or unsubstituted C₃-C₃₀ cycloalkenyl group, a substituted or unsubstituted C₂-C₃₀ alkynyl group, a substituted or unsubstituted C₆-C₃₀ aryl group, or a substituted or unsubstituted C₇-C₃₀ arylalkyl group.

5. The polycarboxylate compound of claim 1, wherein
R₁₁ is selected from compounds represented by Formulae 3-1 to 3-18 below: 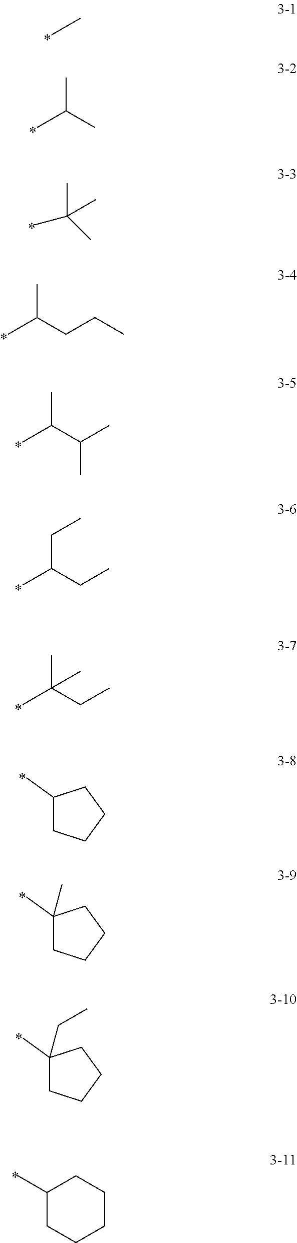 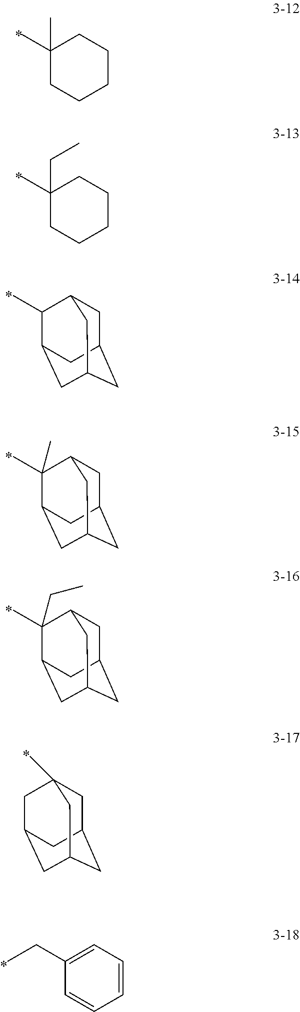
wherein in Formulae 3-1 to 3-18,
* is a binding site with an adjacent atom.

6. The polycarboxylate compound of claim 1, wherein
R₁₂ and R₁₃ are each independently selected from hydrogen; deuterium; a halogen atom; a hydroxyl group; a cyano group; a nitro group; CO₂(Q₁); and a C₁-C₂₀ alkyl group, a C₃-C₂₀ cycloalkyl group, a C₆-C₂₀ aryl group and a C₇-C₃₀ arylalkyl group, unsubstituted or substituted with deuterium, a halogen atom, a hydroxyl group, a cyano group, a nitro group, a carboxylic acid group, an ester moiety, a sulfonate ester moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, a C₇-C₃₀ arylalkyl group, or any combination thereof,
wherein Q₁ is selected from: hydrogen; deuterium; and a C₁-C₂₀ alkyl group, a C₃-C₂₀ cycloalkyl group, a C₆-C₂₀ aryl group and a C₇-C₃₀ arylalkyl group, unsubstituted or substituted with deuterium, a halogen atom, a hydroxyl group, a cyano group, a nitro group, a carboxylic acid group, an ester moiety, a sulfonate ester moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, a C₇-C₃₀ arylalkyl group, or any combination thereof.

7. The polycarboxylate compound of claim 1, wherein 011 and p11 are each independently an integer from 1 to 3.

8. The polycarboxylate compound of claim 1, wherein m11 is an integer from 1 to 3.

9. The polycarboxylate compound of claim 1, wherein n11 is an integer from 1 to 10.

10. The polycarboxylate compound of claim 1, wherein X₁₁ is represented by one of Formulae 2-1 to 2-6 below: 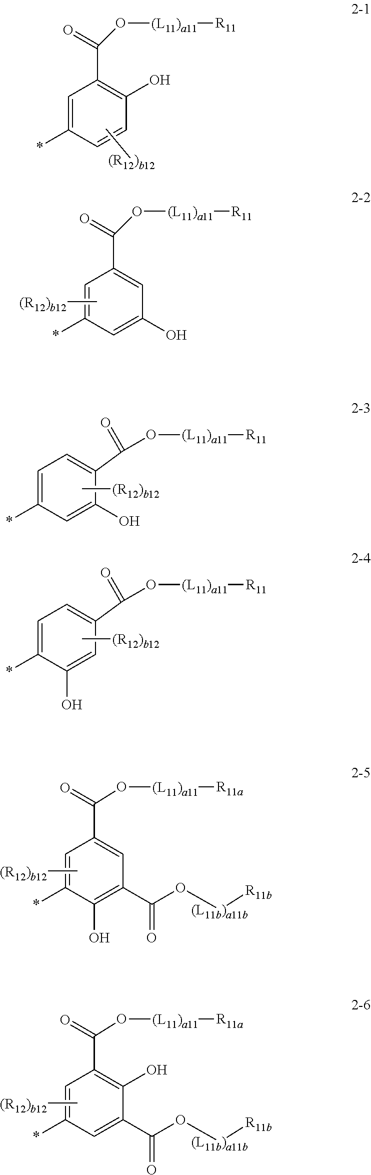
wherein in Formulae 2-1 to 2-6,
definitions of L₁₁, a11, R₁, R₁₂, and b12 are the same as those in Formula 2,
definitions of L₁₁a and L₁₁b are the same as that of L₁₁ in Formula 2,
definitions of a11a and a11b are the same as that of a11 in Formula 2,
definitions of R₁₁ₐ and R_{11b} are the same as that of R₁₁ in Formula 2, and
* is a binding site with an adjacent atom.

11. The polycarboxylate compound of claim 1, wherein X₁₁ is represented by one of Formulae 2-11 to 2-76 below: 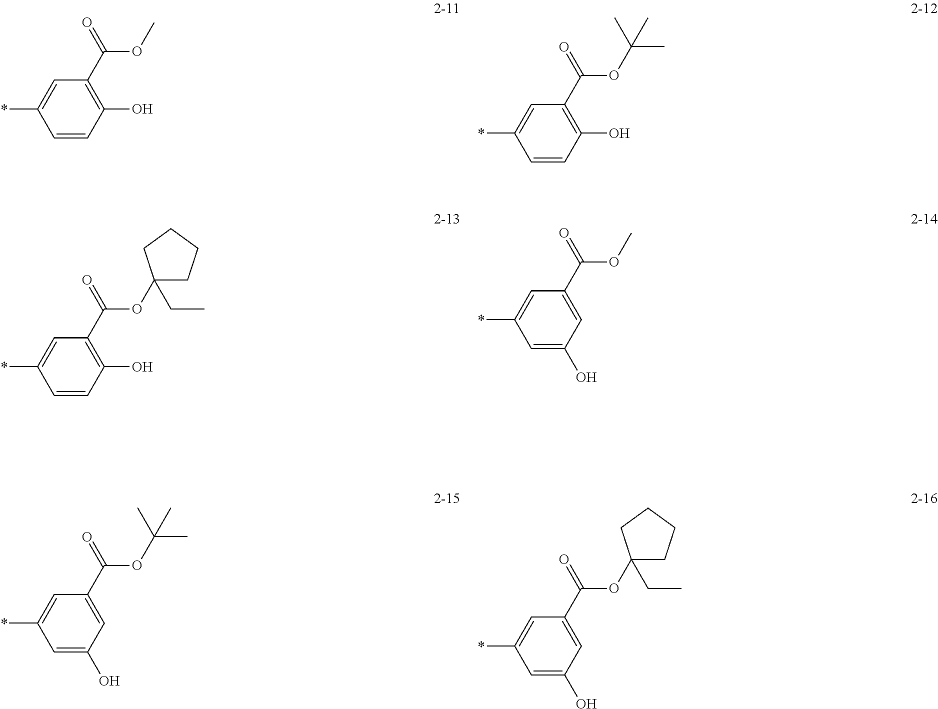 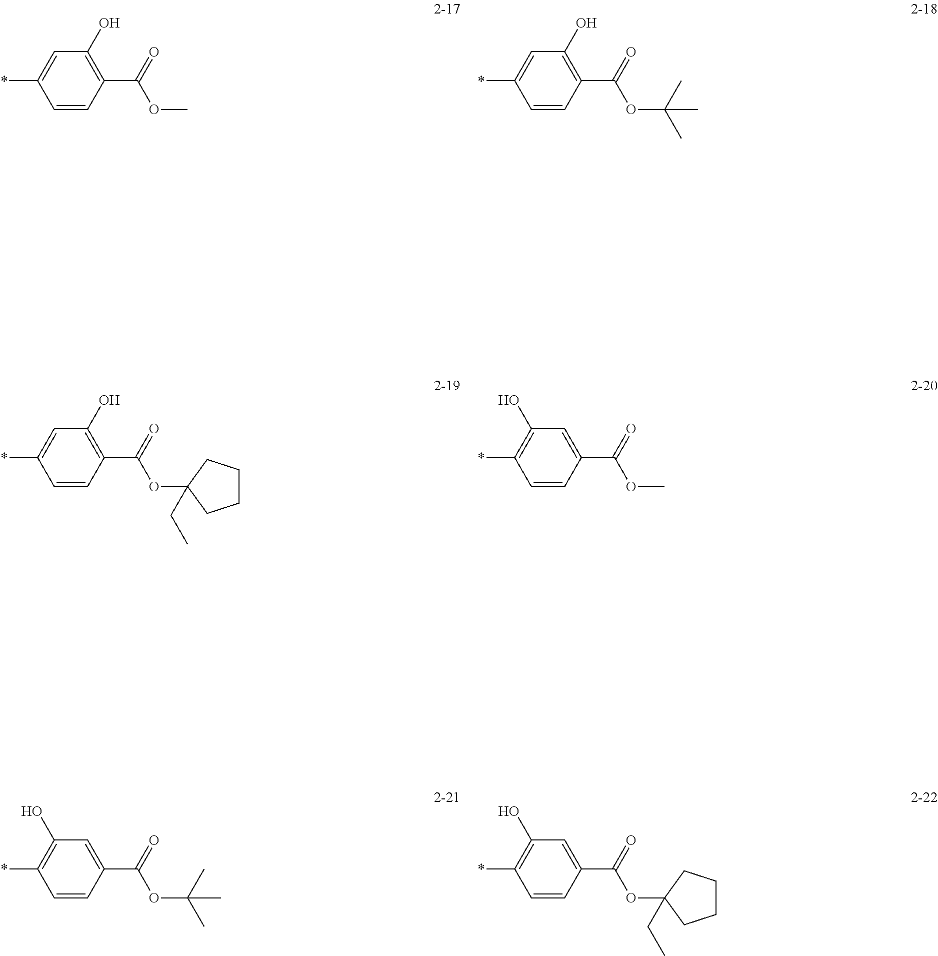 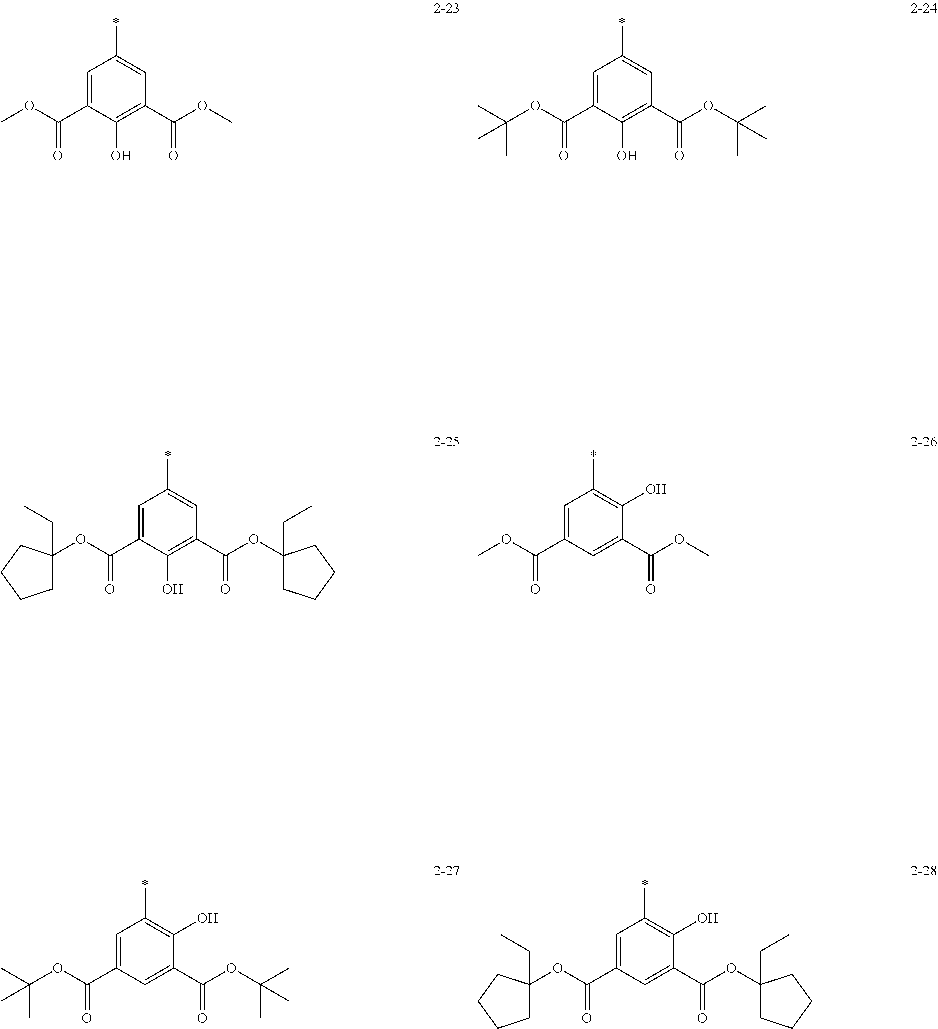 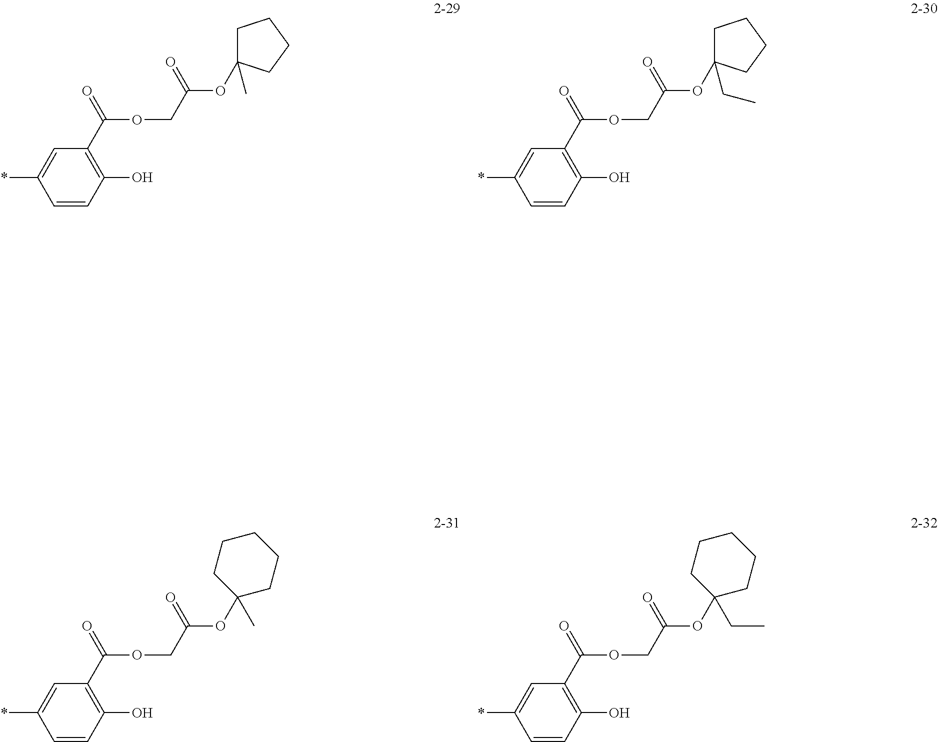 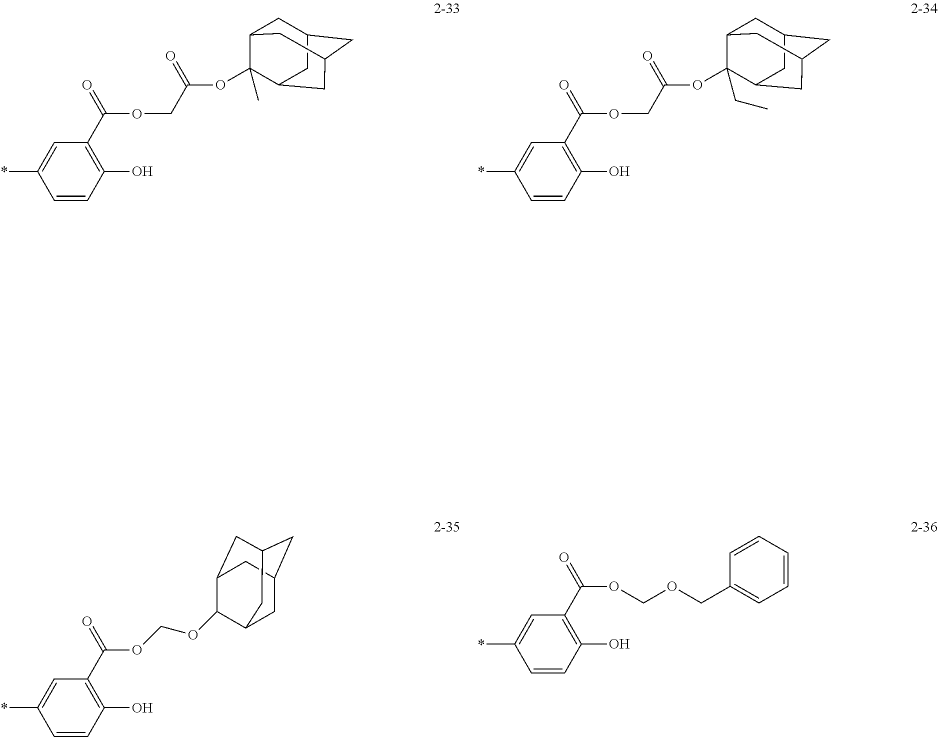 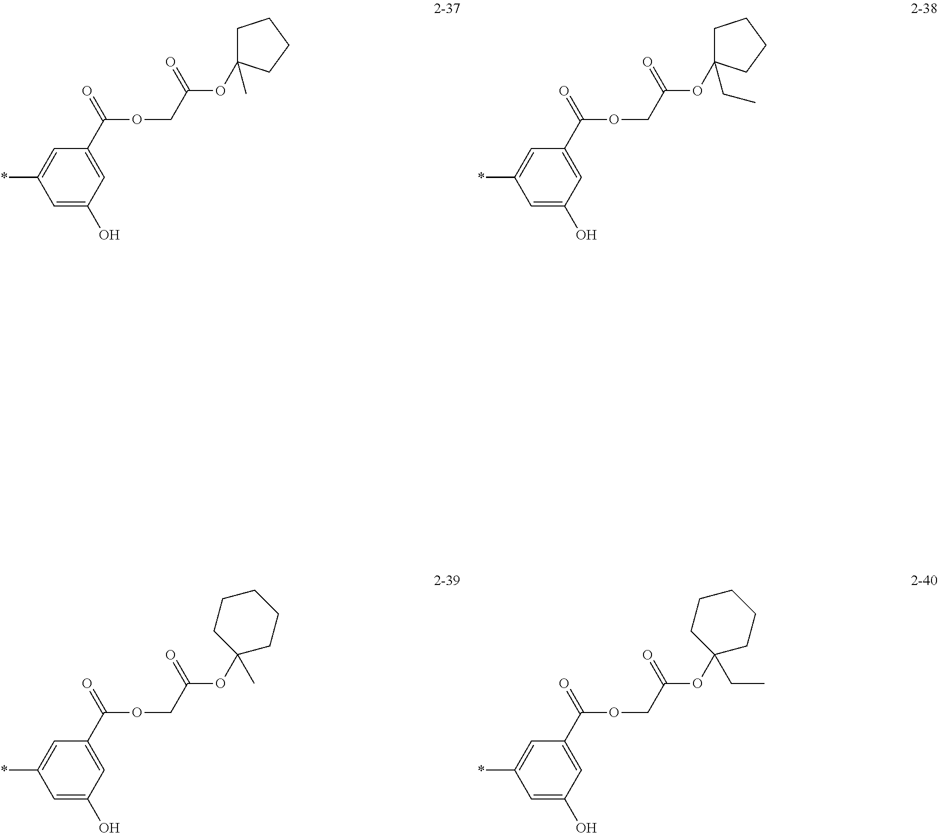 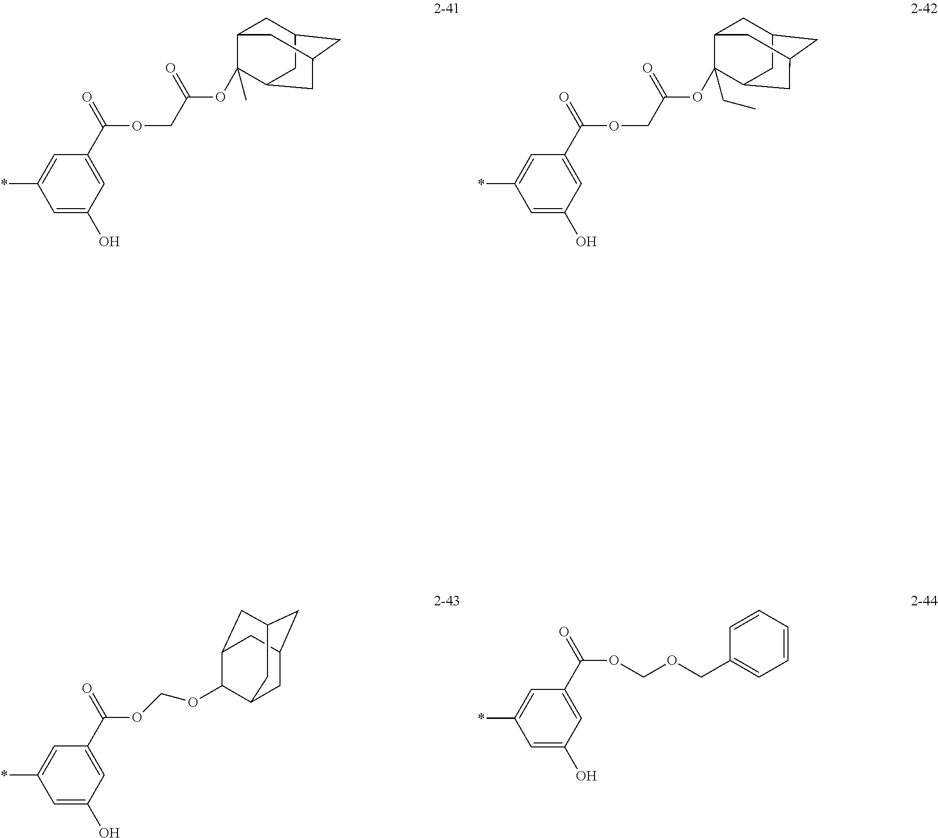 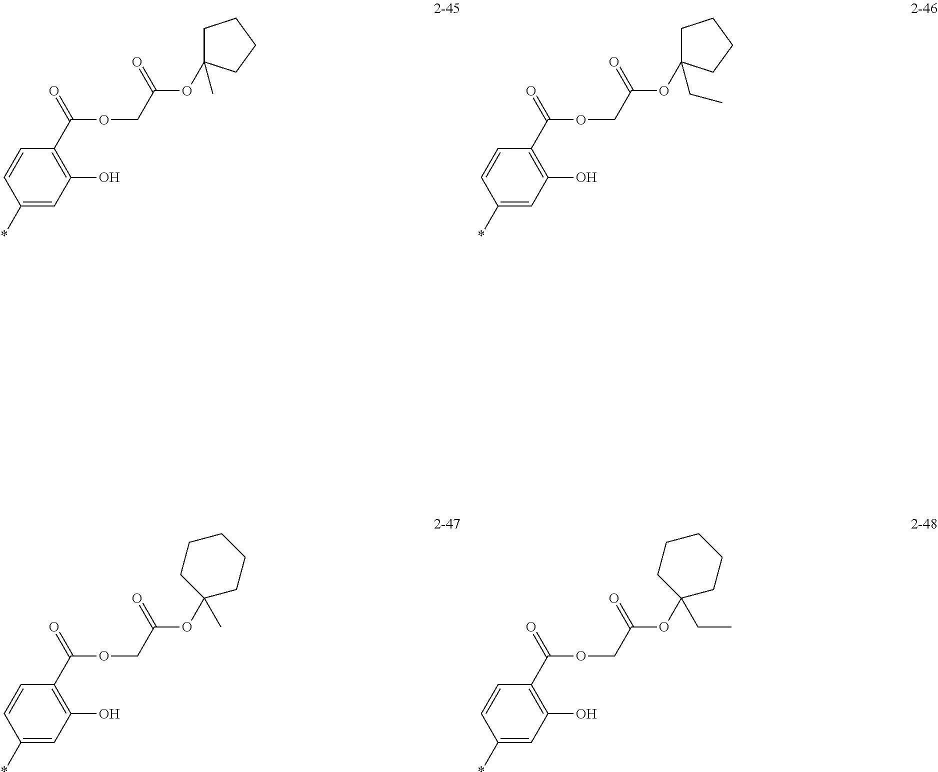 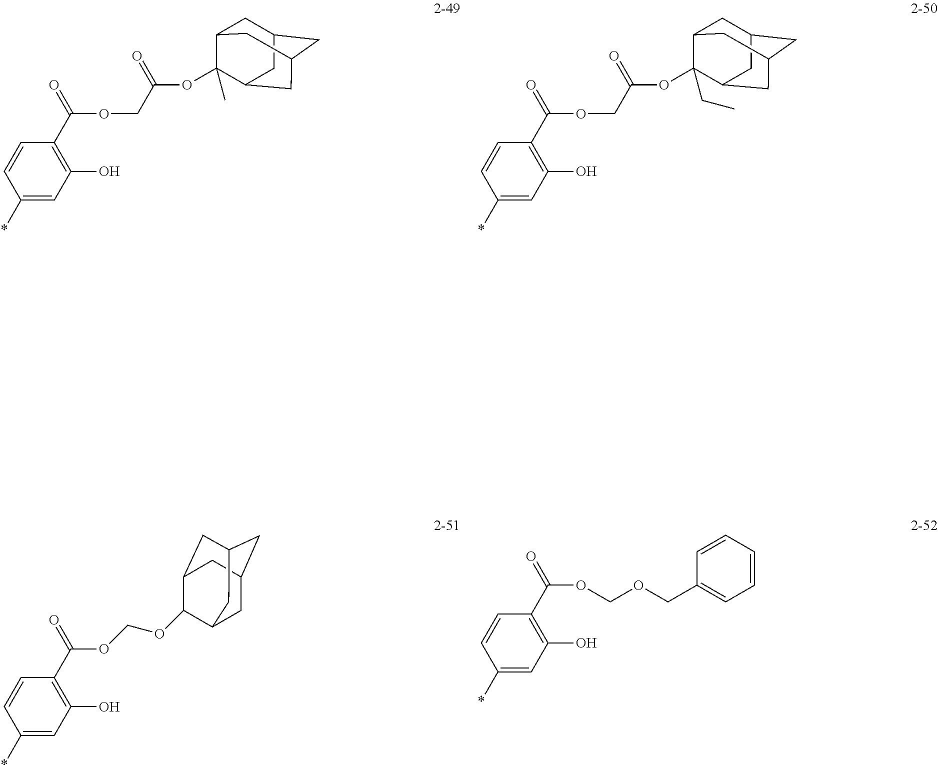 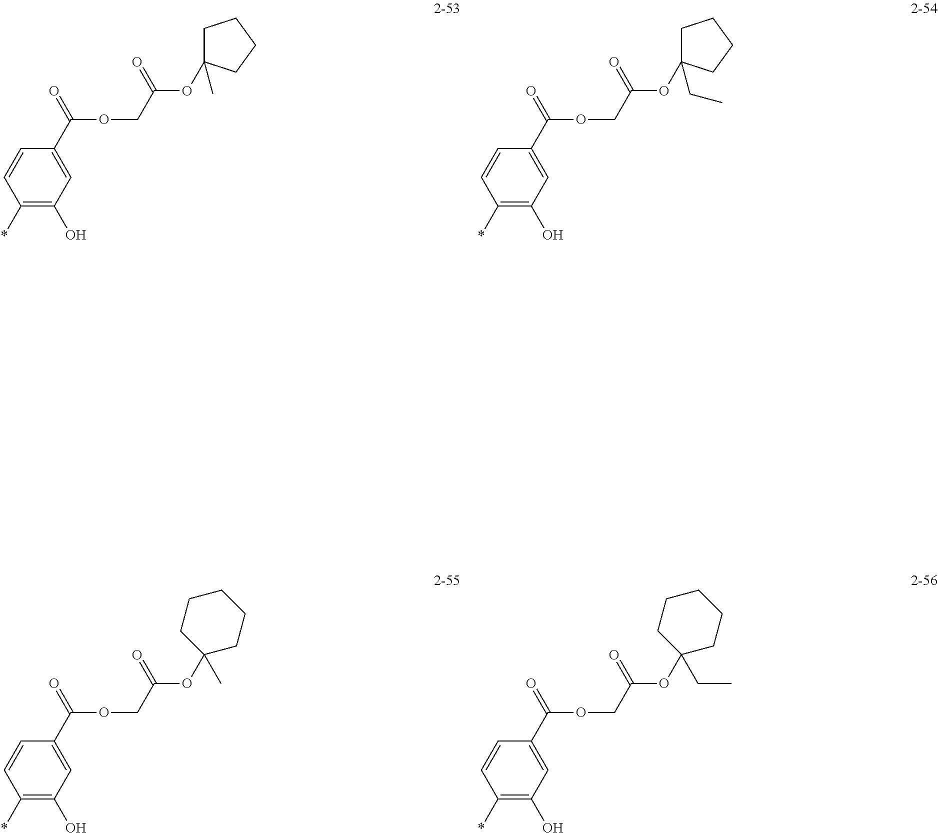 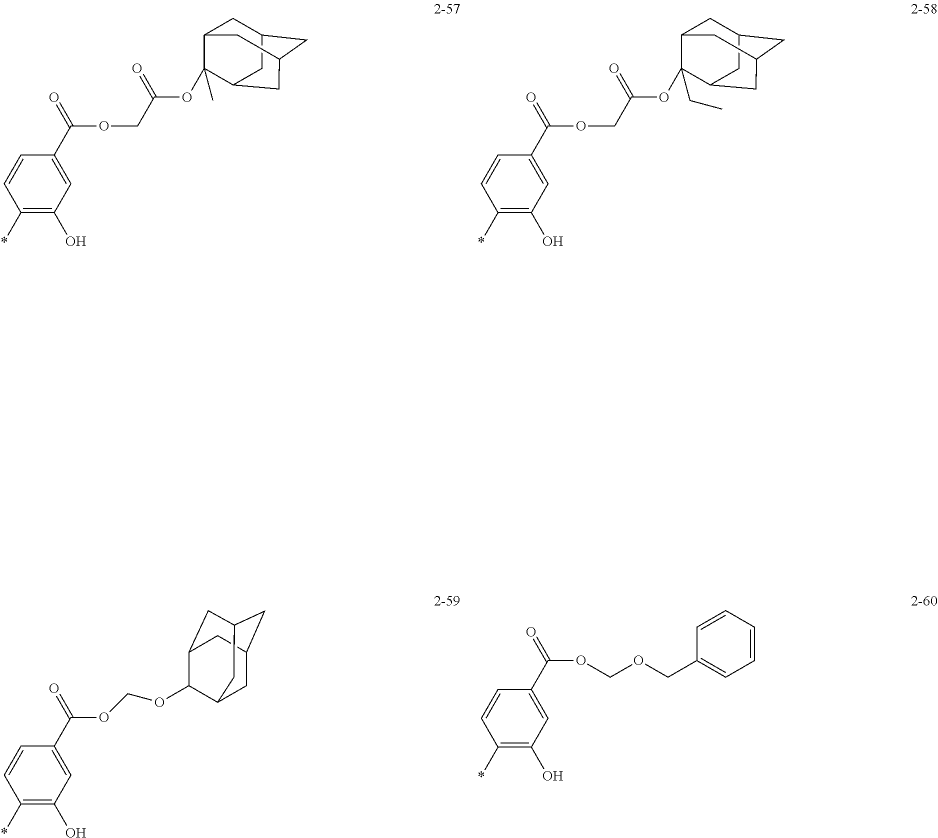 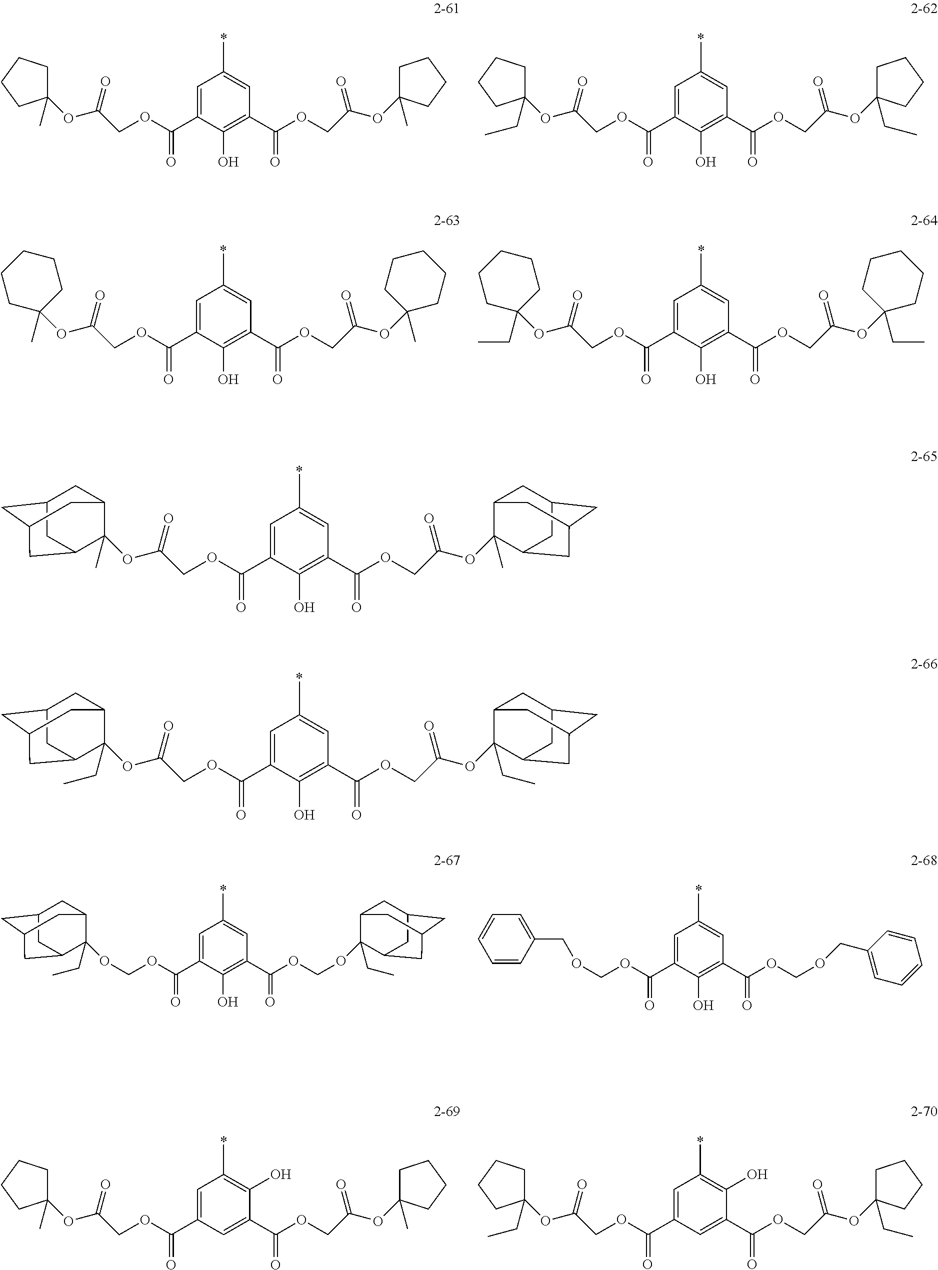 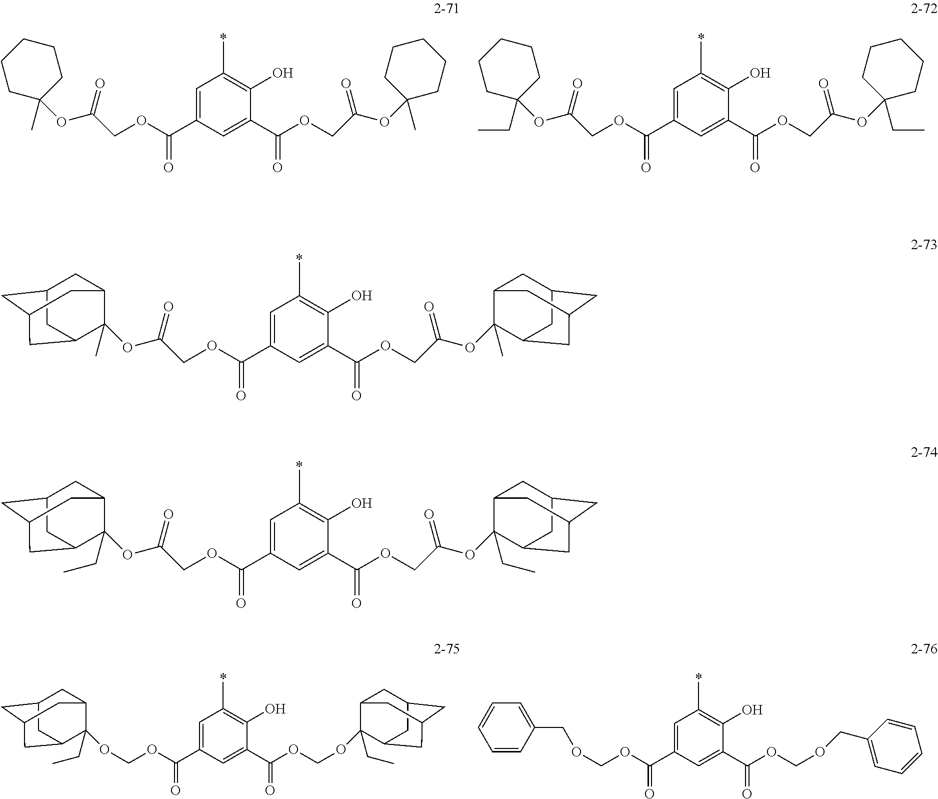
wherein, in Formulae 2-11 to 2-76, and
* is a binding site with an adjacent atom.

12. The polycarboxylate compound of claim 1, wherein the polycarboxylate compound represented by Formula 1 is represented by one of Formulae 1-1 to 1-51 below: 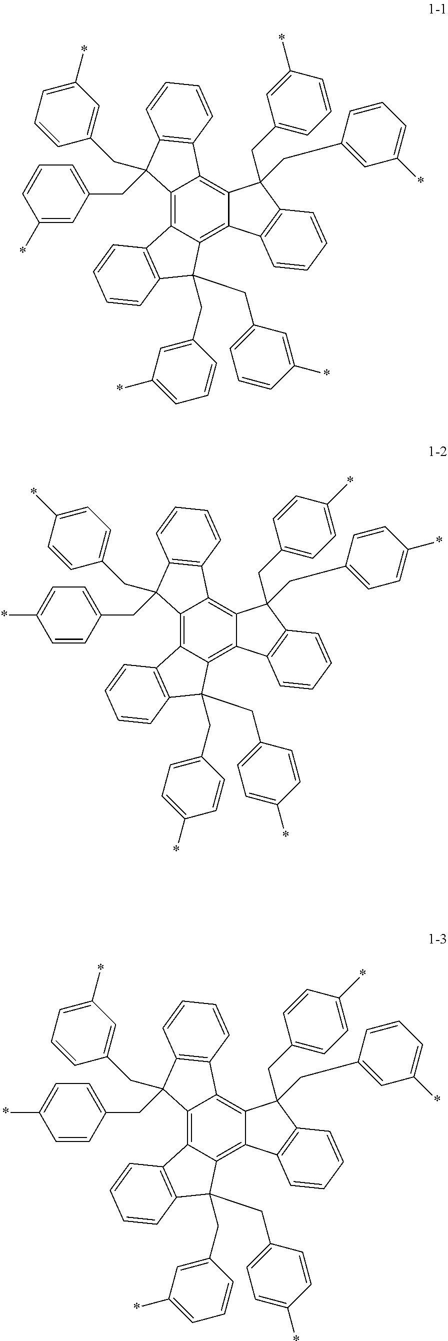 [SMILES: *c1cccc(CC2(Cc3cccc(*)c3)c3ccccc3-c3c2c2c(c4c3C(Cc3cccc(*)c3)(Cc3cccc(*)c3)c3ccccc3-4)C(Cc3cccc(*)c3)(Cc3cccc(*)c3)c3ccccc3-2)c1 ; *c1ccc(CC2(Cc3ccc(*)cc3)c3ccccc3-c3c2c2c(c4c3C(Cc3ccc(*)cc3)(Cc3ccc(*)cc3)c3ccccc3-4)C(Cc3ccc(*)cc3)(Cc3ccc(*)cc3)c3ccccc3-2)cc1 ; *c1ccc(CC2(Cc3cccc(*)c3)c3ccccc3-c3c2c2c(c4c3C(Cc3ccc(*)cc3)(Cc3cccc(*)c3)c3ccccc3-4)C(Cc3ccc(*)cc3)(Cc3cccc(*)c3)c3ccccc3-2)cc1] 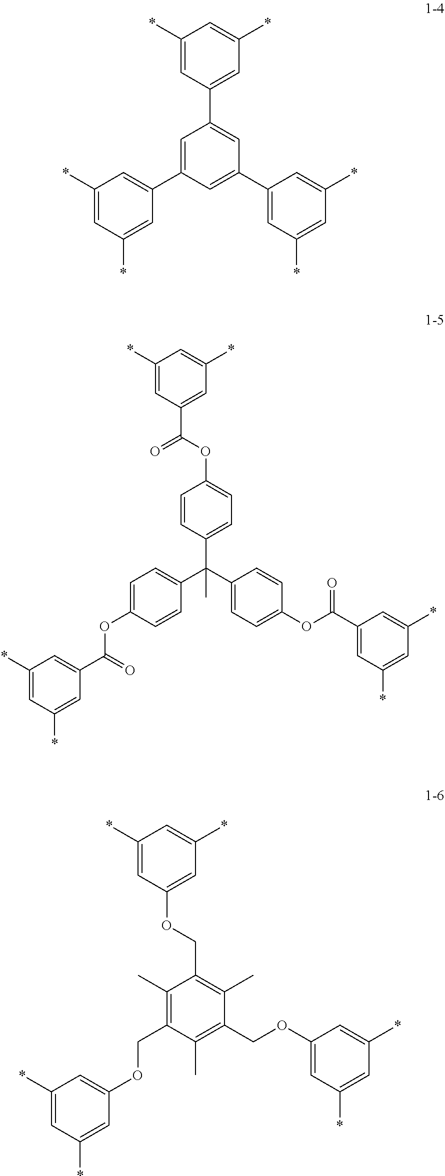 [SMILES: *c1cc(*)cc(-c2cc(-c3cc(*)cc(*)c3)cc(-c3cc(*)cc(*)c3)c2)c1 ; *c1cc(*)cc(C(=O)Oc2ccc(C(C)(c3ccc(OC(=O)c4cc(*)cc(*)c4)cc3)c3ccc(OC(=O)c4cc(*)cc(*)c4)cc3)cc2)c1 ; *c1cc(*)cc(OCc2c(C)c(COc3cc(*)cc(*)c3)c(C)c(COc3cc(*)cc(*)c3)c2C)c1] 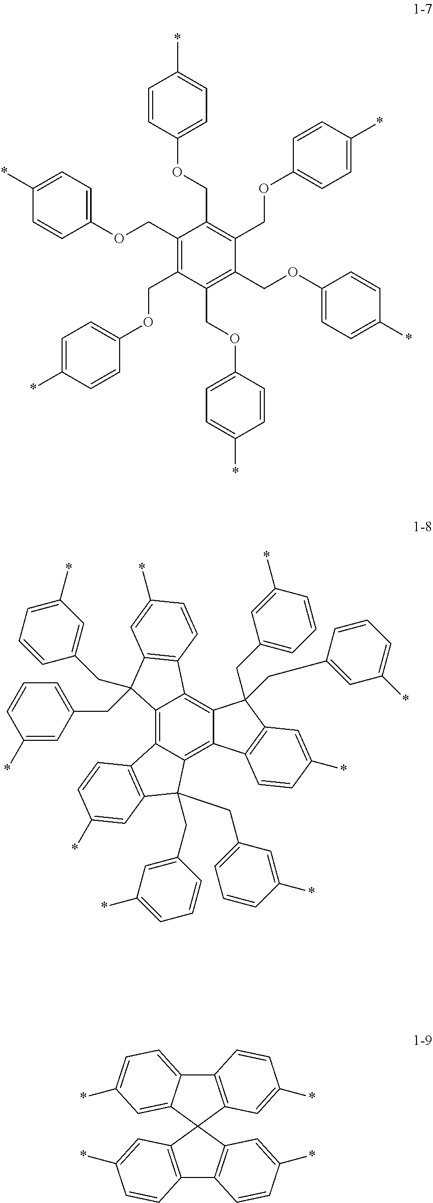 [SMILES: *c1ccc(OCc2c(COc3ccc(*)cc3)c(COc3ccc(*)cc3)c(COc3ccc(*)cc3)c(COc3ccc(*)cc3)c2COc2ccc(*)cc2)cc1 ; *c1cccc(CC2(Cc3cccc(*)c3)c3cc(*)ccc3-c3c2c2c(c4c3C(Cc3cccc(*)c3)(Cc3cccc(*)c3)c3cc(*)ccc3-4)C(Cc3cccc(*)c3)(Cc3cccc(*)c3)c3cc(*)ccc3-2)c1 ; *c1ccc2c(c1)C1(c3cc(*)ccc3-2)c2cc(*)ccc2-c2ccc(*)cc21] 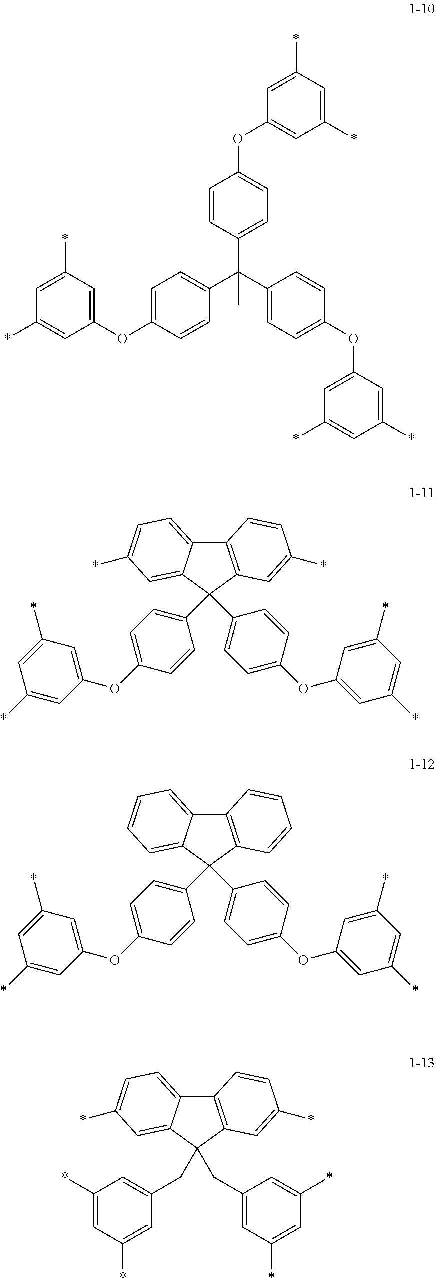 [SMILES: *c1cc(*)cc(Oc2ccc(C(C)(c3ccc(Oc4cc(*)cc(*)c4)cc3)c3ccc(Oc4cc(*)cc(*)c4)cc3)cc2)c1 ; *c1cc(*)cc(Oc2ccc(C3(c4ccc(Oc5cc(*)cc(*)c5)cc4)c4cc(*)ccc4-c4ccc(*)cc43)cc2)c1 ; *c1cc(*)cc(Oc2ccc(C3(c4ccc(Oc5cc(*)cc(*)c5)cc4)c4ccccc4-c4ccccc43)cc2)c1 ; *c1cc(*)cc(CC2(Cc3cc(*)cc(*)c3)c3cc(*)ccc3-c3ccc(*)cc32)c1] 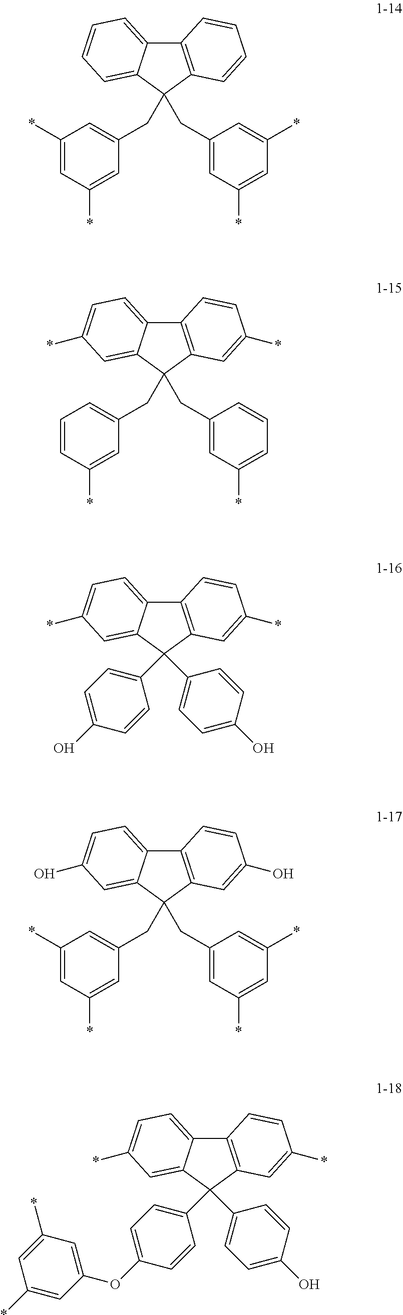 [SMILES: *c1cc(*)cc(CC2(Cc3cc(*)cc(*)c3)c3ccccc3-c3ccccc32)c1 ; *c1cccc(CC2(Cc3cccc(*)c3)c3cc(*)ccc3-c3ccc(*)cc32)c1 ; *c1ccc2c(c1)C(c1ccc(O)cc1)(c1ccc(O)cc1)c1cc(*)ccc1-2 ; *c1cc(*)cc(CC2(Cc3cc(*)cc(*)c3)c3cc(O)ccc3-c3ccc(O)cc32)c1 ; *c1cc(*)cc(Oc2ccc(C3(c4ccc(O)cc4)c4cc(*)ccc4-c4ccc(*)cc43)cc2)c1] 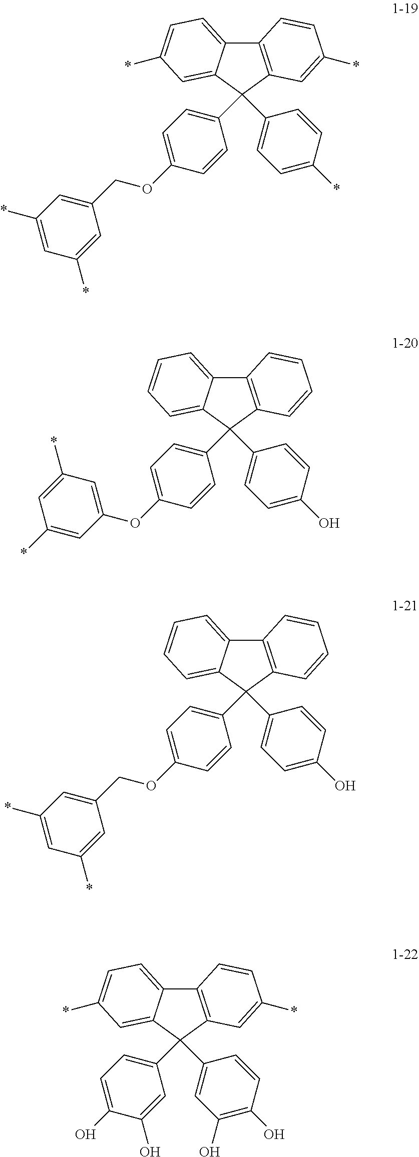 [SMILES: *c1ccc(C2(c3ccc(OCc4cc(*)cc(*)c4)cc3)c3cc(*)ccc3-c3ccc(*)cc32)cc1 ; *c1cc(*)cc(Oc2ccc(C3(c4ccc(O)cc4)c4ccccc4-c4ccccc43)cc2)c1 ; *c1cc(*)cc(COc2ccc(C3(c4ccc(O)cc4)c4ccccc4-c4ccccc43)cc2)c1 ; *c1ccc2c(c1)C(c1ccc(O)c(O)c1)(c1ccc(O)c(O)c1)c1cc(*)ccc1-2] 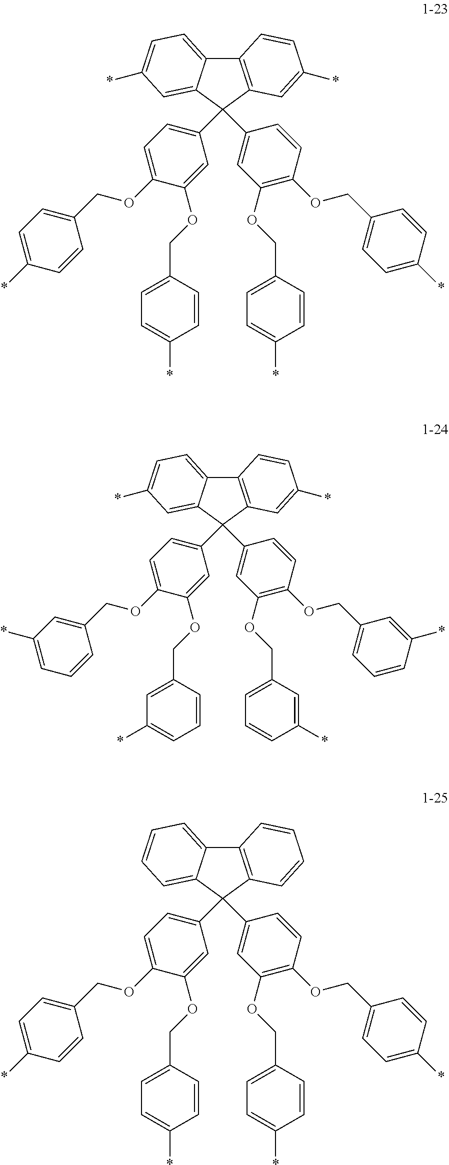 [SMILES: *c1ccc(COc2ccc(C3(c4ccc(OCc5ccc(*)cc5)c(OCc5ccc(*)cc5)c4)c4cc(*)ccc4-c4ccc(*)cc43)cc2OCc2ccc(*)cc2)cc1 ; *c1cccc(COc2ccc(C3(c4ccc(OCc5cccc(*)c5)c(OCc5cccc(*)c5)c4)c4cc(*)ccc4-c4ccc(*)cc43)cc2OCc2cccc(*)c2)c1 ; *c1ccc(COc2ccc(C3(c4ccc(OCc5ccc(*)cc5)c(OCc5ccc(*)cc5)c4)c4ccccc4-c4ccccc43)cc2OCc2ccc(*)cc2)cc1] 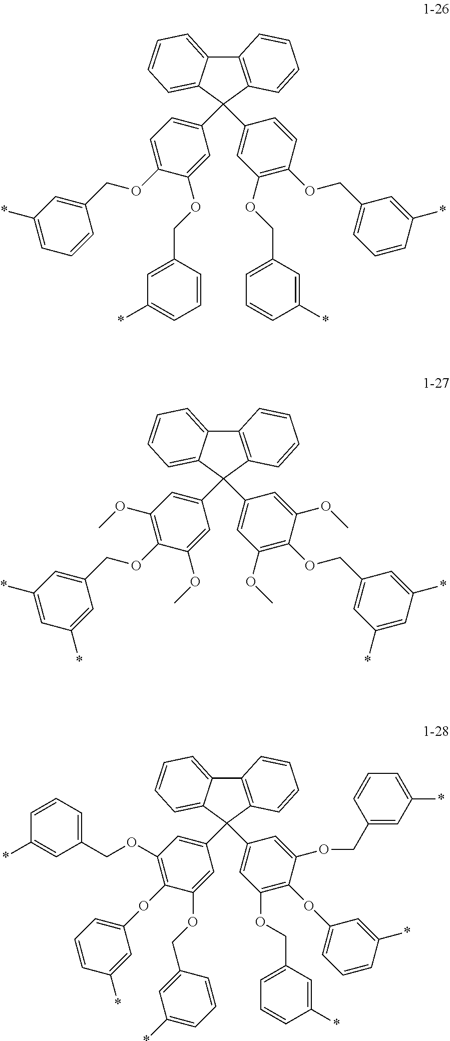 [SMILES: *c1cccc(COc2ccc(C3(c4ccc(OCc5cccc(*)c5)c(OCc5cccc(*)c5)c4)c4ccccc4-c4ccccc43)cc2OCc2cccc(*)c2)c1 ; *c1cc(*)cc(COc2c(OC)cc(C3(c4cc(OC)c(OCc5cc(*)cc(*)c5)c(OC)c4)c4ccccc4-c4ccccc43)cc2OC)c1 ; *c1cccc(COc2cc(C3(c4cc(OCc5cccc(*)c5)c(Oc5cccc(*)c5)c(OCc5cccc(*)c5)c4)c4ccccc4-c4ccccc43)cc(OCc3cccc(*)c3)c2Oc2cccc(*)c2)c1] 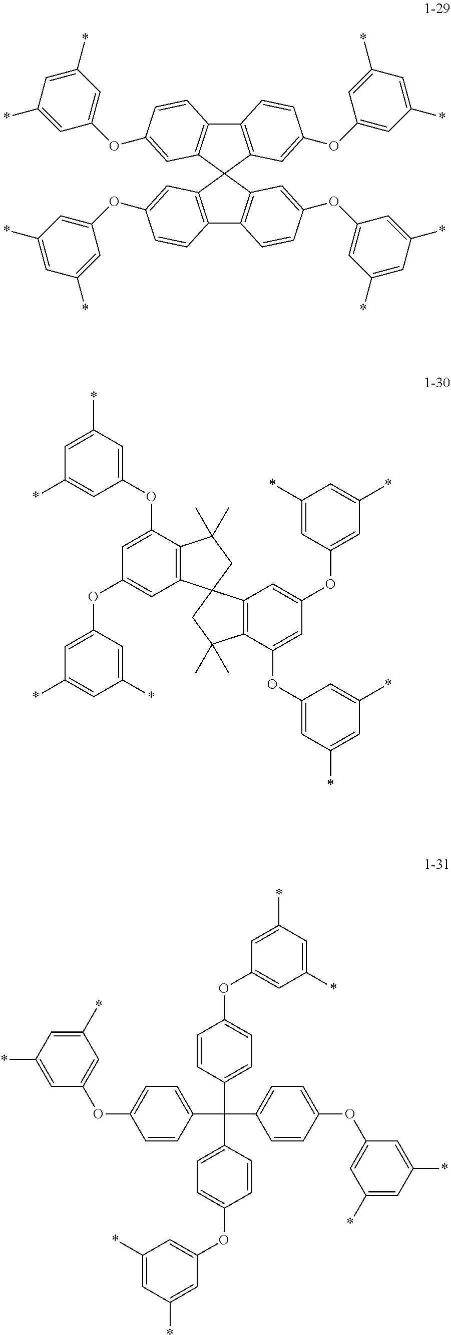 [SMILES: *c1cc(*)cc(Oc2ccc3c(c2)C2(c4cc(Oc5cc(*)cc(*)c5)ccc4-3)c3cc(Oc4cc(*)cc(*)c4)ccc3-c3ccc(Oc4cc(*)cc(*)c4)cc32)c1 ; *c1cc(*)cc(Oc2cc(Oc3cc(*)cc(*)c3)c3c(c2)C2(CC3(C)C)CC(C)(C)c3c(Oc4cc(*)cc(*)c4)cc(Oc4cc(*)cc(*)c4)cc32)c1 ; *c1cc(*)cc(Oc2ccc(C(c3ccc(Oc4cc(*)cc(*)c4)cc3)(c3ccc(Oc4cc(*)cc(*)c4)cc3)c3ccc(Oc4cc(*)cc(*)c4)cc3)cc2)c1] 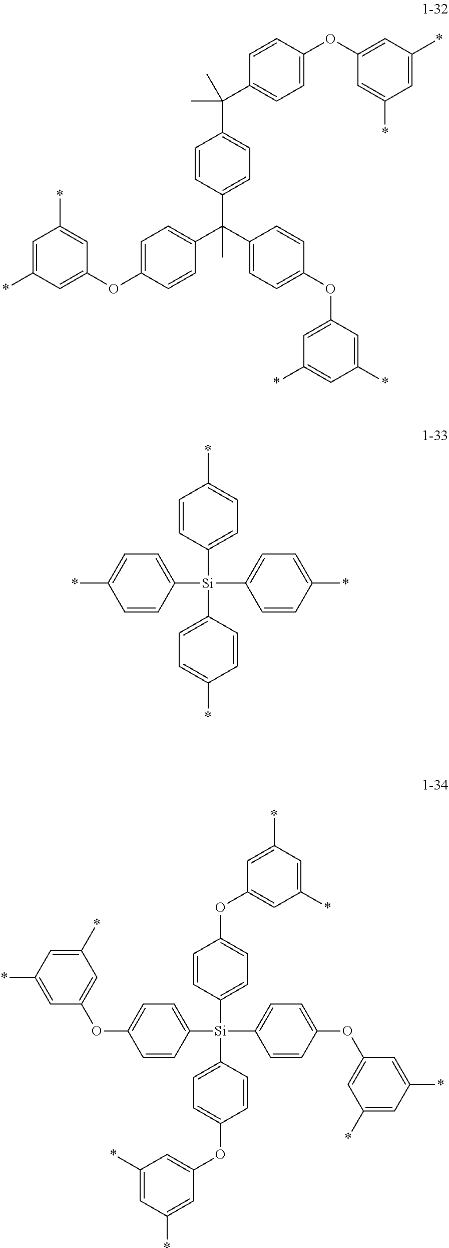 [SMILES: *c1cc(*)cc(Oc2ccc(C(C)(C)c3ccc(C(C)(c4ccc(Oc5cc(*)cc(*)c5)cc4)c4ccc(Oc5cc(*)cc(*)c5)cc4)cc3)cc2)c1 ; *c1ccc([Si](c2ccc(*)cc2)(c2ccc(*)cc2)c2ccc(*)cc2)cc1 ; *c1cc(*)cc(Oc2ccc([Si](c3ccc(Oc4cc(*)cc(*)c4)cc3)(c3ccc(Oc4cc(*)cc(*)c4)cc3)c3ccc(Oc4cc(*)cc(*)c4)cc3)cc2)c1] 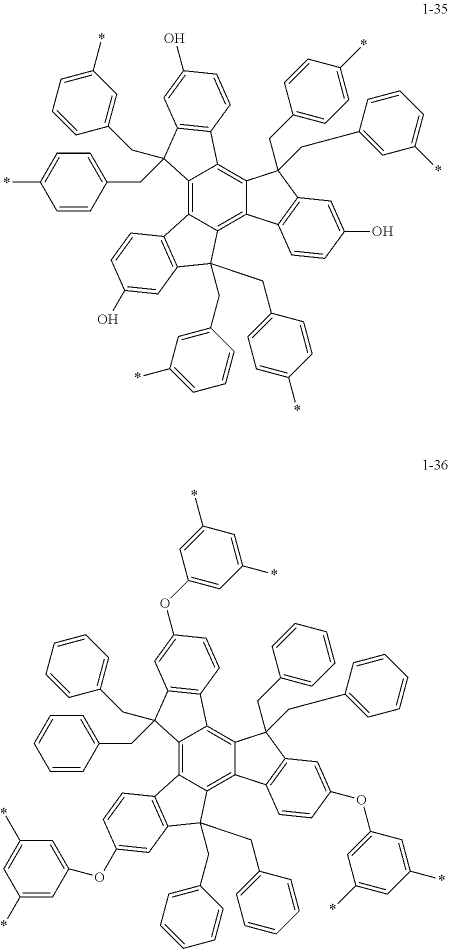 [SMILES: *c1ccc(CC2(Cc3cccc(*)c3)c3cc(O)ccc3-c3c2c2c(c4c3C(Cc3ccc(*)cc3)(Cc3cccc(*)c3)c3cc(O)ccc3-4)C(Cc3ccc(*)cc3)(Cc3cccc(*)c3)c3cc(O)ccc3-2)cc1 ; *c1cc(*)cc(Oc2ccc3c(c2)C(Cc2ccccc2)(Cc2ccccc2)c2c-3c3c(c4c2-c2ccc(Oc5cc(*)cc(*)c5)cc2C4(Cc2ccccc2)Cc2ccccc2)-c2ccc(Oc4cc(*)cc(*)c4)cc2C3(Cc2ccccc2)Cc2ccccc2)c1] 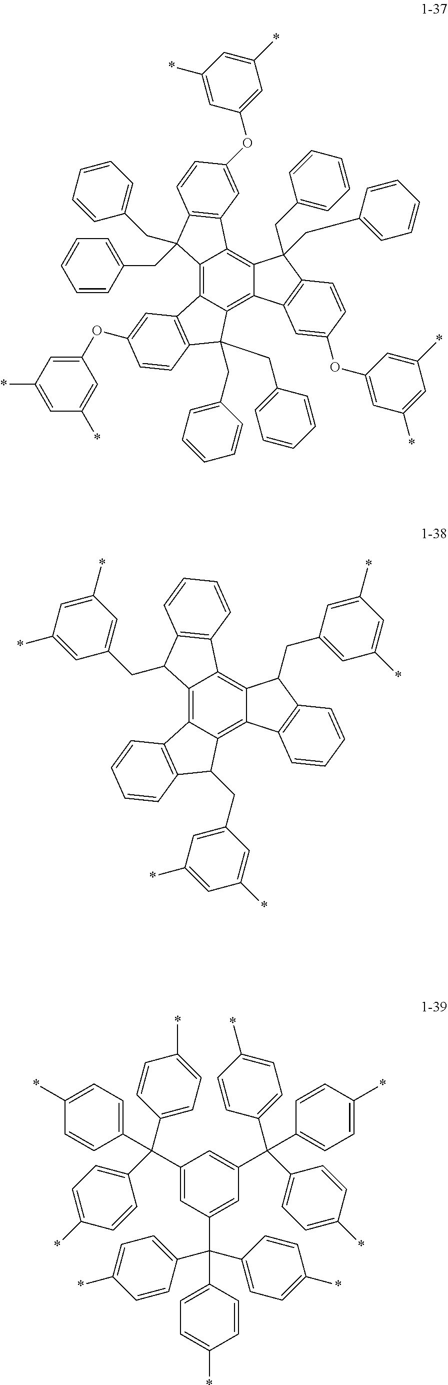 [SMILES: *c1cc(*)cc(Oc2ccc3c(c2)-c2c(c4c(c5c2C(Cc2ccccc2)(Cc2ccccc2)c2ccc(Oc6cc(*)cc(*)c6)cc2-5)C(Cc2ccccc2)(Cc2ccccc2)c2ccc(Oc5cc(*)cc(*)c5)cc2-4)C3(Cc2ccccc2)Cc2ccccc2)c1 ; *c1cc(*)cc(CC2c3ccccc3-c3c2c2c(c4c3C(Cc3cc(*)cc(*)c3)c3ccccc3-4)C(Cc3cc(*)cc(*)c3)c3ccccc3-2)c1 ; *c1ccc(C(c2ccc(*)cc2)(c2ccc(*)cc2)c2cc(C(c3ccc(*)cc3)(c3ccc(*)cc3)c3ccc(*)cc3)cc(C(c3ccc(*)cc3)(c3ccc(*)cc3)c3ccc(*)cc3)c2)cc1] 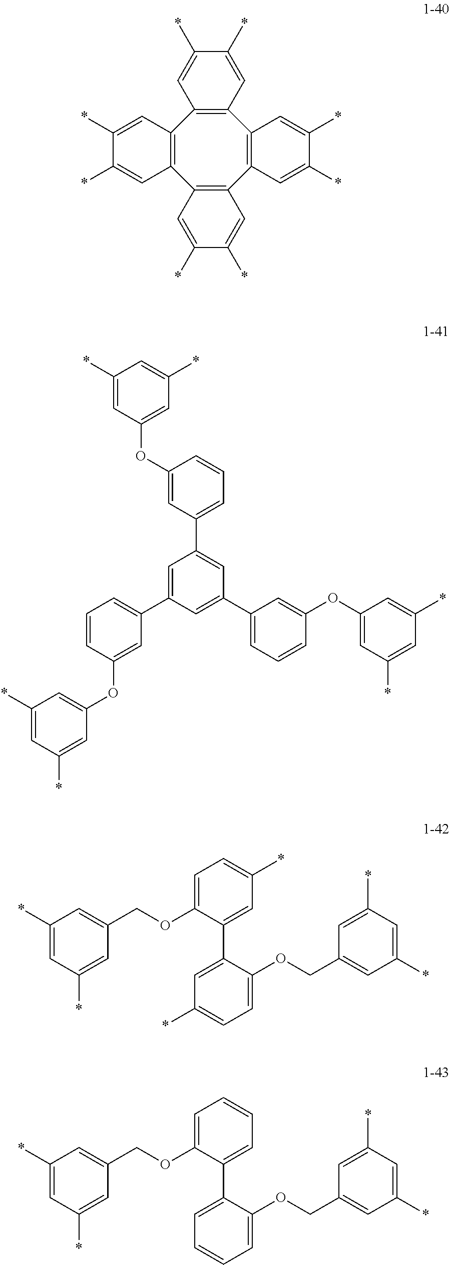 [SMILES: *c1cc2c(cc1*)-c1cc(*)c(*)cc1-c1cc(*)c(*)cc1-c1cc(*)c(*)cc1-2 ; *c1cc(*)cc(Oc2cccc(-c3cc(-c4cccc(Oc5cc(*)cc(*)c5)c4)cc(-c4cccc(Oc5cc(*)cc(*)c5)c4)c3)c2)c1 ; *c1cc(*)cc(COc2ccc(*)cc2-c2cc(*)ccc2OCc2cc(*)cc(*)c2)c1 ; *c1cc(*)cc(COc2ccccc2-c2ccccc2OCc2cc(*)cc(*)c2)c1] 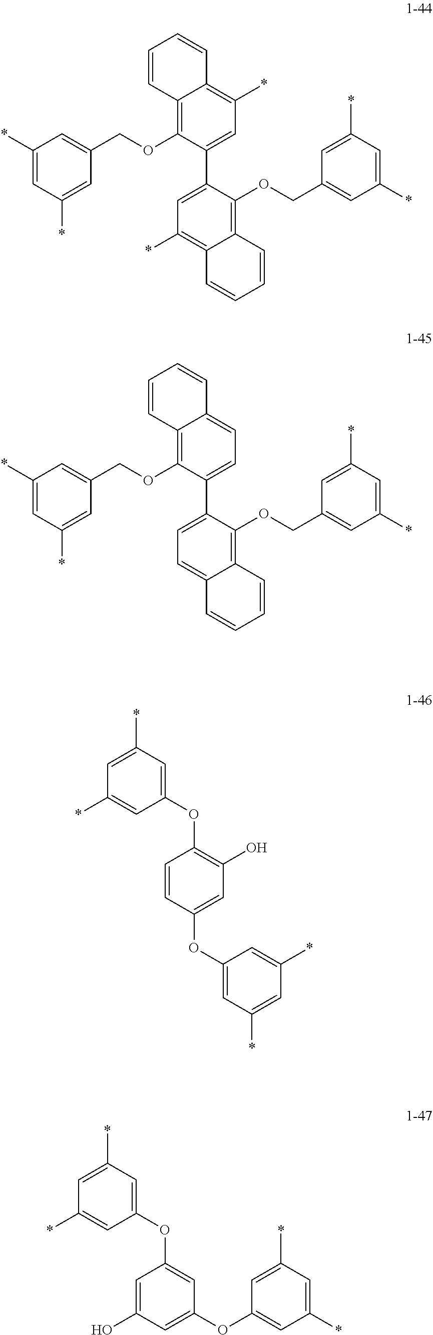 [SMILES: *c1cc(*)cc(COc2c(-c3cc(*)c4ccccc4c3OCc3cc(*)cc(*)c3)cc(*)c3ccccc23)c1 ; *c1cc(*)cc(COc2c(-c3ccc4ccccc4c3OCc3cc(*)cc(*)c3)ccc3ccccc23)c1 ; *c1cc(*)cc(Oc2ccc(Oc3cc(*)cc(*)c3)c(O)c2)c1 ; *c1cc(*)cc(Oc2cc(O)cc(Oc3cc(*)cc(*)c3)c2)c1] 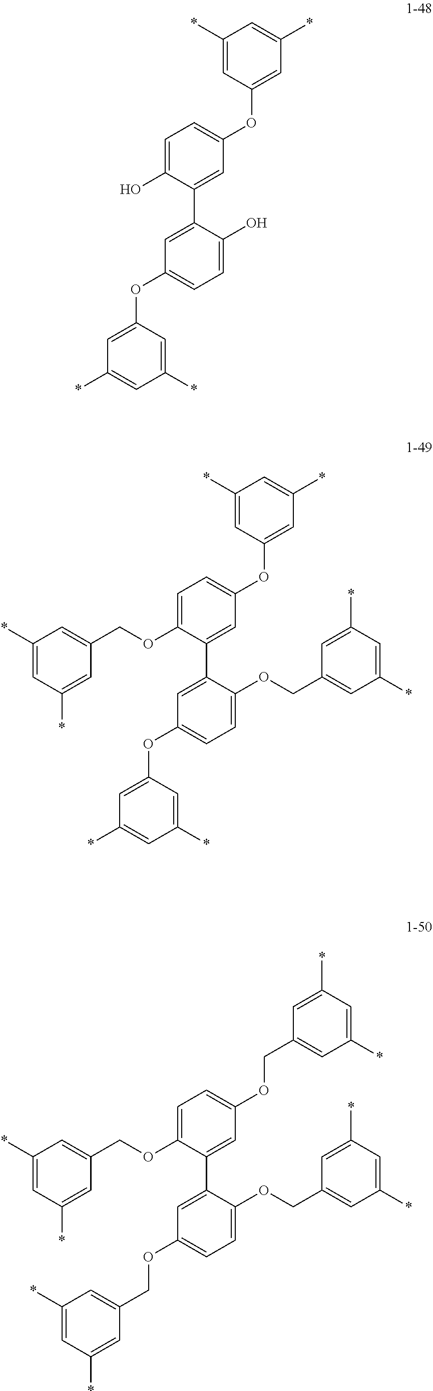 [SMILES: *c1cc(*)cc(Oc2ccc(O)c(-c3cc(Oc4cc(*)cc(*)c4)ccc3O)c2)c1 ; *c1cc(*)cc(COc2ccc(Oc3cc(*)cc(*)c3)cc2-c2cc(Oc3cc(*)cc(*)c3)ccc2OCc2cc(*)cc(*)c2)c1 ; *c1cc(*)cc(COc2ccc(OCc3cc(*)cc(*)c3)c(-c3cc(OCc4cc(*)cc(*)c4)ccc3OCc3cc(*)cc(*)c3)c2)c1] 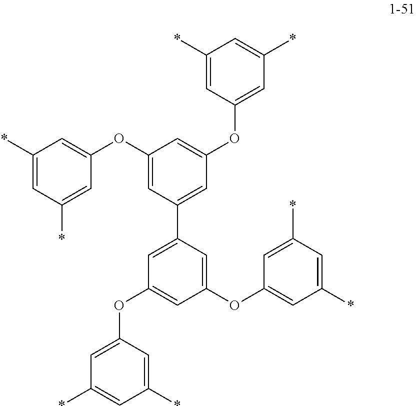 [SMILES: *c1cc(*)cc(Oc2cc(Oc3cc(*)cc(*)c3)cc(-c3cc(Oc4cc(*)cc(*)c4)cc(Oc4cc(*)cc(*)c4)c3)c2)c1]
wherein in Formulae 1-1 to 1-51,
a hydrogen atom is optionally substituted with a substituent, R₁₃, or both the substituent and R₁₃,
* is a binding site with X₁₁.

13. The polycarboxylate compound of claim 12, wherein
X₁₁ is represented by one of Formulae 2-11 to 2-76 below: 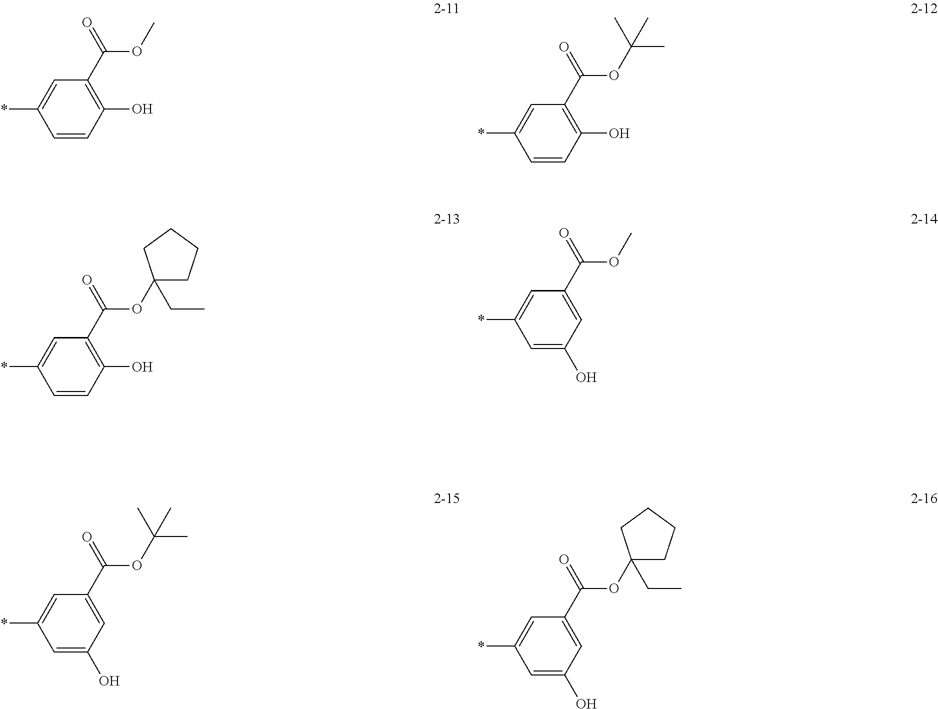 [SMILES: *c1ccc(O)c(C(=O)OC)c1 ; *c1ccc(O)c(C(=O)OC(C)(C)C)c1 ; *c1ccc(O)c(C(=O)OC2(CC)CCCC2)c1 ; *c1cc(O)cc(C(=O)OC)c1 ; *c1cc(O)cc(C(=O)OC(C)(C)C)c1 ; *c1cc(O)cc(C(=O)OC2(CC)CCCC2)c1] 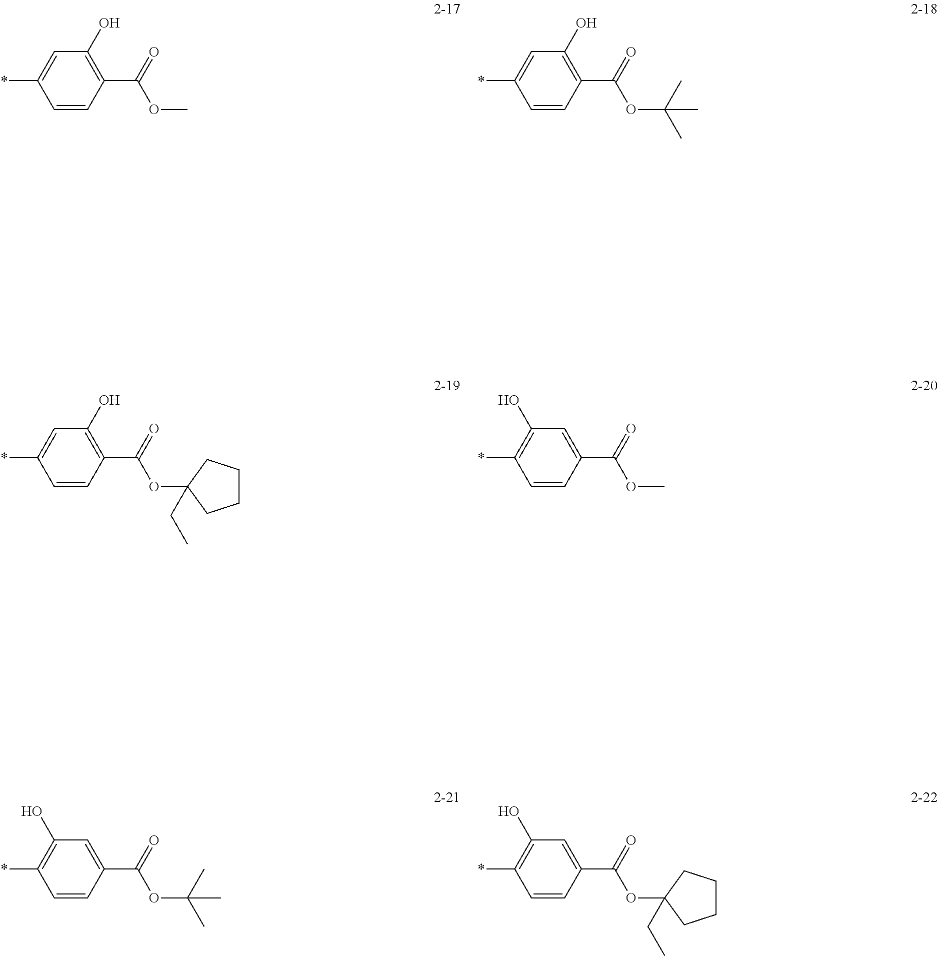 [SMILES: *c1ccc(C(=O)OC)c(O)c1 ; *c1ccc(C(=O)OC(C)(C)C)c(O)c1 ; *c1ccc(C(=O)OC2(CC)CCCC2)c(O)c1 ; *c1ccc(C(=O)OC)cc1O ; *c1ccc(C(=O)OC(C)(C)C)cc1O ; *c1ccc(C(=O)OC2(CC)CCCC2)cc1O] 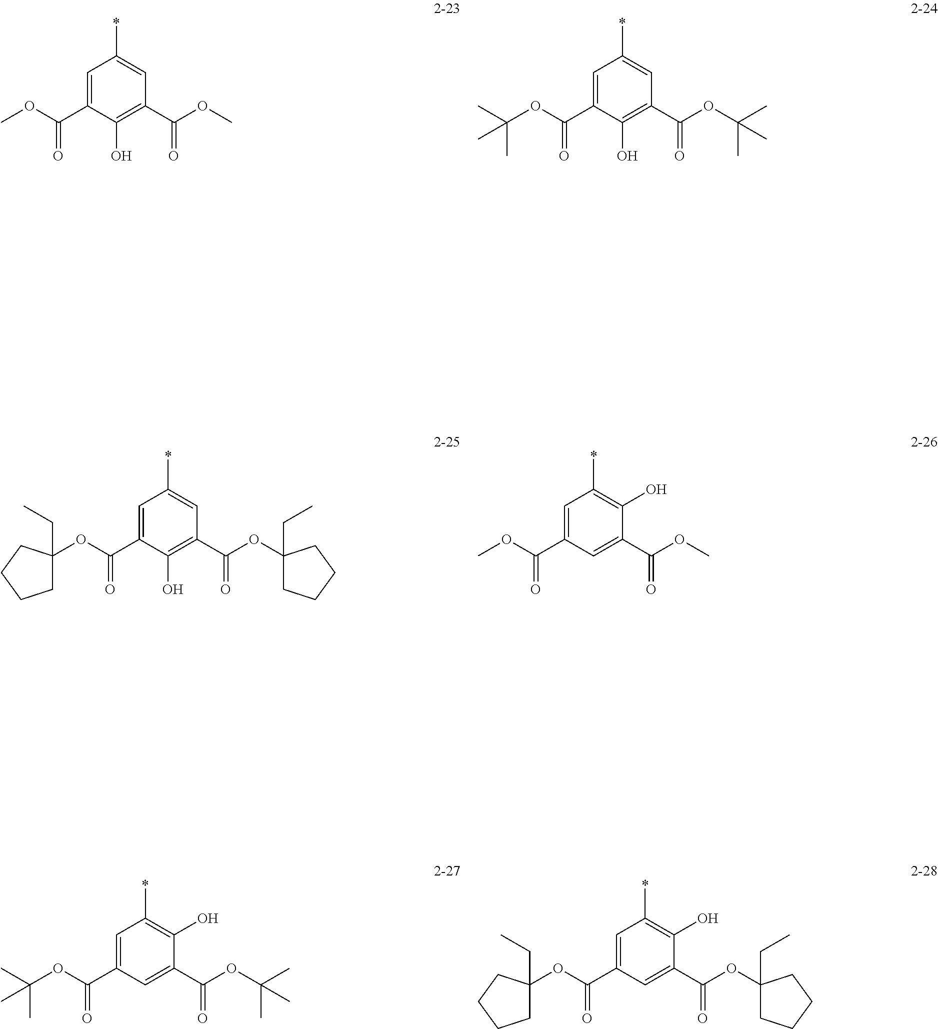 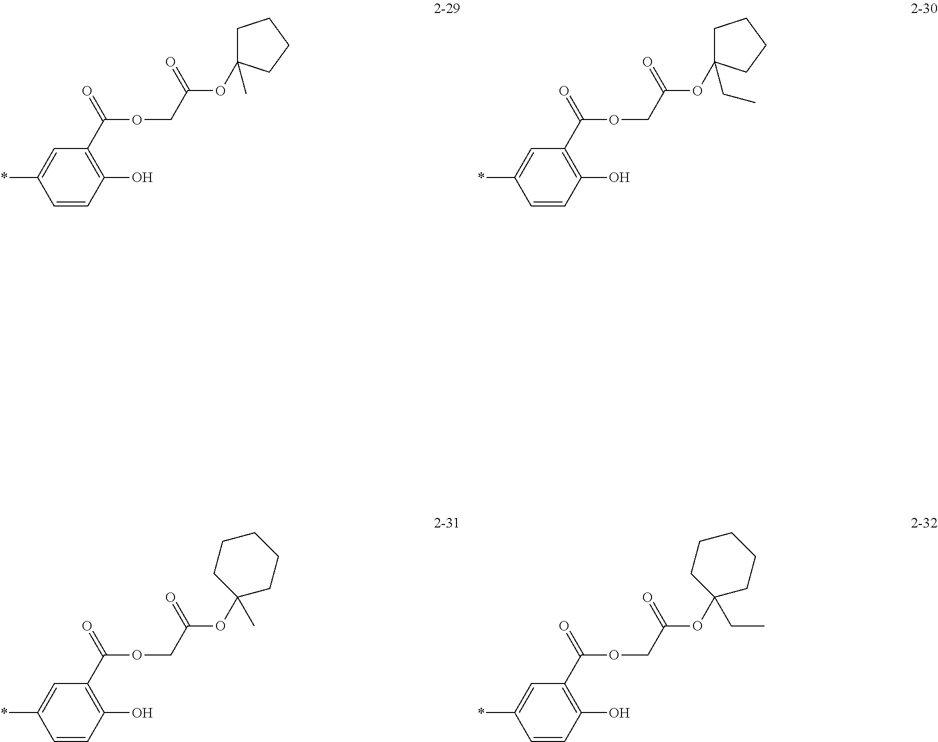 [SMILES: *c1ccc(O)c(C(=O)OCC(=O)OC2(C)CCCC2)c1 ; *c1ccc(O)c(C(=O)OCC(=O)OC2(CC)CCCC2)c1 ; *c1ccc(O)c(C(=O)OCC(=O)OC2(C)CCCCC2)c1 ; *c1ccc(O)c(C(=O)OCC(=O)OC2(CC)CCCCC2)c1] 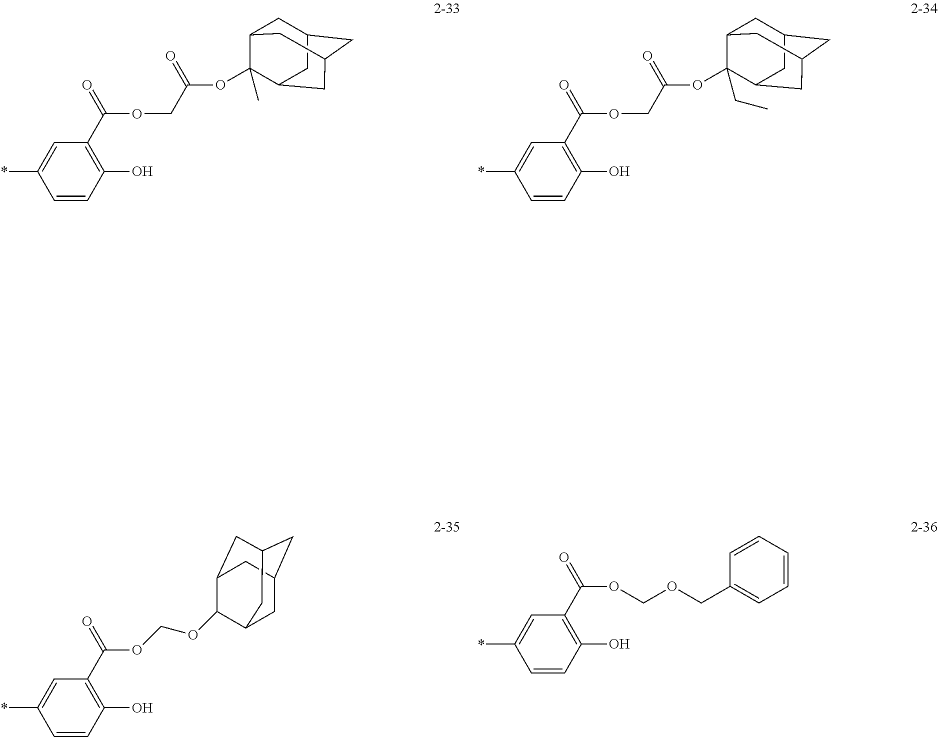 [SMILES: *c1ccc(O)c(C(=O)OCC(=O)OC2(C)C3CC4CC(C3)CC2C4)c1 ; *c1ccc(O)c(C(=O)OCC(=O)OC2(CC)C3CC4CC(C3)CC2C4)c1 ; *c1ccc(O)c(C(=O)OCOC2C3CC4CC(C3)CC2C4)c1 ; *c1ccc(O)c(C(=O)OCOCc2ccccc2)c1] 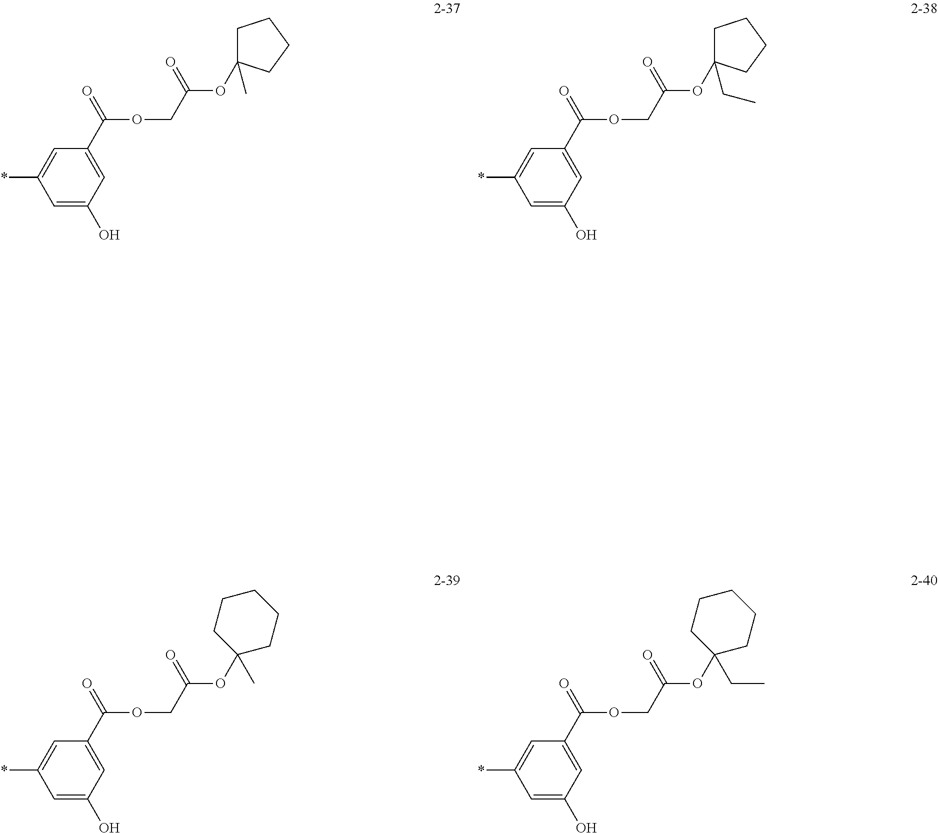 [SMILES: *c1cc(O)cc(C(=O)OCC(=O)OC2(C)CCCC2)c1 ; *c1cc(O)cc(C(=O)OCC(=O)OC2(CC)CCCC2)c1 ; *c1cc(O)cc(C(=O)OCC(=O)OC2(C)CCCCC2)c1 ; *c1cc(O)cc(C(=O)OCC(=O)OC2(CC)CCCCC2)c1] 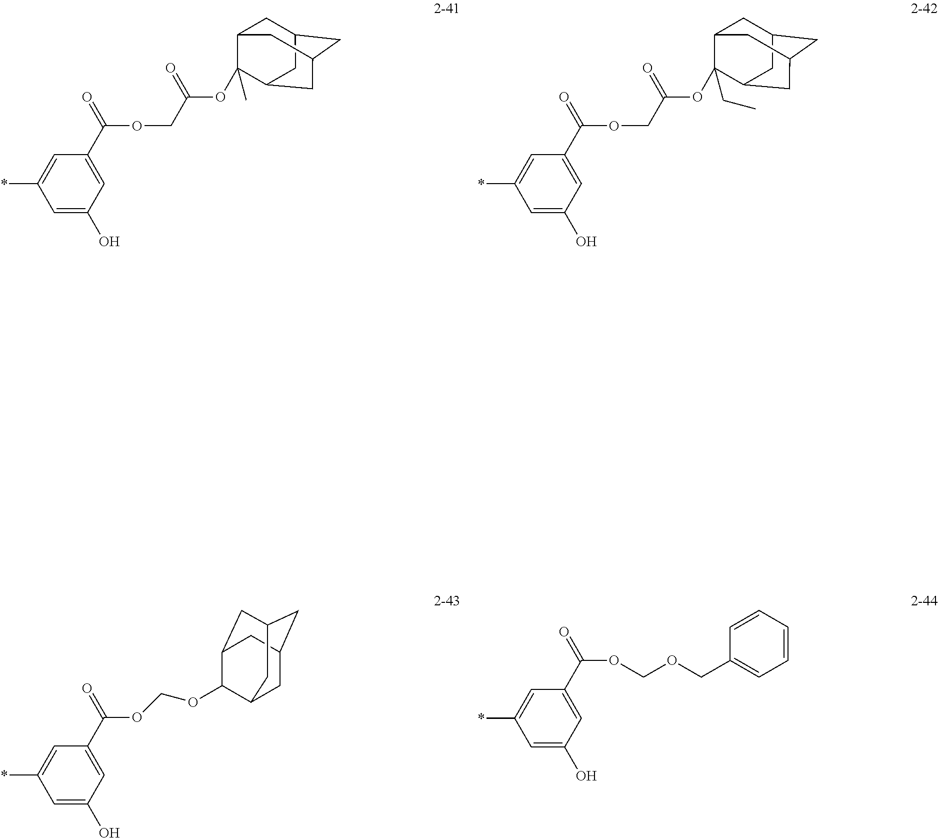 [SMILES: *c1cc(O)cc(C(=O)OCC(=O)OC2(C)C3CC4CC(C3)CC2C4)c1 ; *c1cc(O)cc(C(=O)OCC(=O)OC2(CC)C3CC4CC(C3)CC2C4)c1 ; *c1cc(O)cc(C(=O)OCOC2C3CC4CC(C3)CC2C4)c1 ; *c1cc(O)cc(C(=O)OCOCc2ccccc2)c1] 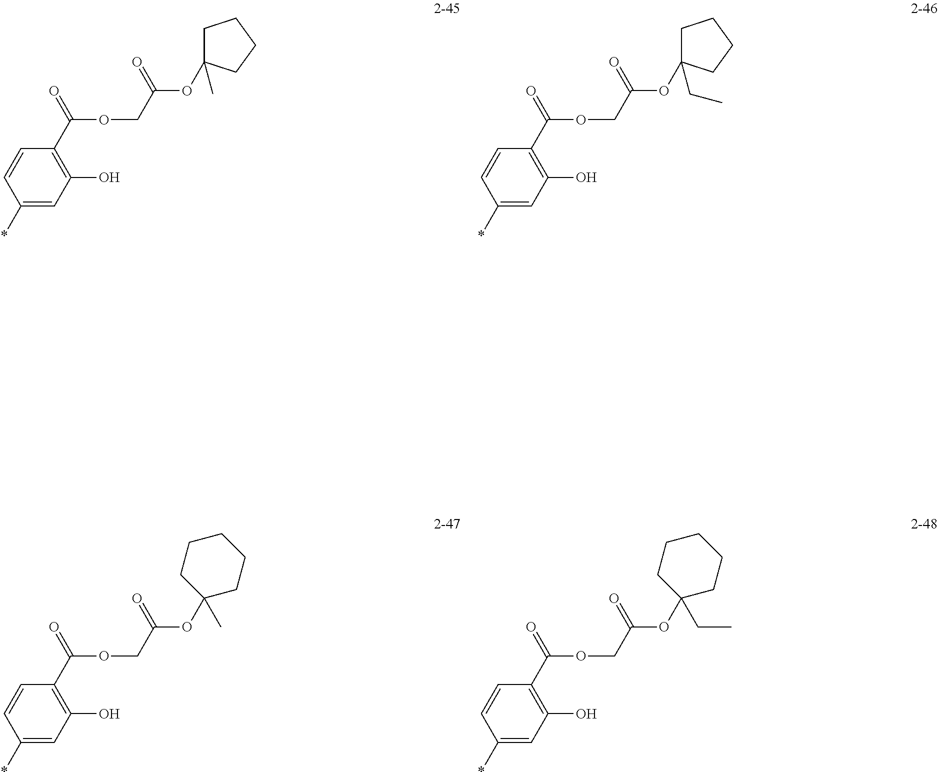 [SMILES: *c1ccc(C(=O)OCC(=O)OC2(C)CCCC2)c(O)c1 ; *c1ccc(C(=O)OCC(=O)OC2(CC)CCCC2)c(O)c1 ; *c1ccc(C(=O)OCC(=O)OC2(C)CCCCC2)c(O)c1 ; *c1ccc(C(=O)OCC(=O)OC2(CC)CCCCC2)c(O)c1] 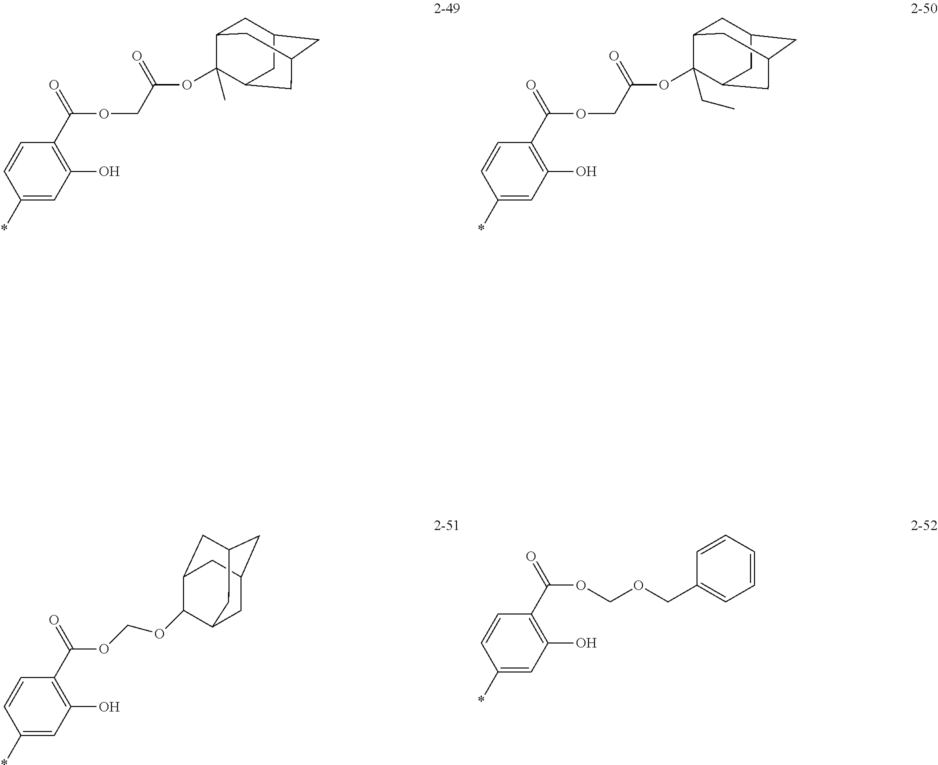 [SMILES: *c1ccc(C(=O)OCC(=O)OC2(C)C3CC4CC(C3)CC2C4)c(O)c1 ; *c1ccc(C(=O)OCC(=O)OC2(CC)C3CC4CC(C3)CC2C4)c(O)c1 ; *c1ccc(C(=O)OCOC2C3CC4CC(C3)CC2C4)c(O)c1 ; *c1ccc(C(=O)OCOCc2ccccc2)c(O)c1] 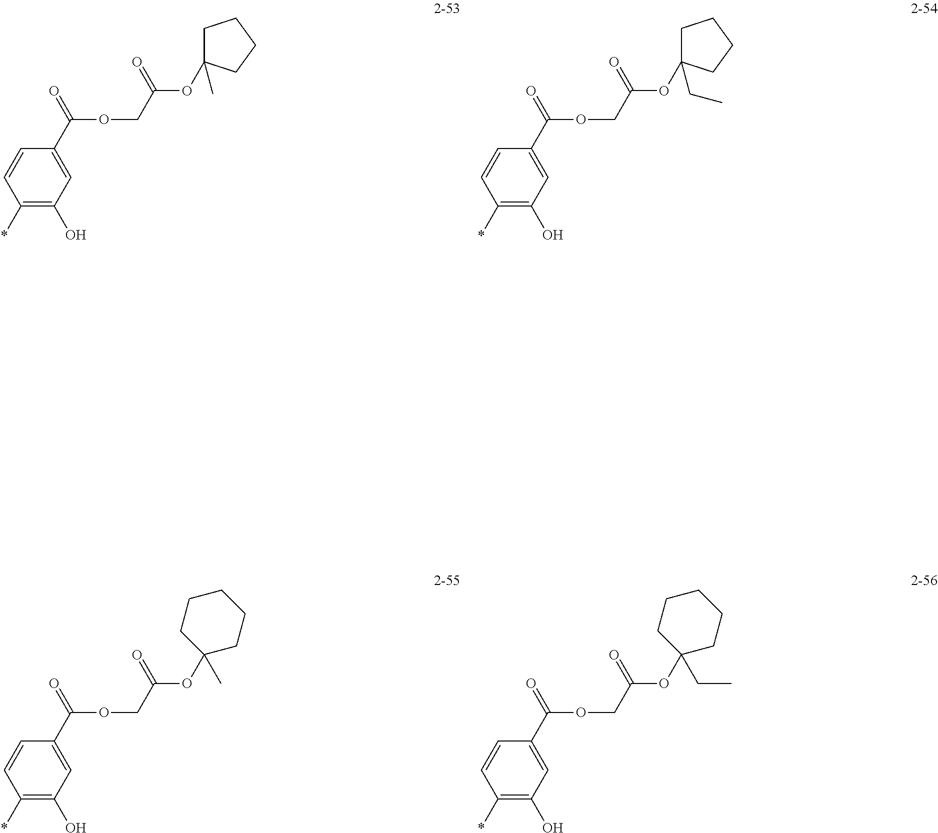 [SMILES: *c1ccc(C(=O)OCC(=O)OC2(C)CCCC2)cc1O ; *c1ccc(C(=O)OCC(=O)OC2(CC)CCCC2)cc1O ; *c1ccc(C(=O)OCC(=O)OC2(C)CCCCC2)cc1O ; *c1ccc(C(=O)OCC(=O)OC2(CC)CCCCC2)cc1O] 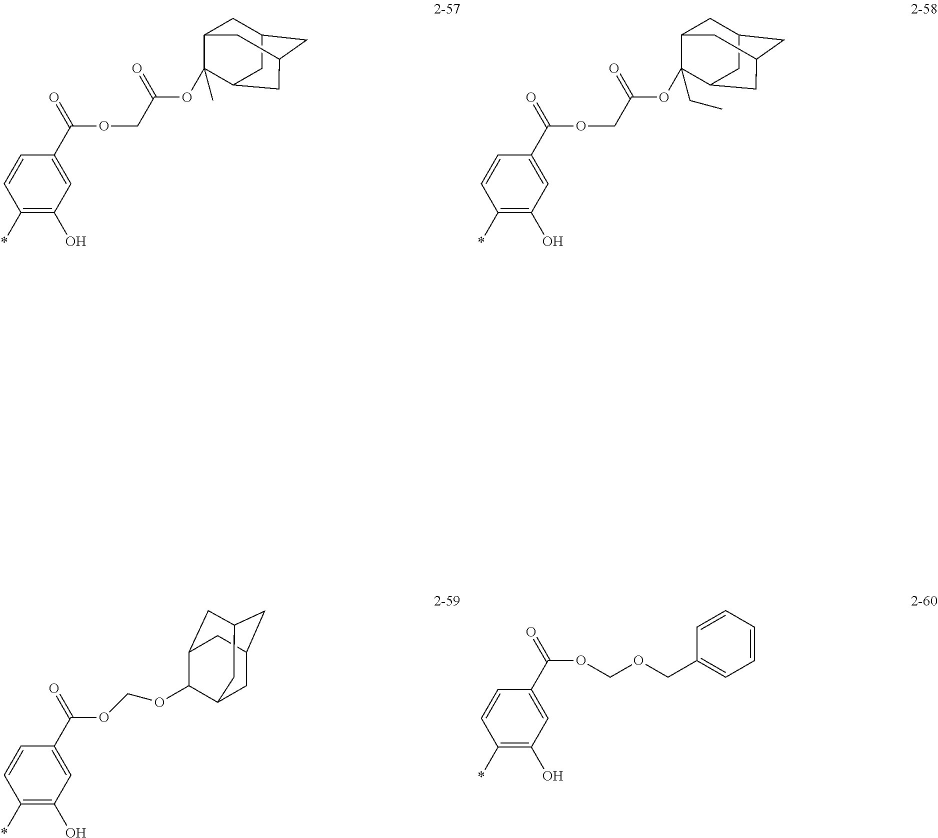 [SMILES: *c1ccc(C(=O)OCC(=O)OC2(C)C3CC4CC(C3)CC2C4)cc1O ; *c1ccc(C(=O)OCC(=O)OC2(CC)C3CC4CC(C3)CC2C4)cc1O ; *c1ccc(C(=O)OCOC2C3CC4CC(C3)CC2C4)cc1O ; *c1ccc(C(=O)OCOCc2ccccc2)cc1O] 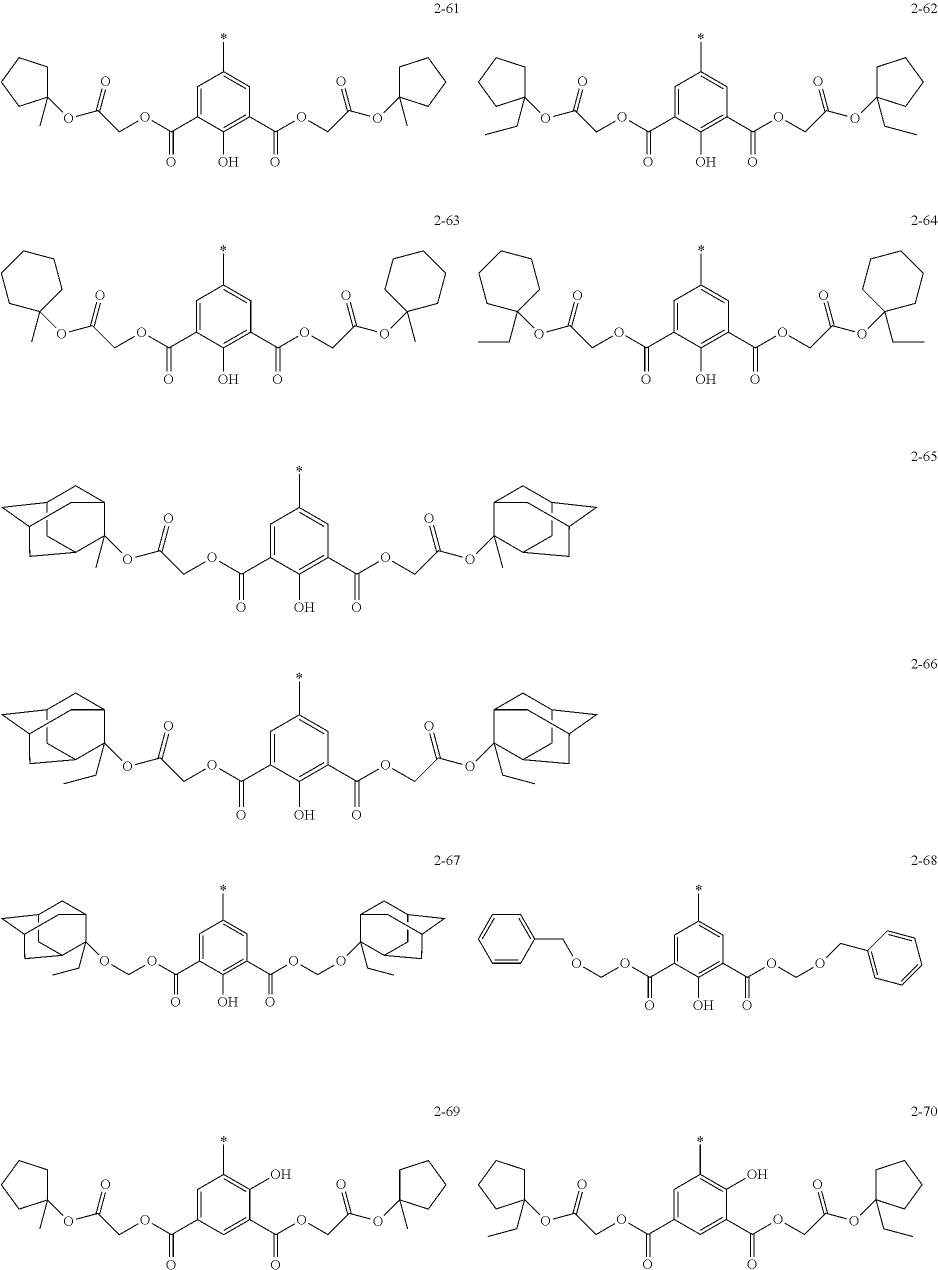 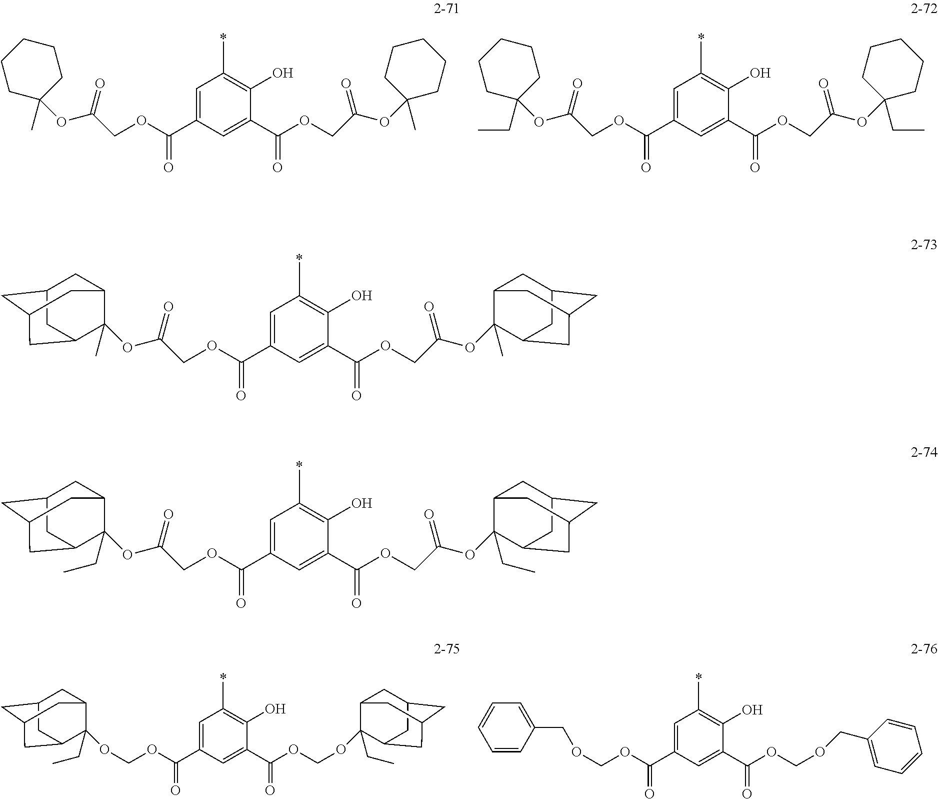
wherein in Formulae 2-11 to 2-76,
* is a binding site with an adjacent atom.

14. The polycarboxylate compound of claim 12, wherein
a hydrogen atom of Formulae 1-1 to 1-51 is unsubstituted.

15. The polycarboxylate compound of claim 1, wherein
the polycarboxylate compound represented by Formula 1 is represented by one of Formulae 1-1 to 1-51 below: 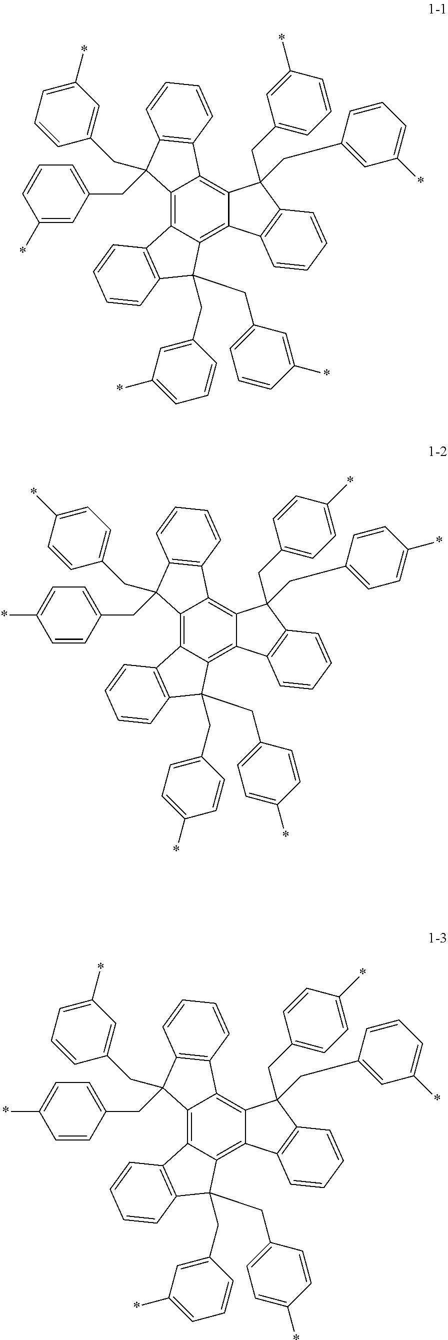 [SMILES: *c1cccc(CC2(Cc3cccc(*)c3)c3ccccc3-c3c2c2c(c4c3C(Cc3cccc(*)c3)(Cc3cccc(*)c3)c3ccccc3-4)C(Cc3cccc(*)c3)(Cc3cccc(*)c3)c3ccccc3-2)c1 ; *c1ccc(CC2(Cc3ccc(*)cc3)c3ccccc3-c3c2c2c(c4c3C(Cc3ccc(*)cc3)(Cc3ccc(*)cc3)c3ccccc3-4)C(Cc3ccc(*)cc3)(Cc3ccc(*)cc3)c3ccccc3-2)cc1 ; *c1ccc(CC2(Cc3cccc(*)c3)c3ccccc3-c3c2c2c(c4c3C(Cc3ccc(*)cc3)(Cc3cccc(*)c3)c3ccccc3-4)C(Cc3ccc(*)cc3)(Cc3cccc(*)c3)c3ccccc3-2)cc1] 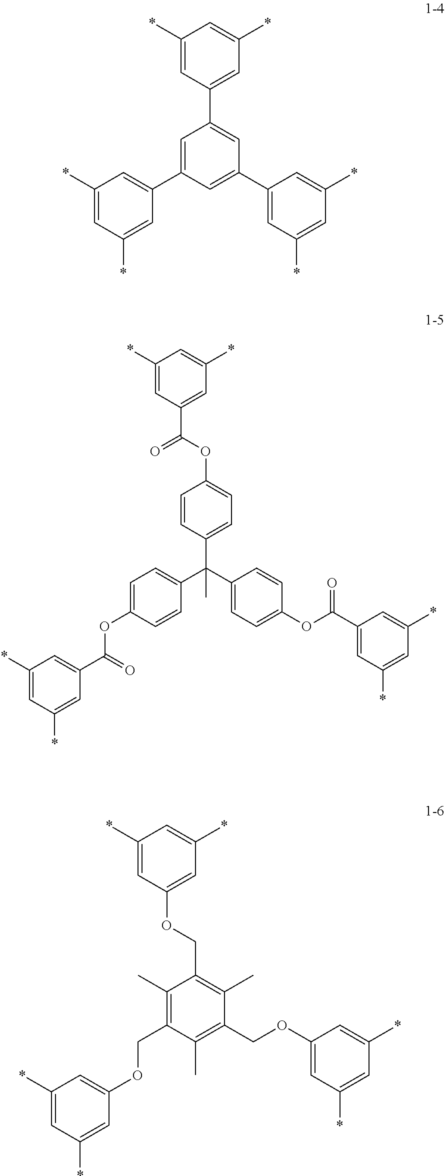 [SMILES: *c1cc(*)cc(-c2cc(-c3cc(*)cc(*)c3)cc(-c3cc(*)cc(*)c3)c2)c1 ; *c1cc(*)cc(C(=O)Oc2ccc(C(C)(c3ccc(OC(=O)c4cc(*)cc(*)c4)cc3)c3ccc(OC(=O)c4cc(*)cc(*)c4)cc3)cc2)c1 ; *c1cc(*)cc(OCc2c(C)c(COc3cc(*)cc(*)c3)c(C)c(COc3cc(*)cc(*)c3)c2C)c1] 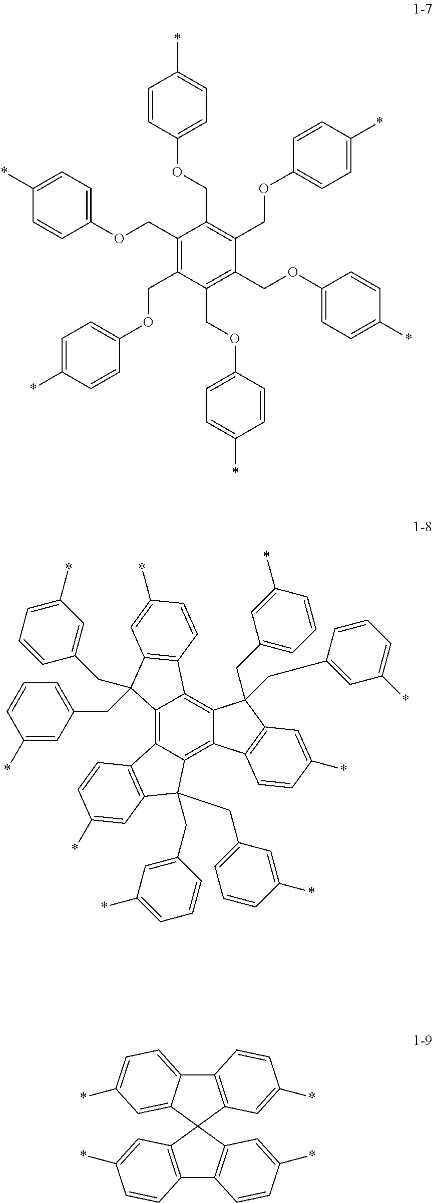 [SMILES: *c1ccc(OCc2c(COc3ccc(*)cc3)c(COc3ccc(*)cc3)c(COc3ccc(*)cc3)c(COc3ccc(*)cc3)c2COc2ccc(*)cc2)cc1 ; *c1cccc(CC2(Cc3cccc(*)c3)c3cc(*)ccc3-c3c2c2c(c4c3C(Cc3cccc(*)c3)(Cc3cccc(*)c3)c3cc(*)ccc3-4)C(Cc3cccc(*)c3)(Cc3cccc(*)c3)c3cc(*)ccc3-2)c1 ; *c1ccc2c(c1)C1(c3cc(*)ccc3-2)c2cc(*)ccc2-c2ccc(*)cc21] 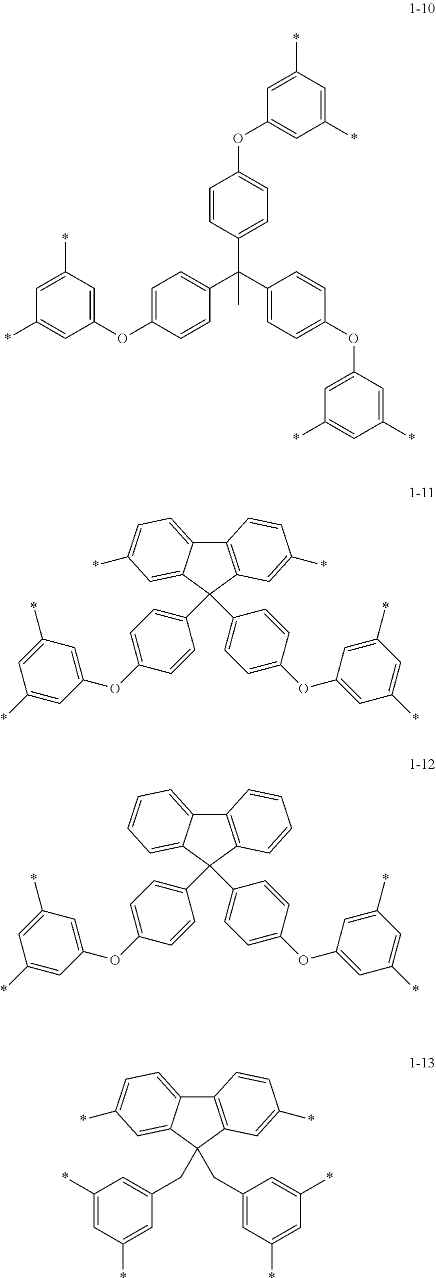 [SMILES: *c1cc(*)cc(Oc2ccc(C(C)(c3ccc(Oc4cc(*)cc(*)c4)cc3)c3ccc(Oc4cc(*)cc(*)c4)cc3)cc2)c1 ; *c1cc(*)cc(Oc2ccc(C3(c4ccc(Oc5cc(*)cc(*)c5)cc4)c4cc(*)ccc4-c4ccc(*)cc43)cc2)c1 ; *c1cc(*)cc(Oc2ccc(C3(c4ccc(Oc5cc(*)cc(*)c5)cc4)c4ccccc4-c4ccccc43)cc2)c1 ; *c1cc(*)cc(CC2(Cc3cc(*)cc(*)c3)c3cc(*)ccc3-c3ccc(*)cc32)c1] 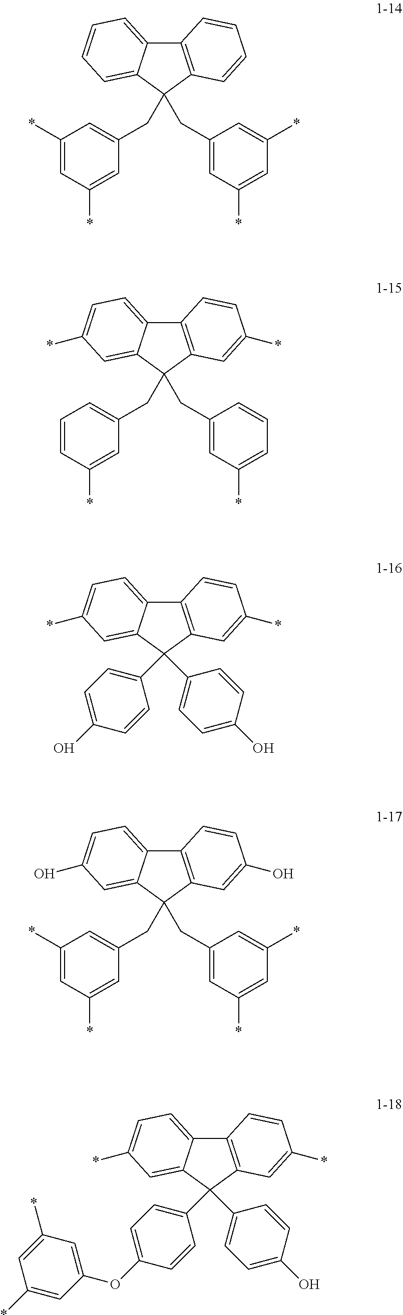 [SMILES: *c1cc(*)cc(CC2(Cc3cc(*)cc(*)c3)c3ccccc3-c3ccccc32)c1 ; *c1cccc(CC2(Cc3cccc(*)c3)c3cc(*)ccc3-c3ccc(*)cc32)c1 ; *c1ccc2c(c1)C(c1ccc(O)cc1)(c1ccc(O)cc1)c1cc(*)ccc1-2 ; *c1cc(*)cc(CC2(Cc3cc(*)cc(*)c3)c3cc(O)ccc3-c3ccc(O)cc32)c1 ; *c1cc(*)cc(Oc2ccc(C3(c4ccc(O)cc4)c4cc(*)ccc4-c4ccc(*)cc43)cc2)c1] 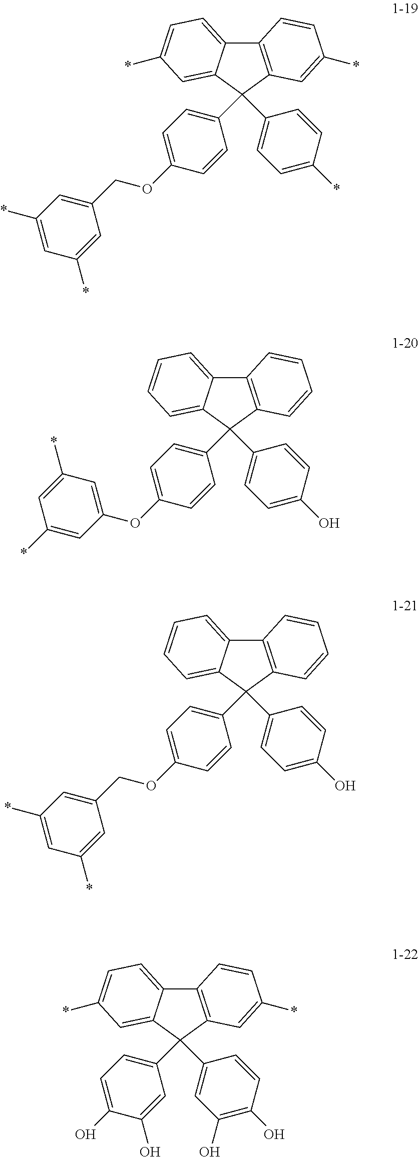 [SMILES: *c1ccc(C2(c3ccc(OCc4cc(*)cc(*)c4)cc3)c3cc(*)ccc3-c3ccc(*)cc32)cc1 ; *c1cc(*)cc(Oc2ccc(C3(c4ccc(O)cc4)c4ccccc4-c4ccccc43)cc2)c1 ; *c1cc(*)cc(COc2ccc(C3(c4ccc(O)cc4)c4ccccc4-c4ccccc43)cc2)c1 ; *c1ccc2c(c1)C(c1ccc(O)c(O)c1)(c1ccc(O)c(O)c1)c1cc(*)ccc1-2] 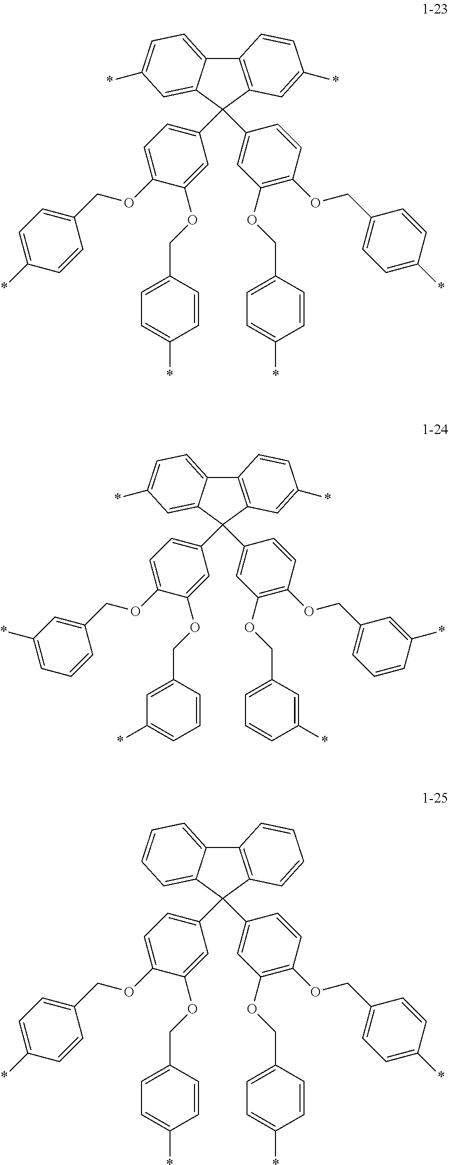 [SMILES: *c1ccc(COc2ccc(C3(c4ccc(OCc5ccc(*)cc5)c(OCc5ccc(*)cc5)c4)c4cc(*)ccc4-c4ccc(*)cc43)cc2OCc2ccc(*)cc2)cc1 ; *c1cccc(COc2ccc(C3(c4ccc(OCc5cccc(*)c5)c(OCc5cccc(*)c5)c4)c4cc(*)ccc4-c4ccc(*)cc43)cc2OCc2cccc(*)c2)c1 ; *c1ccc(COc2ccc(C3(c4ccc(OCc5ccc(*)cc5)c(OCc5ccc(*)cc5)c4)c4ccccc4-c4ccccc43)cc2OCc2ccc(*)cc2)cc1] 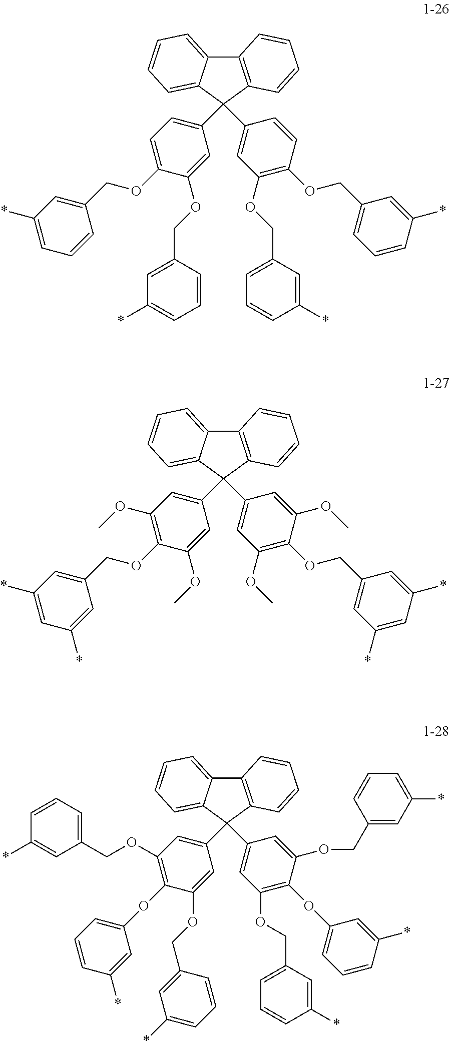 [SMILES: *c1cccc(COc2ccc(C3(c4ccc(OCc5cccc(*)c5)c(OCc5cccc(*)c5)c4)c4ccccc4-c4ccccc43)cc2OCc2cccc(*)c2)c1 ; *c1cc(*)cc(COc2c(OC)cc(C3(c4cc(OC)c(OCc5cc(*)cc(*)c5)c(OC)c4)c4ccccc4-c4ccccc43)cc2OC)c1 ; *c1cccc(COc2cc(C3(c4cc(OCc5cccc(*)c5)c(Oc5cccc(*)c5)c(OCc5cccc(*)c5)c4)c4ccccc4-c4ccccc43)cc(OCc3cccc(*)c3)c2Oc2cccc(*)c2)c1] 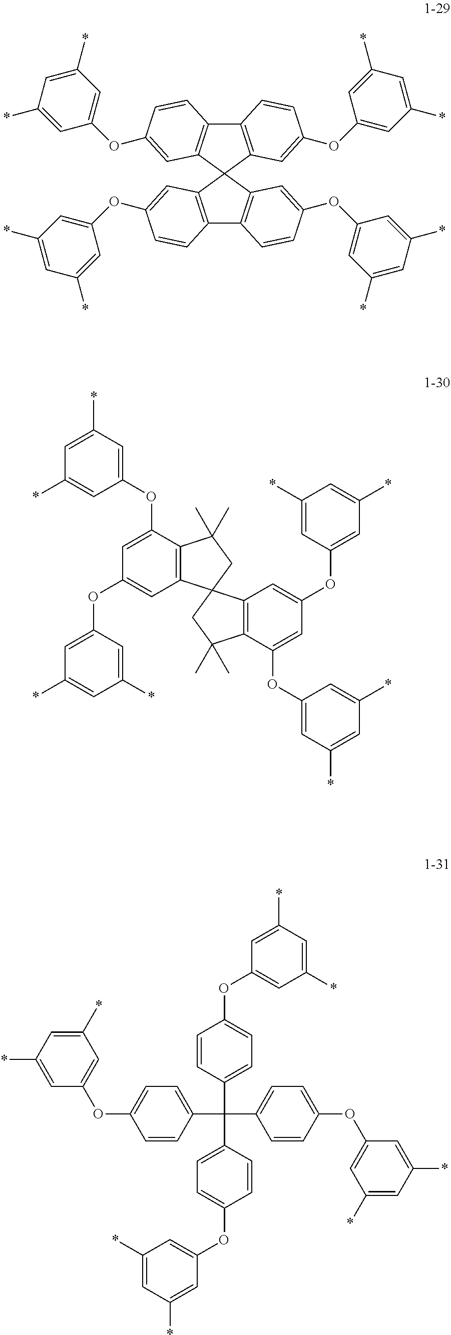 [SMILES: *c1cc(*)cc(Oc2ccc3c(c2)C2(c4cc(Oc5cc(*)cc(*)c5)ccc4-3)c3cc(Oc4cc(*)cc(*)c4)ccc3-c3ccc(Oc4cc(*)cc(*)c4)cc32)c1 ; *c1cc(*)cc(Oc2cc(Oc3cc(*)cc(*)c3)c3c(c2)C2(CC3(C)C)CC(C)(C)c3c(Oc4cc(*)cc(*)c4)cc(Oc4cc(*)cc(*)c4)cc32)c1 ; *c1cc(*)cc(Oc2ccc(C(c3ccc(Oc4cc(*)cc(*)c4)cc3)(c3ccc(Oc4cc(*)cc(*)c4)cc3)c3ccc(Oc4cc(*)cc(*)c4)cc3)cc2)c1] 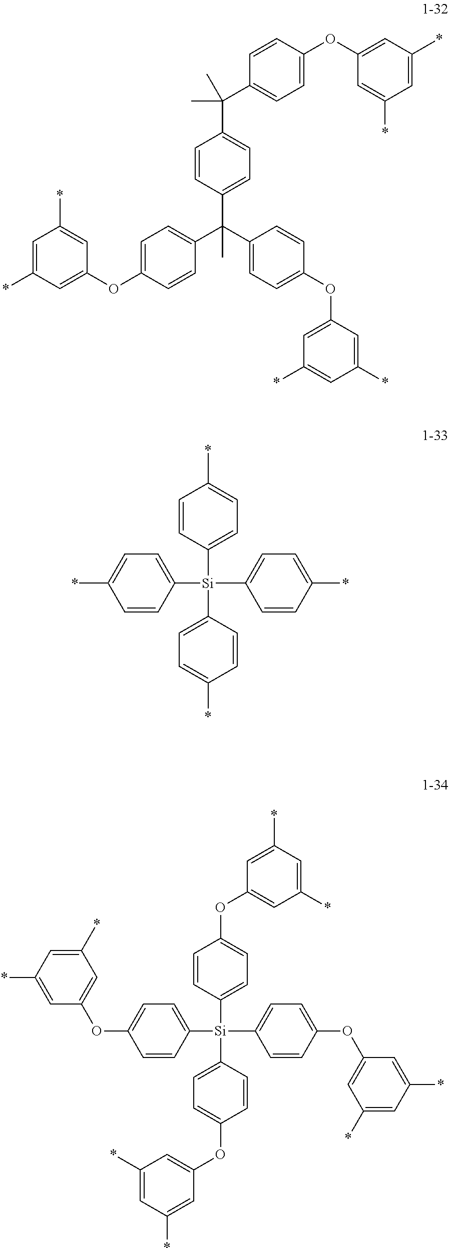 [SMILES: *c1cc(*)cc(Oc2ccc(C(C)(C)c3ccc(C(C)(c4ccc(Oc5cc(*)cc(*)c5)cc4)c4ccc(Oc5cc(*)cc(*)c5)cc4)cc3)cc2)c1 ; *c1ccc([Si](c2ccc(*)cc2)(c2ccc(*)cc2)c2ccc(*)cc2)cc1 ; *c1cc(*)cc(Oc2ccc([Si](c3ccc(Oc4cc(*)cc(*)c4)cc3)(c3ccc(Oc4cc(*)cc(*)c4)cc3)c3ccc(Oc4cc(*)cc(*)c4)cc3)cc2)c1] 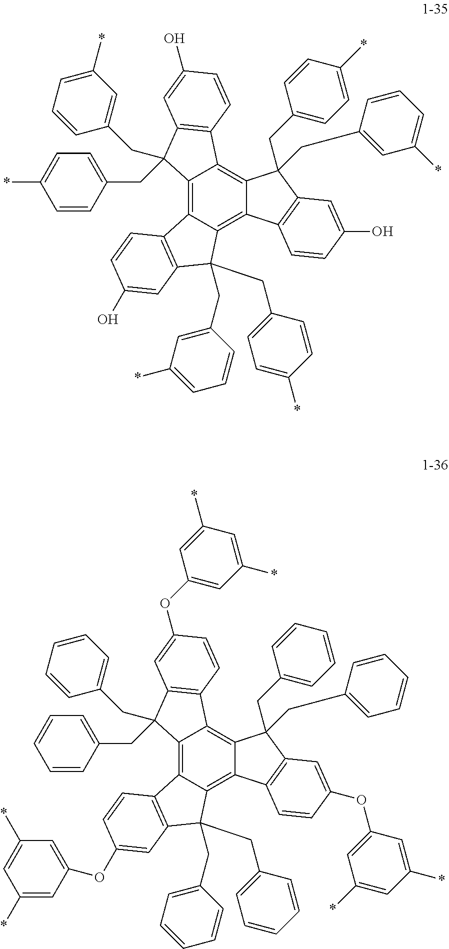 [SMILES: *c1ccc(CC2(Cc3cccc(*)c3)c3cc(O)ccc3-c3c2c2c(c4c3C(Cc3ccc(*)cc3)(Cc3cccc(*)c3)c3cc(O)ccc3-4)C(Cc3ccc(*)cc3)(Cc3cccc(*)c3)c3cc(O)ccc3-2)cc1 ; *c1cc(*)cc(Oc2ccc3c(c2)C(Cc2ccccc2)(Cc2ccccc2)c2c-3c3c(c4c2-c2ccc(Oc5cc(*)cc(*)c5)cc2C4(Cc2ccccc2)Cc2ccccc2)-c2ccc(Oc4cc(*)cc(*)c4)cc2C3(Cc2ccccc2)Cc2ccccc2)c1] 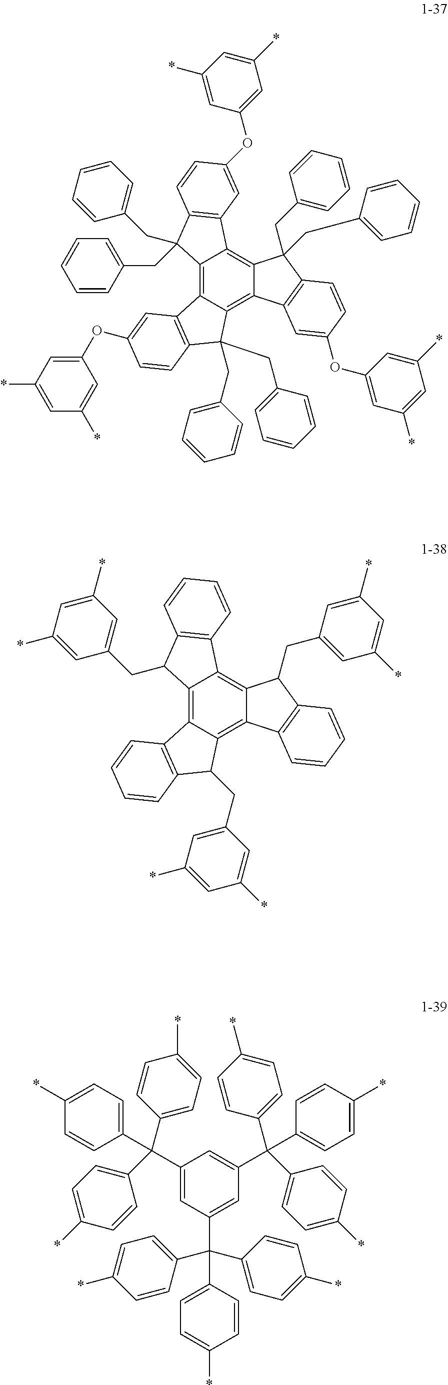 [SMILES: *c1cc(*)cc(Oc2ccc3c(c2)-c2c(c4c(c5c2C(Cc2ccccc2)(Cc2ccccc2)c2ccc(Oc6cc(*)cc(*)c6)cc2-5)C(Cc2ccccc2)(Cc2ccccc2)c2ccc(Oc5cc(*)cc(*)c5)cc2-4)C3(Cc2ccccc2)Cc2ccccc2)c1 ; *c1cc(*)cc(CC2c3ccccc3-c3c2c2c(c4c3C(Cc3cc(*)cc(*)c3)c3ccccc3-4)C(Cc3cc(*)cc(*)c3)c3ccccc3-2)c1 ; *c1ccc(C(c2ccc(*)cc2)(c2ccc(*)cc2)c2cc(C(c3ccc(*)cc3)(c3ccc(*)cc3)c3ccc(*)cc3)cc(C(c3ccc(*)cc3)(c3ccc(*)cc3)c3ccc(*)cc3)c2)cc1] 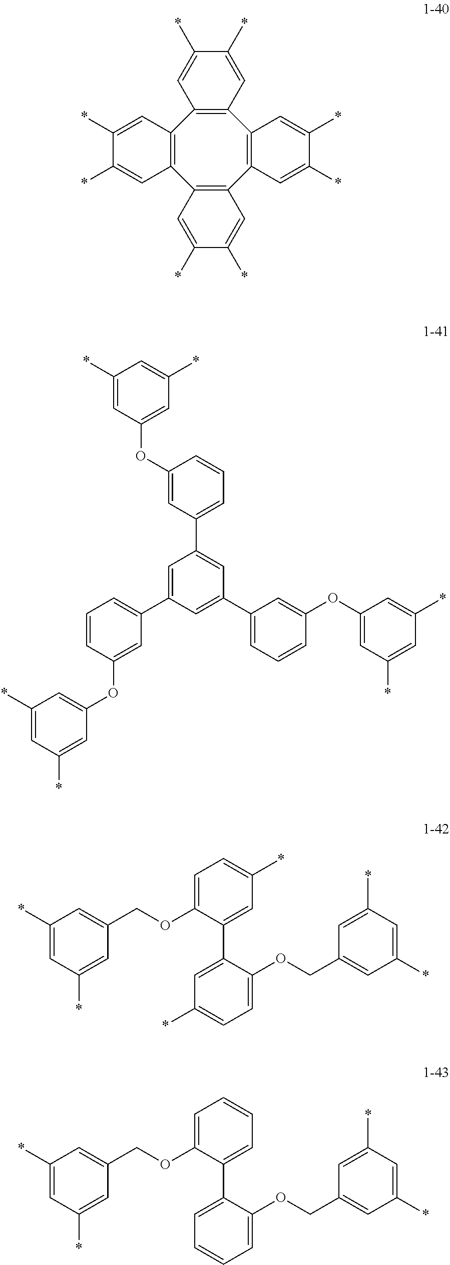 [SMILES: *c1cc2c(cc1*)-c1cc(*)c(*)cc1-c1cc(*)c(*)cc1-c1cc(*)c(*)cc1-2 ; *c1cc(*)cc(Oc2cccc(-c3cc(-c4cccc(Oc5cc(*)cc(*)c5)c4)cc(-c4cccc(Oc5cc(*)cc(*)c5)c4)c3)c2)c1 ; *c1cc(*)cc(COc2ccc(*)cc2-c2cc(*)ccc2OCc2cc(*)cc(*)c2)c1 ; *c1cc(*)cc(COc2ccccc2-c2ccccc2OCc2cc(*)cc(*)c2)c1] 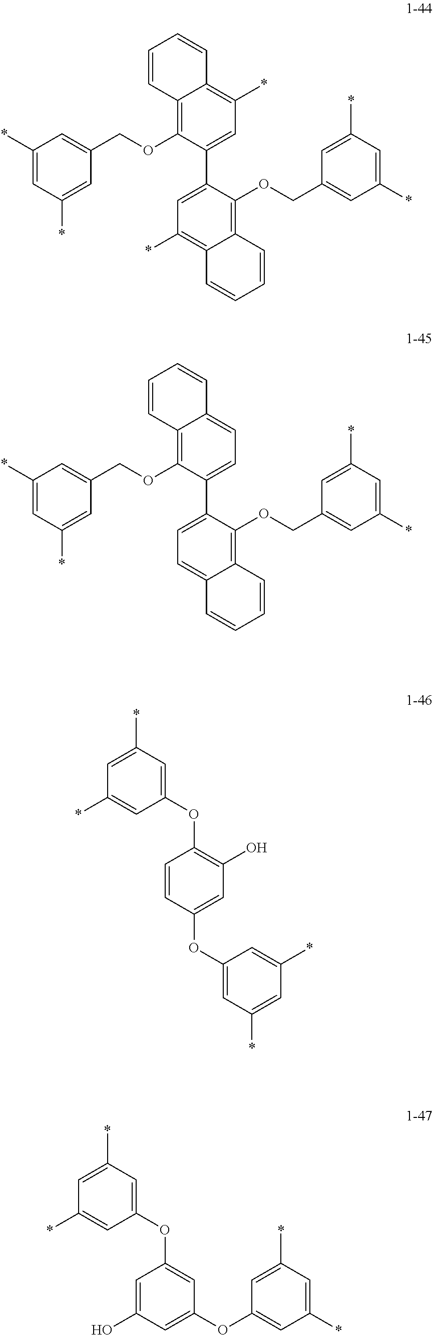 [SMILES: *c1cc(*)cc(COc2c(-c3cc(*)c4ccccc4c3OCc3cc(*)cc(*)c3)cc(*)c3ccccc23)c1 ; *c1cc(*)cc(COc2c(-c3ccc4ccccc4c3OCc3cc(*)cc(*)c3)ccc3ccccc23)c1 ; *c1cc(*)cc(Oc2ccc(Oc3cc(*)cc(*)c3)c(O)c2)c1 ; *c1cc(*)cc(Oc2cc(O)cc(Oc3cc(*)cc(*)c3)c2)c1] 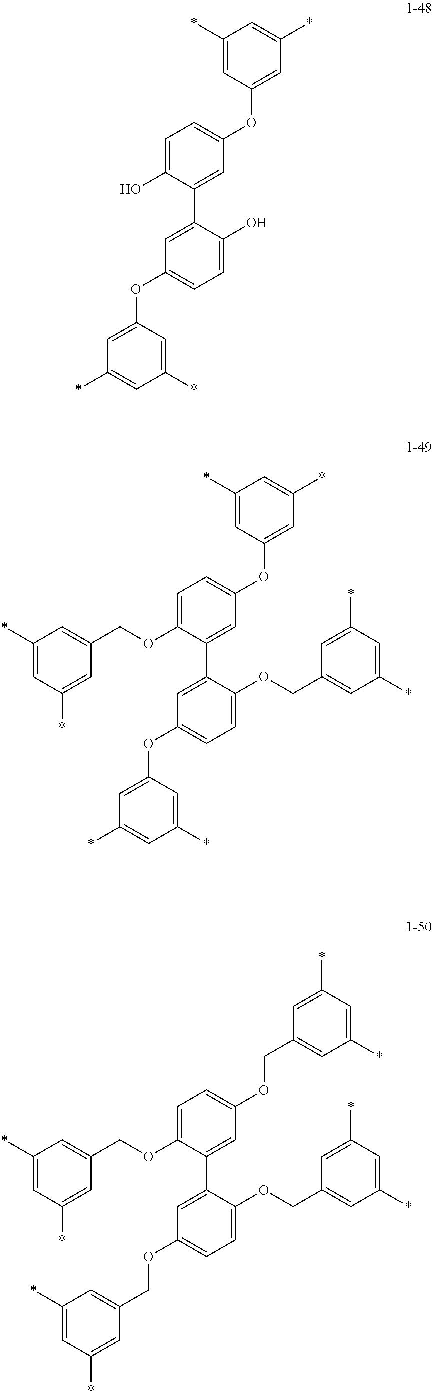 [SMILES: *c1cc(*)cc(Oc2ccc(O)c(-c3cc(Oc4cc(*)cc(*)c4)ccc3O)c2)c1 ; *c1cc(*)cc(COc2ccc(Oc3cc(*)cc(*)c3)cc2-c2cc(Oc3cc(*)cc(*)c3)ccc2OCc2cc(*)cc(*)c2)c1 ; *c1cc(*)cc(COc2ccc(OCc3cc(*)cc(*)c3)c(-c3cc(OCc4cc(*)cc(*)c4)ccc3OCc3cc(*)cc(*)c3)c2)c1] 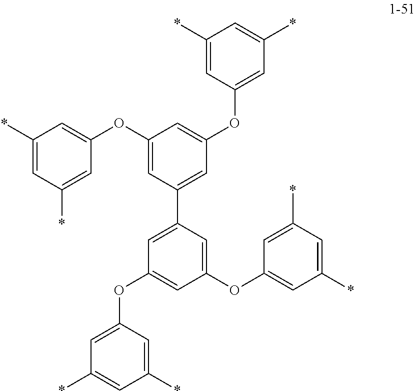 [SMILES: *c1cc(*)cc(Oc2cc(Oc3cc(*)cc(*)c3)cc(-c3cc(Oc4cc(*)cc(*)c4)cc(Oc4cc(*)cc(*)c4)c3)c2)c1]
wherein in Formulae 1-1 to 1-51,
* is a binding site with X₁₁,
wherein X₁₁ is represented by one of Formulae 2-11 to 2-28 below, 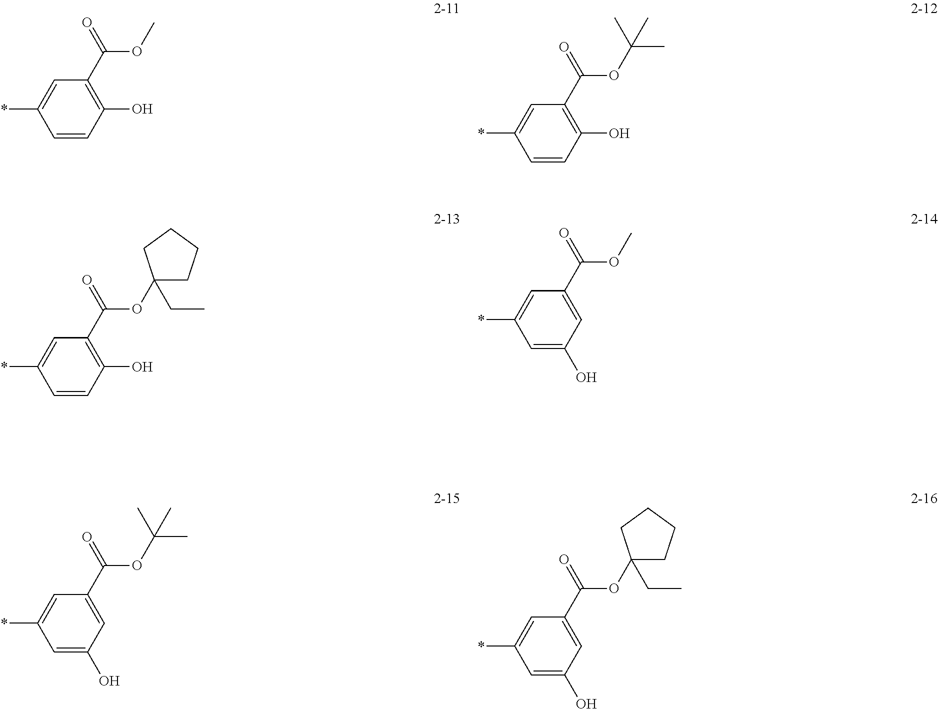 [SMILES: *c1ccc(O)c(C(=O)OC)c1 ; *c1ccc(O)c(C(=O)OC(C)(C)C)c1 ; *c1ccc(O)c(C(=O)OC2(CC)CCCC2)c1 ; *c1cc(O)cc(C(=O)OC)c1 ; *c1cc(O)cc(C(=O)OC(C)(C)C)c1 ; *c1cc(O)cc(C(=O)OC2(CC)CCCC2)c1] 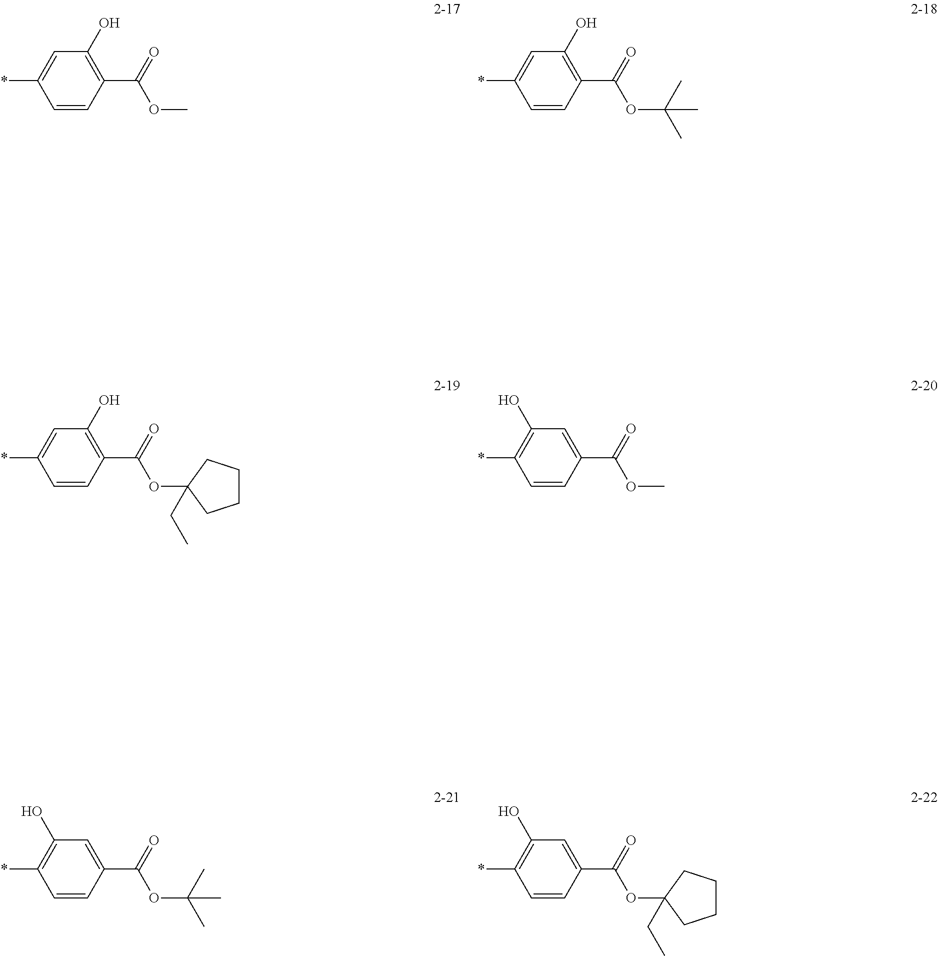 [SMILES: *c1ccc(C(=O)OC)c(O)c1 ; *c1ccc(C(=O)OC(C)(C)C)c(O)c1 ; *c1ccc(C(=O)OC2(CC)CCCC2)c(O)c1 ; *c1ccc(C(=O)OC)cc1O ; *c1ccc(C(=O)OC(C)(C)C)cc1O ; *c1ccc(C(=O)OC2(CC)CCCC2)cc1O] 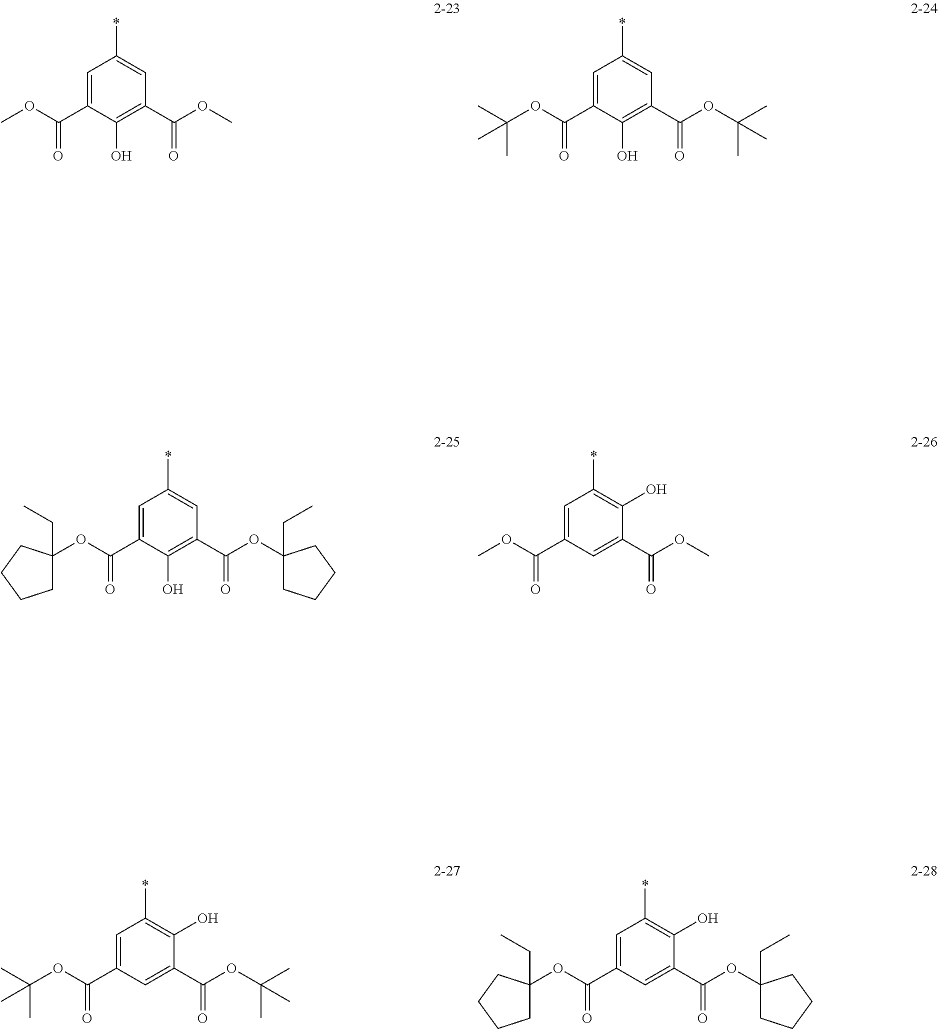
wherein in Formulae 2-11 to 2-28,
* is a binding site with an adjacent atom.

16. The polycarboxylate compound of claim 1, wherein
the polycarboxylate compound represented by Formula 1 is selected from compounds of Group I below:

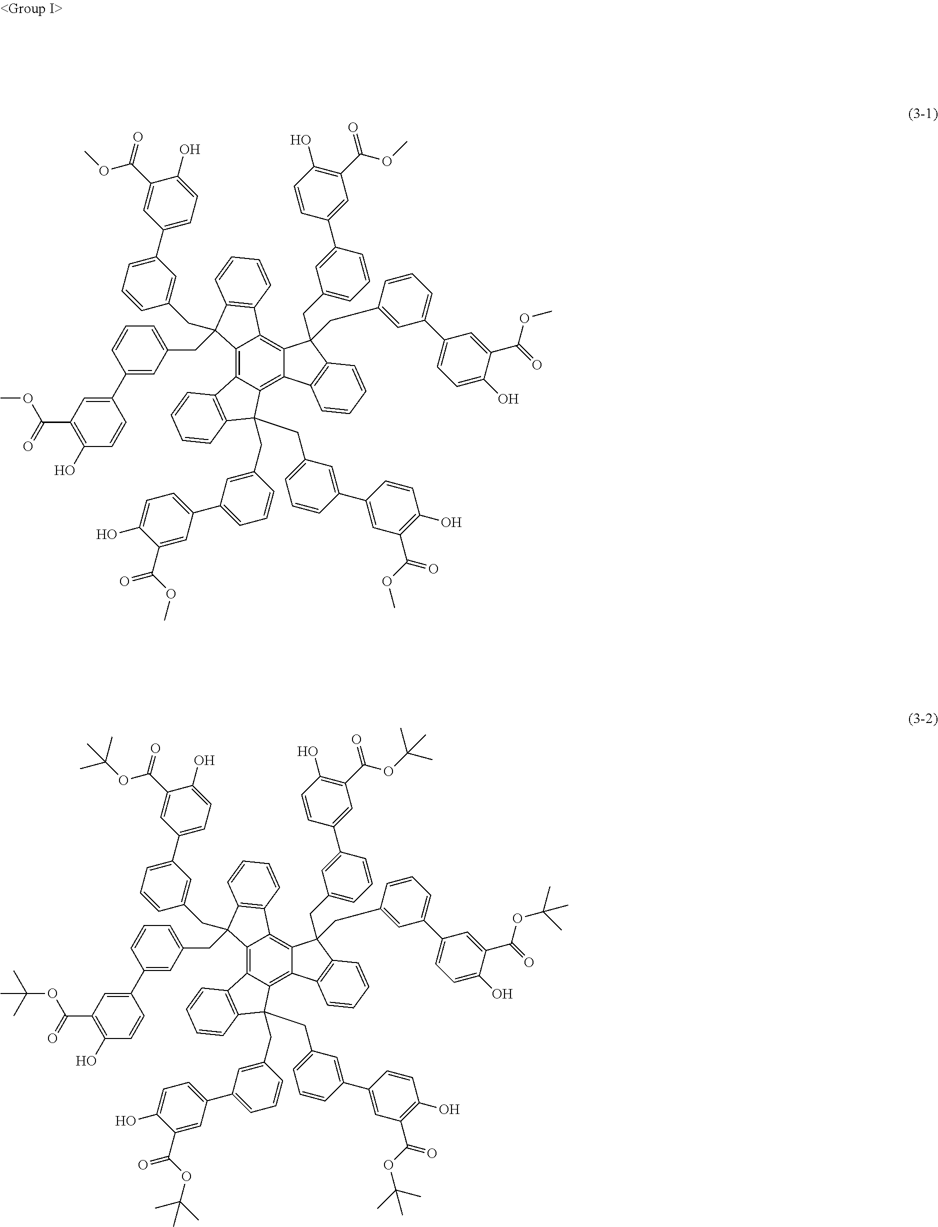

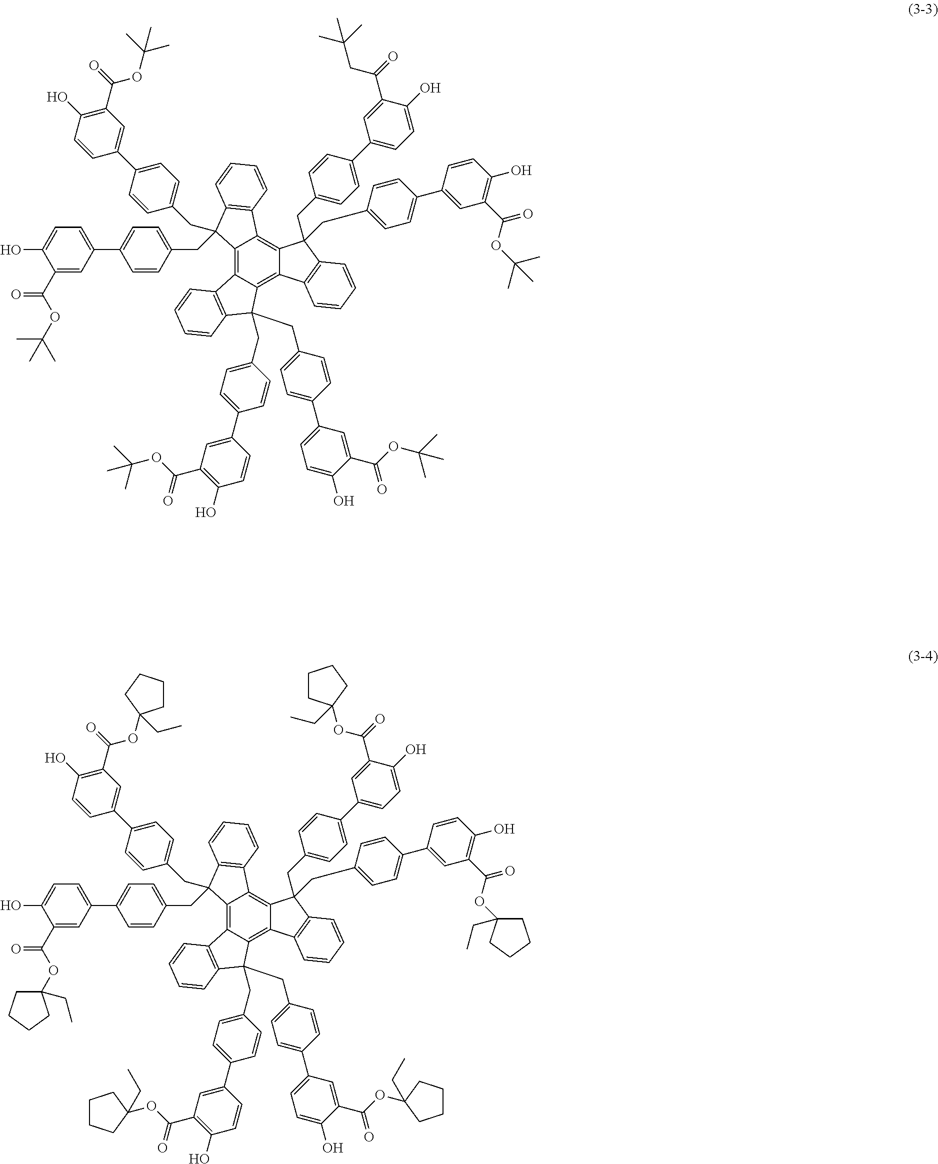

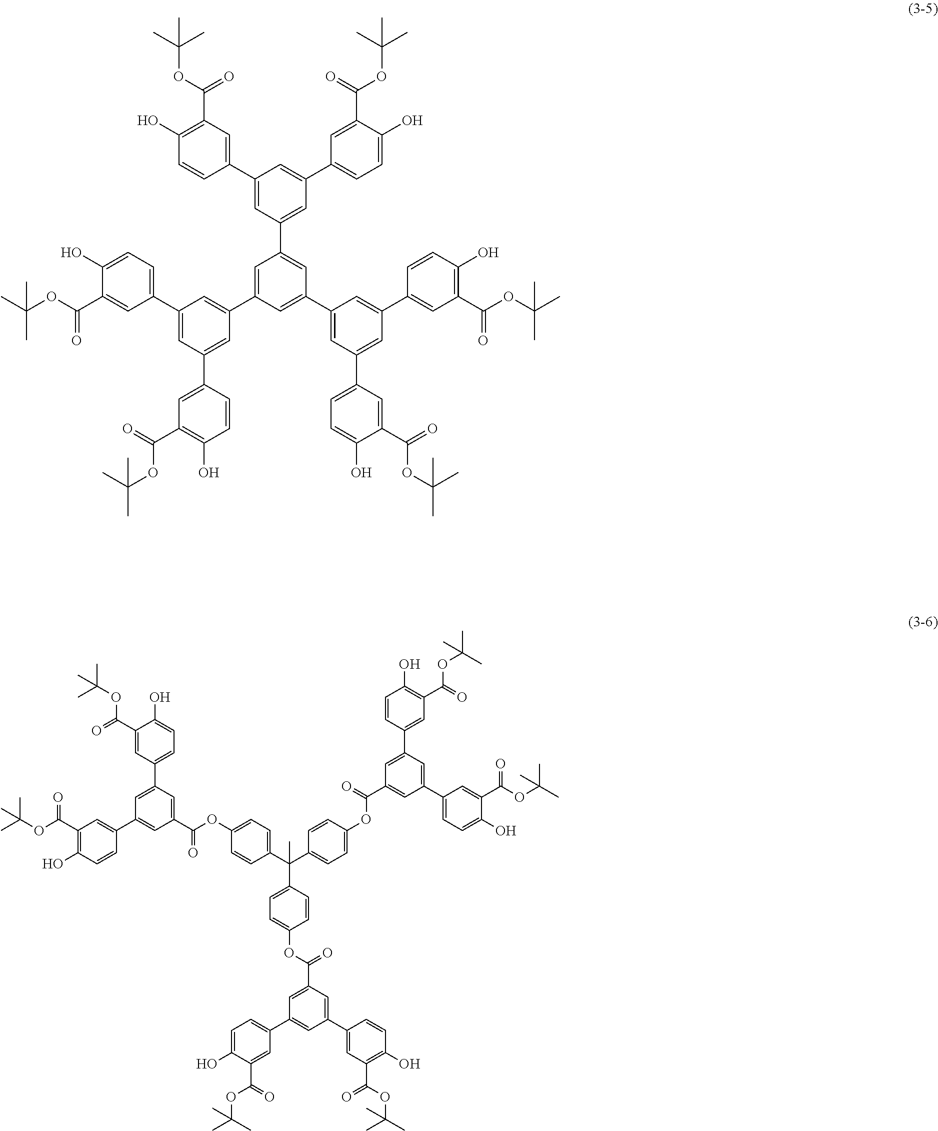

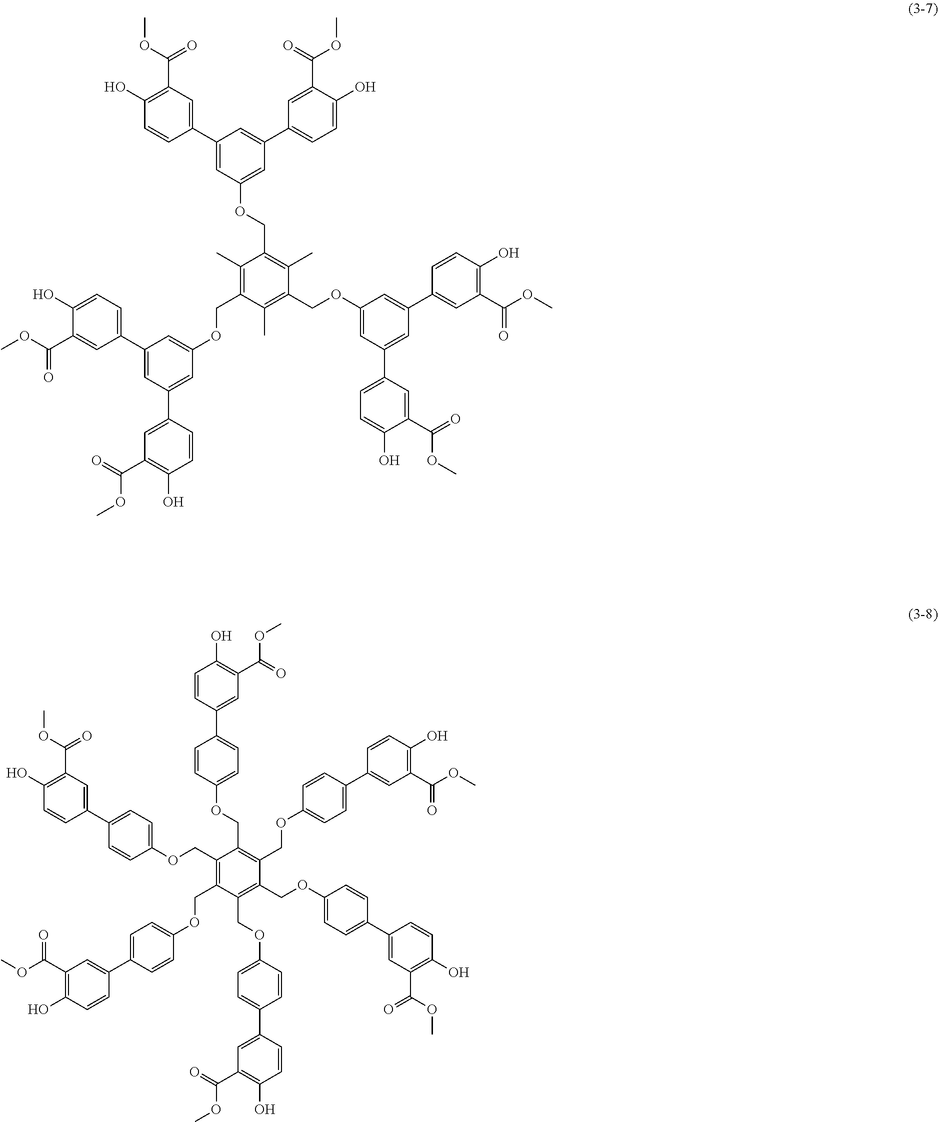

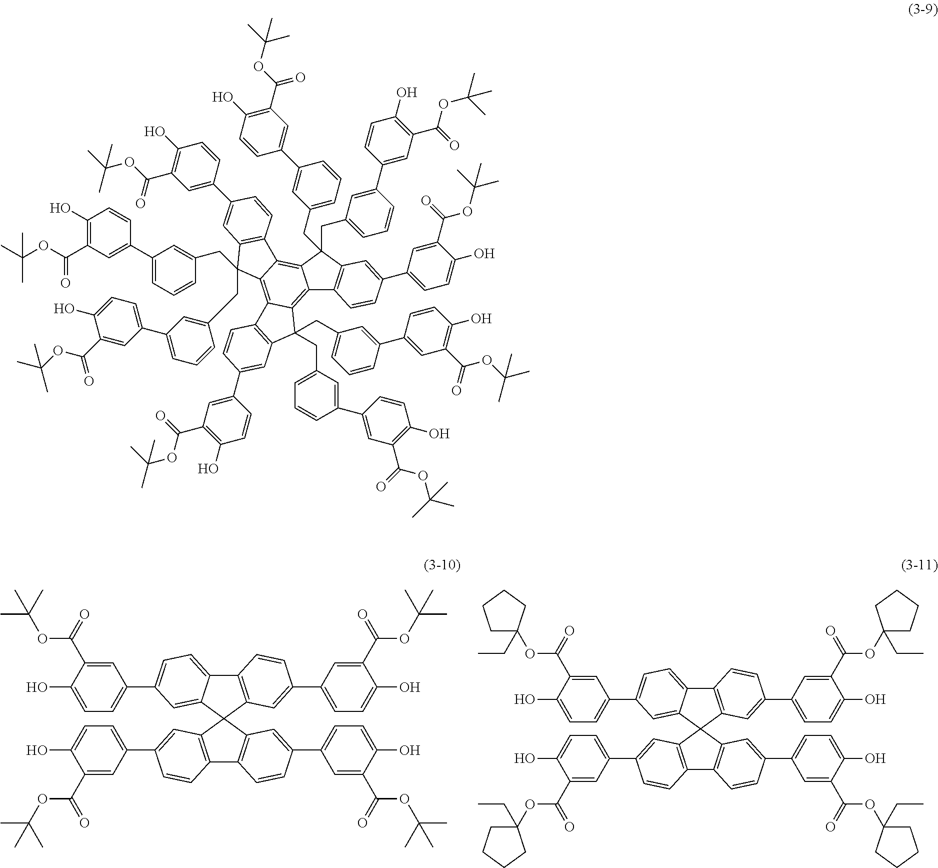

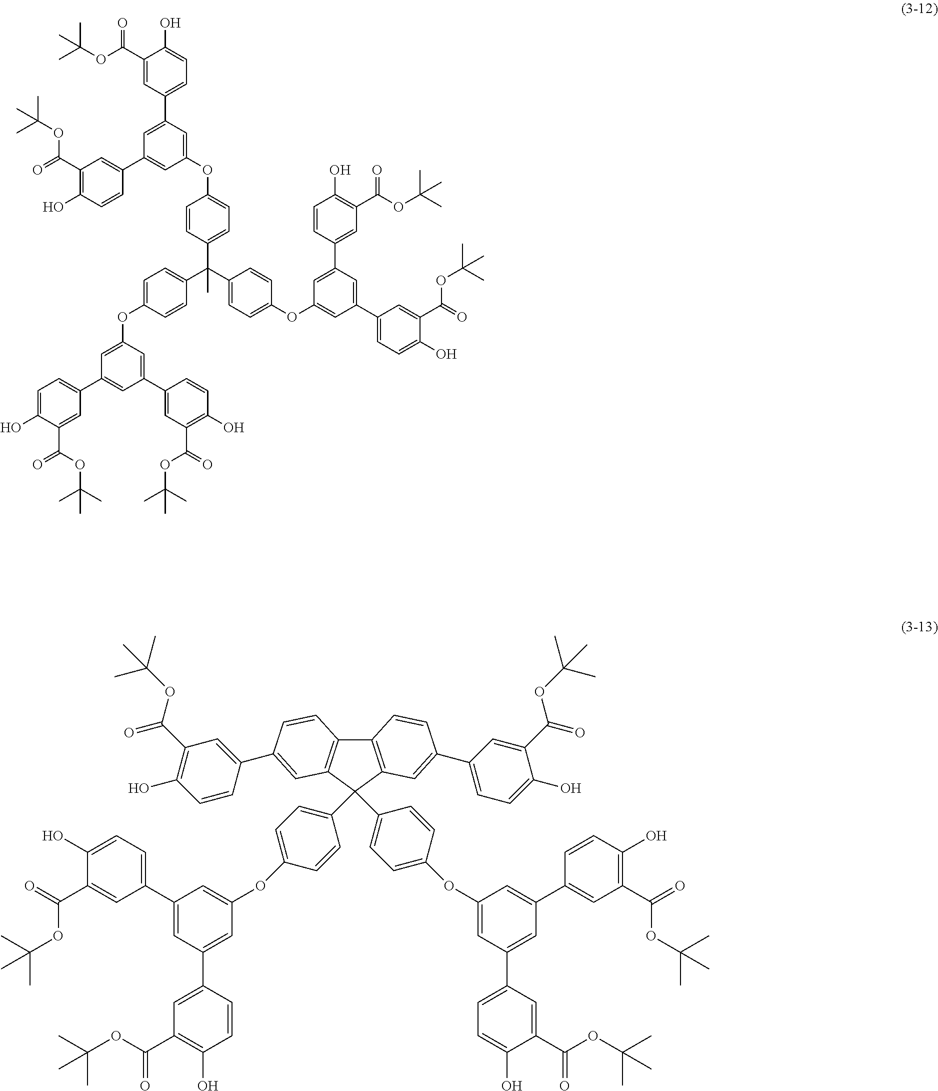

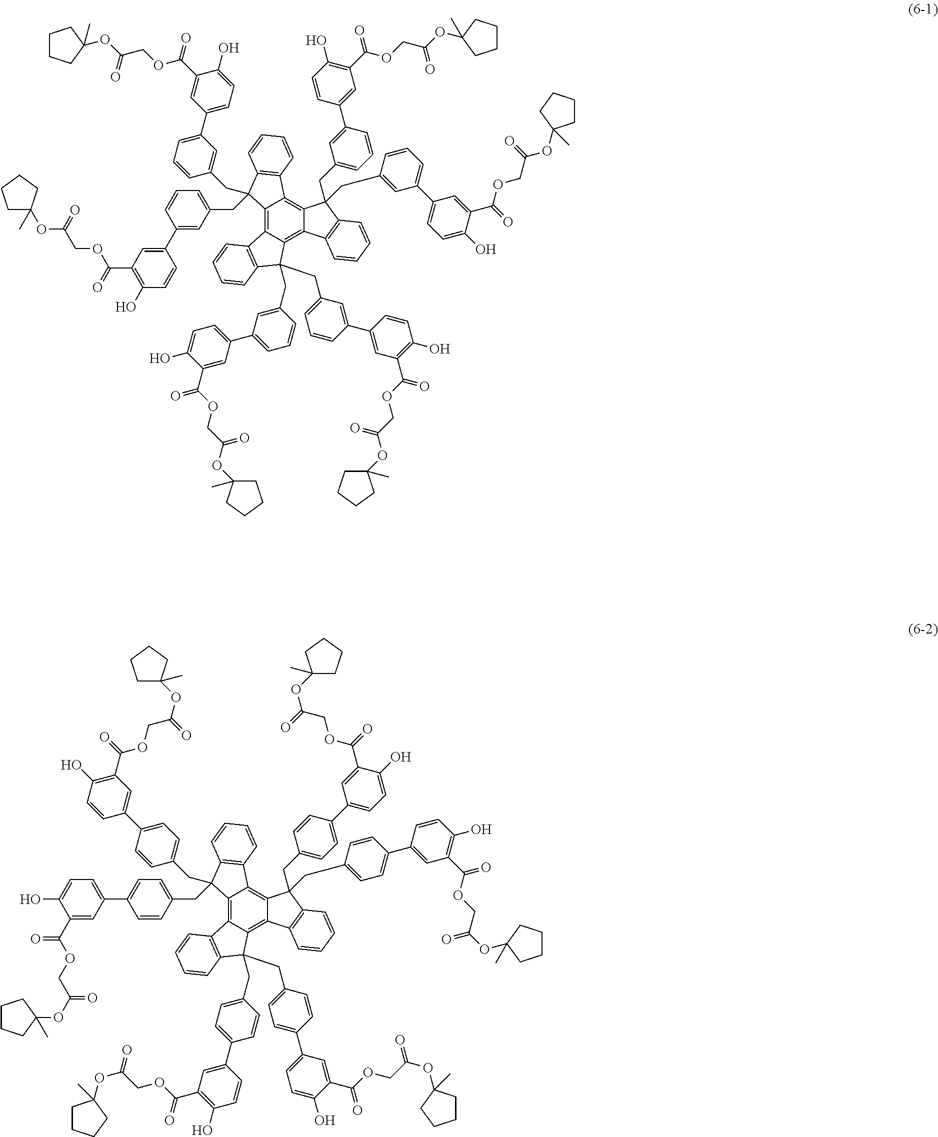

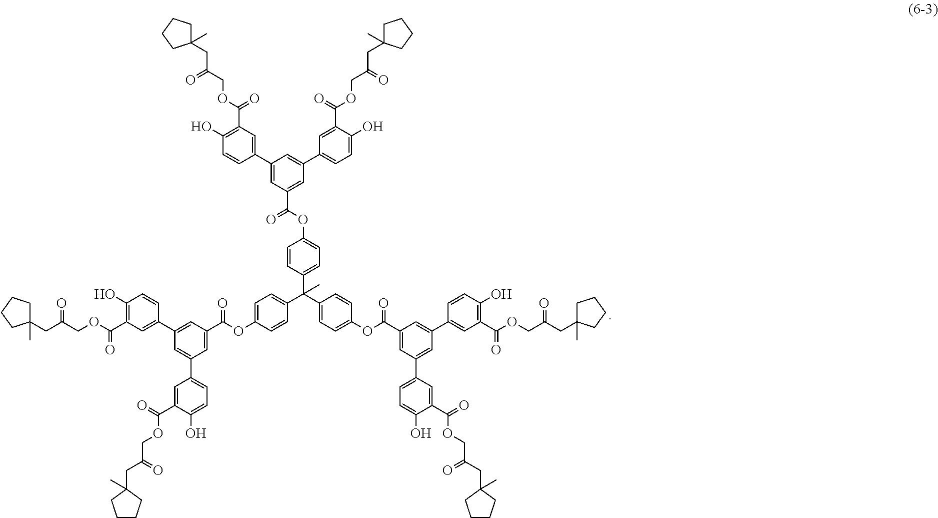 [SMILES: CC1(CC(=O)COC(=O)c2cc(-c3cc(C(=O)Oc4ccc(C(C)(c5ccc(OC(=O)c6cc(-c7ccc(O)c(C(=O)OCC(=O)CC8(C)CCCC8)c7)cc(-c7ccc(O)c(C(=O)OCC(=O)CC8(C)CCCC8)c7)c6)cc5)c5ccc(OC(=O)c6cc(-c7ccc(O)c(C(=O)OCC(=O)CC8(C)CCCC8)c7)cc(-c7ccc(O)c(C(=O)OCC(=O)CC8(C)CCCC8)c7)c6)cc5)cc4)cc(-c4ccc(O)c(C(=O)OCC(=O)CC5(C)CCCC5)c4)c3)ccc2O)CCCC1]

17. A resist composition comprising:
the polycarboxylate compound according to claim 1;
a photoacid generator; and
an organic solvent.

18. The resist composition of claim 17, wherein an amount of the polycarboxylate compound is from about 0.1 parts by weight to about 50 parts by weight based on 100 parts by weight of the resist composition.

19. A method of forming a pattern, the method comprising:
forming a resist film by applying the resist composition according to claim 17 to a substrate;
exposing at least one portion of the resist film to high-energy rays to provide an exposed resist film; and
developing the exposed resist film using a developer.

20. The method of claim 19, wherein the exposing is performed using at least one of ultraviolet (UV) rays, deep ultraviolet (DUV) rays, extreme ultraviolet (EUV) rays, or electron beams (EBs).
